SEQUENCE LISTING (416 sequences)

SEQ ID NO: 1 - protein, 116 aa, synthetic construct
EVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCARGGRAFDIWGQGTMVTVSS

SEQ ID NO: 2 - protein, 8 aa, synthetic construct
GFTFSSYG

SEQ ID NO: 3 - protein, 8 aa, synthetic construct
ISYDGSNK

SEQ ID NO: 4 - protein, 9 aa, synthetic construct
ARGGRAFDI

SEQ ID NO: 5 - protein, 25 aa, synthetic construct
EVQLVESGGGVVQPGRSLRLSCAAS

SEQ ID NO: 6 - protein, 17 aa, synthetic construct
MHWVRQAPGKGLEWVAV

SEQ ID NO: 7 - protein, 38 aa, synthetic construct
YYADSVKGRFTISRDNSKNTLYLQMNSLRAEDTAVYYC

SEQ ID NO: 8 - protein, 11 aa, synthetic construct
WGQGTMVTVSS

SEQ ID NO: 9 - protein, 112 aa, synthetic construct
DVVMTQSPLSLPVTLGQPASISCKSSQSLVNSGGDTFLNWFQQRPGQSPRRLIYKVSNRDSGVPDRFSGSGSGTDFTLEISRVEAEDVGVYYCMQGTHWPRTFGQGTKVEIK

SEQ ID NO: 10 - protein, 11 aa, synthetic construct
QSLVNSGGDTF

SEQ ID NO: 11 - skipped

SEQ ID NO: 12 - protein, 9 aa, synthetic construct
MQGTHWPRT

SEQ ID NO: 13 - protein, 26 aa, synthetic construct
DVVMTQSPLSLPVTLGQPASISCKSS

SEQ ID NO: 14 - protein, 17 aa, synthetic construct
LNWFQQRPGQSPRRLIY

SEQ ID NO: 15 - protein, 36 aa, synthetic construct
NRDSGVPDRFSGSGSGTDFTLEISRVEAEDVGVYYC

SEQ ID NO: 16 - protein, 10 aa, synthetic construct
FGQGTKVEIK

SEQ ID NO: 17 - protein, 116 aa, synthetic construct
QVQLQESGAGLLKPSETLSLTCAVYGGSFSGYYWSWIRQPPGKGLEWIGRIYTSGSTNYNPSLKSRVTISVDTSKNQFSLKLSSVTAADTAVYYCARVGAGAFDIWGQGTMVTVSS

SEQ ID NO: 18 - protein, 8 aa, synthetic construct
GGSFSGYY

SEQ ID NO: 19 - protein, 7 aa, synthetic construct
IYTSGST

SEQ ID NO: 20 - protein, 10 aa, synthetic construct
ARVGAGAFDI

SEQ ID NO: 21 - protein, 25 aa, synthetic construct
QVQLQESGAGLLKPSETLSLTCAVY

SEQ ID NO: 22 - protein, 17 aa, synthetic construct
WSWIRQPPGKGLEWIGR

SEQ ID NO: 23 - protein, 38 aa, synthetic construct
NYNPSLKSRVTISVDTSKNQFSLKLSSVTAADTAVYYC

SEQ ID NO: 24 - protein, 115 aa, synthetic construct
QAVLTQPSSLSASPGASASLTCTLRSDINVGAYRIYWYQQKPGSPPRYLLRYKSDSDKQQGSGVSNRFSASKDASANAGILHISGLHSEDEADYYCLIWHNSVWVFGGGTKLTVL

SEQ ID NO: 25 - protein, 9 aa, synthetic construct
SDINVGAYR

SEQ ID NO: 26 - protein, 7 aa, synthetic construct
YKSDSDK

SEQ ID NO: 27 - protein, 9 aa, synthetic construct
LIWHNSVWV

SEQ ID NO: 28 - protein, 25 aa, synthetic construct
QAVLTQPSSLSASPGASASLTCTLR

SEQ ID NO: 29 - protein, 17 aa, synthetic construct
IYWYQQKPGSPPRYLLR

SEQ ID NO: 30 - protein, 38 aa, synthetic construct
QQGSGVSNRFSASKDASANAGILHISGLHSEDEADYYC

SEQ ID NO: 31 - protein, 10 aa, synthetic construct
FGGGTKLTVL

SEQ ID NO: 32 - protein, 115 aa, synthetic construct
QVQLQESGGGVVRPGGSLRLSCAASGFTFDDYGMSWVRQAPGKGLEWVSGINWNGGSTGYADSVKGRFTISRDNAKNSLYLQMNSLRAEDTAVYYCARGIAVDYWGQGTLVTVSS

SEQ ID NO: 33 - protein, 8 aa, synthetic construct
GFTFDDYG

SEQ ID NO: 34 - protein, 8 aa, synthetic construct
INWNGGST

SEQ ID NO: 35 - protein, 8 aa, synthetic construct
ARGIAVDY

SEQ ID NO: 36 - protein, 25 aa, synthetic construct
QVQLQESGGGVVRPGGSLRLSCAAS

SEQ ID NO: 37 - protein, 17 aa, synthetic construct
MSWVRQAPGKGLEWVSG

SEQ ID NO: 38 - protein, 38 aa, synthetic construct
GYADSVKGRFTISRDNAKNSLYLQMNSLRAEDTAVYYC

SEQ ID NO: 39 - protein, 11 aa, synthetic construct
WGQGTLVTVSS

SEQ ID NO: 40 - protein, 112 aa, synthetic construct
DVVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPWTFGQGTKVEIK

SEQ ID NO: 41 - protein, 11 aa, synthetic construct
QSLLHSNGYNY

SEQ ID NO: 42 - skipped

SEQ ID NO: 43 - protein, 9 aa, synthetic construct
MQALQTPWT

SEQ ID NO: 44 - protein, 26 aa, synthetic construct
DVVMTQSPLSLPVTPGEPASISCRSS

SEQ ID NO: 45 - protein, 17 aa, synthetic construct
LDWYLQKPGQSPQLLIY

SEQ ID NO: 46 - protein, 36 aa, synthetic construct
NRASGVPDRFSGSGSGTDFTLKISRVEAEDVGVYYC

SEQ ID NO: 47 - protein, 126 aa, synthetic construct
QLQLQESGGGVVQPGRSLRLSCAASGFTFSSYAMHWVRQAPGKGLEWVAVISYDGSNKYYADSVKGRFTISRDNSKNTLYLQMNSLRAEDTAVYYCARDIPYGGYAHYYYYGMDVWGQGTTVTVSS

SEQ ID NO: 48 - protein, 8 aa, synthetic construct
GFTFSSYA

SEQ ID NO: 49 - protein, 19 aa, synthetic construct
ARDIPYGGYAHYYYYGMDV

SEQ ID NO: 50 - protein, 25 aa, synthetic construct
QLQLQESGGGVVQPGRSLRLSCAAS

SEQ ID NO: 51 - protein, 11 aa, synthetic construct
WGQGTTVTVSS

SEQ ID NO: 52 - protein, 110 aa, synthetic construct
QTVVTQEPSLTVSPGGTVTLTCTSSTGAVTSASYPNWFQQKPGQAPRKLIYDTNNRHSWTPARFSGSLLGGKAALTLSGAEPEDEAVYYCFLSYSGAYWIFGGGTHLTVL

SEQ ID NO: 53 - protein, 9 aa, synthetic construct
TGAVTSASY

SEQ ID NO: 54 - skipped

SEQ ID NO: 55 - protein, 10 aa, synthetic construct
FLSYSGAYWI

SEQ ID NO: 56 - protein, 25 aa, synthetic construct
QTVVTQEPSLTVSPGGTVTLTCTSS

SEQ ID NO: 57 - protein, 17 aa, synthetic construct
PNWFQQKPGQAPRKLIY

SEQ ID NO: 58 - protein, 36 aa, synthetic construct
NRHSWTPARFSGSLLGGKAALTLSGAEPEDEAVYYC

SEQ ID NO: 59 - protein, 10 aa, synthetic construct
FGGGTHLTVL

SEQ ID NO: 60 - protein, 122 aa, synthetic construct
QVQLVQSGAEVKKPGSSVKVSCKASGGTFSSYAISWVRQAPGQGLEWMGRIIPILGIANYAQKFQGRVTITADKSTSTAYMELSSLRSEDTAVYYCAREERGSYFRAPFDYWGQGTLVTVSS

SEQ ID NO: 61 - protein, 8 aa, synthetic construct
GGTFSSYA

SEQ ID NO: 62 - protein, 8 aa, synthetic construct
IIPILGIA

SEQ ID NO: 63 - protein, 15 aa, synthetic construct
AREERGSYFRAPFDY

SEQ ID NO: 64 - protein, 25 aa, synthetic construct [sequence omitted]

SEQ ID NO: 65 - protein, 17 aa, synthetic construct
ISWVRQAPGQGLEWMGR

SEQ ID NO: 66 - protein, 38 aa, synthetic construct
NYAQKFQGRVTITADKSTSTAYMELSSLRSEDTAVYYC

SEQ ID NO: 67 - protein, 115 aa, synthetic construct
QAVLTQPSSLSASPGASASLTCTLRSDISVGTYRIYWYQQKPGSPPHYLLRYKSDSDKQQGSGVPSRFSGFKDASANAGILLISGLQSEDEADYYCLIWHSNTWVFGGGTKLTVL

SEQ ID NO: 68 - protein, 9 aa, synthetic construct
SDISVGTYR

SEQ ID NO: 69 - protein, 9 aa, synthetic construct
LIWHSNTWV

SEQ ID NO: 70 - protein, 17 aa, synthetic construct
IYWYQQKPGSPPHYLLR

SEQ ID NO: 71 - protein, 38 aa, synthetic construct
QQGSGVPSRFSGFKDASANAGILLISGLQSEDEADYYC

SEQ ID NO: 72 - protein, 115 aa, synthetic construct
QAVLTQPSSLSASPGASASLTCTLRSGINVGTYRIYWYQQKPGSPPQYLLRYKSDSDKQQGSGVPSRFSGSKDASANAGILLISGLQSEDEADYYCMIWHSSAWVFGGGTKLTVL

SEQ ID NO: 73 - protein, 9 aa, synthetic construct
SGINVGTYR

SEQ ID NO: 74 - protein, 9 aa, synthetic construct
MIWHSSAWV

SEQ ID NO: 75 - protein, 17 aa, synthetic construct
IYWYQQKPGSPPQYLLR

SEQ ID NO: 76 - protein, 38 aa, synthetic construct
QQGSGVPSRFSGSKDASANAGILLISGLQSEDEADYYC

SEQ ID NO: 77 - protein, 115 aa, synthetic construct
QAVLTQPSSLSASPGASASLTCTLRSGIHVGTYRIYWYQQKPGSPPQFLLKYKTDSDKQQGSGVPSRFSGSKDASANAGILLISGLQSEDEADYYCMIWHNNAWVFGGGTKLTVL

SEQ ID NO: 78 - protein, 9 aa, synthetic construct
SGIHVGTYR

SEQ ID NO: 79 - protein, 7 aa, synthetic construct
YKTDSDK

SEQ ID NO: 80 - protein, 9 aa, synthetic construct
MIWHNNAWV

SEQ ID NO: 81 - protein, 17 aa, synthetic construct
IYWYQQKPGSPPQFLLK

SEQ ID NO: 82 - protein, 113 aa, synthetic construct
QVTLKESGGGVVQPGRSLRLSCAASGFTFSSYGMHWVRQAPGKGLEWVAVISYDGSNKYYADSVKGRFTISRDNSKNTLYLQMNSLRAEDTAVYYCTQGRDYWGQGTLVTVSS

SEQ ID NO: 83 - protein, 6 aa, synthetic construct
TQGRDY

SEQ ID NO: 84 - protein, 25 aa, synthetic construct
QVTLKESGGGVVQPGRSLRLSCAAS

SEQ ID NO: 85 - protein, 116 aa, synthetic construct
QVQLVQSGGGVVQPGRSLRLSCAASGFTFSSYAMHWVRQAPGKGLEWVAVISYDGSNKYYADSVKGRFTISRDNSKNTLYLQMNSLRAEDTAVYYCARASRGMDVWGQGTTVTVSS

SEQ ID NO: 86 - protein, 9 aa, synthetic construct
ARASRGMDV

SEQ ID NO: 87 - protein, 25 aa, synthetic construct
QVQLVQSGGGVVQPGRSLRLSCAAS

SEQ ID NO: 88 - protein, 112 aa, synthetic construct
DVVMTQSPLSLPVTPGQSASISCRSSQSLVDRDGNTYLTWFHQRPGQSPRRLIFKVSNRDSGVPDRFSGSGSGTDFTLRISRVEAEDVGVYYCMQVTHWPRTFGQGTKVEIK

SEQ ID NO: 89 - protein, 11 aa, synthetic construct
QSLVDRDGNTY

SEQ ID NO: 90 - protein, 9 aa, synthetic construct
MQVTHWPRT

SEQ ID NO: 91 - protein, 26 aa, synthetic construct
DVVMTQSPLSLPVTPGQSASISCRSS

SEQ ID NO: 92 - protein, 17 aa, synthetic construct
LTWFHQRPGQSPRRLIF

SEQ ID NO: 93 - protein, 36 aa, synthetic construct
NRDSGVPDRFSGSGSGTDFTLRISRVEAEDVGVYYC

SEQ ID NO: 94 - protein, 119 aa, synthetic construct
QVQLQQSGAEVKKPGSSVKVSCKASGGTFSSYAISWVRQAPGQGLEWMGGIIPIFGTANYAQKFQGRVTITADKSTSTAYMELSSLRSEDTAVYYCARARSGSYSLHYWGQGTLVTVSS

SEQ ID NO: 95 - protein, 8 aa, synthetic construct
IIPIFGTA

SEQ ID NO: 96 - protein, 12 aa, synthetic construct
ARARSGSYSLHY

SEQ ID NO: 97 - protein, 25 aa, synthetic construct
QVQLQQSGAEVKKPGSSVKVSCKAS

SEQ ID NO: 98 - protein, 17 aa, synthetic construct
ISWVRQAPGQGLEWMGG

SEQ ID NO: 99 - skipped

SEQ ID NO: 100 - protein, 111 aa, synthetic construct
NFMLTQPHSVSESPGKTVTISCTGSSGSIDSNYVQWYQQRPGSAPTTVIFEDSQRPSGVPDRFSGSTDRSSNSASLTISGLKTEDEADYYCQSYDSSNNWVFGGGTKLTVL

SEQ ID NO: 101 - protein, 8 aa, synthetic construct
SGSIDSNY

SEQ ID NO: 102 - skipped

SEQ ID NO: 103 - protein, 10 aa, synthetic construct
QSYDSSNNWV

SEQ ID NO: 104 - protein, 25 aa, synthetic construct
NFMLTQPHSVSESPGKTVTISCTGS

SEQ ID NO: 105 - protein, 17 aa, synthetic construct
VQWYQQRPGSAPTTVIF

SEQ ID NO: 106 - protein, 38 aa, synthetic construct
QRPSGVPDRFSGSTDRSSNSASLTISGLKTEDEADYYC

SEQ ID NO: 107 - protein, 125 aa, synthetic construct
EVQLVQSGAEVKKPGASVKVSCKASGYTFTSYAMHWVRQAPGQRLEWMGWINAGNGNTKYSQKFQGRVTITRDTSASTAYMELSSLRSEDTAVYYCARVLHVLRYFDWPGAFDIWGQGTTVTVSS

SEQ ID NO: 108 - protein, 8 aa, synthetic construct
GYTFTSYA

SEQ ID NO: 109 - protein, 8 aa, synthetic construct
INAGNGNT

SEQ ID NO: 110 - protein, 18 aa, synthetic construct
ARVLHVLRYFDWPGAFDI

SEQ ID NO: 111 - protein, 25 aa, synthetic construct
EVQLVQSGAEVKKPGASVKVSCKAS

SEQ ID NO: 112 - protein, 17 aa, synthetic construct
MHWVRQAPGQRLEWMGW

SEQ ID NO: 113 - protein, 38 aa, synthetic construct
KYSQKFQGRVTITRDTSASTAYMELSSLRSEDTAVYYC

SEQ ID NO: 114 - protein, 110 aa, synthetic construct
QSVLTQPPSASGTPGQRVTISCSGSSSNIGTNYVYWYQQLPGTAPKLLIYINDQRPSGVPDRFSGSKSGTSASLALSGLRSEDEADYYCAAWDDSLSGLVFGGGTKLTVL

SEQ ID NO: 115 - protein, 8 aa, synthetic construct
SSNIGTNY

SEQ ID NO: 116 - skipped

SEQ ID NO: 117 - protein, 11 aa, synthetic construct
AAWDDSLSGLV

SEQ ID NO: 118 - protein, 25 aa, synthetic construct
QSVLTQPPSASGTPGQRVTISCSGS

SEQ ID NO: 119 - protein, 17 aa, synthetic construct
VYWYQQLPGTAPKLLIY

SEQ ID NO: 120 - protein, 36 aa, synthetic construct
QRPSGVPDRFSGSKSGTSASLALSGLRSEDEADYYC

SEQ ID NO: 121 - protein, 128 aa, synthetic construct
QVQLVQSGGGVVQPGRSLRLSCATSGFTFSSYGMHWVRQAPGKGLEWVAVISYDGSNKYYADSVKGRFTISRDNSKNTLYLQMNSLRAEDTAVYYCARDLVDSSGWYGYYYYYGMDVWGQGTTVTVSS

SEQ ID NO: 122 - protein, 21 aa, synthetic construct
ARDLVDSSGWYGYYYYYGMDV

SEQ ID NO: 123 - protein, 25 aa, synthetic construct
QVQLVQSGGGVVQPGRSLRLSCATS

SEQ ID NO: 124 - protein, 107 aa, synthetic construct
EIVLTQSPATLSLSPGERATLSCRASQSVSSYLAWYQQKPGQAPRLLIYDASNRATGIPARFSGSGSGTDFTLTISSLEPEDFAVYFCQQRSNWPGTFGQGTKVEIK

SEQ ID NO: 125 - protein, 6 aa, synthetic construct
QSVSSY

SEQ ID NO: 126 - skipped

SEQ ID NO: 127 - protein, 9 aa, synthetic construct
QQRSNWPGT

SEQ ID NO: 128 - protein, 26 aa, synthetic construct [sequence omitted]

SEQ ID NO: 129 - protein, 17 aa, synthetic construct
LAWYQQKPGQAPRLLIY

SEQ ID NO: 130 - protein, 36 aa, synthetic construct
NRATGIPARFSGSGSGTDFTLTISSLEPEDFAVYFC

SEQ ID NO: 131 - protein, 117 aa, synthetic construct
EVQLVQSGAEVKKPGDSVKVSCKASGYTFTGYYMHWVRQAPGQGLEWMGRINPNSGGTNYAQKFQGRVTMTTDTSTSTAYMELRSLRSDDTAVYYCARDLGGATVYWGQGTLVTVSS

SEQ ID NO: 132 - protein, 8 aa, synthetic construct
GYTFTGYY

SEQ ID NO: 133 - protein, 8 aa, synthetic construct
INPNSGGT

SEQ ID NO: 134 - protein, 10 aa, synthetic construct
ARDLGGATVY

SEQ ID NO: 135 - protein, 25 aa, synthetic construct
EVQLVQSGAEVKKPGDSVKVSCKAS

SEQ ID NO: 136 - protein, 17 aa, synthetic construct
MHWVRQAPGQGLEWMGR

SEQ ID NO: 137 - protein, 38 aa, synthetic construct
NYAQKFQGRVTMTTDTSTSTAYMELRSLRSDDTAVYYC

SEQ ID NO: 138 - protein, 115 aa, synthetic construct
QAVLTQPSSLSASPGASASLTCTLHSGVNVGTQRIYWYQQKPGSPPRYLVRYKSDSNKQQGSGVPSRFSGSKDASANAGILIISGLQSDDEADYYCMIWHNNAWVFGGGTTLTVL

SEQ ID NO: 139 - protein, 9 aa, synthetic construct
SGVNVGTQR

SEQ ID NO: 140 - protein, 7 aa, synthetic construct
YKSDSNK

SEQ ID NO: 141 - protein, 25 aa, synthetic construct
QAVLTQPSSLSASPGASASLTCTLH

SEQ ID NO: 142 - protein, 17 aa, synthetic construct
IYWYQQKPGSPPRYLVR

SEQ ID NO: 143 - protein, 38 aa, synthetic construct
QQGSGVPSRFSGSKDASANAGILIISGLQSDDEADYYC

SEQ ID NO: 144 - protein, 10 aa, synthetic construct
FGGGTTLTVL

SEQ ID NO: 145 - protein, 120 aa, synthetic construct
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARNGAVAGKYFDYWGQGTLVTVSS

SEQ ID NO: 146 - protein, 8 aa, synthetic construct
GFTFSSYS

SEQ ID NO: 147 - protein, 8 aa, synthetic construct
ISSSSSYI

SEQ ID NO: 148 - protein, 13 aa, synthetic construct
ARNGAVAGKYFDY

SEQ ID NO: 149 - protein, 25 aa, synthetic construct
EVQLVESGGGLVKPGGSLRLSCAAS

SEQ ID NO: 150 - protein, 17 aa, synthetic construct
MNWVRQAPGKGLEWVSS

SEQ ID NO: 151 - protein, 38 aa, synthetic construct
YYADSVKGRFTISRDNAKNSLYLQMNSLRAEDTAVYYC

SEQ ID NO: 152 - protein, 112 aa, synthetic construct
DVVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPLTFGGGTKVEIK

SEQ ID NO: 153 - protein, 9 aa, synthetic construct
MQALQTPLT

SEQ ID NO: 154 - protein, 10 aa, synthetic construct
FGGGTKVEIK

SEQ ID NO: 155 - protein, 124 aa, synthetic construct
QVQLVQSGGGVVQPGRSLRLSCAASGFTFSSYAMHWVRQAPGKGLEWVAVISYDGSNKYYADSVKGRFTISRDNSKNTLYLQMNSLRAEDTAVYYCARDFYPFIVGATSFFDYWGQGTLVTVSS

SEQ ID NO: 156 - protein, 17 aa, synthetic construct
ARDFYPFIVGATSFFDY

SEQ ID NO: 157 - protein, 107 aa, synthetic construct
DIQLTQSPSSLSASVGDRVTITCRASQSISTYINWYQQKPGKAPKLLIYFASSLQSGVPSRFSGGGSGTDFTLTISSLQPEDFATYYCQQSYNIPVTFGQGTKVEVT

SEQ ID NO: 158 - protein, 6 aa, synthetic construct
QSISTY

SEQ ID NO: 159 - skipped

SEQ ID NO: 160 - protein, 9 aa, synthetic construct
QQSYNIPVT

SEQ ID NO: 161 - protein, 26 aa, synthetic construct [sequence omitted]

SEQ ID NO: 162 - protein, 17 aa, synthetic construct
INWYQQKPGKAPKLLIY

SEQ ID NO: 163 - protein, 36 aa, synthetic construct
SLQSGVPSRFSGGGSGTDFTLTISSLQPEDFATYYC

SEQ ID NO: 164 - protein, 19 aa, synthetic construct
QQSYNIPVTFGQGTKVEVT

SEQ ID NO: 165 - protein, 119 aa, synthetic construct
QVQLVQSGGGVVQPGRSLRLSCAASGFTVSSNYMSWVRQAPGKGLEWVSVIYSGGSTYYADSVKGRFTISRDNSKNTLYLQMNSLRAEDTAVYYCARGSRAVAGAFDYWGQGTLVTVSS

SEQ ID NO: 166 - protein, 8 aa, synthetic construct
GFTVSSNY

SEQ ID NO: 167 - protein, 7 aa, synthetic construct
IYSGGST

SEQ ID NO: 168 - protein, 13 aa, synthetic construct
ARGSRAVAGAFDY

SEQ ID NO: 169 - protein, 17 aa, synthetic construct
MSWVRQAPGKGLEWVSV

SEQ ID NO: 170 - protein, 108 aa, synthetic construct
SSELTQDPAVSVALGQTVSITCQGDSLRNYFAAWYQQKPGQAPLLVIYGKNNRPSGIPDRFSGSSSGNTASLTITGAEAEDEADYYCGSRDSSGNHPVFGGGTKVTVL

SEQ ID NO: 171 - protein, 6 aa, synthetic construct
SLRNYF

SEQ ID NO: 172 - skipped

SEQ ID NO: 173 - protein, 11 aa, synthetic construct
GSRDSSGNHPV

SEQ ID NO: 174 - protein, 25 aa, synthetic construct
SSELTQDPAVSVALGQTVSITCQGD

SEQ ID NO: 175 - protein, 17 aa, synthetic construct
AAWYQQKPGQAPLLVIY

SEQ ID NO: 176 - protein, 36 aa, synthetic construct
NRPSGIPDRFSGSSSGNTASLTITGAEAEDEADYYC

SEQ ID NO: 177 - protein, 10 aa, synthetic construct
FGGGTKVTVL

SEQ ID NO: 178 - protein, 108 aa, synthetic construct
YSELTQDPAVSVALGQTVRITCQGDSLRTYYANWYQQKPGQAPVLVMYAKNHRPSGIPDRFSGSSSGNTASLTITGAQAEDEADYYCGSRDSSGNHPVFGGGTKVTVL

SEQ ID NO: 179 - protein, 6 aa, synthetic construct
SLRTYY

SEQ ID NO: 180 - skipped

SEQ ID NO: 181 - protein, 25 aa, synthetic construct
YSELTQDPAVSVALGQTVRITCQGD

SEQ ID NO: 182 - protein, 17 aa, synthetic construct
ANWYQQKPGQAPVLVMY

SEQ ID NO: 183 - protein, 36 aa, synthetic construct
HRPSGIPDRFSGSSSGNTASLTITGAQAEDEADYYC

SEQ ID NO: 184 - protein, 122 aa, synthetic construct
QVQLQQSGPGLVKPSQTLSVTCAISGDSVSSNSAAWNWFRQSPSRGLEWLGRTYYRSKWYSDYAVSVKSRITINPDTSKNQFSLQLNSVTPEDTAVYYCARDVRAARAFDIWGQGTMVTVSS

SEQ ID NO: 185 - protein, 10 aa, synthetic construct
SVSSNSAAWN

SEQ ID NO: 186 - protein, 7 aa, synthetic construct
YRSKWYS

SEQ ID NO: 187 - protein, 12 aa, synthetic construct
ARDVRAARAFDI

SEQ ID NO: 188 - protein, 27 aa, synthetic construct
QVQLQQSGPGLVKPSQTLSVTCAISGD

SEQ ID NO: 189 - protein, 17 aa, synthetic construct
WFRQSPSRGLEWLGRTY

SEQ ID NO: 190 - protein, 38 aa, synthetic construct
DYAVSVKSRITINPDTSKNQFSLQLNSVTPEDTAVYYC

SEQ ID NO: 191 - protein, 115 aa, synthetic construct
QAVLTQPSSLSASPGASASLTCTLRSGINVGTYRIYWYQQKPGSPPQYLLRYKSDSDKQQGSGVPSRFSGSKDASANAGILVISGLQSEDEADYYCLIWHNRGWVFGGGTKVTVL

SEQ ID NO: 192-193 - skipped (2 entries)

SEQ ID NO: 194 - protein, 9 aa, synthetic construct
LIWHNRGWV

SEQ ID NO: 195-196 - skipped (2 entries)

SEQ ID NO: 197 - protein, 38 aa, synthetic construct
QQGSGVPSRFSGSKDASANAGILVISGLQSEDEADYYC

SEQ ID NO: 198 - protein, 117 aa, synthetic construct
EVQLVESGGGLVQPGRPLRLSCAASGFTFSSYAMSWVRQAPGKGLEWVSSISSGGSTYYADSVRGRFTISRDDSKDTLYLQMNSLRAEDTAVYYCARTGVGGAFDIWGQGTMVTVSS

SEQ ID NO: 199 - protein, 7 aa, synthetic construct
ISSGGST

SEQ ID NO: 200 - protein, 11 aa, synthetic construct
ARTGVGGAFDI

SEQ ID NO: 201 - protein, 25 aa, synthetic construct
EVQLVESGGGLVQPGRPLRLSCAAS

SEQ ID NO: 202 - protein, 17 aa, synthetic construct
MSWVRQAPGKGLEWVSS

SEQ ID NO: 203 - protein, 38 aa, synthetic construct
YYADSVRGRFTISRDDSKDTLYLQMNSLRAEDTAVYYC

SEQ ID NO: 204 - protein, 112 aa, synthetic construct
DVVMTQSPLSLPVTPGEPASISCRSSHSLLYDNRYNYLDWYLQRPGQSPQLLIYLGSTRASGVPDRFSGSGSGTDFTLKISRVEAEDIGVYYCMQAVQTPIIFGQGTRLEIK

SEQ ID NO: 205 - protein, 11 aa, synthetic construct
HSLLYDNRYNY

SEQ ID NO: 206 - protein, 9 aa, synthetic construct
MQAVQTPII

SEQ ID NO: 207 - protein, 17 aa, synthetic construct
LDWYLQRPGQSPQLLIY

SEQ ID NO: 208 - protein, 36 aa, synthetic construct
TRASGVPDRFSGSGSGTDFTLKISRVEAEDIGVYYC

SEQ ID NO: 209 - protein, 10 aa, synthetic construct
FGQGTRLEIK

SEQ ID NO: 210 - protein, 116 aa, synthetic construct
EVQLLESGGGLVQPGGSLRLSCAASGFTFSSYAMSWVRQAPGKGLEWVSTIYYAGSNTYYADSVKGRFTISRDNSKNTLYLQMNSLRAEDTAVYYCAKGYYTFDYWGQGTLVTVSS

SEQ ID NO: 211 - protein, 8 aa, synthetic construct
IYYAGSNT

SEQ ID NO: 212 - protein, 9 aa, synthetic construct
AKGYYTFDY

SEQ ID NO: 213 - protein, 25 aa, synthetic construct
EVQLLESGGGLVQPGGSLRLSCAAS

SEQ ID NO: 214 - protein, 17 aa, synthetic construct
MSWVRQAPGKGLEWVST

SEQ ID NO: 215 - protein, 104 aa, synthetic construct
QDPDVSVALGQTVRITCHGDSLRSSYASWYQQKPGQAPVLVFFGKNNRPSGIPDRFSGSSSGDTASLTITGAQAEDEADYYCHSRDTSDNHLRVFGGGTKLTVL

SEQ ID NO: 216 - protein, 6 aa, synthetic construct
SLRSSY

SEQ ID NO: 217 - protein, 12 aa, synthetic construct
HSRDTSDNHLRV

SEQ ID NO: 218 - protein, 20 aa, synthetic construct
QDPDVSVALGQTVRITCHGD

SEQ ID NO: 219 - protein, 17 aa, synthetic construct
ASWYQQKPGQAPVLVFF

SEQ ID NO: 220 - protein, 36 aa, synthetic construct
NRPSGIPDRFSGSSSGDTASLTITGAQAEDEADYYC

SEQ ID NO: 221 - protein, 117 aa, synthetic construct
EVQLVESGGGLVRPGRSLRLSCRASGLTLSDYGMHWVRQAPGKGLEWVSGISGSGSNTYYTDSVKGRFTISRDNAKNSLYLQMNSLRAEDTAVYFCARSVRGAFDNWGQGTMVTVSS

SEQ ID NO: 222 - protein, 8 aa, synthetic construct
GLTLSDYG

SEQ ID NO: 223 - protein, 8 aa, synthetic construct
ISGSGSNT

SEQ ID NO: 224 - protein, 10 aa, synthetic construct
ARSVRGAFDN

SEQ ID NO: 225 - protein, 25 aa, synthetic construct
EVQLVESGGGLVRPGRSLRLSCRAS

SEQ ID NO: 226 - protein, 17 aa, synthetic construct
MHWVRQAPGKGLEWVSG

SEQ ID NO: 227 - protein, 38 aa, synthetic construct
YYTDSVKGRFTISRDNAKNSLYLQMNSLRAEDTAVYFC

SEQ ID NO: 228 - protein, 108 aa, synthetic construct
SSELTQDPAVSVALGQTVRITCQGDSLRRFFASWYQQRPGQAPILVIYGKNNRPSGIPDRFSGSRSGNTASLTIVGAQADDEADYYCSSRDSSRDRLVFGGGTKLTVL

SEQ ID NO: 229 - protein, 6 aa, synthetic construct
SLRRFF

SEQ ID NO: 230 - protein, 11 aa, synthetic construct
SSRDSSRDRLV

SEQ ID NO: 231 - protein, 25 aa, synthetic construct
SSELTQDPAVSVALGQTVRITCQGD

SEQ ID NO: 232 - protein, 17 aa, synthetic construct
ASWYQQRPGQAPILVIY

SEQ ID NO: 233 - protein, 36 aa, synthetic construct
NRPSGIPDRFSGSRSGNTASLTIVGAQADDEADYYC

SEQ ID NO: 234 - protein, 110 aa, synthetic construct
QSVLTQPPSVSAAPGQKVTISCSGSSSNIGDNYVSWYQQLPGTAPKVLIYGNNNRPSGIPDRFSGSKSGTSASLAISGLQSEDEADYYCATWDDSLKGYVFGTGTQLTVL

SEQ ID NO: 235 - protein, 8 aa, synthetic construct
SSNIGDNY

SEQ ID NO: 236 - skipped

SEQ ID NO: 237 - protein, 11 aa, synthetic construct
ATWDDSLKGYV

SEQ ID NO: 238 - protein, 25 aa, synthetic construct
QSVLTQPPSVSAAPGQKVTISCSGS

SEQ ID NO: 239 - protein, 17 aa, synthetic construct
VSWYQQLPGTAPKVLIY

SEQ ID NO: 240 - protein, 36 aa, synthetic construct
NRPSGIPDRFSGSKSGTSASLAISGLQSEDEADYYC

SEQ ID NO: 241 - protein, 10 aa, synthetic construct
FGTGTQLTVL

SEQ ID NO: 242 - protein, 120 aa, synthetic construct
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCARGIVGAFDYWGQGTLVTVSS

SEQ ID NO: 243 - protein, 7 aa, synthetic construct
YRSKWYN

SEQ ID NO: 244 - protein, 10 aa, synthetic construct
ARGIVGAFDY

SEQ ID NO: 245 - protein, 27 aa, synthetic construct
QVQLQQSGPGLVKPSQTLSLTCAISGD

SEQ ID NO: 246 - protein, 17 aa, synthetic construct
WIRQSPSRGLEWLGRTY

SEQ ID NO: 247 - protein, 109 aa, synthetic construct
SSELTQDPAVSVALGQTVRITCQGDSLRSYHASWYQQKPGQAPVLVIYGRNNRPSGIPDRFSGSTSGNTASLTITGAQAEDEADYYCKSRDSSGSLSYVFGTGTKVTVL

SEQ ID NO: 248 - protein, 6 aa, synthetic construct
SLRSYH

SEQ ID NO: 249 - skipped

SEQ ID NO: 250 - protein, 12 aa, synthetic construct
KSRDSSGSLSYV

SEQ ID NO: 251 - protein, 17 aa, synthetic construct
ASWYQQKPGQAPVLVIY

SEQ ID NO: 252 - protein, 36 aa, synthetic construct
NRPSGIPDRFSGSTSGNTASLTITGAQAEDEADYYC

SEQ ID NO: 253 - protein, 10 aa, synthetic construct
FGTGTKVTVL

SEQ ID NO: 254 - protein, 330 aa, Homo sapiens [sequence omitted]

SEQ ID NO: 255 - protein, 98 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 256 - protein, 12 aa, synthetic construct
EPKSCDKTHTCP

SEQ ID NO: 257 - protein, 113 aa, synthetic construct
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 258 - protein, 107 aa, synthetic construct
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 259 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 260 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 261 - protein, 98 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 262 - protein, 107 aa, synthetic construct
GQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 263 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 264 - protein, 220 aa, Homo sapiens
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 265 - protein, 219 aa, Homo sapiens
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 266 - protein, 217 aa, Homo sapiens
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 267 - protein, 107 aa, synthetic construct [sequence omitted]

SEQ ID NO: 268 - protein, 106 aa, synthetic construct
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 269 - protein, 106 aa, synthetic construct
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 270 - protein, 106 aa, synthetic construct
GQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 271 - protein, 106 aa, synthetic construct
GQPKAAPSVTLFPPSSEELQANKATLVCLISDFYPGAVKVAWKADGSPVNTGVETTTPSKQSNNKYAASSYLSLTPEQWKSHRSYSCQVTHEGSTVEKTVAPAECS

SEQ ID NO: 272 - protein, 106 aa, synthetic construct
GQPKAAPSVTLFPPSSEELQANKATLVCLVSDFNPGAVTVAWKADGSPVKVGVETTKPSKQSNNKYAASSYLSLTPEQWKSHRSYSCRVTHEGSTVEKTVAPAECS

SEQ ID NO: 273 - protein, 446 aa, synthetic construct
EVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCARGGRAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 274 - protein, 443 aa, synthetic construct
EVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCARGGRAFDIWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 275 - protein, 219 aa, synthetic construct
DVVMTQSPLSLPVTLGQPASISCKSSQSLVNSGGDTFLNWFQQRPGQSPRRLIYKVSNRDSGVPDRFSGSGSGTDFTLEISRVEAEDVGVYYCMQGTHWPRTFGQGTKVEIKRTVAAPSVFIFSPSDEQLKSGTASVVCLLNNFYPREAKVQWKVDNALQSGNSQESVTEQDSKDSTYSLSSTLTLSKADYEKHKLYACEVTHQGLSSPVTKSFNRGEC

SEQ ID NO: 276 - protein, 446 aa, synthetic construct
QVQLQESGAGLLKPSETLSLTCAVYGGSFSGYYWSWIRQPPGKGLEWIGRIYTSGSTNYNPSLKSRVTISVDTSKNQFSLKLSSVTAADTAVYYCARVGAGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 277 - protein, 443 aa, synthetic construct
QVQLQESGAGLLKPSETLSLTCAVYGGSFSGYYWSWIRQPPGKGLEWIGRIYTSGSTNYNPSLKSRVTISVDTSKNQFSLKLSSVTAADTAVYYCARVGAGAFDIWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 278 - protein, 221 aa, synthetic construct
QAVLTQPSSLSASPGASASLTCTLRSDINVGAYRIYWYQQKPGSPPRYLLRYKSDSDKQQGSGVSNRFSASKDASANAGILHISGLHSEDEADYYCLIWHNSVWVFGGGTKLTVLSQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPAECS

SEQ ID NO: 279 - protein, 445 aa, synthetic construct
QVQLQESGGGVVRPGGSLRLSCAASGFTFDDYGMSWVRQAPGKGLEWVSGINWNGGSTGYADSVKGRFTISRDNAKNSLYLQMNSLRAEDTAVYYCARGIAVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 280 - protein, 442 aa, synthetic construct
QVQLQESGGGVVRPGGSLRLSCAASGFTFDDYGMSWVRQAPGKGLEWVSGINWNGGSTGYADSVKGRFTISRDNAKNSLYLQMNSLRAEDTAVYYCARGIAVDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 281 - protein, 219 aa, synthetic construct
DVVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 282 - protein, 456 aa, synthetic construct
QLQLQESGGGVVQPGRSLRLSCAASGFTFSSYAMHWVRQAPGKGLEWVAVISYDGSNKYYADSVKGRFTISRDNSKNTLYLQMNSLRAEDTAVYYCARDIPYGGYAHYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 283 - protein, 453 aa, synthetic construct
QLQLQESGGGVVQPGRSLRLSCAASGFTFSSYAMHWVRQAPGKGLEWVAVISYDGSNKYYADSVKGRFTISRDNSKNTLYLQMNSLRAEDTAVYYCARDIPYGGYAHYYYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 284 - protein, 216 aa, synthetic construct
QTVVTQEPSLTVSPGGTVTLTCTSSTGAVTSASYPNWFQQKPGQAPRKLIYDTNNRHSWTPARFSGSLLGGKAALTLSGAEPEDEAVYYCFLSYSGAYWIFGGGTHLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 285 - protein, 452 aa, synthetic construct
QVQLVQSGAEVKKPGSSVKVSCKASGGTFSSYAISWVRQAPGQGLEWMGRIIPILGIANYAQKFQGRVTITADKSTSTAYMELSSLRSEDTAVYYCAREERGSYFRAPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 286 - protein, 449 aa, synthetic construct
QVQLVQSGAEVKKPGSSVKVSCKASGGTFSSYAISWVRQAPGQGLEWMGRIIPILGIANYAQKFQGRVTITADKSTSTAYMELSSLRSEDTAVYYCAREERGSYFRAPFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 287 - protein, 221 aa, synthetic construct
QAVLTQPSSLSASPGASASLTCTLRSDISVGTYRIYWYQQKPGSPPHYLLRYKSDSDKQQGSGVPSRFSGFKDASANAGILLISGLQSEDEADYYCLIWHSNTWVFGGGTKLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 288 - protein, 221 aa, synthetic construct
QAVLTQPSSLSASPGASASLTCTLRSGINVGTYRIYWYQQKPGSPPQYLLRYKSDSDKQQGSGVPSRFSGSKDASANAGILLISGLQSEDEADYYCMIWHSSAWVFGGGTKLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 289 - protein, 221 aa, synthetic construct
QAVLTQPSSLSASPGASASLTCTLRSGIHVGTYRIYWYQQKPGSPPQFLLKYKTDSDKQQGSGVPSRFSGSKDASANAGILLISGLQSEDEADYYCMIWHNNAWVFGGGTKLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 290 - protein, 443 aa, synthetic construct
QVTLKESGGGVVQPGRSLRLSCAASGFTFSSYGMHWVRQAPGKGLEWVAVISYDGSNKYYADSVKGRFTISRDNSKNTLYLQMNSLRAEDTAVYYCTQGRDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 291 - protein, 440 aa, synthetic construct
QVTLKESGGGVVQPGRSLRLSCAASGFTFSSYGMHWVRQAPGKGLEWVAVISYDGSNKYYADSVKGRFTISRDNSKNTLYLQMNSLRAEDTAVYYCTQGRDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 292 - protein, 446 aa, synthetic construct
QVQLVQSGGGVVQPGRSLRLSCAASGFTFSSYAMHWVRQAPGKGLEWVAVISYDGSNKYYADSVKGRFTISRDNSKNTLYLQMNSLRAEDTAVYYCARASRGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 293 - protein, 443 aa, synthetic construct
QVQLVQSGGGVVQPGRSLRLSCAASGFTFSSYAMHWVRQAPGKGLEWVAVISYDGSNKYYADSVKGRFTISRDNSKNTLYLQMNSLRAEDTAVYYCARASRGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 294 - protein, 219 aa, synthetic construct
DVVMTQSPLSLPVTPGQSASISCRSSQSLVDRDGNTYLTWFHQRPGQSPRRLIFKVSNRDSGVPDRFSGSGSGTDFTLRISRVEAEDVGVYYCMQVTHWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 295 - protein, 449 aa, synthetic construct
QVQLQQSGAEVKKPGSSVKVSCKASGGTFSSYAISWVRQAPGQGLEWMGGIIPIFGTANYAQKFQGRVTITADKSTSTAYMELSSLRSEDTAVYYCARARSGSYSLHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 296 - protein, 446 aa, synthetic construct
QVQLQQSGAEVKKPGSSVKVSCKASGGTFSSYAISWVRQAPGQGLEWMGGIIPIFGTANYAQKFQGRVTITADKSTSTAYMELSSLRSEDTAVYYCARARSGSYSLHYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 297 - protein, 217 aa, synthetic construct
NFMLTQPHSVSESPGKTVTISCTGSSGSIDSNYVQWYQQRPGSAPTTVIFEDSQRPSGVPDRFSGSTDRSSNSASLTISGLKTEDEADYYCQSYDSSNNWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPAECS

SEQ ID NO: 298 - protein, 455 aa, synthetic construct
EVQLVQSGAEVKKPGASVKVSCKASGYTFTSYAMHWVRQAPGQRLEWMGWINAGNGNTKYSQKFQGRVTITRDTSASTAYMELSSLRSEDTAVYYCARVLHVLRYFDWPGAFDIWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 299 - protein, 452 aa, synthetic construct
EVQLVQSGAEVKKPGASVKVSCKASGYTFTSYAMHWVRQAPGQRLEWMGWINAGNGNTKYSQKFQGRVTITRDTSASTAYMELSSLRSEDTAVYYCARVLHVLRYFDWPGAFDIWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 300 - protein, 216 aa, synthetic construct
QSVLTQPPSASGTPGQRVTISCSGSSSNIGTNYVYWYQQLPGTAPKLLIYINDQRPSGVPDRFSGSKSGTSASLALSGLRSEDEADYYCAAWDDSLSGLVFGGGTKLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPAECS

SEQ ID NO: 301 - protein, 458 aa, synthetic construct
QVQLVQSGGGVVQPGRSLRLSCATSGFTFSSYGMHWVRQAPGKGLEWVAVISYDGSNKYYADSVKGRFTISRDNSKNTLYLQMNSLRAEDTAVYYCARDLVDSSGWYGYYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 302 - protein, 455 aa, synthetic construct
QVQLVQSGGGVVQPGRSLRLSCATSGFTFSSYGMHWVRQAPGKGLEWVAVISYDGSNKYYADSVKGRFTISRDNSKNTLYLQMNSLRAEDTAVYYCARDLVDSSGWYGYYYYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 303 - protein, 214 aa, synthetic construct
EIVLTQSPATLSLSPGERATLSCRASQSVSSYLAWYQQKPGQAPRLLIYDASNRATGIPARFSGSGSGTDFTLTISSLEPEDFAVYFCQQRSNWPGTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 304 - protein, 447 aa, synthetic construct
EVQLVQSGAEVKKPGDSVKVSCKASGYTFTGYYMHWVRQAPGQGLEWMGRINPNSGGTNYAQKFQGRVTMTTDTSTSTAYMELRSLRSDDTAVYYCARDLGGATVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 305 - protein, 444 aa, synthetic construct
EVQLVQSGAEVKKPGDSVKVSCKASGYTFTGYYMHWVRQAPGQGLEWMGRINPNSGGTNYAQKFQGRVTMTTDTSTSTAYMELRSLRSDDTAVYYCARDLGGATVYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 306 - protein, 221 aa, synthetic construct
QAVLTQPSSLSASPGASASLTCTLHSGVNVGTQRIYWYQQKPGSPPRYLVRYKSDSNKQQGSGVPSRFSGSKDASANAGILIISGLQSDDEADYYCMIWHNNAWVFGGGTTLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKAVAPTECS

SEQ ID NO: 307 - protein, 450 aa, synthetic construct
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARNGAVAGKYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 308 - protein, 447 aa, synthetic construct
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARNGAVAGKYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 309 - protein, 219 aa, synthetic construct
DVVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGEC

SEQ ID NO: 310 - protein, 454 aa, synthetic construct
QVQLVQSGGGVVQPGRSLRLSCAASGFTFSSYAMHWVRQAPGKGLEWVAVISYDGSNKYYADSVKGRFTISRDNSKNTLYLQMNSLRAEDTAVYYCARDFYPFIVGATSFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 311 - protein, 451 aa, synthetic construct
QVQLVQSGGGVVQPGRSLRLSCAASGFTFSSYAMHWVRQAPGKGLEWVAVISYDGSNKYYADSVKGRFTISRDNSKNTLYLQMNSLRAEDTAVYYCARDFYPFIVGATSFFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 312 - protein, 214 aa, synthetic construct
DIQLTQSPSSLSASVGDRVTITCRASQSISTYINWYQQKPGKAPKLLIYFASSLQSGVPSRFSGGGSGTDFTLTISSLQPEDFATYYCQQSYNIPVTFGQGTKVEVTRTVAAPSIFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 313 - protein, 449 aa, synthetic construct
QVQLVQSGGGVVQPGRSLRLSCAASGFTVSSNYMSWVRQAPGKGLEWVSVIYSGGSTYYADSVKGRFTISRDNSKNTLYLQMNSLRAEDTAVYYCARGSRAVAGAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 314 - protein, 446 aa, synthetic construct
QVQLVQSGGGVVQPGRSLRLSCAASGFTVSSNYMSWVRQAPGKGLEWVSVIYSGGSTYYADSVKGRFTISRDNSKNTLYLQMNSLRAEDTAVYYCARGSRAVAGAFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 315 - protein, 214 aa, synthetic construct
SSELTQDPAVSVALGQTVSITCQGDSLRNYFAAWYQQKPGQAPLLVIYGKNNRPSGIPDRFSGSSSGNTASLTITGAEAEDEADYYCGSRDSSGNHPVFGGGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 316 - protein, 214 aa, synthetic construct
YSELTQDPAVSVALGQTVRITCQGDSLRTYYANWYQQKPGQAPVLVMYAKNHRPSGIPDRFSGSSSGNTASLTITGAQAEDEADYYCGSRDSSGNHPVFGGGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 317 - protein, 452 aa, synthetic construct
QVQLQQSGPGLVKPSQTLSVTCAISGDSVSSNSAAWNWFRQSPSRGLEWLGRTYYRSKWYSDYAVSVKSRITINPDTSKNQFSLQLNSVTPEDTAVYYCARDVRAARAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 318 - protein, 449 aa, synthetic construct
QVQLQQSGPGLVKPSQTLSVTCAISGDSVSSNSAAWNWFRQSPSRGLEWLGRTYYRSKWYSDYAVSVKSRITINPDTSKNQFSLQLNSVTPEDTAVYYCARDVRAARAFDIWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 319 - protein, 221 aa, synthetic construct
QAVLTQPSSLSASPGASASLTCTLRSGINVGTYRIYWYQQKPGSPPQYLLRYKSDSDKQQGSGVPSRFSGSKDASANAGILVISGLQSEDEADYYCLIWHNRGWVFGGGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 320 - protein, 447 aa, synthetic construct
EVQLVESGGGLVQPGRPLRLSCAASGFTFSSYAMSWVRQAPGKGLEWVSSISSGGSTYYADSVRGRFTISRDDSKDTLYLQMNSLRAEDTAVYYCARTGVGGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 321 - protein, 444 aa, synthetic construct
EVQLVESGGGLVQPGRPLRLSCAASGFTFSSYAMSWVRQAPGKGLEWVSSISSGGSTYYADSVRGRFTISRDDSKDTLYLQMNSLRAEDTAVYYCARTGVGGAFDIWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 322 - protein, 219 aa, synthetic construct
DVVMTQSPLSLPVTPGEPASISCRSSHSLLYDNRYNYLDWYLQRPGQSPQLLIYLGSTRASGVPDRFSGSGSGTDFTLKISRVEAEDIGVYYCMQAVQTPIIFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 323 - protein, 446 aa, synthetic construct
EVQLLESGGGLVQPGGSLRLSCAASGFTFSSYAMSWVRQAPGKGLEWVSTIYYAGSNTYYADSVKGRFTISRDNSKNTLYLQMNSLRAEDTAVYYCAKGYYTFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 324 - protein, 443 aa, synthetic construct
EVQLLESGGGLVQPGGSLRLSCAASGFTFSSYAMSWVRQAPGKGLEWVSTIYYAGSNTYYADSVKGRFTISRDNSKNTLYLQMNSLRAEDTAVYYCAKGYYTFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 325 - protein, 210 aa, synthetic construct
QDPDVSVALGQTVRITCHGDSLRSSYASWYQQKPGQAPVLVFFGKNNRPSGIPDRFSGSSSGDTASLTITGAQAEDEADYYCHSRDTSDNHLRVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 326 - protein, 447 aa, synthetic construct
EVQLVESGGGLVRPGRSLRLSCRASGLTLSDYGMHWVRQAPGKGLEWVSGISGSGSNTYYTDSVKGRFTISRDNAKNSLYLQMNSLRAEDTAVYFCARSVRGAFDNWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 327 - protein, 444 aa, synthetic construct
EVQLVESGGGLVRPGRSLRLSCRASGLTLSDYGMHWVRQAPGKGLEWVSGISGSGSNTYYTDSVKGRFTISRDNAKNSLYLQMNSLRAEDTAVYFCARSVRGAFDNWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 328 - protein, 214 aa, synthetic construct
SSELTQDPAVSVALGQTVRITCQGDSLRRFFASWYQQRPGQAPILVIYGKNNRPSGIPDRFSGSRSGNTASLTIVGAQADDEADYYCSSRDSSRDRLVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 329 - protein, 216 aa, synthetic construct
QSVLTQPPSVSAAPGQKVTISCSGSSSNIGDNYVSWYQQLPGTAPKVLIYGNNNRPSGIPDRFSGSKSGTSASLAISGLQSEDEADYYCATWDDSLKGYVFGTGTQLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 330 - protein, 450 aa, synthetic construct
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCARGIVGAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 331 - protein, 447 aa, synthetic construct
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCARGIVGAFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 332 - protein, 215 aa, synthetic construct
SSELTQDPAVSVALGQTVRITCQGDSLRSYHASWYQQKPGQAPVLVIYGRNNRPSGIPDRFSGSTSGNTASLTITGAQAEDEADYYCKSRDSSGSLSYVFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 333 - protein, 592 aa, Homo sapiens
MEGKWLLCMLLVLGTAIVEAHDGHDDDVIDIEDDLDDVIEEVEDSKPDTTAPPSSPKVTYKAPVPTGEVYFADSFDRGTLSGWILSKAKKDDTDDEIAKYDGKWEVEEMKESKLPGDKGLVLMSRAKHHAISAKLNKPFLFDTKPLIVQYEVNFQNGIECGGAYVKLLSKTPELNLDQFHDKTPYTIMFGPDKCGEDYKLHFIFRHKNPKTGIYEEKHAKRPDADLKTYFTDKKTHLYTLILNPDNSFEILVDQSVVNSGNLLNDMTPPVNPSREIEDPEDRKPEDWDERPKIPDPEAVKPDDWDEDAPAKIPDEEATKPEGWLDDEPEYVPDPDAEKPEDWDEDMDGEWEAPQIANPRCESAPGCGVWQRPVIDNPNYKGKWKPPMIDNPSYQGIWKPRKIPNPDFFEDLEPFRMTPFSAIGLELWSMTSDIFFDNFIICADRRIVDDWANDGWGLKKAADGAAEPGVVGQMIEAAEERPWLWVVYILTVALPVFLVILFCCSGKKQTSGMEYKKTDAPQPDVKEEEEEKEEEKDKGDEEEEGEEKLEEKQKSDAEEDGGTVSQEEEDRKPKAEEDEILNRSPRNRKPRRE

SEQ ID NO: 334 - protein, 627 aa, Homo sapiens
MADRRTPTPFAGCRLPRQRRARDASQVSAPGTRRIMEGKWLLCMLLVLGTAIVEAHDGHDDDVIDIEDDLDDVIEEVEDSKPDTTAPPSSPKVTYKAPVPTGEVYFADSFDRGTLSGWILSKAKKDDTDDEIAKYDGKWEVEEMKESKLPGDKGLVLMSRAKHHAISAKLNKPFLFDTKPLIVQYEVNFQNGIECGGAYVKLLSKTPELNLDQFHDKTPYTIMFGPDKCGEDYKLHFIFRHKNPKTGIYEEKHAKRPDADLKTYFTDKKTHLYTLILNPDNSFEILVDQSVVNSGNLLNDMTPPVNPSREIEDPEDRKPEDWDERPKIPDPEAVKPDDWDEDAPAKIPDEEATKPEGWLDDEPEYVPDPDAEKPEDWDEDMDGEWEAPQIANPRCESAPGCGVWQRPVIDNPNYKGKWKPPMIDNPSYQGIWKPRKIPNPDFFEDLEPFRMTPFSAIGLELWSMTSDIFFDNFIICADRRIVDDWANDGWGLKKAADGAAEPGVVGQMIEAAEERPWLWVVYILTVALPVFLVILFCCSGKKQTSGMEYKKTDAPQPDVKEEEEEKEEEKDKGDEEEEGEEKLEEKQKSDAEEDGGTVSQEEEDRKPKAEEDEILNRSPRNRKPRRE

SEQ ID NO: 335 - protein, 484 aa, Homo sapiens
MKESKLPGDKGLVLMSRAKHHAISAKLNKPFLFDTKPLIVQYEVNFQNGIECGGAYVKLLSKTPELNLDQFHDKTPYTIMFGPDKCGEDYKLHFIFRHKNPKTGIYEEKHAKRPDADLKTYFTDKKTHLYTLILNPDNSFEILVDQSVVNSGNLLNDMTPPVNPSREIEDPEDRKPEDWDERPKIPDPEAVKPDDWDEDAPAKIPDEEATKPEGWLDDEPEYVPDPDAEKPEDWDEDMDGEWEAPQIANPRCESAPGCGVWQRPVIDNPNYKGKWKPPMIDNPSYQGIWKPRKIPNPDFFEDLEPFRMTPFSAIGLELWSMTSDIFFDNFIICADRRIVDDWANDGWGLKKAADGAAEPGVVGQMIEAAEERPWLWVVYILTVALPVFLVILFCCSGKKQTSGMEYKKTDAPQPDVKEEEEEKEEEKDKGDEEEEGEEKLEEKQKSDAEEDGGTVSQEEEDRKPKAEEDEILNRSPRNRKPRRE

SEQ ID NO: 336 - protein, 20 aa, Homo sapiens
MEGKWLLCMLLVLGTAIVEA

SEQ ID NO: 337 - protein, 461 aa, Homo sapiens
HDGHDDDVIDIEDDLDDVIEEVEDSKPDTTAPPSSPKVTYKAPVPTGEVYFADSFDRGTLSGWILSKAKKDDTDDEIAKYDGKWEVEEMKESKLPGDKGLVLMSRAKHHAISAKLNKPFLFDTKPLIVQYEVNFQNGIECGGAYVKLLSKTPELNLDQFHDKTPYTIMFGPDKCGEDYKLHFIFRHKNPKTGIYEEKHAKRPDADLKTYFTDKKTHLYTLILNPDNSFEILVDQSVVNSGNLLNDMTPPVNPSREIEDPEDRKPEDWDERPKIPDPEAVKPDDWDEDAPAKIPDEEATKPEGWLDDEPEYVPDPDAEKPEDWDEDMDGEWEAPQIANPRCESAPGCGVWQRPVIDNPNYKGKWKPPMIDNPSYQGIWKPRKIPNPDFFEDLEPFRMTPFSAIGLELWSMTSDIFFDNFIICADRRIVDDWANDGWGLKKAADGAAEPGVVGQMIEAAEERP

SEQ ID NO: 338 - protein, 21 aa, Homo sapiens
WLWVVYILTVALPVFLVILFC

SEQ ID NO: 339 - protein, 90 aa, Homo sapiens
CSGKKQTSGMEYKKTDAPQPDVKEEEEEKEEEKDKGDEEEEGEEKLEEKQKSDAEEDGGTVSQEEEDRKPKAEEDEILNRSPRNRKPRRE

SEQ ID NO: 340 - protein, 211 aa, Homo sapiens
KAPVPTGEVYFADSFDRGTLSGWILSKAKKDDTDDEIAKYDGKWEVEEMKESKLPGDKGLVLMSRAKHHAISAKLNKPFLFDTKPLIVQYEVNFQNGIECGGAYVKLLSKTPELNLDQFHDKTPYTIMFGPDKCGEDYKLHFIFRHKNPKTGIYEEKHAKRPDADLKTYFTDKKTHLYTLILNPDNSFEILVDQSVVNSGNLLNDMTPPVN

SEQ ID NO: 341 - protein, 140 aa, Homo sapiens
PSREIEDPEDRKPEDWDERPKIPDPEAVKPDDWDEDAPAKIPDEEATKPEGWLDDEPEYVPDPDAEKPEDWDEDMDGEWEAPQIANPRCESAPGCGVWQRPVIDNPNYKGKWKPPMIDNPSYQGIWKPRKIPNPDFFEDL

SEQ ID NO: 342 - protein, 46 aa, Homo sapiens
EPFRMTPFSAIGLELWSMTSDIFFDNFIICADRRIVDDWANDGWGL

SEQ ID NO: 343 - protein, 572 aa, Homo sapiens
HDGHDDDVIDIEDDLDDVIEEVEDSKPDTTAPPSSPKVTYKAPVPTGEVYFADSFDRGTLSGWILSKAKKDDTDDEIAKYDGKWEVEEMKESKLPGDKGLVLMSRAKHHAISAKLNKPFLFDTKPLIVQYEVNFQNGIECGGAYVKLLSKTPELNLDQFHDKTPYTIMFGPDKCGEDYKLHFIFRHKNPKTGIYEEKHAKRPDADLKTYFTDKKTHLYTLILNPDNSFEILVDQSVVNSGNLLNDMTPPVNPSREIEDPEDRKPEDWDERPKIPDPEAVKPDDWDEDAPAKIPDEEATKPEGWLDDEPEYVPDPDAEKPEDWDEDMDGEWEAPQIANPRCESAPGCGVWQRPVIDNPNYKGKWKPPMIDNPSYQGIWKPRKIPNPDFFEDLEPFRMTPFSAIGLELWSMTSDIFFDNFIICADRRIVDDWANDGWGLKKAADGAAEPGVVGQMIEAAEERPWLWVVYILTVALPVFLVILFCCSGKKQTSGMEYKKTDAPQPDVKEEEEEKEEEKDKGDEEEEGEEKLEEKQKSDAEEDGGTVSQEEEDRKPKAEEDEILNRSPRNRKPRRE

SEQ ID NO: 344 - protein, 591 aa, Mus musculus
MEGKWLLCLLLVLGTAAVEAHDGHDDDAIDIEDDLDDVIEEVEDSKSKSDASTPPSPKVTYKAPVPTGEVYFADSFDRGSLSGWILSKAKKDDTDDEIAKYDGKWEVDEMKETKLPGDKGLVLMSRAKHHAISAKLNKPFLFDTKPLIVQYEVNFQNGIECGGAYVKLLSKTAELSLDQFHDKTPYTIMFGPDKCGEDYKLHFIFRHKNPKTGVYEEKHAKRPDADLKTYFTDKKTHLYTLILNPDNSFEILVDQSVVNSGNLLNDMTPPVNPSREIEDPEDRKPEDWDERPKIADPDAVKPDDWDEDAPSKIPDEEATKPEGWLDDEPEYIPDPDAEKPEDWDEDMDGEWEAPQIANPKCESAPGCGVWQRPMIDNPNYKGKWKPPMIDNPNYQGIWKPRKIPNPDFFEDLEPFKMTPFSAIGLELWSMTSDIFFDNFIISGDRRVVDDWANDGWGLKKAADGAAEPGVVLQMLEAAEERPWLWVVYILTVALPVFLVILFCCSGKKQSNAMEYKKTDAPQPDVKDEEGKEEEKNKRDEEEEEEKLEEKQKSDAEEDGVTGSQDEEDSKPKAEEDEILNRSPRNRKPRRE

SEQ ID NO: 345 - protein, 20 aa, Mus musculus
MEGKWLLCLLLVLGTAAVEA

SEQ ID NO: 346 - protein, 462 aa, Mus musculus
HDGHDDDAIDIEDDLDDVIEEVEDSKSKSDASTPPSPKVTYKAPVPTGEVYFADSFDRGSLSGWILSKAKKDDTDDEIAKYDGKWEVDEMKETKLPGDKGLVLMSRAKHHAISAKLNKPFLFDTKPLIVQYEVNFQNGIECGGAYVKLLSKTAELSLDQFHDKTPYTIMFGPDKCGEDYKLHFIFRHKNPKTGVYEEKHAKRPDADLKTYFTDKKTHLYTLILNPDNSFEILVDQSVVNSGNLLNDMTPPVNPSREIEDPEDRKPEDWDERPKIADPDAVKPDDWDEDAPSKIPDEEATKPEGWLDDEPEYIPDPDAEKPEDWDEDMDGEWEAPQIANPKCESAPGCGVWQRPMIDNPNYKGKWKPPMIDNPNYQGIWKPRKIPNPDFFEDLEPFKMTPFSAIGLELWSMTSDIFFDNFIISGDRRVVDDWANDGWGLKKAADGAAEPGVVLQMLEAAEERP

SEQ ID NO: 347 - protein, 21 aa, Mus musculus
WLWVVYILTVALPVFLVILFC

SEQ ID NO: 348 - protein, 88 aa, Mus musculus
CSGKKQSNAMEYKKTDAPQPDVKDEEGKEEEKNKRDEEEEEEKLEEKQKSDAEEDGVTGSQDEEDSKPKAEEDEILNRSPRNRKPRRE

SEQ ID NO: 349 - protein, 211 aa, Mus musculus
KAPVPTGEVYFADSFDRGSLSGWILSKAKKDDTDDEIAKYDGKWEVDEMKETKLPGDKGLVLMSRAKHHAISAKLNKPFLFDTKPLIVQYEVNFQNGIECGGAYVKLLSKTAELSLDQFHDKTPYTIMFGPDKCGEDYKLHFIFRHKNPKTGVYEEKHAKRPDADLKTYFTDKKTHLYTLILNPDNSFEILVDQSVVNSGNLLNDMTPPVN

SEQ ID NO: 350 - protein, 140 aa, Mus musculus
PSREIEDPEDRKPEDWDERPKIADPDAVKPDDWDEDAPSKIPDEEATKPEGWLDDEPEYIPDPDAEKPEDWDEDMDGEWEAPQIANPKCESAPGCGVWQRPMIDNPNYKGKWKPPMIDNPNYQGIWKPRKIPNPDFFEDL

SEQ ID NO: 351 - protein, 46 aa, Mus musculus
EPFKMTPFSAIGLELWSMTSDIFFDNFIISGDRRVVDDWANDGWGL

SEQ ID NO: 352 - protein, 571 aa, Mus musculus
HDGHDDDAIDIEDDLDDVIEEVEDSKSKSDASTPPSPKVTYKAPVPTGEVYFADSFDRGSLSGWILSKAKKDDTDDEIAKYDGKWEVDEMKETKLPGDKGLVLMSRAKHHAISAKLNKPFLFDTKPLIVQYEVNFQNGIECGGAYVKLLSKTAELSLDQFHDKTPYTIMFGPDKCGEDYKLHFIFRHKNPKTGVYEEKHAKRPDADLKTYFTDKKTHLYTLILNPDNSFEILVDQSVVNSGNLLNDMTPPVNPSREIEDPEDRKPEDWDERPKIADPDAVKPDDWDEDAPSKIPDEEATKPEGWLDDEPEYIPDPDAEKPEDWDEDMDGEWEAPQIANPKCESAPGCGVWQRPMIDNPNYKGKWKPPMIDNPNYQGIWKPRKIPNPDFFEDLEPFKMTPFSAIGLELWSMTSDIFFDNFIISGDRRVVDDWANDGWGLKKAADGAAEPGVVLQMLEAAEERPWLWVVYILTVALPVFLVILFCCSGKKQSNAMEYKKTDAPQPDVKDEEGKEEEKNKRDEEEEEEKLEEKQKSDAEEDGVTGSQDEEDSKPKAEEDEILNRSPRNRKPRRE

SEQ ID NO: 353 - protein, 417 aa, Homo sapiens
MLLSVPLLLGLLGLAVAEPAVYFKEQFLDGDGWTSRWIESKHKSDFGKFVLSSGKFYGDEEKDKGLQTSQDARFYALSASFEPFSNKGQTLVVQFTVKHEQNIDCGGGYVKLFPNSLDQTDMHGDSEYNIMFGPDICGPGTKKVHVIFNYKGKNVLINKDIRCKDDEFTHLYTLIVRPDNTYEVKIDNSQVESGSLEDDWDFLPPKKIKDPDASKPEDWDERAKIDDPTDSKPEDWDKPEHIPDPDAKKPEDWDEEMDGEWEPPVIQNPEYKGEWKPRQIDNPDYKGTWIHPEIDNPEYSPDPSIYAYDNFGVLGLDLWQVKSGTIFDNFLITNDEAYAEEFGNETWGVTKAAEKQMKDKQDEEQRLKEEEEDKKRKEEEEAEDKEDDEDKDEDEEDEEDKEEDEEEDVPGQAKDEL

SEQ ID NO: 354 - protein, 17 aa, Homo sapiens
MLLSVPLLLGLLGLAVA

SEQ ID NO: 355 - protein, 339 aa, Homo sapiens
EPAVYFKEQFLDGDGWTSRWIESKHKSDFGKFVLSSGKFYGDEEKDKGLQTSQDARFYALSASFEPFSNKGQTLVVQFTVKHEQNIDCGGGYVKLFPNSLDQTDMHGDSEYNIMFGPDICGPGTKKVHVIFNYKGKNVLINKDIRCKDDEFTHLYTLIVRPDNTYEVKIDNSQVESGSLEDDWDFLPPKKIKDPDASKPEDWDERAKIDDPTDSKPEDWDKPEHIPDPDAKKPEDWDEEMDGEWEPPVIQNPEYKGEWKPRQIDNPDYKGTWIHPEIDNPEYSPDPSIYAYDNFGVLGLDLWQVKSGTIFDNFLITNDEAYAEEFGNETWGVTKAAEKQ

SEQ ID NO: 356 - protein, 61 aa, Homo sapiens [sequence omitted]

SEQ ID NO: 357 - protein, 184 aa, Homo sapiens
EPAVYFKEQFLDGDGWTSRWIESKHKSDFGKFVLSSGKFYGDEEKDKGLQTSQDARFYALSASFEPFSNKGQTLVVQFTVKHEQNIDCGGGYVKLFPNSLDQTDMHGDSEYNIMFGPDICGPGTKKVHVIFNYKGKNVLINKDIRCKDDEFTHLYTLIVRPDNTYEVKIDNSQVESGSLEDDWD

SEQ ID NO: 358 - protein, 55 aa, Homo sapiens
DPSIYAYDNFGVLGLDLWQVKSGTIFDNFLITNDEAYAEEFGNETWGVTKAAEKQ

SEQ ID NO: 359 - protein, 100 aa, Homo sapiens
FLPPKKIKDPDASKPEDWDERAKIDDPTDSKPEDWDKPEHIPDPDAKKPEDWDEEMDGEWEPPVIQNPEYKGEWKPRQIDNPDYKGTWIHPEIDNPEYSP

SEQ ID NO: 360 - protein, 400 aa, Homo sapiens
EPAVYFKEQFLDGDGWTSRWIESKHKSDFGKFVLSSGKFYGDEEKDKGLQTSQDARFYALSASFEPFSNKGQTLVVQFTVKHEQNIDCGGGYVKLFPNSLDQTDMHGDSEYNIMFGPDICGPGTKKVHVIFNYKGKNVLINKDIRCKDDEFTHLYTLIVRPDNTYEVKIDNSQVESGSLEDDWDFLPPKKIKDPDASKPEDWDERAKIDDPTDSKPEDWDKPEHIPDPDAKKPEDWDEEMDGEWEPPVIQNPEYKGEWKPRQIDNPDYKGTWIHPEIDNPEYSPDPSIYAYDNFGVLGLDLWQVKSGTIFDNFLITNDEAYAEEFGNETWGVTKAAEKQMKDKQDEEQRLKEEEEDKKRKEEEEAEDKEDDEDKDEDEEDEEDKEEDEEEDVPGQAKDEL

SEQ ID NO: 361 - protein, 15 aa, synthetic construct
YEVNFQNGIECGGAY

SEQ ID NO: 362 - protein, 11 aa, synthetic construct
WEAPQIANPRC

SEQ ID NO: 363 - protein, 211 aa, synthetic construct
YEVNFQNGIECGGAYVKLLSKTPELNLDQFHDKTPYTIMFGPDKCGEDYKLHFIFRHKNPKTGIYEEKHAKRPDADLKTYFTDKKTHLYTLILNPDNSFEILVDQSVVNSGNLLNDMTPPVNPSREIEDPEDRKPEDWDERPKIPDPEAVKPDDWDEDAPAKIPDEEATKPEGWLDDEPEYVPDPDAEKPEDWDEDMDGEWEAPQIANPRC

SEQ ID NO: 364 - protein, 14 aa, synthetic construct
VYFADSFDRGTLSG

SEQ ID NO: 365 - protein, 25 aa, synthetic construct
ADSFDRGTLSGWILSKAKKDDTDDE

SEQ ID NO: 366 - protein, 24 aa, synthetic construct
VDQSVVNSGNLLNDMTPPVNPSRE

SEQ ID NO: 367 - protein, 29 aa, synthetic construct
DERPKIPDPEAVKPDDWDEDAPAKIPDEE

SEQ ID NO: 368 - protein, 19 aa, synthetic construct
WEAPQIANPRCESAPGCGV

SEQ ID NO: 369 - protein, 39 aa, synthetic construct
WQRPVIDNPNYKGKWKPPMIDNPSYQGIWKPRKIPNPDF

SEQ ID NO: 370 - protein, 26 aa, synthetic construct
WANDGWGLKKAADGAAEPGVVGQMIE

SEQ ID NO: 371 - protein, 407 aa, synthetic construct
VYFADSFDRGTLSGWILSKAKKDDTDDEIAKYDGKWEVEEMKESKLPGDKGLVLMSRAKHHAISAKLNKPFLFDTKPLIVQYEVNFQNGIECGGAYVKLLSKTPELNLDQFHDKTPYTIMFGPDKCGEDYKLHFIFRHKNPKTGIYEEKHAKRPDADLKTYFTDKKTHLYTLILNPDNSFEILVDQSVVNSGNLLNDMTPPVNPSREIEDPEDRKPEDWDERPKIPDPEAVKPDDWDEDAPAKIPDEEATKPEGWLDDEPEYVPDPDAEKPEDWDEDMDGEWEAPQIANPRCESAPGCGVWQRPVIDNPNYKGKWKPPMIDNPSYQGIWKPRKIPNPDFFEDLEPFRMTPFSAIGLELWSMTSDIFFDNFIICADRRIVDDWANDGWGLKKAADGAAEPGVVGQMIE

SEQ ID NO: 372 - protein, 28 aa, synthetic construct
VYFADSFDRGTLSGWILSKAKKDDTDDE

SEQ ID NO: 373 - protein, 58 aa, synthetic construct
WEAPQIANPRCESAPGCGVWQRPVIDNPNYKGKWKPPMIDNPSYQGIWKPRKIPNPDF

SEQ ID NO: 374 - DNA, 9522 bp, synthetic construct
  source 1..9522: mol_type = other DNA
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatcgataagcttgggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatccctaccggtgatatcctcgagaccatggctacaggctcccggacgtccctgctcctggcttttggcctgctctgcctgccctggcttcaagagggcagtgccttcccaaccattcccttatccgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcacctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaaggtctatatcaccgccgacaagcagaagaacggcatcaaggtgaacttcaagacccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggctcgagcccatcaacaagtttgtacaaaaaagcaggctccgataacgggaagcctatccctaaccctctcctcggtctcgattctacggagttaagggttccagaccatcaggatatagcttttggggccttgcagcagggaactaactgcctcgacactttgggacactttgctgatggtgtggttggagtttatgaatgtcacaatgctgggggaaaccaggaatgggccttgacgaaggagaagtcggtgaagcacatggatttgtgccttactgtggtggaccgggcaccgggctctcttataaagctgcagggctgccgagaaaatgacagcagacagaaatgggaacagatcgagggcaactccaagctgaggcacgtgggcagcaacctgtgcctggacagtcgcacggccaagagcgggggcctaagcgtggaggtgtgtggcccggccctttcgcagcagtggaagttcacgctcaacctgcagcaggcacagggagcccaagggagatccgataacgggaagcctatccctaaccctctcctcggtctcgattctacggagttaagggttccagaccatcaggatatagcttttggggccttgcagcagggaactaactgcctcgacactttgggacactttgctgatggtgtggttggagtttatgaatgtcacaatgctgggggaaaccaggaatgggccttgacgaaggagaagtcggtgaagcacatggatttgtgccttactgtggtggaccgggcaccgggctctcttataaagctgcagggctgccgagaaaatgacagcagacagaaatgggaacagatcgagggcaactccaagctgaggcacgtgggcagcaacctgtgcctggacagtcgcacggccaagagcgggggcctaagcgtggaggtgtgtggcccggccctttcgcagcagtggaagttcacgctcaacctgcagcagaaagacgagctgtaggacccagctttcttgtacaaagtggttgatatccagcacagtggcggccgctcgagtctagagggcccgcggttcgaaggtaagcctatccctaaccctctcctcggtctcgattctacgcgtaccggttagtaatgatcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggcgatggtaccggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagcacaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 375 - RNA, 21 nt, synthetic construct
  source 1..21: mol_type = other RNA
  ncRNA 1..21: ncRNA_class = siRNA
caagagtggtcctaggagatt

SEQ ID NO: 376 - RNA, 19 nt, synthetic construct
  source 1..19: mol_type = other RNA
  ncRNA 1..19: ncRNA_class = siRNA
ggaatagtcccattagcaa

SEQ ID NO: 377 - RNA, 19 nt, synthetic construct
  source 1..19: mol_type = other RNA
  ncRNA 1..19: ncRNA_class = siRNA
gggcaaggacttacttatt

SEQ ID NO: 378 - RNA, 19 nt, synthetic construct
  source 1..19: mol_type = other RNA
  ncRNA 1..19: ncRNA_class = siRNA
agacccaaatatcgtcata

SEQ ID NO: 379 - RNA, 18 nt, synthetic construct
  source 1..18: mol_type = other RNA
  ncRNA 1..18: ncRNA_class = siRNA
aggagttctcgcgtgatg

SEQ ID NO: 380 - RNA, 19 nt, synthetic construct
  source 1..19: mol_type = other RNA
  ncRNA 1..19: ncRNA_class = siRNA
gaacgagtatgatgataat

SEQ ID NO: 381 - RNA, 19 nt, synthetic construct
  source 1..19: mol_type = other RNA
  ncRNA 1..19: ncRNA_class = siRNA
ggacaagactgtggcatat

SEQ ID NO: 382 - RNA, 19 nt, synthetic construct
  source 1..19: mol_type = other RNA
  ncRNA 1..19: ncRNA_class = siRNA
ggcaaggacttacttattg

SEQ ID NO: 383 - RNA, 19 nt, synthetic construct
  source 1..19: mol_type = other RNA
  ncRNA 1..19: ncRNA_class = siRNA
tgataaagatgcctctata

SEQ ID NO: 384 - protein, 13 aa, synthetic construct
VQSGGGVVQPGRS

SEQ ID NO: 385 - protein, 9 aa, synthetic construct
YADSVKGRF

SEQ ID NO: 386 - protein, 18 aa, synthetic construct
SRAVAGAFDYWGQGTLVT

SEQ ID NO: 387 - protein, 27 aa, synthetic construct
VMYAKNHRPSGIPDRFSGSSSGNTASL

SEQ ID NO: 388 - protein, 24 aa, synthetic construct
YYVGSRDSSGNHPVFGGGTKVTLV

SEQ ID NO: 389 - protein, 11 aa, synthetic construct
ADSFDRGTLSG

SEQ ID NO: 390 - protein, 13 aa, synthetic construct
YVKLLSKTPELNL

SEQ ID NO: 391 - protein, 18 aa, synthetic construct
DEDAPAKIPDEEATKPEG

SEQ ID NO: 392 - protein, 27 aa, synthetic construct
WVRQAPGKGLEWVSGINWNGGSTGYAD

SEQ ID NO: 393 - protein, 22 aa, synthetic construct
WVSIGNWNGGSTGYADSVKGRF

SEQ ID NO: 394 - protein, 30 aa, synthetic construct
VTVSSASTKGPSVFPLAPSSKSTSGGTAAL

SEQ ID NO: 395 - protein, 29 aa, synthetic construct
VKDYFPEPVTVSWNSGALTSGVHTFPAVL

SEQ ID NO: 396 - protein, 15 aa, synthetic construct
VVDVSHEDPEVKFNW

SEQ ID NO: 397 - protein, 27 aa, synthetic construct
TVLHQDWLNGKEYKCKVSNKALPAPIE

SEQ ID NO: 398 - protein, 17 aa, synthetic construct
VLHQDWLNGKEYKCKVS

SEQ ID NO: 399 - protein, 32 aa, synthetic construct
KTISKAKGQPREPQVYTLPPSRDELTKNQVSL

SEQ ID NO: 400 - protein, 16 aa, synthetic construct
YSKLTVDKSRWQQGNV

SEQ ID NO: 401 - protein, 13 aa, synthetic construct
TLKISRVEAEDVG

SEQ ID NO: 402 - protein, 17 aa, synthetic construct
VFIFPPSDEQLKSGTAS

SEQ ID NO: 403 - protein, 29 aa, synthetic construct
VCLLNNFYPREAKVQWKVDNALQSGNSQE

SEQ ID NO: 404 - protein, 20 aa, synthetic construct
EVTHQGLSSPVTKSFNRGEC

SEQ ID NO: 405 - protein, 18 aa, synthetic construct
WEAPQIANPRCESAPCGV

SEQ ID NO: 406 - protein, 27 aa, synthetic construct
WANDGWGLKKAADDGAAEPGVVGQMIE

SEQ ID NO: 407 - protein, 33 aa, synthetic construct
QNGIECGGAYYVKLLSKTPELNLDQFHDKTPYT

SEQ ID NO: 408 - protein, 16 aa, synthetic construct
TPTTVGPTTVGSTTVG

SEQ ID NO: 409 - protein, 15 aa, synthetic construct
GGGGSGGGGSGGGGS

SEQ ID NO: 410 - protein, 4 aa, synthetic construct
  REGION >4: note = The entire sequence of amino acids 1-4 can be repeated two to six times.
GGGS

SEQ ID NO: 411 - protein, 5 aa, synthetic construct
  REGION >5: note = The entire sequence of amino acids 1-5 can be repeated two to six times.
GGGGS

SEQ ID NO: 412 - protein, 5 aa, synthetic construct
  REGION >4: note = The entire sequence of amino acids 1-4 can be repeated two to six times
  REGION >5: note = The amino acid 5 can be repeated one to three times or absent.
GGGSG

SEQ ID NO: 413 - protein, 6 aa, synthetic construct
  REGION >5: note = The entire sequence of amino acids 1-5 can be repeated two to six times
  REGION >6: note = The amino acid 6 can be repeated one to three times or absent
GGGGSG

SEQ ID NO: 414 - protein, 5 aa, synthetic construct
GGGGS

SEQ ID NO: 415 - protein, 20 aa, synthetic construct
GGGGSGGGGSGGGGSGGGGS

SEQ ID NO: 416 - protein, 30 aa, synthetic construct
GGGGSGGGGSGGGGSGGGGSGGGGSGGGGS

(19) United States
(12) Patent Application Publication
(10) Pub. No.: US 2026/0250378 A1
(43) Pub. Date: Aug. 27, 2026

(54) CNX ANTIGEN-BINDING MOLECULES

(71) Applicants: Agency for Science, Technology and Research, Singapore (SG); Albatroz Therapeutics Pte. Ltd., Singapore (SG)

(72) Inventors: Frederic Bard, Singapore (SG); Cheng-I Wang, Singapore (SG); Zhi Hui Joanne Chia, Singapore (SG); Eve Ngoh, Singapore (SG); Xavier Le Guezennec, Singapore (SG); Tham Keit Min, Singapore (SG); Anh Tuan Nguyen, Singapore (SG); Rebecca Bennion, Singapore (SG)

(73) Assignees: Agency for Science, Technology and Research, Singapore (SG); Albatroz Therapeutics Pte. Ltd., Singapore (SG)

(21) Appl. No.: 18/992,129

(22) PCT Filed: Jul. 7, 2023
(86) PCT No.: PCT/EP2023/068932

§ 371 Date: Jan. 7, 2025

Related U.S. Application Data
(60) Provisional application No. 63/359,499, filed on Jul. 8, 2022.

(51) Int. Cl. C07K 16/28; A61K 39/00; A61K 40/31; A61K 40/33; A61K 47/68; A61P 35/00; C07K 16/18; G01N 33/575

(52) U.S. Cl. CPC C07K 16/28; A61K 40/31; A61K 40/33; A61K 47/6889; A61P 35/00; C07K 16/18; G01N 33/5759; A61K 2039/505; A61K 2239/13; C07K 2317/31; C07K 2317/565; C07K 2317/76; G01N 2333/705

(57) ABSTRACT

Antigen binding molecules capable of binding to calnexin (CNX) are disclosed herein. Also disclosed are chimeric antigen receptors, antibody-drug conjugates, and compositions comprising such antigen binding molecules, as well as nucleic acids, vectors and cells. Uses and methods involving antigen binding molecules capable of binding to calnexin (CNX) are also disclosed.

This application claims priority from U.S. 63/359,499 filed 8 Jul. 2022, the contents and elements of which are herein incorporated by reference for all purposes.

### TECHNICAL FIELD

The present disclosure relates to the fields of molecular biology, more specifically antibody technology. The present disclosure also relates to methods of medical treatment and prophylaxis.

### BACKGROUND

CNX is an endoplasmic reticulum (ER)-resident lectin chaperone protein, which binds to N-glycoproteins bearing monoglucosylated glycans, and recruits various other chaperones that mediate protein disulfide formation, proline isomerisation, and protein folding.

Recent studies have implicated CNX and CNX-containing complexes (e.g. CNX:ERp57) in the pathology of diseases/conditions including cancers, particularly through their ECM degrading activity (see Ros et al. Nat. Cell Biol. (2020) 22(11):1371-1381).

Ros et al. Nat. Cell Biol. (2020) 22(11):1371-1381 discloses anti-CNX antibodies ab10286 and ab22595. Abcam's ab10286 and ab22595 are each rabbit polyclonal antibody preparations to human CNX. There remains a need to develop antibodies to CNX suitable for use in methods of medical treatment and prophylaxis.

### SUMMARY

In a first aspect, the present disclosure provides an antigen-binding molecule, optionally isolated, which binds to CNX.

In some embodiments, the antigen-binding molecule inhibits extracellular matrix (ECM) degradation.

In some embodiments, the antigen-binding molecule comprises:

(a)
(i) a heavy chain variable (VH) region incorporating the following CDRs:
   HC-CDR1 having the amino acid sequence of SEQ ID NO:166
   HC-CDR2 having the amino acid sequence of SEQ ID NO:167
   HC-CDR3 having the amino acid sequence of SEQ ID NO:168; and
(ii) a light chain variable (VL) region incorporating the following CDRs:
   LC-CDR1 having the amino acid sequence of SEQ ID NO:179
   LC-CDR2 having the amino acid sequence of SEQ ID NO:180
   LC-CDR3 having the amino acid sequence of SEQ ID NO:173; or
      (b)
(i) a heavy chain variable (VH) region incorporating the following CDRs:
   HC-CDR1 having the amino acid sequence of SEQ ID NO:33
   HC-CDR2 having the amino acid sequence of SEQ ID NO:34
   HC-CDR3 having the amino acid sequence of SEQ ID NO:35; and
(ii) a light chain variable (VL) region incorporating the following CDRs:
   LC-CDR1 having the amino acid sequence of SEQ ID NO:41
   LC-CDR2 having the amino acid sequence of SEQ ID NO:42
   LC-CDR3 having the amino acid sequence of SEQ ID NO:43; or
      (c)
(i) a heavy chain variable (VH) region incorporating the following CDRs:
   HC-CDR1 having the amino acid sequence of SEQ ID NO:2
   HC-CDR2 having the amino acid sequence of SEQ ID NO:3
   HC-CDR3 having the amino acid sequence of SEQ ID NO:4; and
(ii) a light chain variable (VL) region incorporating the following CDRs:
   LC-CDR1 having the amino acid sequence of SEQ ID NO:10
   LC-CDR2 having the amino acid sequence of SEQ ID NO:11
   LC-CDR3 having the amino acid sequence of SEQ ID NO:12; or
      (d)
(i) a heavy chain variable (VH) region incorporating the following CDRs:
   HC-CDR1 having the amino acid sequence of SEQ ID NO:18
   HC-CDR2 having the amino acid sequence of SEQ ID NO:19
   HC-CDR3 having the amino acid sequence of SEQ ID NO:20; and
(ii) a light chain variable (VL) region incorporating the following CDRs:
   LC-CDR1 having the amino acid sequence of SEQ ID NO:25
   LC-CDR2 having the amino acid sequence of SEQ ID NO:26
   LC-CDR3 having the amino acid sequence of SEQ ID NO:27; or
      (e)
(i) a heavy chain variable (VH) region incorporating the following CDRs:
   HC-CDR1 having the amino acid sequence of SEQ ID NO:48
   HC-CDR2 having the amino acid sequence of SEQ ID NO:3
   HC-CDR3 having the amino acid sequence of SEQ ID NO:49; and
(ii) a light chain variable (VL) region incorporating the following CDRs:
   LC-CDR1 having the amino acid sequence of SEQ ID NO:53
   LC-CDR2 having the amino acid sequence of SEQ ID NO:54
   LC-CDR3 having the amino acid sequence of SEQ ID NO:55; or
      (f)
(i) a heavy chain variable (VH) region incorporating the following CDRs:
   HC-CDR1 having the amino acid sequence of SEQ ID NO:61
   HC-CDR2 having the amino acid sequence of SEQ ID NO:62
   HC-CDR3 having the amino acid sequence of SEQ ID NO:63; and
(ii) a light chain variable (VL) region incorporating the following CDRs:
   LC-CDR1 having the amino acid sequence of SEQ ID NO:68
   LC-CDR2 having the amino acid sequence of SEQ ID NO:26
   LC-CDR3 having the amino acid sequence of SEQ ID NO:69; or
      (g)
(i) a heavy chain variable (VH) region incorporating the following CDRs:
   HC-CDR1 having the amino acid sequence of SEQ ID NO:61
   HC-CDR2 having the amino acid sequence of SEQ ID NO:62
   HC-CDR3 having the amino acid sequence of SEQ ID NO:63; and
(ii) a light chain variable (VL) region incorporating the following CDRs:
   LC-CDR1 having the amino acid sequence of SEQ ID NO:73
   LC-CDR2 having the amino acid sequence of SEQ ID NO:26
   LC-CDR3 having the amino acid sequence of SEQ ID NO:74; or
      (h)
(i) a heavy chain variable (VH) region incorporating the following CDRs:
   HC-CDR1 having the amino acid sequence of SEQ ID NO:61
   HC-CDR2 having the amino acid sequence of SEQ ID NO:62
   HC-CDR3 having the amino acid sequence of SEQ ID NO:63; and
(ii) a light chain variable (VL) region incorporating the following CDRs:
   LC-CDR1 having the amino acid sequence of SEQ ID NO:78
   LC-CDR2 having the amino acid sequence of SEQ ID NO:79
   LC-CDR3 having the amino acid sequence of SEQ ID NO:80; or
      (i)
(i) a heavy chain variable (VH) region incorporating the following CDRs:
   HC-CDR1 having the amino acid sequence of SEQ ID NO:2
   HC-CDR2 having the amino acid sequence of SEQ ID NO:3
   HC-CDR3 having the amino acid sequence of SEQ ID NO:83; and
(ii) a light chain variable (VL) region incorporating the following CDRs:
   LC-CDR1 having the amino acid sequence of SEQ ID NO:73
   LC-CDR2 having the amino acid sequence of SEQ ID NO:26
   LC-CDR3 having the amino acid sequence of SEQ ID NO:74; or
      (j)
(i) a heavy chain variable (VH) region incorporating the following CDRs:
   HC-CDR1 having the amino acid sequence of SEQ ID NO:48
   HC-CDR2 having the amino acid sequence of SEQ ID NO:3
   HC-CDR3 having the amino acid sequence of SEQ ID NO:86; and
(ii) a light chain variable (VL) region incorporating the following CDRs:
   LC-CDR1 having the amino acid sequence of SEQ ID NO:89
   LC-CDR2 having the amino acid sequence of SEQ ID NO:11
   LC-CDR3 having the amino acid sequence of SEQ ID NO:90; or
      (k)
(i) a heavy chain variable (VH) region incorporating the following CDRs:
   HC-CDR1 having the amino acid sequence of SEQ ID NO:61
   HC-CDR2 having the amino acid sequence of SEQ ID NO:95
   HC-CDR3 having the amino acid sequence of SEQ ID NO:96; and
(ii) a light chain variable (VL) region incorporating the following CDRs:
   LC-CDR1 having the amino acid sequence of SEQ ID NO:101
   LC-CDR2 having the amino acid sequence of SEQ ID NO:102
   LC-CDR3 having the amino acid sequence of SEQ ID NO:103; or
      (l)
(i) a heavy chain variable (VH) region incorporating the following CDRs:
   HC-CDR1 having the amino acid sequence of SEQ ID NO:108
   HC-CDR2 having the amino acid sequence of SEQ ID NO:109
   HC-CDR3 having the amino acid sequence of SEQ ID NO:110; and
(ii) a light chain variable (VL) region incorporating the following CDRs:
   LC-CDR1 having the amino acid sequence of SEQ ID NO:115
   LC-CDR2 having the amino acid sequence of SEQ ID NO:116
   LC-CDR3 having the amino acid sequence of SEQ ID NO:117; or
      (m)
(i) a heavy chain variable (VH) region incorporating the following CDRs:
   HC-CDR1 having the amino acid sequence of SEQ ID NO:2
   HC-CDR2 having the amino acid sequence of SEQ ID NO:3
   HC-CDR3 having the amino acid sequence of SEQ ID NO:122; and
(ii) a light chain variable (VL) region incorporating the following CDRs:
   LC-CDR1 having the amino acid sequence of SEQ ID NO:125
   LC-CDR2 having the amino acid sequence of SEQ ID NO:126
   LC-CDR3 having the amino acid sequence of SEQ ID NO:127; or
      (n)
(i) a heavy chain variable (VH) region incorporating the following CDRs:
   HC-CDR1 having the amino acid sequence of SEQ ID NO:132
   HC-CDR2 having the amino acid sequence of SEQ ID NO:133
   HC-CDR3 having the amino acid sequence of SEQ ID NO:134; and
(ii) a light chain variable (VL) region incorporating the following CDRs:
   LC-CDR1 having the amino acid sequence of SEQ ID NO:139
   LC-CDR2 having the amino acid sequence of SEQ ID NO:140
   LC-CDR3 having the amino acid sequence of SEQ ID NO:80; or
      (o)
(i) a heavy chain variable (VH) region incorporating the following CDRs:
   HC-CDR1 having the amino acid sequence of SEQ ID NO:146
   HC-CDR2 having the amino acid sequence of SEQ ID NO:147
   HC-CDR3 having the amino acid sequence of SEQ ID NO:148; and
(ii) a light chain variable (VL) region incorporating the following CDRs:
   LC-CDR1 having the amino acid sequence of SEQ ID NO:41
   LC-CDR2 having the amino acid sequence of SEQ ID NO:42
   LC-CDR3 having the amino acid sequence of SEQ ID NO:153; or
      (p)
(i) a heavy chain variable (VH) region incorporating the following CDRs:
   HC-CDR1 having the amino acid sequence of SEQ ID NO:48
   HC-CDR2 having the amino acid sequence of SEQ ID NO:3
   HC-CDR3 having the amino acid sequence of SEQ ID NO:156; and
(ii) a light chain variable (VL) region incorporating the following CDRs:
   LC-CDR1 having the amino acid sequence of SEQ ID NO:158
   LC-CDR2 having the amino acid sequence of SEQ ID NO:159
   LC-CDR3 having the amino acid sequence of SEQ ID NO:160; or
      (q)
(i) a heavy chain variable (VH) region incorporating the following CDRs:
   HC-CDR1 having the amino acid sequence of SEQ ID NO:166
   HC-CDR2 having the amino acid sequence of SEQ ID NO:167
   HC-CDR3 having the amino acid sequence of SEQ ID NO:168; and
(ii) a light chain variable (VL) region incorporating the following CDRs:
   LC-CDR1 having the amino acid sequence of SEQ ID NO:171
   LC-CDR2 having the amino acid sequence of SEQ ID NO:172
   LC-CDR3 having the amino acid sequence of SEQ ID NO:173; or
      (r)
(i) a heavy chain variable (VH) region incorporating the following CDRs:
   HC-CDR1 having the amino acid sequence of SEQ ID NO:185
   HC-CDR2 having the amino acid sequence of SEQ ID NO:186
   HC-CDR3 having the amino acid sequence of SEQ ID NO:187; and
(ii) a light chain variable (VL) region incorporating the following CDRs:
   LC-CDR1 having the amino acid sequence of SEQ ID NO:73
   LC-CDR2 having the amino acid sequence of SEQ ID NO:26
   LC-CDR3 having the amino acid sequence of SEQ ID NO:194; or
      (s)
(i) a heavy chain variable (VH) region incorporating the following CDRs:
   HC-CDR1 having the amino acid sequence of SEQ ID NO:48
   HC-CDR2 having the amino acid sequence of SEQ ID NO:199
   HC-CDR3 having the amino acid sequence of SEQ ID NO:200; and
(ii) a light chain variable (VL) region incorporating the following CDRs:
   LC-CDR1 having the amino acid sequence of SEQ ID NO:205
   LC-CDR2 having the amino acid sequence of SEQ ID NO:42
   LC-CDR3 having the amino acid sequence of SEQ ID NO:206; or
      (t)
(i) a heavy chain variable (VH) region incorporating the following CDRs:
   HC-CDR1 having the amino acid sequence of SEQ ID NO:48
   HC-CDR2 having the amino acid sequence of SEQ ID NO:211
   HC-CDR3 having the amino acid sequence of SEQ ID NO:212; and
(ii) a light chain variable (VL) region incorporating the following CDRs:
   LC-CDR1 having the amino acid sequence of SEQ ID NO:216
   LC-CDR2 having the amino acid sequence of SEQ ID NO:172
   LC-CDR3 having the amino acid sequence of SEQ ID NO:217; or
      (u)
(i) a heavy chain variable (VH) region incorporating the following CDRs:
   HC-CDR1 having the amino acid sequence of SEQ ID NO:222
   HC-CDR2 having the amino acid sequence of SEQ ID NO:223
   HC-CDR3 having the amino acid sequence of SEQ ID NO:224; and
(ii) a light chain variable (VL) region incorporating the following CDRs:
   LC-CDR1 having the amino acid sequence of SEQ ID NO:229
   LC-CDR2 having the amino acid sequence of SEQ ID NO:172
   LC-CDR3 having the amino acid sequence of SEQ ID NO:230; or
      (v)
(i) a heavy chain variable (VH) region incorporating the following CDRs:
   HC-CDR1 having the amino acid sequence of SEQ ID NO:48
   HC-CDR2 having the amino acid sequence of SEQ ID NO:199
   HC-CDR3 having the amino acid sequence of SEQ ID NO:200; and
(ii) a light chain variable (VL) region incorporating the following CDRs:
   LC-CDR1 having the amino acid sequence of SEQ ID NO:235
   LC-CDR2 having the amino acid sequence of SEQ ID NO:236
   LC-CDR3 having the amino acid sequence of SEQ ID NO:237; or
      (w)
(i) a heavy chain variable (VH) region incorporating the following CDRs:
   HC-CDR1 having the amino acid sequence of SEQ ID NO:185
   HC-CDR2 having the amino acid sequence of SEQ ID NO:243
   HC-CDR3 having the amino acid sequence of SEQ ID NO:244; and
(ii) a light chain variable (VL) region incorporating the following CDRs:
   LC-CDR1 having the amino acid sequence of SEQ ID NO:248
   LC-CDR2 having the amino acid sequence of SEQ ID NO:249
   LC-CDR3 having the amino acid sequence of SEQ ID NO:250.

In some embodiments, the antigen-binding molecule comprises:
a VH region comprising an amino acid sequence having at least 70% sequence identity to the amino acid sequence of SEQ ID NO:165, 32, 1, 17, 47, 60, 82, 85, 94, 107, 121, 131, 154, 155, 184, 198, 210, 221 or 242; and
a VL region comprising an amino acid sequence having at least 70% sequence identity to the amino acid sequence of SEQ ID NO:178, 40, 9, 24, 52, 67, 72, 77, 88, 100, 114, 124, 138, 152, 157, 170, 191, 204, 215, 228, 234 or 247.

In some embodiments, the antigen-binding molecule comprises:
(i) a VH region comprising an amino acid sequence having at least 70% sequence identity to the amino acid sequence of SEQ ID NO:165; and
   a VL region comprising an amino acid sequence having at least 70% sequence identity to the amino acid sequence of SEQ ID NO:178;
      or
(ii) a VH region comprising an amino acid sequence having at least 70% sequence identity to the amino acid sequence of SEQ ID NO:32; and
   a VL region comprising an amino acid sequence having at least 70% sequence identity to the amino acid sequence of SEQ ID NO:40;
      or
(iii) a VH region comprising an amino acid sequence having at least 70% sequence identity to the amino acid sequence of SEQ ID NO:1; and
   a VL region comprising an amino acid sequence having at least 70% sequence identity to the amino acid sequence of SEQ ID NO:9;
      or
(iv) a VH region comprising an amino acid sequence having at least 70% sequence identity to the amino acid sequence of SEQ ID NO:17; and
   a VL region comprising an amino acid sequence having at least 70% sequence identity to the amino acid sequence of SEQ ID NO:24;
      or
(v) a VH region comprising an amino acid sequence having at least 70% sequence identity to the amino acid sequence of SEQ ID NO:47; and
   a VL region comprising an amino acid sequence having at least 70% sequence identity to the amino acid sequence of SEQ ID NO:52;
      or
(vi) a VH region comprising an amino acid sequence having at least 70% sequence identity to the amino acid sequence of SEQ ID NO:60; and
   a VL region comprising an amino acid sequence having at least 70% sequence identity to the amino acid sequence of SEQ ID NO:67;
      or
(vii) a VH region comprising an amino acid sequence having at least 70% sequence identity to the amino acid sequence of SEQ ID NO:60; and
   a VL region comprising an amino acid sequence having at least 70% sequence identity to the amino acid sequence of SEQ ID NO:72;
      or
(viii) a VH region comprising an amino acid sequence having at least 70% sequence identity to the amino acid sequence of SEQ ID NO:60; and
   a VL region comprising an amino acid sequence having at least 70% sequence identity to the amino acid sequence of SEQ ID NO:77;
      or
(ix) a VH region comprising an amino acid sequence having at least 70% sequence identity to the amino acid sequence of SEQ ID NO:; and
   a VL region comprising an amino acid sequence having at least 70% sequence identity to the amino acid sequence of SEQ ID NO:;
      or
(x) a VH region comprising an amino acid sequence having at least 70% sequence identity to the amino acid sequence of SEQ ID NO:82; and
   a VL region comprising an amino acid sequence having at least 70% sequence identity to the amino acid sequence of SEQ ID NO:72;
      or
(xi) a VH region comprising an amino acid sequence having at least 70% sequence identity to the amino acid sequence of SEQ ID NO:85; and
   a VL region comprising an amino acid sequence having at least 70% sequence identity to the amino acid sequence of SEQ ID NO:88;
      or
(xii) a VH region comprising an amino acid sequence having at least 70% sequence identity to the amino acid sequence of SEQ ID NO:94; and
   a VL region comprising an amino acid sequence having at least 70% sequence identity to the amino acid sequence of SEQ ID NO:100;
      or
(xiii) a VH region comprising an amino acid sequence having at least 70% sequence identity to the amino acid sequence of SEQ ID NO:107; and
   a VL region comprising an amino acid sequence having at least 70% sequence identity to the amino acid sequence of SEQ ID NO:114;
      or
(xiv) a VH region comprising an amino acid sequence having at least 70% sequence identity to the amino acid sequence of SEQ ID NO:121; and
   a VL region comprising an amino acid sequence having at least 70% sequence identity to the amino acid sequence of SEQ ID NO:124;
      or
(xv) a VH region comprising an amino acid sequence having at least 70% sequence identity to the amino acid sequence of SEQ ID NO:131; and
   a VL region comprising an amino acid sequence having at least 70% sequence identity to the amino acid sequence of SEQ ID NO:138;
      or
(xvi) a VH region comprising an amino acid sequence having at least 70% sequence identity to the amino acid sequence of SEQ ID NO:145; and
   a VL region comprising an amino acid sequence having at least 70% sequence identity to the amino acid sequence of SEQ ID NO:152;
      or
(xvii) a VH region comprising an amino acid sequence having at least 70% sequence identity to the amino acid sequence of SEQ ID NO:155; and
   a VL region comprising an amino acid sequence having at least 70% sequence identity to the amino acid sequence of SEQ ID NO:157;
      or
(xviii) a VH region comprising an amino acid sequence having at least 70% sequence identity to the amino acid sequence of SEQ ID NO:165; and
   a VL region comprising an amino acid sequence having at least 70% sequence identity to the amino acid sequence of SEQ ID NO:170;
      or
(xix) a VH region comprising an amino acid sequence having at least 70% sequence identity to the amino acid sequence of SEQ ID NO:184; and
   a VL region comprising an amino acid sequence having at least 70% sequence identity to the amino acid sequence of SEQ ID NO:191;
      or
(xx) a VH region comprising an amino acid sequence having at least 70% sequence identity to the amino acid sequence of SEQ ID NO:198; and
   a VL region comprising an amino acid sequence having at least 70% sequence identity to the amino acid sequence of SEQ ID NO:204;
      or
(xxi) a VH region comprising an amino acid sequence having at least 70% sequence identity to the amino acid sequence of SEQ ID NO:210; and
   a VL region comprising an amino acid sequence having at least 70% sequence identity to the amino acid sequence of SEQ ID NO:215;
      or
(xxii) a VH region comprising an amino acid sequence having at least 70% sequence identity to the amino acid sequence of SEQ ID NO:221; and
   a VL region comprising an amino acid sequence having at least 70% sequence identity to the amino acid sequence of SEQ ID NO:228;
      or
(xxiii) a VH region comprising an amino acid sequence having at least 70% sequence identity to the amino acid sequence of SEQ ID NO:198; and
   a VL region comprising an amino acid sequence having at least 70% sequence identity to the amino acid sequence of SEQ ID NO:234;
      or
(xxiv) a VH region comprising an amino acid sequence having at least 70% sequence identity to the amino acid sequence of SEQ ID NO:242; and
   a VL region comprising an amino acid sequence having at least 70% sequence identity to the amino acid sequence of SEQ ID NO:247.

In some embodiments, the antigen-binding molecule binds to CNX via contact with: (a) one or more amino acid residues of the region of CNX corresponding to the region shown in SEQ ID NO:363, optionally wherein the antigen-binding molecule binds to CNX via contact with one or more amino acid residues of the region of CNX corresponding to the region shown in SEQ ID NO:361 or 362; or (b) one or more amino acid residues of the region of CNX corresponding to the region shown in SEQ ID NO:371, optionally wherein the antigen-binding molecule binds to CNX via contact with one or more amino acid residues of the region of CNX corresponding to the region shown in SEQ ID NO:364, 365, 366, 367, 368, 369, 370, 372, or 373.

In some embodiments, the antigen-binding molecule binds to CRT.

In some embodiments, the antigen-binding molecule binds to human CNX and mouse CNX.

In some embodiments, the antigen-binding molecule is a multispecific antigen-binding molecule, and wherein the antigen-binding molecule further comprises an antigen-binding domain which binds to an antigen other than CNX. In some embodiments, the multispecific antigen-binding molecule is a bispecific T cell engager (BiTE).

The present disclosure also provides a chimeric antigen receptor (CAR) comprising an antigen-binding molecule according to the present disclosure.

The present disclosure also provides an antibody-drug conjugate (ADC) comprising an antigen-binding molecule according to the present disclosure and a drug moiety.

The present disclosure also provides a nucleic acid, or a plurality of nucleic acids, optionally isolated, encoding an antigen-binding molecule or CAR according to the present disclosure.

The present disclosure also provides an expression vector, or a plurality of expression vectors, comprising a nucleic acid or a plurality of nucleic acids according to the present disclosure.

The present disclosure also provides a cell comprising an antigen-binding molecule, CAR, nucleic acid or plurality of nucleic acids, expression vector or a plurality of expression vectors according to the present disclosure.

The present disclosure also provides a method comprising culturing a cell according to the present disclosure under conditions suitable for expression of an antigen-binding molecule or CAR by the cell.

The present disclosure also provides a composition comprising an antigen-binding molecule, CAR, nucleic acid or plurality of nucleic acids, expression vector or a plurality of expression vectors, or cell according to the present disclosure, and a pharmaceutically acceptable carrier, diluent, excipient or adjuvant.

The present disclosure also provides an antigen-binding molecule, CAR, nucleic acid or plurality of nucleic acids, expression vector or a plurality of expression vectors, cell or composition according to the present disclosure, for use in a method of medical treatment or prophylaxis.

The present disclosure also provides an antigen-binding molecule, CAR, nucleic acid or plurality of nucleic acids, expression vector or a plurality of expression vectors, cell or composition according to the present disclosure, for use in a method of treatment or prevention of a disease/condition characterised by extracellular matrix (ECM) degradation.

The present disclosure also provides an antigen-binding molecule, CAR, nucleic acid or plurality of nucleic acids, expression vector or a plurality of expression vectors, cell or composition according to the present disclosure, for use in a method of treatment or prevention of a cancer.

In some embodiments, the cancer is selected from: liver cancer, breast cancer, oral cancer, oral squamous cell carcinoma, sarcoma, lung cancer, prostate cancer, bladder cancer, renal cancer, melanoma, pancreatic cancer, endometrial cancer, colorectal cancer, and thyroid cancer.

The present disclosure also provides an antigen-binding molecule, CAR, nucleic acid or plurality of nucleic acids, expression vector or a plurality of expression vectors, cell or composition according to the present disclosure, for use in a method of treatment or prevention of cartilage degradation, or a disease/condition characterised by cartilage degradation.

In some embodiments, the disease/condition characterised by cartilage degradation is selected from: a joint disorder, arthritis, osteoarthritis, psoriasis arthritis, rheumatoid arthritis, juvenile arthritis, post-traumatic arthritis, gout, chondrocalcinosis, fibromyalgia, costochondritis, osteochondritis dissecans, cartilage damage and polychondritis.

The present disclosure also provides the use of antigen-binding molecule according to the present disclosure to deplete or increase killing of cells expressing CNX.

The present disclosure also provides an in vitro complex, optionally isolated, comprising an antigen-binding molecule according to the present disclosure bound to CNX.

The present disclosure also provides a method for detecting CNX in a sample, comprising contacting a sample containing, or suspected to contain, CNX with an antigen-binding molecule according to the present disclosure, and detecting the formation of a complex of the antigen-binding molecule with CNX.

The present disclosure also provides method of selecting or stratifying a subject for treatment with a CNX-targeted agent, the method comprising contacting, in vitro, a sample from the subject with an antigen-binding molecule according to the present disclosure and detecting the formation of a complex of the antigen-binding molecule with CNX.

The present disclosure also provides the use of an antigen-binding molecule according to the present disclosure as an in vitro or in vivo diagnostic or prognostic agent.

### DESCRIPTION

The present disclosure provides antigen-binding molecules that bind to CNX, having novel biophysical and/or functional properties as compared to antigen-binding molecules disclosed in the prior art.

### CNX and CRT

The present disclosure relates to CNX-specific antigen-binding molecules.

Human CNX (also known as CNX, CANX or IP90) is the protein identified by UniProt P27824. Alternative splicing of mRNA encoded by the human CANX gene yields three main CNX isoforms: isoform 1 (SEQ ID NO:333), isoform 2 (SEQ ID NO:334) and isoform 3 (SEQ ID NO:335). Isoform 2 differs from isoform 1 by insertion of a 35 amino acid sequence after position 1 of SEQ ID NO:333. Positions 1 to 108 of SEQ ID NO:333 are absent from isoform 3.

Human CNX isoform 1 comprises an N-terminal signal peptide (SEQ ID NO:336), followed by a calcium-binding lumenal domain (SEQ ID NO:337), a single-pass transmembrane domain (SEQ ID NO:338) and an acidic cytoplasmic domain (SEQ ID NO:339) at the C-terminus. The lumenal domain comprises a globular lectin domain (SEQ ID NO:340), followed by an arm-like, proline-rich P-domain (SEQ ID NO:341) and a second lectin domain (SEQ ID NO:342). The mature form of human CNX isoform 1 is shown in SEQ ID NO:343.

In this specification ‘CNX’ refers to CNX from any species, and includes isoforms, fragments, variants or homologues from any species. In some embodiments CNX is CNX from a mammal (e.g. a therian, placental, epitherian, preptotheria, archontan, primate (rhesus, cynomolgous, non-human primate or human)). In some embodiments, the CNX is human CNX or mouse CNX.

As used herein, a ‘fragment’, ‘variant’, ‘isoform’ or ‘homologue’ of a given protein may optionally be characterised as having at least 60%, preferably one of 70%, 75%, 80%, 85%, 90%, 91%, 92%, 93%, 94%, 95%, 96%, 97%, 98%, 99% or greater amino acid sequence identity to the amino acid sequence of the reference protein (e.g. a reference isoform).

A ‘fragment’ generally refers to a fraction of the reference protein. A ‘variant’ generally refers to a protein having an amino acid sequence comprising one or more amino acid substitutions, insertions, deletions or other modifications relative to the amino acid sequence of the reference protein, but retaining a considerable degree of sequence identity (e.g. at least 60%) to the amino acid sequence of the reference protein. An ‘isoform’ generally refers to a variant of the reference protein expressed by the same species as the species of the reference protein (e.g. human CNX isoform 1, isoform 2 and isoform 3 are all isoforms of one another). A ‘homologue’ generally refers to a variant of the reference protein produced by a different species as compared to the species of the reference protein. For example, human CNX isoform 1 (UniProt: P27824-1, v2; SEQ ID NO:333) and mouse CNX (UniProt: P35564-1, v1; SEQ ID NO:344) are homologues of one another. Homologues include orthologues.

Isoforms, fragments, variants or homologues of CNX according to the present disclosure may optionally be characterised as having at least 70%, preferably one of 80%, 85%, 90%, 91%, 92%, 93%, 94%, 95%, 96%, 97%, 98%, 99% or 100% amino acid sequence identity to the amino acid sequence of an immature or mature CNX isoform from a given species, e.g. human.

Isoforms, fragments, variants or homologues may optionally be functional isoforms, fragments, variants or homologues, e.g. having a functional property/activity of the reference CNX (e.g. human CNX isoform 1), as determined by analysis by a suitable assay for the functional property/activity. For example, an isoform, fragment, variant or homologue of CNX may display binding to a monoglucosylated glycan-bearing N-glycoprotein, and/or association with ERp57, cyclophilin B and/or ERp29.

In some embodiments, the CNX comprises, or consists of, an amino acid sequence having at least 70%, preferably one of 80%, 85%, 90%, 91%, 92%, 93%, 94%, 95%, 96%, 97%, 98%, 99% or 100% amino acid sequence identity to SEQ ID NO:333, 334, 335 or 343.

In some embodiments, the CNX comprises, or consists of, an amino acid sequence having at least 70%, preferably one of 80%, 85%, 90%, 91%, 92%, 93%, 94%, 95%, 96%, 97%, 98%, 99% or 100% amino acid sequence identity to SEQ ID NO:344 or 352.

A ‘fragment’ of a reference protein may be of any length (by number of amino acids), although may optionally be at least 25% of the length of the reference protein (that is, the protein from which the fragment is derived) and may have a maximum length of one of 50%, 75%, 80%, 85%, 90%, 91%, 92%, 93%, 94%, 95%, 96%, 97%, 98%, or 99% of the length of the reference protein.

A fragment of CNX may have a minimum length of one of 10, 20, 30, 40, 50, 100, 150, 200, 250, 300, 350, 400, 450, 500, 550 or 600 amino acids, and may have a maximum length of one of 20, 30, 40, 50, 100, 150, 200, 250, 300, 350, 400, 450, 500, 550 or 600 amino acids.

In some embodiments, a fragment of CNX comprises, or consists of, an amino acid sequence having at least 70%, preferably one of 80%, 85%, 90%, 91%, 92%, 93%, 94%, 95%, 96%, 97%, 98%, 99% or 100% amino acid sequence identity to SEQ ID NO:343, 337, 338, 339, 340, 341 or 342.

In some embodiments, a fragment of CNX comprises, or consists of, an amino acid sequence having at least 70%, preferably one of 80%, 85%, 90%, 91%, 92%, 93%, 94%, 95%, 96%, 97%, 98%, 99% or 100% amino acid sequence identity to SEQ ID NO:352, 346, 347, 348, 349, 350 or 351.

In some embodiments, the antigen-binding molecules of the present disclosure display binding to Calreticulin (CRT).

Inventors observed that CNX-depleted cells can compensate for CNX loss through the action of CRT. Therefore, the inventors identified antigen-binding molecules capable of binding both CNX and CRT.

In some embodiments, the antigen-binding molecule is cross-reactive for human CNX and CRT. In some embodiments, the antigen-binding molecule reduces an activity of CNX and an activity of CRT. In some embodiments, the antigen-binding molecule reduces CNX activity and CRT activity.

As used herein, a ‘cross-reactive’ antigen-binding molecule/domain binds to the target antigens for which the antigen-binding molecule/domain is cross-reactive. For example, an antigen-binding molecule/domain/polypeptide which is cross-reactive for CNX and CRT binds to CNX and is also capable of binding to CRT. Cross-reactive antigen-binding molecules/domains/polypeptides may display specific binding to each of the target antigens.

Human CRT (also known as calreticulin, calregulin or ERp60) is the protein identified by UniProt P27797. Human CRT has the amino acid sequence shown in SEQ ID NO:353. Human CRT comprises an N-terminal signal peptide (SEQ ID NO:354), followed by a calcium-binding N-domain (SEQ ID NO:355), and an acidic C-domain (SEQ ID NO:356) at the C-terminus. The N-domain comprises a globular lectin domain (SEQ ID NO:357), followed by an arm-like, proline-rich P-domain (SEQ ID NO:359) and a second lectin domain (SEQ ID NO:358). The mature form of human CRT is shown in SEQ ID NO:360.

In this specification ‘CRT’ refers to CRT from any species, and includes isoforms, fragments, variants or homologues from any species. In some embodiments CRT is CRT from a mammal (e.g. a therian, placental, epitherian, preptotheria, archontan, primate (rhesus, cynomolgous, non-human primate or human)). In some embodiments, the CRT is human CRT or mouse CRT.

Isoforms, fragments, variants or homologues of CRT according to the present disclosure may optionally be characterised as having at least 70%, preferably one of 80%, 85%, 90%, 91%, 92%, 93%, 94%, 95%, 96%, 97%, 98%, 99% or 100% amino acid sequence identity to the amino acid sequence of an immature or mature CRT isoform from a given species, e.g. human.

Isoforms, fragments, variants or homologues of CRT may optionally be functional isoforms, fragments, variants or homologues, e.g. having a functional property/activity of the reference CRT (e.g. human CRT), as determined by analysis by a suitable assay for the functional property/activity. For example, an isoform, fragment, variant or homologue of CRT may display binding to a monoglucosylated glycan-bearing N-glycoprotein, and/or association with ERp57, cyclophilin B and/or ERp29.

In some embodiments, the CRT comprises, or consists of, an amino acid sequence having at least 70%, preferably one of 80%, 85%, 90%, 91%, 92%, 93%, 94%, 95%, 96%, 97%, 98%, 99% or 100% amino acid sequence identity to SEQ ID NO:353 or 360.

A fragment of CRT may have a minimum length of one of 10, 20, 30, 40, 50, 100, 150, 200, 250, 300, 350, or 400 amino acids, and may have a maximum length of one of 20, 30, 40, 50, 100, 150, 200, 250, 300, 350, or 400 amino acids.

In some embodiments, a fragment of CRT comprises, or consists of, an amino acid sequence having at least 70%, preferably one of 80%, 85%, 90%, 91%, 92%, 93%, 94%, 95%, 96%, 97%, 98%, 99% or 100% amino acid sequence identity to SEQ ID NO:360, 355, 356, 357, 358 or 359.

The structure and function of CNX and CRT is reviewed e.g. in Kozlov and Gehring, FEBS J. (2020) 287(20):4322-4340, which is hereby incorporated by reference in its entirety.

CNX and CRT are endoplasmic reticulum (ER)-resident lectin chaperone proteins. CNX/CRT bind N-glycoproteins bearing monoglucosylated glycans, and recruit various other chaperones which mediate protein disulfide formation, proline isomerisation, and protein folding. CNX/CRT are able to associate with the protein folding enzyme ERp57 to catalyse glycoprotein-specific disulfide bond formation. CNX:ERp57 complexes have also been shown to translocate to the surface of cancer cells, where they reduce disulfide bridges in the extracellular matrix (Ros et al., Nat. Cell Biol. 22, 1371-1381, 2020). The reduction of disulfide bridges has been shown to be essential for the effective activity of matrix metalloproteinases (MMPs), and thus for the degradation of the extracellular matrix in cancer. CNX/CRT also associate with the peptidyl-prolyl cis-trans isomerase cyclophilin B (CypB), for the proline isomerisation of peptide bonds. CNX/CRT have also been reported to associate with ERp29 to form CNX/CRT:ERp29 complexes, which have a general chaperone function. CNX also functions as a chaperone for the folding of MHC class I α-chain in the membrane of the ER.

Processing by glucosidase II removes the glucose residue of the monoglucosylated N-glycan required for interaction of the glycoprotein with CNX/CRT, resulting in liberation of the mature, processed glycoprotein from CNX/CRT. For proteins that have not yet folded properly, UDP-glucose:glycoprotein glucosyltransferase (UGGT) acts as a checkpoint by re-adding a glucose residue back onto the N-glycan, reconstituting the interaction site for CNX/CRT. In this way, misfolded proteins re-associate with CNX/CRT for additional rounds of chaperone-mediated refolding, and their exit from the ER and progression to the Golgi is prevented. If multiple folding cycles are unsuccessful, terminally misfolded proteins are transported to the cytoplasm for degradation via the ER-associated protein degradation (ERAD) pathway.

### Antigen-Binding Molecules

The present disclosure provides antigen-binding molecules capable of binding to CNX. An antigen-binding molecule that is capable of binding to a given target antigen may also be described as an antigen-binding molecule that binds to the given target antigen.

An ‘antigen-binding molecule’ refers to a molecule that binds to a given target antigen. Antigen-binding molecules include antibodies (i.e. immunoglobulins (Igs)) and antigen-binding fragments thereof. As used herein, ‘antibodies’ include monoclonal antibodies, polyclonal antibodies, monospecific and multispecific (e.g., bispecific, trispecific, etc.) antibodies, and antibody-derived antigen-binding molecules such as scFv, scFab, diabodies, triabodies, scFv-Fc, minibodies, single domain antibodies (e.g. VhH), etc.). Antigen-binding fragments of antibodies include e.g. Fv, Fab, F(ab′)₂ and F(ab′) fragments. In some embodiments, an antigen-binding molecule may be an antibody or an antigen-binding fragment thereof.

Antigen-binding molecules according to the present disclosure also include antibody-derived molecules, e.g. molecules comprising an antigen-binding region/domain derived from an antibody. Antibody-derived antigen-binding molecules may comprise an antigen-binding region/domain that comprises, or consists of, the antigen-binding region of an antibody (e.g. an antigen-binding fragment of an antibody). In some embodiments, the antigen-binding region/domain of an antibody-derived antigen-binding molecule may be or comprise the Fv (e.g. provided as an scFv) or the Fab region of an antibody, or the whole antibody. For example, antigen-binding molecules according to the present disclosure include antibody-drug conjugates (ADCs) comprising a (cytotoxic) drug moiety (e.g. as described hereinbelow). Antigen-binding molecules according to the present disclosure also include multispecific antigen-binding molecules such as immune cell engager molecules comprising a domain for recruiting (effector) immune cells (reviewed e.g. in Goebeler and Bargou, Nat. Rev. Clin. Oncol. (2020) 17: 418-434 and Ellerman, Methods (2019) 154:102-117, both of which are hereby incorporated by reference in their entirety), including BiTEs, BiKEs and TriKEs. Antigen-binding molecules according to the present disclosure also include chimeric antigen receptors (CARs), which are recombinant receptors providing both antigen-binding and T cell activating functions (CAR structure, function and engineering is reviewed e.g. in Dotti et al., Immunol Rev (2014) 257(1), which is hereby incorporated by reference in its entirety).

The antigen-binding molecule of the present disclosure comprises a moiety or moieties capable of binding to a target antigen(s). In some embodiments, the moiety capable of binding to a target antigen comprises an antibody heavy chain variable region (VH) and an antibody light chain variable region (VL) of an antibody capable of specific binding to the target antigen. In some embodiments, the moiety capable of binding to a target antigen comprises or consists of an aptamer capable of binding to the target antigen, e.g. a nucleic acid aptamer (reviewed, for example, in Zhou and Rossi Nat Rev Drug Discov. 2017 16(3):181-202). In some embodiments, the moiety capable of binding to a target antigen comprises or consists of a antigen-binding peptide/polypeptide, e.g. a peptide aptamer, thioredoxin, monobody, anticalin, Kunitz domain, avimer, knottin, fynomer, atrimer, DARPin, affibody, nanobody (i.e. a single-domain antibody (sdAb)), affilin, armadillo repeat protein (ArmRP), OBody or fibronectin—reviewed e.g. in Reverdatto et al., Curr Top Med Chem. 2015; 15(12): 1082-1101, which is hereby incorporated by reference in its entirety (see also e.g. Boersma et al., J Biol Chem (2011) 286:41273-85 and Emanuel et al., Mabs (2011) 3:38-48).

As used herein, a ‘peptide’ refers to a chain of two or more amino acid monomers linked by peptide bonds. A peptide typically has a length in the region of about 2 to 50 amino acids. A ‘polypeptide’ is a polymer chain of two or more peptides. Polypeptides typically have a length greater than about 50 amino acids.

The antigen-binding molecules of the present disclosure generally comprise an antigen-binding domain comprising a VH and a VL of an antibody capable of specific binding to the target antigen. The antigen-binding domain formed by a VH and a VL may also be referred to herein as an Fv region.

An antigen-binding molecule may be, or may comprise, an antigen-binding polypeptide, or an antigen-binding polypeptide complex. An antigen-binding molecule may comprise more than one polypeptide which together form an antigen-binding domain. The polypeptides may associate covalently or non-covalently. In some embodiments, the polypeptides form part of a larger polypeptide comprising the polypeptides (e.g. in the case of scFv comprising VH and VL, or in the case of scFab comprising VH-CH1 and VL-CL).

An antigen-binding molecule may refer to a non-covalent or covalent complex of more than one polypeptide (e.g. 2, 3, 4, 6, or 8 polypeptides), e.g. an IgG-like antigen-binding molecule comprising two heavy chain polypeptides and two light chain polypeptides.

The antigen-binding molecules of the present disclosure may be designed and prepared using the sequences of monoclonal antibodies (mAbs) capable of binding to CNX. Antigen-binding regions of antibodies, such as single chain variable fragment (scFv), Fab and F(ab′)₂ fragments may also be used/provided. An ‘antigen-binding region’ is any fragment of an antibody that binds to the target for which the given antibody is specific.

Antibodies generally comprise six complementarity-determining regions CDRs; three in the heavy chain variable (VH) region: HC-CDR1, HC-CDR2 and HC-CDR3, and three in the light chain variable (VL) region: LC-CDR1, LC-CDR2, and LC-CDR3. The six CDRs together define the paratope of the antibody, which is the part of the antibody that binds to the target antigen.

The VH region and VL region comprise framework regions (FRs) either side of each CDR, which provide a scaffold for the CDRs. From N-terminus to C-terminus, VH regions comprise the following structure: N term-[HC-FR1]-[HC-CDR1]-[HC-FR2]-[HC-CDR2]-[HC-FR3]-[HC-CDR3]-[HC-FR4]-C term; and VL regions comprise the following structure: N term-[LC-FR1]-[LC-CDR1]-[LC-FR2]-[LC-CDR2]-[LC-FR3]-[LC-CDR3]-[LC-FR4]-C term.

There are several different conventions for defining antibody CDRs and FRs, such as those described in Kabat et al., Sequences of Proteins of Immunological Interest, 5th Ed. Public Health Service, National Institutes of Health, Bethesda, MD (1991), Chothia et al., J. Mol. Biol. 196:901-917 (1987), and VBASE2, as described in Retter et al., Nucl. Acids Res. (2005) 33 (suppl 1): D671-D674. The CDRs and FRs of the VH regions and VL regions of the antibody clones described herein were defined according to the international IMGT (ImMunoGeneTics) information system (LeFranc et al., Nucleic Acids Res. (2015) 43 (Database issue):D413-22), which uses the IMGT V-DOMAIN numbering rules as described in Lefranc et al., Dev. Comp. Immunol. (2003) 27:55-77. In preferred embodiments, the CDRs and FRs of antigen-binding molecules referred to herein are defined according to the IMGT information system.

In some embodiments, the antigen-binding molecule comprises the CDRs of an antigen-binding molecule that binds to CNX. In some embodiments, the antigen-binding molecule comprises the FRs of an antigen-binding molecule that binds to CNX. In some embodiments, the antigen-binding molecule comprises the CDRs and the FRs of an antigen-binding molecule that binds to CNX. That is, In some embodiments, the antigen-binding molecule comprises the VH region and the VL region of an antigen-binding molecule that binds to CNX.

In some embodiments, the antigen-binding molecule comprises the CDRs, FRs and/or the VH and/or VL regions of a CNX-binding antibody clone described herein, or CDRs, FRs and/or VH and/or VL regions which are derived from those of a CNX-binding antibody clone described herein. In some embodiments, a CNX-binding antibody clone is selected from: 1D3, 1D6, 1E1, 1E6, 2C6, 2H6, 3D1, 2G9, 2G12, 2H5, 3F8, 3F9, 4G9, 5A3, 5E8, 0001, C008, C010, C023, C025, C040, C046 and C117.

In some embodiments, the antigen-binding molecule comprises a VH region according to one of (1) to (19) below:
(1) a VH region incorporating the following CDRs:
   HC-CDR1 having the amino acid sequence of SEQ ID NO:2
   HC-CDR2 having the amino acid sequence of SEQ ID NO:3
   HC-CDR3 having the amino acid sequence of SEQ ID NO:4,
   or a variant thereof in which 1 or 2 or 3 amino acids in HC-CDR1, and/or in which 1 or 2 or 3 amino acids in HC-CDR2, and/or in which 1 or 2 or 3 amino acids in HC-CDR3 are substituted with another amino acid.
(2) a VH region incorporating the following CDRs:
   HC-CDR1 having the amino acid sequence of SEQ ID NO:18
   HC-CDR2 having the amino acid sequence of SEQ ID NO:19
   HC-CDR3 having the amino acid sequence of SEQ ID NO:20,
   or a variant thereof in which 1 or 2 or 3 amino acids in HC-CDR1, and/or in which 1 or 2 or 3 amino acids in HC-CDR2, and/or in which 1 or 2 or 3 amino acids in HC-CDR3 are substituted with another amino acid.
(3) a VH region incorporating the following CDRs:
   HC-CDR1 having the amino acid sequence of SEQ ID NO:33
   HC-CDR2 having the amino acid sequence of SEQ ID NO:34
   HC-CDR3 having the amino acid sequence of SEQ ID NO:35,
   or a variant thereof in which 1 or 2 or 3 amino acids in HC-CDR1, and/or in which 1 or 2 or 3 amino acids in HC-CDR2, and/or in which 1 or 2 or 3 amino acids in HC-CDR3 are substituted with another amino acid.
(4) a VH region incorporating the following CDRs:
   HC-CDR1 having the amino acid sequence of SEQ ID NO:48
   HC-CDR2 having the amino acid sequence of SEQ ID NO:3
   HC-CDR3 having the amino acid sequence of SEQ ID NO:49,
   or a variant thereof in which 1 or 2 or 3 amino acids in HC-CDR1, and/or in which 1 or 2 or 3 amino acids in HC-CDR2, and/or in which 1 or 2 or 3 amino acids in HC-CDR3 are substituted with another amino acid.
(5) a VH region incorporating the following CDRs:
   HC-CDR1 having the amino acid sequence of SEQ ID NO:61
   HC-CDR2 having the amino acid sequence of SEQ ID NO:62
   HC-CDR3 having the amino acid sequence of SEQ ID NO:63,
   or a variant thereof in which 1 or 2 or 3 amino acids in HC-CDR1, and/or in which 1 or 2 or 3 amino acids in HC-CDR2, and/or in which 1 or 2 or 3 amino acids in HC-CDR3 are substituted with another amino acid.
(6) a VH region incorporating the following CDRs:
   HC-CDR1 having the amino acid sequence of SEQ ID NO:2
   HC-CDR2 having the amino acid sequence of SEQ ID NO:3
   HC-CDR3 having the amino acid sequence of SEQ ID NO:83,
   or a variant thereof in which 1 or 2 or 3 amino acids in HC-CDR1, and/or in which 1 or 2 or 3 amino acids in HC-CDR2, and/or in which 1 or 2 or 3 amino acids in HC-CDR3 are substituted with another amino acid.
(6) a VH region incorporating the following CDRs:
   HC-CDR1 having the amino acid sequence of SEQ ID NO:2
   HC-CDR2 having the amino acid sequence of SEQ ID NO:3
   HC-CDR3 having the amino acid sequence of SEQ ID NO:83,
   or a variant thereof in which 1 or 2 or 3 amino acids in HC-CDR1, and/or in which 1 or 2 or 3 amino acids in HC-CDR2, and/or in which 1 or 2 or 3 amino acids in HC-CDR3 are substituted with another amino acid.
(7) a VH region incorporating the following CDRs:
   HC-CDR1 having the amino acid sequence of SEQ ID NO:48
   HC-CDR2 having the amino acid sequence of SEQ ID NO:3
   HC-CDR3 having the amino acid sequence of SEQ ID NO:86,
   or a variant thereof in which 1 or 2 or 3 amino acids in HC-CDR1, and/or in which 1 or 2 or 3 amino acids in HC-CDR2, and/or in which 1 or 2 or 3 amino acids in HC-CDR3 are substituted with another amino acid.
(8) a VH region incorporating the following CDRs:
   HC-CDR1 having the amino acid sequence of SEQ ID NO:61
   HC-CDR2 having the amino acid sequence of SEQ ID NO:95
   HC-CDR3 having the amino acid sequence of SEQ ID NO:96,
   or a variant thereof in which 1 or 2 or 3 amino acids in HC-CDR1, and/or in which 1 or 2 or 3 amino acids in HC-CDR2, and/or in which 1 or 2 or 3 amino acids in HC-CDR3 are substituted with another amino acid.
(9) a VH region incorporating the following CDRs:
   HC-CDR1 having the amino acid sequence of SEQ ID NO:108
   HC-CDR2 having the amino acid sequence of SEQ ID NO:109
   HC-CDR3 having the amino acid sequence of SEQ ID NO:110,
   or a variant thereof in which 1 or 2 or 3 amino acids in HC-CDR1, and/or in which 1 or 2 or 3 amino acids in HC-CDR2, and/or in which 1 or 2 or 3 amino acids in HC-CDR3 are substituted with another amino acid.
(10) a VH region incorporating the following CDRs:
   HC-CDR1 having the amino acid sequence of SEQ ID NO:2
   HC-CDR2 having the amino acid sequence of SEQ ID NO:3
   HC-CDR3 having the amino acid sequence of SEQ ID NO:122,
   or a variant thereof in which 1 or 2 or 3 amino acids in HC-CDR1, and/or in which 1 or 2 or 3 amino acids in HC-CDR2, and/or in which 1 or 2 or 3 amino acids in HC-CDR3 are substituted with another amino acid.
(11) a VH region incorporating the following CDRs:
   HC-CDR1 having the amino acid sequence of SEQ ID NO:132
   HC-CDR2 having the amino acid sequence of SEQ ID NO:133
   HC-CDR3 having the amino acid sequence of SEQ ID NO:134,
   or a variant thereof in which 1 or 2 or 3 amino acids in HC-CDR1, and/or in which 1 or 2 or 3 amino acids in HC-CDR2, and/or in which 1 or 2 or 3 amino acids in HC-CDR3 are substituted with another amino acid.
(12) a VH region incorporating the following CDRs:
   HC-CDR1 having the amino acid sequence of SEQ ID NO:146
   HC-CDR2 having the amino acid sequence of SEQ ID NO:147
   HC-CDR3 having the amino acid sequence of SEQ ID NO:148,
   or a variant thereof in which 1 or 2 or 3 amino acids in HC-CDR1, and/or in which 1 or 2 or 3 amino acids in HC-CDR2, and/or in which 1 or 2 or 3 amino acids in HC-CDR3 are substituted with another amino acid.
(13) a VH region incorporating the following CDRs:
   HC-CDR1 having the amino acid sequence of SEQ ID NO:48
   HC-CDR2 having the amino acid sequence of SEQ ID NO:3
   HC-CDR3 having the amino acid sequence of SEQ ID NO:156,
   or a variant thereof in which 1 or 2 or 3 amino acids in HC-CDR1, and/or in which 1 or 2 or 3 amino acids in HC-CDR2, and/or in which 1 or 2 or 3 amino acids in HC-CDR3 are substituted with another amino acid.
(14) a VH region incorporating the following CDRs:
   HC-CDR1 having the amino acid sequence of SEQ ID NO:166
   HC-CDR2 having the amino acid sequence of SEQ ID NO:167
   HC-CDR3 having the amino acid sequence of SEQ ID NO:168,
   or a variant thereof in which 1 or 2 or 3 amino acids in HC-CDR1, and/or in which 1 or 2 or 3 amino acids in HC-CDR2, and/or in which 1 or 2 or 3 amino acids in HC-CDR3 are substituted with another amino acid.
(15) a VH region incorporating the following CDRs:
   HC-CDR1 having the amino acid sequence of SEQ ID NO:185
   HC-CDR2 having the amino acid sequence of SEQ ID NO:186
   HC-CDR3 having the amino acid sequence of SEQ ID NO:187,
   or a variant thereof in which 1 or 2 or 3 amino acids in HC-CDR1, and/or in which 1 or 2 or 3 amino acids in HC-CDR2, and/or in which 1 or 2 or 3 amino acids in HC-CDR3 are substituted with another amino acid.
(16) a VH region incorporating the following CDRs:
   HC-CDR1 having the amino acid sequence of SEQ ID NO:48
   HC-CDR2 having the amino acid sequence of SEQ ID NO:199
   HC-CDR3 having the amino acid sequence of SEQ ID NO:200,
   or a variant thereof in which 1 or 2 or 3 amino acids in HC-CDR1, and/or in which 1 or 2 or 3 amino acids in HC-CDR2, and/or in which 1 or 2 or 3 amino acids in HC-CDR3 are substituted with another amino acid.
(17) a VH region incorporating the following CDRs:
   HC-CDR1 having the amino acid sequence of SEQ ID NO:48
   HC-CDR2 having the amino acid sequence of SEQ ID NO:211
   HC-CDR3 having the amino acid sequence of SEQ ID NO:212,
   or a variant thereof in which 1 or 2 or 3 amino acids in HC-CDR1, and/or in which 1 or 2 or 3 amino acids in HC-CDR2, and/or in which 1 or 2 or 3 amino acids in HC-CDR3 are substituted with another amino acid.
(18) a VH region incorporating the following CDRs:
   HC-CDR1 having the amino acid sequence of SEQ ID NO:222
   HC-CDR2 having the amino acid sequence of SEQ ID NO:223
   HC-CDR3 having the amino acid sequence of SEQ ID NO:224,
   or a variant thereof in which 1 or 2 or 3 amino acids in HC-CDR1, and/or in which 1 or 2 or 3 amino acids in HC-CDR2, and/or in which 1 or 2 or 3 amino acids in HC-CDR3 are substituted with another amino acid.
(19) a VH region incorporating the following CDRs:
   HC-CDR1 having the amino acid sequence of SEQ ID NO:185
   HC-CDR2 having the amino acid sequence of SEQ ID NO:243
   HC-CDR3 having the amino acid sequence of SEQ ID NO:244,
   or a variant thereof in which 1 or 2 or 3 amino acids in HC-CDR1, and/or in which 1 or 2 or 3 amino acids in HC-CDR2, and/or in which 1 or 2 or 3 amino acids in HC-CDR3 are substituted with another amino acid.

In some embodiments, the antigen-binding molecule comprises a VH region according to one of (20) to (37) below:
(20) a VH region incorporating the following FRs:
   HC-FR1 having the amino acid sequence of SEQ ID NO:5
   HC-FR2 having the amino acid sequence of SEQ ID NO:6
   HC-FR3 having the amino acid sequence of SEQ ID NO:7
   HC-FR4 having the amino acid sequence of SEQ ID NO:8,
   or a variant thereof in which 1 or 2 or 3 amino acids in HC-FR1, and/or in which 1 or 2 or 3 amino acids in HC-FR2, and/or in which 1 or 2 or 3 amino acids in HC-FR3, and/or in which 1 or 2 or 3 amino acids in HC-FR4 are substituted with another amino acid.
(21) a VH region incorporating the following FRs:
   HC-FR1 having the amino acid sequence of SEQ ID NO:21
   HC-FR2 having the amino acid sequence of SEQ ID NO:22
   HC-FR3 having the amino acid sequence of SEQ ID NO:23
   HC-FR4 having the amino acid sequence of SEQ ID NO:8,
   or a variant thereof in which 1 or 2 or 3 amino acids in HC-FR1, and/or in which 1 or 2 or 3 amino acids in HC-FR2, and/or in which 1 or 2 or 3 amino acids in HC-FR3, and/or in which 1 or 2 or 3 amino acids in HC-FR4 are substituted with another amino acid.
(22) a VH region incorporating the following FRs:
   HC-FR1 having the amino acid sequence of SEQ ID NO:36
   HC-FR2 having the amino acid sequence of SEQ ID NO:37
   HC-FR3 having the amino acid sequence of SEQ ID NO:38
   HC-FR4 having the amino acid sequence of SEQ ID NO:39,
   or a variant thereof in which 1 or 2 or 3 amino acids in HC-FR1, and/or in which 1 or 2 or 3 amino acids in HC-FR2, and/or in which 1 or 2 or 3 amino acids in HC-FR3, and/or in which 1 or 2 or 3 amino acids in HC-FR4 are substituted with another amino acid.
(23) a VH region incorporating the following FRs:
   HC-FR1 having the amino acid sequence of SEQ ID NO:50
   HC-FR2 having the amino acid sequence of SEQ ID NO:6
   HC-FR3 having the amino acid sequence of SEQ ID NO:7
   HC-FR4 having the amino acid sequence of SEQ ID NO:51,
   or a variant thereof in which 1 or 2 or 3 amino acids in HC-FR1, and/or in which 1 or 2 or 3 amino acids in HC-FR2, and/or in which 1 or 2 or 3 amino acids in HC-FR3, and/or in which 1 or 2 or 3 amino acids in HC-FR4 are substituted with another amino acid.
(24) a VH region incorporating the following FRs:
   HC-FR1 having the amino acid sequence of SEQ ID NO:64
   HC-FR2 having the amino acid sequence of SEQ ID NO:65
   HC-FR3 having the amino acid sequence of SEQ ID NO:66
   HC-FR4 having the amino acid sequence of SEQ ID NO:39,
   or a variant thereof in which 1 or 2 or 3 amino acids in HC-FR1, and/or in which 1 or 2 or 3 amino acids in HC-FR2, and/or in which 1 or 2 or 3 amino acids in HC-FR3, and/or in which 1 or 2 or 3 amino acids in HC-FR4 are substituted with another amino acid.
(25) a VH region incorporating the following FRs:
   HC-FR1 having the amino acid sequence of SEQ ID NO:84
   HC-FR2 having the amino acid sequence of SEQ ID NO:6
   HC-FR3 having the amino acid sequence of SEQ ID NO:7
   HC-FR4 having the amino acid sequence of SEQ ID NO:39,
   or a variant thereof in which 1 or 2 or 3 amino acids in HC-FR1, and/or in which 1 or 2 or 3 amino acids in HC-FR2, and/or in which 1 or 2 or 3 amino acids in HC-FR3, and/or in which 1 or 2 or 3 amino acids in HC-FR4 are substituted with another amino acid.
(26) a VH region incorporating the following FRs:
   HC-FR1 having the amino acid sequence of SEQ ID NO:87
   HC-FR2 having the amino acid sequence of SEQ ID NO:6
   HC-FR3 having the amino acid sequence of SEQ ID NO:7
   HC-FR4 having the amino acid sequence of SEQ ID NO:51,
   or a variant thereof in which 1 or 2 or 3 amino acids in HC-FR1, and/or in which 1 or 2 or 3 amino acids in HC-FR2, and/or in which 1 or 2 or 3 amino acids in HC-FR3, and/or in which 1 or 2 or 3 amino acids in HC-FR4 are substituted with another amino acid.
(27) a VH region incorporating the following FRs:
   HC-FR1 having the amino acid sequence of SEQ ID NO:97
   HC-FR2 having the amino acid sequence of SEQ ID NO:98
   HC-FR3 having the amino acid sequence of SEQ ID NO:66
   HC-FR4 having the amino acid sequence of SEQ ID NO:39,
   or a variant thereof in which 1 or 2 or 3 amino acids in HC-FR1, and/or in which 1 or 2 or 3 amino acids in HC-FR2, and/or in which 1 or 2 or 3 amino acids in HC-FR3, and/or in which 1 or 2 or 3 amino acids in HC-FR4 are substituted with another amino acid.
(28) a VH region incorporating the following FRs:
   HC-FR1 having the amino acid sequence of SEQ ID NO: 11
   HC-FR2 having the amino acid sequence of SEQ ID NO:112
   HC-FR3 having the amino acid sequence of SEQ ID NO:113
   HC-FR4 having the amino acid sequence of SEQ ID NO:51,
   or a variant thereof in which 1 or 2 or 3 amino acids in HC-FR1, and/or in which 1 or 2 or 3 amino acids in HC-FR2, and/or in which 1 or 2 or 3 amino acids in HC-FR3, and/or in which 1 or 2 or 3 amino acids in HC-FR4 are substituted with another amino acid.
(29) a VH region incorporating the following FRs:
   HC-FR1 having the amino acid sequence of SEQ ID NO:123
   HC-FR2 having the amino acid sequence of SEQ ID NO:6
   HC-FR3 having the amino acid sequence of SEQ ID NO:7
   HC-FR4 having the amino acid sequence of SEQ ID NO:51,
   or a variant thereof in which 1 or 2 or 3 amino acids in HC-FR1, and/or in which 1 or 2 or 3 amino acids in HC-FR2, and/or in which 1 or 2 or 3 amino acids in HC-FR3, and/or in which 1 or 2 or 3 amino acids in HC-FR4 are substituted with another amino acid.
(30) a VH region incorporating the following FRs:
   HC-FR1 having the amino acid sequence of SEQ ID NO:135
   HC-FR2 having the amino acid sequence of SEQ ID NO:136
   HC-FR3 having the amino acid sequence of SEQ ID NO:137
   HC-FR4 having the amino acid sequence of SEQ ID NO:39,
   or a variant thereof in which 1 or 2 or 3 amino acids in HC-FR1, and/or in which 1 or 2 or 3 amino acids in HC-FR2, and/or in which 1 or 2 or 3 amino acids in HC-FR3, and/or in which 1 or 2 or 3 amino acids in HC-FR4 are substituted with another amino acid.
(31) a VH region incorporating the following FRs:
   HC-FR1 having the amino acid sequence of SEQ ID NO:149
   HC-FR2 having the amino acid sequence of SEQ ID NO:150
   HC-FR3 having the amino acid sequence of SEQ ID NO:151
   HC-FR4 having the amino acid sequence of SEQ ID NO:39,
   or a variant thereof in which 1 or 2 or 3 amino acids in HC-FR1, and/or in which 1 or 2 or 3 amino acids in HC-FR2, and/or in which 1 or 2 or 3 amino acids in HC-FR3, and/or in which 1 or 2 or 3 amino acids in HC-FR4 are substituted with another amino acid.
(32) a VH region incorporating the following FRs:
   HC-FR1 having the amino acid sequence of SEQ ID NO:87
   HC-FR2 having the amino acid sequence of SEQ ID NO:169
   HC-FR3 having the amino acid sequence of SEQ ID NO:168
   HC-FR4 having the amino acid sequence of SEQ ID NO:39,
   or a variant thereof in which 1 or 2 or 3 amino acids in HC-FR1, and/or in which 1 or 2 or 3 amino acids in HC-FR2, and/or in which 1 or 2 or 3 amino acids in HC-FR3, and/or in which 1 or 2 or 3 amino acids in HC-FR4 are substituted with another amino acid.
(33) a VH region incorporating the following FRs:
   HC-FR1 having the amino acid sequence of SEQ ID NO:188
   HC-FR2 having the amino acid sequence of SEQ ID NO:189
   HC-FR3 having the amino acid sequence of SEQ ID NO:7
   HC-FR4 having the amino acid sequence of SEQ ID NO:8,
   or a variant thereof in which 1 or 2 or 3 amino acids in HC-FR1, and/or in which 1 or 2 or 3 amino acids in HC-FR2, and/or in which 1 or 2 or 3 amino acids in HC-FR3, and/or in which 1 or 2 or 3 amino acids in HC-FR4 are substituted with another amino acid.
(34) a VH region incorporating the following FRs:
   HC-FR1 having the amino acid sequence of SEQ ID NO:201
   HC-FR2 having the amino acid sequence of SEQ ID NO:202
   HC-FR3 having the amino acid sequence of SEQ ID NO:203
   HC-FR4 having the amino acid sequence of SEQ ID NO:8,
   or a variant thereof in which 1 or 2 or 3 amino acids in HC-FR1, and/or in which 1 or 2 or 3 amino acids in HC-FR2, and/or in which 1 or 2 or 3 amino acids in HC-FR3, and/or in which 1 or 2 or 3 amino acids in HC-FR4 are substituted with another amino acid.
(35) a VH region incorporating the following FRs:
   HC-FR1 having the amino acid sequence of SEQ ID NO:213
   HC-FR2 having the amino acid sequence of SEQ ID NO:214
   HC-FR3 having the amino acid sequence of SEQ ID NO:7
   HC-FR4 having the amino acid sequence of SEQ ID NO:39,
   or a variant thereof in which 1 or 2 or 3 amino acids in HC-FR1, and/or in which 1 or 2 or 3 amino acids in HC-FR2, and/or in which 1 or 2 or 3 amino acids in HC-FR3, and/or in which 1 or 2 or 3 amino acids in HC-FR4 are substituted with another amino acid.
(36) a VH region incorporating the following FRs:
   HC-FR1 having the amino acid sequence of SEQ ID NO:225
   HC-FR2 having the amino acid sequence of SEQ ID NO:226
   HC-FR3 having the amino acid sequence of SEQ ID NO:227
   HC-FR4 having the amino acid sequence of SEQ ID NO:8,
   or a variant thereof in which 1 or 2 or 3 amino acids in HC-FR1, and/or in which 1 or 2 or 3 amino acids in HC-FR2, and/or in which 1 or 2 or 3 amino acids in HC-FR3, and/or in which 1 or 2 or 3 amino acids in HC-FR4 are substituted with another amino acid.
(37) a VH region incorporating the following FRs:
   HC-FR1 having the amino acid sequence of SEQ ID NO:245
   HC-FR2 having the amino acid sequence of SEQ ID NO:246
   HC-FR3 having the amino acid sequence of SEQ ID NO:190
   HC-FR4 having the amino acid sequence of SEQ ID NO:39,
   or a variant thereof in which 1 or 2 or 3 amino acids in HC-FR1, and/or in which 1 or 2 or 3 amino acids in HC-FR2, and/or in which 1 or 2 or 3 amino acids in HC-FR3, and/or in which 1 or 2 or 3 amino acids in HC-FR4 are substituted with another amino acid.

In some embodiments, the antigen-binding molecule comprises a VH region comprising the CDRs according to any one of (1) to (19) above, and the FRs according to any one of (20) to (37) above.

In some embodiments, the antigen-binding molecule comprises a VH region according to one of (38) to (56) below:
(38) a VH region comprising the CDRs according to (1) and the FRs according to (20).
(39) a VH region comprising the CDRs according to (2) and the FRs according to (21).
(40) a VH region comprising the CDRs according to (3) and the FRs according to (22).
(41) a VH region comprising the CDRs according to (4) and the FRs according to (23).
(42) a VH region comprising the CDRs according to (5) and the FRs according to (24).
(43) a VH region comprising the CDRs according to (6) and the FRs according to (25).
(44) a VH region comprising the CDRs according to (7) and the FRs according to (26).
(45) a VH region comprising the CDRs according to (8) and the FRs according to (27).
(46) a VH region comprising the CDRs according to (9) and the FRs according to (28).
(47) a VH region comprising the CDRs according to (10) and the FRs according to (29).
(48) a VH region comprising the CDRs according to (11) and the FRs according to (30).
(49) a VH region comprising the CDRs according to (12) and the FRs according to (31).
(50) a VH region comprising the CDRs according to (13) and the FRs according to (26).
(51) a VH region comprising the CDRs according to (14) and the FRs according to (32).
(52) a VH region comprising the CDRs according to (15) and the FRs according to (33).
(53) a VH region comprising the CDRs according to (16) and the FRs according to (34).
(54) a VH region comprising the CDRs according to (17) and the FRs according to (35).
(55) a VH region comprising the CDRs according to (18) and the FRs according to (36).
(56) a VH region comprising the CDRs according to (19) and the FRs according to (37).

In some embodiments, the antigen-binding molecule comprises a VH region according to one of (57) to (75) below:
(57) a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:1.
(58) a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:17.
(59) a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:32.
(60) a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:47.
(61) a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:60.
(62) a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:82.
(63) a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:85.
(64) a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:94.
(65) a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:107.
(66) a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:121.
(67) a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:131.
(68) a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:145.
(69) a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:155.
(70) a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:165.
(71) a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:184.
(72) a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:198.
(73) a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:210.
(74) a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:221.
(75) a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:242.

In some embodiments, the antigen-binding molecule comprises a VL region according to one of (76) to (97) below:
(76) a VL region incorporating the following CDRs:
   LC-CDR1 having the amino acid sequence of SEQ ID NO:10
   LC-CDR2 having the amino acid sequence of SEQ ID NO:11
   LC-CDR3 having the amino acid sequence of SEQ ID NO:12,
   or a variant thereof in which 1 or 2 or 3 amino acids in LC-CDR1, and/or in which 1 or 2 or 3 amino acids in LC-CDR2, and/or in which 1 or 2 or 3 amino acids in LC-CDR3 are substituted with another amino acid.
(77) a VL region incorporating the following CDRs:
   LC-CDR1 having the amino acid sequence of SEQ ID NO:25
   LC-CDR2 having the amino acid sequence of SEQ ID NO:26
   LC-CDR3 having the amino acid sequence of SEQ ID NO:27,
   or a variant thereof in which 1 or 2 or 3 amino acids in LC-CDR1, and/or in which 1 or 2 or 3 amino acids in LC-CDR2, and/or in which 1 or 2 or 3 amino acids in LC-CDR3 are substituted with another amino acid.
(78) a VL region incorporating the following CDRs:
   LC-CDR1 having the amino acid sequence of SEQ ID NO:41
   LC-CDR2 having the amino acid sequence of SEQ ID NO:42
   LC-CDR3 having the amino acid sequence of SEQ ID NO:43,
   or a variant thereof in which 1 or 2 or 3 amino acids in LC-CDR1, and/or in which 1 or 2 or 3 amino acids in LC-CDR2, and/or in which 1 or 2 or 3 amino acids in LC-CDR3 are substituted with another amino acid.
(79) a VL region incorporating the following CDRs:
   LC-CDR1 having the amino acid sequence of SEQ ID NO:53
   LC-CDR2 having the amino acid sequence of SEQ ID NO:54
   LC-CDR3 having the amino acid sequence of SEQ ID NO:55,
   or a variant thereof in which 1 or 2 or 3 amino acids in LC-CDR1, and/or in which 1 or 2 or 3 amino acids in LC-CDR2, and/or in which 1 or 2 or 3 amino acids in LC-CDR3 are substituted with another amino acid.
(80) a VL region incorporating the following CDRs:
   LC-CDR1 having the amino acid sequence of SEQ ID NO:68
   LC-CDR2 having the amino acid sequence of SEQ ID NO:26
   LC-CDR3 having the amino acid sequence of SEQ ID NO:69,
   or a variant thereof in which 1 or 2 or 3 amino acids in LC-CDR1, and/or in which 1 or 2 or 3 amino acids in LC-CDR2, and/or in which 1 or 2 or 3 amino acids in LC-CDR3 are substituted with another amino acid.
(81) a VL region incorporating the following CDRs:
   LC-CDR1 having the amino acid sequence of SEQ ID NO:73
   LC-CDR2 having the amino acid sequence of SEQ ID NO:26
   LC-CDR3 having the amino acid sequence of SEQ ID NO:74,
   or a variant thereof in which 1 or 2 or 3 amino acids in LC-CDR1, and/or in which 1 or 2 or 3 amino acids in LC-CDR2, and/or in which 1 or 2 or 3 amino acids in LC-CDR3 are substituted with another amino acid.
(82) a VL region incorporating the following CDRs:
   LC-CDR1 having the amino acid sequence of SEQ ID NO:78
   LC-CDR2 having the amino acid sequence of SEQ ID NO:79
   LC-CDR3 having the amino acid sequence of SEQ ID NO:80,
   or a variant thereof in which 1 or 2 or 3 amino acids in LC-CDR1, and/or in which 1 or 2 or 3 amino acids in LC-CDR2, and/or in which 1 or 2 or 3 amino acids in LC-CDR3 are substituted with another amino acid.
(83) a VL region incorporating the following CDRs:
   LC-CDR1 having the amino acid sequence of SEQ ID NO:89
   LC-CDR2 having the amino acid sequence of SEQ ID NO:11
   LC-CDR3 having the amino acid sequence of SEQ ID NO:90,
   or a variant thereof in which 1 or 2 or 3 amino acids in LC-CDR1, and/or in which 1 or 2 or 3 amino acids in LC-CDR2, and/or in which 1 or 2 or 3 amino acids in LC-CDR3 are substituted with another amino acid.
(84) a VL region incorporating the following CDRs:
   LC-CDR1 having the amino acid sequence of SEQ ID NO:101
   LC-CDR2 having the amino acid sequence of SEQ ID NO:102
   LC-CDR3 having the amino acid sequence of SEQ ID NO:103,
   or a variant thereof in which 1 or 2 or 3 amino acids in LC-CDR1, and/or in which 1 or 2 or 3 amino acids in LC-CDR2, and/or in which 1 or 2 or 3 amino acids in LC-CDR3 are substituted with another amino acid.
(85) a VL region incorporating the following CDRs:
   LC-CDR1 having the amino acid sequence of SEQ ID NO:115
   LC-CDR2 having the amino acid sequence of SEQ ID NO:116
   LC-CDR3 having the amino acid sequence of SEQ ID NO:117,
   or a variant thereof in which 1 or 2 or 3 amino acids in LC-CDR1, and/or in which 1 or 2 or 3 amino acids in LC-CDR2, and/or in which 1 or 2 or 3 amino acids in LC-CDR3 are substituted with another amino acid.
(86) a VL region incorporating the following CDRs:
   LC-CDR1 having the amino acid sequence of SEQ ID NO:125
   LC-CDR2 having the amino acid sequence of SEQ ID NO:126
   LC-CDR3 having the amino acid sequence of SEQ ID NO:127,
   or a variant thereof in which 1 or 2 or 3 amino acids in LC-CDR1, and/or in which 1 or 2 or 3 amino acids in LC-CDR2, and/or in which 1 or 2 or 3 amino acids in LC-CDR3 are substituted with another amino acid.
(87) a VL region incorporating the following CDRs:
   LC-CDR1 having the amino acid sequence of SEQ ID NO:139
   LC-CDR2 having the amino acid sequence of SEQ ID NO:140
   LC-CDR3 having the amino acid sequence of SEQ ID NO:80,
   or a variant thereof in which 1 or 2 or 3 amino acids in LC-CDR1, and/or in which 1 or 2 or 3 amino acids in LC-CDR2, and/or in which 1 or 2 or 3 amino acids in LC-CDR3 are substituted with another amino acid.
(88) a VL region incorporating the following CDRs:
   LC-CDR1 having the amino acid sequence of SEQ ID NO:41
   LC-CDR2 having the amino acid sequence of SEQ ID NO:42
   LC-CDR3 having the amino acid sequence of SEQ ID NO:153,
   or a variant thereof in which 1 or 2 or 3 amino acids in LC-CDR1, and/or in which 1 or 2 or 3 amino acids in LC-CDR2, and/or in which 1 or 2 or 3 amino acids in LC-CDR3 are substituted with another amino acid.
(89) a VL region incorporating the following CDRs:
   LC-CDR1 having the amino acid sequence of SEQ ID NO:158
   LC-CDR2 having the amino acid sequence of SEQ ID NO:159
   LC-CDR3 having the amino acid sequence of SEQ ID NO:160,
   or a variant thereof in which 1 or 2 or 3 amino acids in LC-CDR1, and/or in which 1 or 2 or 3 amino acids in LC-CDR2, and/or in which 1 or 2 or 3 amino acids in LC-CDR3 are substituted with another amino acid.
(90) a VL region incorporating the following CDRs:
   LC-CDR1 having the amino acid sequence of SEQ ID NO:171
   LC-CDR2 having the amino acid sequence of SEQ ID NO:172
   LC-CDR3 having the amino acid sequence of SEQ ID NO:173,
   or a variant thereof in which 1 or 2 or 3 amino acids in LC-CDR1, and/or in which 1 or 2 or 3 amino acids in LC-CDR2, and/or in which 1 or 2 or 3 amino acids in LC-CDR3 are substituted with another amino acid.
(91) a VL region incorporating the following CDRs:
   LC-CDR1 having the amino acid sequence of SEQ ID NO:179
   LC-CDR2 having the amino acid sequence of SEQ ID NO:180
   LC-CDR3 having the amino acid sequence of SEQ ID NO:173,
   or a variant thereof in which 1 or 2 or 3 amino acids in LC-CDR1, and/or in which 1 or 2 or 3 amino acids in LC-CDR2, and/or in which 1 or 2 or 3 amino acids in LC-CDR3 are substituted with another amino acid.
(92) a VL region incorporating the following CDRs:
   LC-CDR1 having the amino acid sequence of SEQ ID NO:73
   LC-CDR2 having the amino acid sequence of SEQ ID NO:26
   LC-CDR3 having the amino acid sequence of SEQ ID NO:194,
   or a variant thereof in which 1 or 2 or 3 amino acids in LC-CDR1, and/or in which 1 or 2 or 3 amino acids in LC-CDR2, and/or in which 1 or 2 or 3 amino acids in LC-CDR3 are substituted with another amino acid.
(93) a VL region incorporating the following CDRs:
   LC-CDR1 having the amino acid sequence of SEQ ID NO:205
   LC-CDR2 having the amino acid sequence of SEQ ID NO:42
   LC-CDR3 having the amino acid sequence of SEQ ID NO:206,
   or a variant thereof in which 1 or 2 or 3 amino acids in LC-CDR1, and/or in which 1 or 2 or 3 amino acids in LC-CDR2, and/or in which 1 or 2 or 3 amino acids in LC-CDR3 are substituted with another amino acid.
(94) a VL region incorporating the following CDRs:
   LC-CDR1 having the amino acid sequence of SEQ ID NO:216
   LC-CDR2 having the amino acid sequence of SEQ ID NO:172
   LC-CDR3 having the amino acid sequence of SEQ ID NO:217,
   or a variant thereof in which 1 or 2 or 3 amino acids in LC-CDR1, and/or in which 1 or 2 or 3 amino acids in LC-CDR2, and/or in which 1 or 2 or 3 amino acids in LC-CDR3 are substituted with another amino acid.
(95) a VL region incorporating the following CDRs:
   LC-CDR1 having the amino acid sequence of SEQ ID NO:229
   LC-CDR2 having the amino acid sequence of SEQ ID NO:172
   LC-CDR3 having the amino acid sequence of SEQ ID NO:230,
   or a variant thereof in which 1 or 2 or 3 amino acids in LC-CDR1, and/or in which 1 or 2 or 3 amino acids in LC-CDR2, and/or in which 1 or 2 or 3 amino acids in LC-CDR3 are substituted with another amino acid.
(96) a VL region incorporating the following CDRs:
   LC-CDR1 having the amino acid sequence of SEQ ID NO:235
   LC-CDR2 having the amino acid sequence of SEQ ID NO:236
   LC-CDR3 having the amino acid sequence of SEQ ID NO:237,
   or a variant thereof in which 1 or 2 or 3 amino acids in LC-CDR1, and/or in which 1 or 2 or 3 amino acids in LC-CDR2, and/or in which 1 or 2 or 3 amino acids in LC-CDR3 are substituted with another amino acid.
(97) a VL region incorporating the following CDRs:
   LC-CDR1 having the amino acid sequence of SEQ ID NO:248
   LC-CDR2 having the amino acid sequence of SEQ ID NO:249
   LC-CDR3 having the amino acid sequence of SEQ ID NO:250,
   or a variant thereof in which 1 or 2 or 3 amino acids in LC-CDR1, and/or in which 1 or 2 or 3 amino acids in LC-CDR2, and/or in which 1 or 2 or 3 amino acids in LC-CDR3 are substituted with another amino acid.

In some embodiments, the antigen-binding molecule comprises a VL region according to one of (98) to (119) below:
(98) a VL region incorporating the following FRs:
   LC-FR1 having the amino acid sequence of SEQ ID NO:13
   LC-FR2 having the amino acid sequence of SEQ ID NO:14
   LC-FR3 having the amino acid sequence of SEQ ID NO:15
   LC-FR4 having the amino acid sequence of SEQ ID NO:16,
   or a variant thereof in which 1 or 2 or 3 amino acids in LC-FR1, and/or in which 1 or 2 or 3 amino acids in LC-FR2, and/or in which 1 or 2 or 3 amino acids in LC-FR3, and/or in which 1 or 2 or 3 amino acids in LC-FR4 are substituted with another amino acid.
(99) a VL region incorporating the following FRs:
   LC-FR1 having the amino acid sequence of SEQ ID NO:28
   LC-FR2 having the amino acid sequence of SEQ ID NO:29
   LC-FR3 having the amino acid sequence of SEQ ID NO:30
   LC-FR4 having the amino acid sequence of SEQ ID NO:31,
   or a variant thereof in which 1 or 2 or 3 amino acids in LC-FR1, and/or in which 1 or 2 or 3 amino acids in LC-FR2, and/or in which 1 or 2 or 3 amino acids in LC-FR3, and/or in which 1 or 2 or 3 amino acids in LC-FR4 are substituted with another amino acid.
(100) a VL region incorporating the following FRs:
   LC-FR1 having the amino acid sequence of SEQ ID NO:44
   LC-FR2 having the amino acid sequence of SEQ ID NO:45
   LC-FR3 having the amino acid sequence of SEQ ID NO:46
   LC-FR4 having the amino acid sequence of SEQ ID NO:16,
   or a variant thereof in which 1 or 2 or 3 amino acids in LC-FR1, and/or in which 1 or 2 or 3 amino acids in LC-FR2, and/or in which 1 or 2 or 3 amino acids in LC-FR3, and/or in which 1 or 2 or 3 amino acids in LC-FR4 are substituted with another amino acid.
(101) a VL region incorporating the following FRs:
   LC-FR1 having the amino acid sequence of SEQ ID NO:56
   LC-FR2 having the amino acid sequence of SEQ ID NO:57
   LC-FR3 having the amino acid sequence of SEQ ID NO:58
   LC-FR4 having the amino acid sequence of SEQ ID NO:59,
   or a variant thereof in which 1 or 2 or 3 amino acids in LC-FR1, and/or in which 1 or 2 or 3 amino acids in LC-FR2, and/or in which 1 or 2 or 3 amino acids in LC-FR3, and/or in which 1 or 2 or 3 amino acids in LC-FR4 are substituted with another amino acid.
(102) a VL region incorporating the following FRs:
   LC-FR1 having the amino acid sequence of SEQ ID NO:28
   LC-FR2 having the amino acid sequence of SEQ ID NO:70
   LC-FR3 having the amino acid sequence of SEQ ID NO:71
   LC-FR4 having the amino acid sequence of SEQ ID NO:31,
   or a variant thereof in which 1 or 2 or 3 amino acids in LC-FR1, and/or in which 1 or 2 or 3 amino acids in LC-FR2, and/or in which 1 or 2 or 3 amino acids in LC-FR3, and/or in which 1 or 2 or 3 amino acids in LC-FR4 are substituted with another amino acid.
(103) a VL region incorporating the following FRs:
   LC-FR1 having the amino acid sequence of SEQ ID NO:28
   LC-FR2 having the amino acid sequence of SEQ ID NO:75
   LC-FR3 having the amino acid sequence of SEQ ID NO:76
   LC-FR4 having the amino acid sequence of SEQ ID NO:31,
   or a variant thereof in which 1 or 2 or 3 amino acids in LC-FR1, and/or in which 1 or 2 or 3 amino acids in LC-FR2, and/or in which 1 or 2 or 3 amino acids in LC-FR3, and/or in which 1 or 2 or 3 amino acids in LC-FR4 are substituted with another amino acid.
(104) a VL region incorporating the following FRs:
   LC-FR1 having the amino acid sequence of SEQ ID NO:28
   LC-FR2 having the amino acid sequence of SEQ ID NO:81
   LC-FR3 having the amino acid sequence of SEQ ID NO:76
   LC-FR4 having the amino acid sequence of SEQ ID NO:31,
   or a variant thereof in which 1 or 2 or 3 amino acids in LC-FR1, and/or in which 1 or 2 or 3 amino acids in LC-FR2, and/or in which 1 or 2 or 3 amino acids in LC-FR3, and/or in which 1 or 2 or 3 amino acids in LC-FR4 are substituted with another amino acid.
(105) a VL region incorporating the following FRs:
   LC-FR1 having the amino acid sequence of SEQ ID NO:91
   LC-FR2 having the amino acid sequence of SEQ ID NO:92
   LC-FR3 having the amino acid sequence of SEQ ID NO:93
   LC-FR4 having the amino acid sequence of SEQ ID NO:16,
   or a variant thereof in which 1 or 2 or 3 amino acids in LC-FR1, and/or in which 1 or 2 or 3 amino acids in LC-FR2, and/or in which 1 or 2 or 3 amino acids in LC-FR3, and/or in which 1 or 2 or 3 amino acids in LC-FR4 are substituted with another amino acid.
(106) a VL region incorporating the following FRs:
   LC-FR1 having the amino acid sequence of SEQ ID NO:104
   LC-FR2 having the amino acid sequence of SEQ ID NO:105
   LC-FR3 having the amino acid sequence of SEQ ID NO:106
   LC-FR4 having the amino acid sequence of SEQ ID NO:31,
   or a variant thereof in which 1 or 2 or 3 amino acids in LC-FR1, and/or in which 1 or 2 or 3 amino acids in LC-FR2, and/or in which 1 or 2 or 3 amino acids in LC-FR3, and/or in which 1 or 2 or 3 amino acids in LC-FR4 are substituted with another amino acid.
(107) a VL region incorporating the following FRs:
   LC-FR1 having the amino acid sequence of SEQ ID NO:118
   LC-FR2 having the amino acid sequence of SEQ ID NO:119
   LC-FR3 having the amino acid sequence of SEQ ID NO:120
   LC-FR4 having the amino acid sequence of SEQ ID NO:31,
   or a variant thereof in which 1 or 2 or 3 amino acids in LC-FR1, and/or in which 1 or 2 or 3 amino acids in LC-FR2, and/or in which 1 or 2 or 3 amino acids in LC-FR3, and/or in which 1 or 2 or 3 amino acids in LC-FR4 are substituted with another amino acid.
(108) a VL region incorporating the following FRs:
   LC-FR1 having the amino acid sequence of SEQ ID NO:128
   LC-FR2 having the amino acid sequence of SEQ ID NO:129
   LC-FR3 having the amino acid sequence of SEQ ID NO:130
   LC-FR4 having the amino acid sequence of SEQ ID NO:16,
   or a variant thereof in which 1 or 2 or 3 amino acids in LC-FR1, and/or in which 1 or 2 or 3 amino acids in LC-FR2, and/or in which 1 or 2 or 3 amino acids in LC-FR3, and/or in which 1 or 2 or 3 amino acids in LC-FR4 are substituted with another amino acid.
(109) a VL region incorporating the following FRs:
   LC-FR1 having the amino acid sequence of SEQ ID NO:141
   LC-FR2 having the amino acid sequence of SEQ ID NO:142
   LC-FR3 having the amino acid sequence of SEQ ID NO:143
   LC-FR4 having the amino acid sequence of SEQ ID NO:144,
   or a variant thereof in which 1 or 2 or 3 amino acids in LC-FR1, and/or in which 1 or 2 or 3 amino acids in LC-FR2, and/or in which 1 or 2 or 3 amino acids in LC-FR3, and/or in which 1 or 2 or 3 amino acids in LC-FR4 are substituted with another amino acid.
(110) a VL region incorporating the following FRs:
   LC-FR1 having the amino acid sequence of SEQ ID NO:44
   LC-FR2 having the amino acid sequence of SEQ ID NO:45
   LC-FR3 having the amino acid sequence of SEQ ID NO:46
   LC-FR4 having the amino acid sequence of SEQ ID NO:154,
   or a variant thereof in which 1 or 2 or 3 amino acids in LC-FR1, and/or in which 1 or 2 or 3 amino acids in LC-FR2, and/or in which 1 or 2 or 3 amino acids in LC-FR3, and/or in which 1 or 2 or 3 amino acids in LC-FR4 are substituted with another amino acid.
(111) a VL region incorporating the following FRs:
   LC-FR1 having the amino acid sequence of SEQ ID NO:161
   LC-FR2 having the amino acid sequence of SEQ ID NO:162
   LC-FR3 having the amino acid sequence of SEQ ID NO:163
   LC-FR4 having the amino acid sequence of SEQ ID NO:164,
   or a variant thereof in which 1 or 2 or 3 amino acids in LC-FR1, and/or in which 1 or 2 or 3 amino acids in LC-FR2, and/or in which 1 or 2 or 3 amino acids in LC-FR3, and/or in which 1 or 2 or 3 amino acids in LC-FR4 are substituted with another amino acid.
(112) a VL region incorporating the following FRs:
   LC-FR1 having the amino acid sequence of SEQ ID NO:174
   LC-FR2 having the amino acid sequence of SEQ ID NO:175
   LC-FR3 having the amino acid sequence of SEQ ID NO:176
   LC-FR4 having the amino acid sequence of SEQ ID NO:177,
   or a variant thereof in which 1 or 2 or 3 amino acids in LC-FR1, and/or in which 1 or 2 or 3 amino acids in LC-FR2, and/or in which 1 or 2 or 3 amino acids in LC-FR3, and/or in which 1 or 2 or 3 amino acids in LC-FR4 are substituted with another amino acid.
(113) a VL region incorporating the following FRs:
   LC-FR1 having the amino acid sequence of SEQ ID NO:181
   LC-FR2 having the amino acid sequence of SEQ ID NO:182
   LC-FR3 having the amino acid sequence of SEQ ID NO:183
   LC-FR4 having the amino acid sequence of SEQ ID NO:177,
   or a variant thereof in which 1 or 2 or 3 amino acids in LC-FR1, and/or in which 1 or 2 or 3 amino acids in LC-FR2, and/or in which 1 or 2 or 3 amino acids in LC-FR3, and/or in which 1 or 2 or 3 amino acids in LC-FR4 are substituted with another amino acid.
(114) a VL region incorporating the following FRs:
   LC-FR1 having the amino acid sequence of SEQ ID NO:28
   LC-FR2 having the amino acid sequence of SEQ ID NO:75
   LC-FR3 having the amino acid sequence of SEQ ID NO:197
   LC-FR4 having the amino acid sequence of SEQ ID NO:177,
   or a variant thereof in which 1 or 2 or 3 amino acids in LC-FR1, and/or in which 1 or 2 or 3 amino acids in LC-FR2, and/or in which 1 or 2 or 3 amino acids in LC-FR3, and/or in which 1 or 2 or 3 amino acids in LC-FR4 are substituted with another amino acid.
(115) a VL region incorporating the following FRs:
   LC-FR1 having the amino acid sequence of SEQ ID NO:44
   LC-FR2 having the amino acid sequence of SEQ ID NO:207
   LC-FR3 having the amino acid sequence of SEQ ID NO:208
   LC-FR4 having the amino acid sequence of SEQ ID NO:209,
   or a variant thereof in which 1 or 2 or 3 amino acids in LC-FR1, and/or in which 1 or 2 or 3 amino acids in LC-FR2, and/or in which 1 or 2 or 3 amino acids in LC-FR3, and/or in which 1 or 2 or 3 amino acids in LC-FR4 are substituted with another amino acid.
(116) a VL region incorporating the following FRs:
   LC-FR1 having the amino acid sequence of SEQ ID NO:218
   LC-FR2 having the amino acid sequence of SEQ ID NO:219
   LC-FR3 having the amino acid sequence of SEQ ID NO:220
   LC-FR4 having the amino acid sequence of SEQ ID NO:31,
   or a variant thereof in which 1 or 2 or 3 amino acids in LC-FR1, and/or in which 1 or 2 or 3 amino acids in LC-FR2, and/or in which 1 or 2 or 3 amino acids in LC-FR3, and/or in which 1 or 2 or 3 amino acids in LC-FR4 are substituted with another amino acid.
(117) a VL region incorporating the following FRs:
   LC-FR1 having the amino acid sequence of SEQ ID NO:231
   LC-FR2 having the amino acid sequence of SEQ ID NO:232
   LC-FR3 having the amino acid sequence of SEQ ID NO:233
   LC-FR4 having the amino acid sequence of SEQ ID NO:31,
   or a variant thereof in which 1 or 2 or 3 amino acids in LC-FR1, and/or in which 1 or 2 or 3 amino acids in LC-FR2, and/or in which 1 or 2 or 3 amino acids in LC-FR3, and/or in which 1 or 2 or 3 amino acids in LC-FR4 are substituted with another amino acid.
(118) a VL region incorporating the following FRs:
   LC-FR1 having the amino acid sequence of SEQ ID NO:238
   LC-FR2 having the amino acid sequence of SEQ ID NO:239
   LC-FR3 having the amino acid sequence of SEQ ID NO:240
   LC-FR4 having the amino acid sequence of SEQ ID NO:241,
   or a variant thereof in which 1 or 2 or 3 amino acids in LC-FR1, and/or in which 1 or 2 or 3 amino acids in LC-FR2, and/or in which 1 or 2 or 3 amino acids in LC-FR3, and/or in which 1 or 2 or 3 amino acids in LC-FR4 are substituted with another amino acid.
(119) a VL region incorporating the following FRs:
   LC-FR1 having the amino acid sequence of SEQ ID NO:231
   LC-FR2 having the amino acid sequence of SEQ ID NO:251
   LC-FR3 having the amino acid sequence of SEQ ID NO:252
   LC-FR4 having the amino acid sequence of SEQ ID NO:253,
   or a variant thereof in which 1 or 2 or 3 amino acids in LC-FR1, and/or in which 1 or 2 or 3 amino acids in LC-FR2, and/or in which 1 or 2 or 3 amino acids in LC-FR3, and/or in which 1 or 2 or 3 amino acids in LC-FR4 are substituted with another amino acid.

In some embodiments, the antigen-binding molecule comprises a VL region comprising the CDRs according to any one of (76) to (97) above, and the FRs according to any one of (98) to (119) above.

In some embodiments, the antigen-binding molecule comprises a VL region according to one of (120) to (142) below:
(120) a VL region comprising the CDRs according to (76) and the FRs according to (98).
(121) a VL region comprising the CDRs according to (77) and the FRs according to (99).
(123) a VL region comprising the CDRs according to (78) and the FRs according to (100).
(124) a VL region comprising the CDRs according to (79) and the FRs according to (101).
(125) a VL region comprising the CDRs according to (80) and the FRs according to (102).
(126) a VL region comprising the CDRs according to (81) and the FRs according to (103).
(127) a VL region comprising the CDRs according to (82) and the FRs according to (104).
(128) a VL region comprising the CDRs according to (83) and the FRs according to (105).
(129) a VL region comprising the CDRs according to (84) and the FRs according to (106).
(130) a VL region comprising the CDRs according to (85) and the FRs according to (107).
(131) a VL region comprising the CDRs according to (86) and the FRs according to (108).
(132) a VL region comprising the CDRs according to (87) and the FRs according to (109).
(133) a VL region comprising the CDRs according to (88) and the FRs according to (110).
(134) a VL region comprising the CDRs according to (89) and the FRs according to (111).
(135) a VL region comprising the CDRs according to (90) and the FRs according to (112).
(136) a VL region comprising the CDRs according to (91) and the FRs according to (113).
(137) a VL region comprising the CDRs according to (92) and the FRs according to (114).
(138) a VL region comprising the CDRs according to (93) and the FRs according to (115).
(139) a VL region comprising the CDRs according to (94) and the FRs according to (116).
(140) a VL region comprising the CDRs according to (95) and the FRs according to (117).
(141) a VL region comprising the CDRs according to (96) and the FRs according to (118).
(142) a VL region comprising the CDRs according to (97) and the FRs according to (119).

In some embodiments, the antigen-binding molecule comprises a VL region according to one of (143) to (164) below:
(143) a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:9.
(144) a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:24.
(145) a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:40.
(146) a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:52.
(147) a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:67.
(148) a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:72.
(149) a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:77.
(150) a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:88.
(151) a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:100.
(152) a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:114.
(153) a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:124.
(154) a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:138.
(155) a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:152.
(156) a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:157.
(157) a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:170.
(158) a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:178.
(159) a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:191.
(160) a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:204.
(161) a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:215.
(162) a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:228.
(163) a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:234.
(164) a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:247.

In some embodiments, the antigen-binding molecule comprises a VH region according to any one of (1) to (75) above, and a VL region according to any one of (76) to (164) above.

In embodiments in accordance with the present disclosure, one or more amino acids are substituted with another amino acid. A substitution comprises substitution of an amino acid residue with a non-identical ‘replacement’ amino acid residue. A replacement amino acid residue of a substitution according to the present disclosure may be a naturally-occurring amino acid residue (i.e. encoded by the genetic code) which is non-identical to the amino acid residue at the relevant position of the equivalent, unsubstituted amino acid sequence, selected from: alanine (Ala), arginine (Arg), asparagine (Asn), aspartic acid (Asp), cysteine (Cys), glutamine (Gln), glutamic acid (Glu), glycine (Gly), histidine (His), isoleucine (Ile): leucine (Leu), lysine (Lys), methionine (Met), phenylalanine (Phe), proline (Pro), serine (Ser), threonine (Thr), tryptophan (Trp), tyrosine (Tyr), and valine (Val). In some embodiments, a replacement amino acid may be a non-naturally occurring amino acid residue—i.e. an amino acid residue other than those recited in the preceding sentence. Examples of non-naturally occurring amino acid residues include norleucine, ornithine, norvaline, homoserine, aib, and other amino acid residue analogues such as those described in Ellman, et al., Meth. Enzym. 202 (1991) 301-336.

In some embodiments, a substitution may be biochemically conservative. In some embodiments, where an amino acid to be substituted is provided in one of rows 1 to 5 of the table below, the replacement amino acid of the substitution is another, non-identical amino acid provided in the same row:

| Row | Shared property | Amino acids |
|---|---|---|
| 1 | Hydrophobic | Met, Ala, Val, Leu, Ile, Trp, |
| | | Tyr, Phe, Norleucine |
| 2 | Neutral hydrophilic | Cys, Ser, Thr, Asn, Gln |
| 3 | Acidic or negatively-charged | Asp, Glu |
| 4 | Basic or positively-charged | His, Lys, Arg |
| 5 | Orientation influencing | Gly, Pro |

By way of illustration, in some embodiments wherein substitution is of a Met residue, the replacement amino acid may be selected from Ala, Val, Leu, lie, Trp, Tyr, Phe and Norleucine.

In some embodiments, a replacement amino acid in a substitution may have the same side chain polarity as the amino acid residue it replaces. In some embodiments, a replacement amino acid in a substitution may have the same side chain charge (at pH 7.4) as the amino acid residue it replaces:

| | | Side-chain charge |
|---|---|---|
| Amino Acid | Side-chain polarity | (pH 7.4) |
| Alanine | nonpolar | neutral |
| Arginine | basic polar | positive |
| Asparagine | polar | neutral |
| Aspartic acid | acidic polar | negative |
| Cysteine | nonpolar | neutral |
| Glutamic acid | acidic polar | negative |
| Glutamine | polar | neutral |
| Glycine | nonpolar | neutral |
| Histidine | basic polar | positive (10%) |
| | | neutral (90%) |
| Isoleucine | nonpolar | neutral |
| Leucine | nonpolar | neutral |
| Lysine | basic polar | positive |
| Methionine | nonpolar | neutral |
| Phenylalanine | nonpolar | neutral |
| Proline | nonpolar | neutral |
| Serine | polar | neutral |
| Threonine | polar | neutral |
| Tryptophan | nonpolar | neutral |
| Tyrosine | polar | neutral |
| Valine | nonpolar | neutral |

That is, in some embodiments, a nonpolar amino acid is substituted with another, non-identical nonpolar amino acid. In some embodiments, a polar amino acid is substituted with another, non-identical polar amino acid. In some embodiments, an acidic polar amino acid is substituted with another, non-identical acidic polar amino acid. In some embodiments, a basic polar amino acid is substituted with another, non-identical basic polar amino acid. In some embodiments, a neutral amino acid is substituted with another, non-identical neutral amino acid. In some embodiments, a positive amino acid is substituted with another, non-identical positive amino acid. In some embodiments, a negative amino acid is substituted with another, non-identical negative amino acid.

In some embodiments, substitution(s) may be functionally conservative. That is, In some embodiments, the substitution may not affect (or may not substantially affect) one or more functional properties (e.g. target binding) of the antigen-binding molecule comprising the substitution as compared to the equivalent unsubstituted molecule.

The VH and VL region of an antigen-binding region of an antibody together constitute the Fv region. In some embodiments, the antigen-binding molecule according to the present disclosure comprises, or consists of, an Fv region that binds to CNX. In some embodiments, the VH and VL regions of the Fv are provided as single polypeptide joined by a linker region, i.e. a single chain Fv (scFv).

The VL and light chain constant (CL) region, and the VH region and heavy chain constant 1 (CH1) region of an antigen-binding region of an antibody together constitute the Fab region. In some embodiments, the antigen-binding molecule comprises a Fab region comprising a VH, a CH1, a VL and a CL (e.g. CK or CA). In some embodiments, the Fab region comprises a polypeptide comprising a VH and a CH1 (e.g. a VH-CH1 fusion polypeptide), and a polypeptide comprising a VL and a CL (e.g. a VL-CL fusion polypeptide). In some embodiments, the Fab region comprises a polypeptide comprising a VH and a CL (e.g. a VH-CL fusion polypeptide) and a polypeptide comprising a VL and a CH (e.g. a VL-CH1 fusion polypeptide); that is, In some embodiments, the Fab region is a CrossFab region. In some embodiments, the VH, CH1, VL and CL regions of the Fab or CrossFab are provided as single polypeptide joined by linker regions, i.e. as a single chain Fab (scFab) or a single chain CrossFab (scCrossFab).

In some embodiments, the antigen-binding molecule described herein comprises, or consists of, a whole antibody that binds to CNX. As used herein, ‘whole antibody’ refers to an antibody having a structure which is substantially similar to the structure of an immunoglobulin (Ig). Different kinds of immunoglobulins and their structures are described e.g. in Schroeder and Cavacini J Allergy Clin Immunol. (2010) 125(202): S41-S52, which is hereby incorporated by reference in its entirety.

Immunoglobulins of type G (i.e. IgG) are ~150 kDa glycoproteins comprising two heavy chains and two light chains. From N- to C-terminus, the heavy chains comprise a VH followed by a heavy chain constant region comprising three constant domains (CH1, CH2, and CH3), and similarly the light chains comprise a VL followed by a CL. Depending on the heavy chain, immunoglobulins may be classed as IgG (e.g. IgG1, IgG2, IgG3, IgG4), IgA (e.g. IgA1, IgA2), IgD, IgE, or IgM. The light chain may be kappa (κ) or lambda (λ).

In some embodiments, the antigen-binding molecule described herein comprises, or consists of, an IgG (e.g. IgG1, IgG2, IgG3, IgG4), IgA (e.g. IgA1, IgA2), IgD, IgE, or IgM that binds to CNX.

In some embodiments, the antigen-binding molecule of the present disclosure comprises one or more regions (e.g. CH1, CH2, CH3, etc.) of an immunoglobulin heavy chain constant sequence. In some embodiments, the immunoglobulin heavy chain constant sequence is, or is derived from, the heavy chain constant sequence of an IgG (e.g. IgG1, IgG2, IgG3, IgG4), IgA (e.g. IgA1, IgA2), IgD, IgE or IgM, e.g. a human IgG (e.g. hIgG1, hIgG2, hIgG3, hIgG4), hIgA (e.g. hIgA1, hIgA2), hIgD, hIgE or hIgM. In some embodiments, the immunoglobulin heavy chain constant sequence is, or is derived from, the heavy chain constant sequence of a human IgG1 allotype (e.g. G1 m1, G1m2, G1m3 or G1m17).

In some embodiments, the antigen-binding molecule comprises an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:254, 259, 260 or 263.

In some embodiments, the antigen-binding molecule comprises a CH1 region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:255 or 261. In some embodiments, the antigen-binding molecule comprises a CH2 region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:257. In some embodiments, the antigen-binding molecule comprises a CH3 region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:258 or 262.

In some embodiments, the antigen-binding molecule comprises a hinge region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:256, 264, 265 or 266.

It will be appreciated that CH2 and/or CH3 regions may be provided with further substitutions in accordance with modification to an Fc region of the antigen-binding molecule as described herein.

In some embodiments, the antigen-binding molecule of the present disclosure comprises one or more regions of an immunoglobulin light chain constant sequence. In some embodiments, the immunoglobulin light chain constant sequence is human immunoglobulin kappa constant (IGKC; Cκ). In some embodiments, the immunoglobulin light chain constant sequence is a human immunoglobulin lambda constant (IGLC; Cλ), e.g. IGLC1, IGLC2, IGLC3, IGLC6 or IGLC7.

In some embodiments, the antigen-binding molecule comprises an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:267, 268, 269, 270, 271 or 272.

In some embodiments, the antigen-binding molecule is or comprises a monoclonal antibody, or an antigen-binding fragment thereof.

In some embodiments, the antigen-binding molecule is or comprises a fully human antibody/antibody fragment. A fully human antibody/antibody fragment may be encoded by human nucleic acid sequence(s). A fully human antibody/antibody fragment may be devoid of non-human amino acid sequences. Commonly employed techniques for the production of fully human antibodies include (i) phage display, in which human antibody genes are expressed in phage display libraries, and (ii) production of antibodies in transgenic mice engineered to have human antibody genes (described in Park and Smolen, Advances in Protein Chemistry (2001) 56: 369-421). Briefly, in the human antibody gene-phage display technique, genes encoding the VH and VL chains are generated by PCR amplification and cloning from ‘naive’ human lymphocytes, and assembled into a library from which they can be expressed either as disulfide-linked Fab fragments or as single-chain Fv (scFv) fragments. The Fab- or scFv-encoding genes are fused to a surface coat protein of filamentous bacteriophage and Fab or scFv capable of binding to the target of interest can then be identified by screening the library with antigen. Molecular evolution or affinity maturation procedures can be employed to enhance the affinity of the Fab/scFv fragment. In the transgenic mouse technique, mice in which the endogenous murine Ig gene loci have been replaced by homologous recombination with their human homologues are immunized with antigen, and monoclonal antibody is prepared by conventional hybridoma technology, to yield a fully human monoclonal antibody.

In some embodiments, the antigen-binding molecule of the present disclosure is a mouse antibody/antibody fragment. In some embodiments, the antibody/antibody fragment is obtained from phage display using a human naïve antibody gene library.

In some embodiments, the antigen-binding molecule is a mouse/human chimeric antibody/antibody fragment (i.e. an antigen-binding molecule comprising mouse antibody variable domains and human antibody constant regions). In some embodiments, the antigen-binding molecule is a humanised antibody/antibody fragment. In some embodiments, the antigen-binding molecule comprises mouse antibody CDRs and human antibody framework and constant regions.

Mouse/human chimeric antigen-binding molecules can be prepared from mouse antibodies by the process of chimerisation, e.g. as described in Human Monoclonal Antibodies: Methods and Protocols, Michael Steinitz (Editor), Methods in Molecular Biology 1060, Springer Protocols, Humana Press (2014), in Chapter 8 thereof, in particular section 3 of Chapter 8.

Humanised antigen-binding molecules can be prepared from mouse antibodies by the process of humanisation, e.g. as described in Human Monoclonal Antibodies: Methods and Protocols, Michael Steinitz (Editor), Methods in Molecular Biology 1060, Springer Protocols, Humana Press (2014), in Chapter 7 thereof, in particular section 3.1 of Chapter 7 entitled ‘Antibody Humanization’. Techniques for antibody humanisation are also described e.g. in Safdari et al., Biotechnol Genet Eng Rev (2013) 29:175-86.

Aspects of the present disclosure relate to multispecific antigen-binding molecules. By ‘multispecific’ it is meant that the antigen-binding molecule displays specific binding to more than one target. In some embodiments, the antigen-binding molecule is a bispecific antigen-binding molecule. In some embodiments, the antigen-binding molecule comprises at least two different antigen-binding domains (i.e. at least two antigen-binding domains, e.g. comprising non-identical VHs and VLs).

In some embodiments, the antigen-binding molecule binds to CNX and another target (e.g. an antigen other than CNX), and so is at least bispecific. The term ‘bispecific’ means that the antigen-binding molecule is able to bind specifically to at least two distinct antigenic determinants.

It will be appreciated that an antigen-binding molecule according to the present disclosure (e.g. a multispecific antigen-binding molecule) may comprise antigen-binding molecules capable of binding to the targets for which the antigen-binding molecule is specific. For example, an antigen-binding molecule that binds to CNX and an antigen other than CNX may comprise: (i) an antigen-binding molecule that binds to CNX, and (ii) an antigen-binding molecule that binds to an antigen other than CNX.

It will also be appreciated that an antigen-binding molecule according to the present disclosure (e.g. a multispecific antigen-binding molecule) may comprise antigen-binding polypeptides or antigen-binding polypeptide complexes capable of binding to the targets for which the antigen-binding molecule is specific.

In some embodiments, a component antigen-binding molecule of a larger antigen-binding molecule (e.g. a multispecific antigen-binding molecule) may be referred to e.g. as an ‘antigen-binding domain’ or ‘antigen-binding region’ of the larger antigen-binding molecule.

In some embodiments, the antigen other than CNX in a multispecific antigen-binding molecule is an immune cell surface molecule. In some embodiments, the antigen is a cancer cell antigen. In some embodiments, the antigen is a receptor molecule, e.g. a cell surface receptor. In some embodiments, the antigen is a cell signalling molecule, e.g. a cytokine, chemokine, interferon, interleukin or lymphokine. In some embodiments, the antigen is a growth factor or a hormone.

A cancer cell antigen is an antigen which is expressed or over-expressed by a cancer cell. A cancer cell antigen may be any peptide/polypeptide, glycoprotein, lipoprotein, glycan, glycolipid, lipid, or fragment thereof. A cancer cell antigen's expression may be associated with a cancer. A cancer cell antigen may be abnormally expressed by a cancer cell (e.g. the cancer cell antigen may be expressed with abnormal localisation), or may be expressed with an abnormal structure by a cancer cell. A cancer cell antigen may be capable of eliciting an immune response. In some embodiments, the antigen is expressed at the cell surface of the cancer cell (i.e. the cancer cell antigen is a cancer cell surface antigen). In some embodiments, the part of the antigen which is bound by the antigen-binding molecule described herein is displayed on the external surface of the cancer cell (i.e. is extracellular). The cancer cell antigen may be a cancer-associated antigen. In some embodiments, the cancer cell antigen is an antigen whose expression is associated with the development, progression or severity of symptoms of a cancer. The cancer-associated antigen may be associated with the cause or pathology of the cancer, or may be expressed abnormally as a consequence of the cancer. In some embodiments, the cancer cell antigen is an antigen whose expression is upregulated (e.g. at the RNA and/or protein level) by cells of a cancer, e.g. as compared to the level of expression by comparable non-cancerous cells (e.g. non-cancerous cells derived from the same tissue/cell type). In some embodiments, the cancer-associated antigen may be preferentially expressed by cancerous cells, and not expressed by comparable non-cancerous cells (e.g. non-cancerous cells derived from the same tissue/cell type). In some embodiments, the cancer-associated antigen may be the product of a mutated oncogene or mutated tumor suppressor gene. In some embodiments, the cancer-associated antigen may be the product of an overexpressed cellular protein, a cancer antigen produced by an oncogenic virus, an oncofetal antigen, or a cell surface glycolipid or glycoprotein.

An immune cell surface molecule may be any peptide/polypeptide, glycoprotein, lipoprotein, glycan, glycolipid, lipid, or fragment thereof expressed at or on the cell surface of an immune cell. In some embodiments, the part of the immune cell surface molecule which is bound by the antigen-binding molecule of the present disclosure is on the external surface of the immune cell (i.e. is extracellular). The immune cell surface molecule may be expressed at the cell surface of any immune cell. In some embodiments, the immune cell may be a cell of hematopoietic origin, e.g. a neutrophil, eosinophil, basophil, dendritic cell, lymphocyte, or monocyte. The lymphocyte may be e.g. a T cell, B cell, natural killer (NK) cell, NKT cell or innate lymphoid cell (ILC), or a precursor thereof (e.g. a thymocyte or pre-B cell).

In some embodiments, the antigen-binding molecule is an immune cell engager. Immune cell engagers are reviewed e.g. in Goebeler and Bargou, Nat. Rev. Clin. Oncol. (2020) 17: 418-434 and Ellerman, Methods (2019) 154:102-117, both of which are hereby incorporated by reference in their entirety. Immune cell engager molecules comprise an antigen-binding region for a target antigen of interest, and an antigen-binding region for recruiting/engaging an immune cell of interest. Immune cell engagers recruit/engage immune cells through an antigen-binding region specific for an immune cell surface molecule.

The best studied immune cells engagers are bispecific T cell engagers (BiTEs), which comprise a target antigen binding domain, and a CD3 polypeptide (typically CD3ε)-binding domain, through which the BiTE recruits T cells. Binding of the BiTE to its target antigen and to the CD3 polypeptide expressed by the T cell results in activation of the T cell, and ultimately directs T cell effector activity against cells expressing the target antigen. Other kinds of immune cell engagers are well known in the art, and include natural killer cell engagers such as bispecific killer engagers (BiKEs), which recruit and activate NK cells.

In some embodiments, multispecific antigen-binding molecules described herein display at least monovalent binding with respect to CNX, and also display at least monovalent binding with respect to a CD3 polypeptide (e.g. CD3ε, CD3δ, CD3γ or CD3ζ; preferably CD3ε, CD3δ or CD3γ; or more preferably CD3ε). In some embodiments, the antigen-binding molecule comprises one binding site for CNX and one binding site for a CD3 polypeptide.

In some embodiments, the antigen-binding molecule comprises the CDRs of an antigen-binding molecule that binds to a CD3 polypeptide (e.g. CD3ε, CD3δ, CD3γ or CD3ζ; preferably CD3ε, CD3δ or CD3γ; or more preferably CD3ε). In some embodiments, the antigen-binding molecule comprises the FRs of an antigen-binding molecule that binds to a CD3 polypeptide (e.g. CD3ε, CD3δ, CD3γ or CD3ζ; preferably CD3ε, CD3δ or CD3γ; or more preferably CD3ε). In some embodiments, the antigen-binding molecule comprises the CDRs and the FRs of an antigen-binding molecule that binds to a CD3 polypeptide (e.g. CD3ε, CD3δ, CD3γ or CD3ζ; preferably CD3ε, CD3δ or CD3γ; or more preferably CD3ε). That is, In some embodiments, the antigen-binding molecule comprises the VH region and the VL region of an antigen-binding molecule that binds to a CD3 polypeptide (e.g. CD3ε, CD3δ, CD3γ or CD3ζ; preferably CD3ε, CD3δ or CD3γ; or more preferably CD3ε).

In some embodiments, the antigen-binding molecule comprises the CDRs, FRs and/or the VH and/or VL regions of a CD3 polypeptide-binding antibody clone, or CDRs, FRs and/or VH and/or VL regions which are derived from those of a CD3 polypeptide-binding antibody clone.

In some embodiments, a CD3 polypeptide-binding antibody clone is selected from: OKT3 (in Kjer-Nielsen et al., PNAS (2004) 101(20):7675-80), SP34 (described e.g. in WO 2014/122143 A1), UCHT1 (described e.g. in WO 2000/041474 A1) HIT3a (Invitrogen Cat #16-0039-85), and clone SK7 (Invitrogen Cat #16-0036-81).

In some embodiments, the immune cell engaged by the immune cell engager is a T cell or an NK cell. In some embodiments, the immune cell engager is a T cell-engager.

Multispecific antigen-binding molecules according to the present disclosure may be provided in any suitable format, such as those formats described in described in Brinkmann and Kontermann, MAbs (2017) 9(2): 182-212, which is hereby incorporated by reference in its entirety. Suitable formats include those shown in FIG. 2 of Brinkmann and Kontermann, MAbs (2017) 9(2): 182-212: antibody conjugates, e.g. IgG2, F(ab′)2 or CovX-Body; IgG or IgG-like molecules, e.g. IgG, chimeric IgG, KA-body common HC; CH1/CL fusion proteins, e.g. scFv2-CH1/CL, VHH2-CH1/CL; ‘variable domain only’ bispecific antigen-binding molecules, e.g. tandem scFv (taFV), triplebodies, diabodies (Db), dsDb, Db(kih), DART, scDB, dsFv-dsFv, tandAbs, triple heads, tandem dAb/VHH, tertravalent dAb.VHH; Non-Ig fusion proteins, e.g. scFv₂-albumin, scDb-albumin, taFv-albumin, taFv-toxin, miniantibody, DNL-Fab₂, DNL-Fab₂-scFv, DNL-Fab₂-IgG-cytokine₂, ImmTAC (TCR-scFv); modified Fc and CH3 fusion proteins, e.g. scFv-Fc(kih), scFv-Fc(CH3 charge pairs), scFv-Fc (EW-RVT), scFv-fc (HA-TF), scFv-Fc (SEEDbody), taFv-Fc(kih), scFv-Fc(kih)-Fv, Fab-Fc(kih)-scFv, Fab-scFv-Fc(kih), Fab-scFv-Fc(BEAT), Fab-scFv-Fc (SEEDbody), DART-Fc, scFv-CH3(kih), TriFabs; Fc fusions, e.g. Di-diabody, scDb-Fc, taFv-Fc, scFv-Fc-scFv, HCAb-VHH, Fab-scFv-Fc, scFv4-Ig, scFv2-Fcab; CH3 fusions, e.g. Dia-diabody, scDb-CH3; IgE/IgM CH2 fusions, e.g. scFv-EHD2-scFv, scFvMHD2-scFv; Fab fusion proteins, e.g. Fab-scFv (bibody), Fab-scFv₂ (tribody), Fab-Fv, Fab-dsFv, Fab-VHH, orthogonal Fab-Fab; non-Ig fusion proteins, e.g. DNL-Fab3, DNL-Fab2-scFv, DNL-Fab2-IgG-cytokine2; asymmetric IgG or IgG-like molecules, e.g. IgG(kih), IgG(kih) common LC, ZW1 IgG common LC, Biclonics common LC, CrossMab, CrossMab(kih), scFab-IgG(kih), Fab-scFab-IgG(kih), orthogonal Fab IgG(kih), DuetMab, CH3 charge pairs+CH1/CL charge pairs, hinge/CH3 charge pairs, SEED-body, Duobody, four-in-one-CrossMab(kih), LUZ-Y common LC; LUZ-Y scFab-IgG, FcFc*; appended and Fc-modified IgGs, e.g. IgG(kih)-Fv, IgG HA-TF-Fv, IgG(kih)scFab, scFab-Fc(kih)-scFv2, scFab-Fc(kih)-scFv, half DVD-Ig, DVI-Ig (four-in-one), CrossMab-Fab; modified Fc and CH3 fusion proteins, e.g. Fab-Fc(kih)-scFv, Fab-scFv-Fc(kih), Fab-scFv-Fc(BEAT), Fab-scFv-Fc-SEEDbody, TriFab; appended IgGs-HC fusions, e.g. IgG-HC, scFv, IgG-dAb, IgG-taFV, IgG-CrossFab, IgG-orthogonal Fab, IgG-(CaCp) Fab, scFv-HC-IgG, tandem Fab-IgG (orthogonal Fab), Fab-IgG(CaCp Fab), Fab-IgG(CR3), Fab-hinge-IgG(CR3); appended IgGs-LC fusions, e.g. IgG-scFv(LC), scFv(LC)-IgG, dAb-IgG; appended IgGs-HC and LC fusions, e.g. DVD-Ig, TVD-Ig, CODV-Ig, scFv4-IgG, Zybody; Fc fusions, e.g. Fab-scFv-Fc, scFv₄-Ig; F(ab′)2 fusions, e.g. F(ab′)₂-scFv₂; CH1/CL fusion proteins e.g. scFv₂-CH1-hinge/CL; modified IgGs, e.g. DAF (two-in one-IgG), DutaMab, Mab²; and non-Ig fusions, e.g. DNL-Fab₄-IgG.

The skilled person is able to design and prepare bispecific antigen-binding molecules. Methods for producing multispecific antigen-binding molecules include chemically crosslinking antigen-binding molecules or antibody fragments, e.g. with reducible disulphide or non-reducible thioether bonds, for example as described in Segal and Bast, 2001. Production of Bispecific Antigen-binding molecules. Current Protocols in Immunology. 14:IV:2.13:2.13.1-2.13.16, which is hereby incorporated by reference in its entirety. For example, N-succinimidyl-3-(-2-pyridyldithio)-propionate (SPDP) can be used to chemically crosslink e.g. Fab fragments via hinge region SH— groups, to create disulfide-linked bispecific F(ab)₂ heterodimers.

Other methods for producing multispecific antigen-binding molecules include fusing antibody-producing hybridomas e.g. with polyethylene glycol, to produce a quadroma cell capable of secreting bispecific antibody, for example as described in D. M. and Bast, B. J. 2001. Production of Bispecific Antigen-binding molecules. Current Protocols in Immunology. 14:IV:2.13:2.13.1-2.13.16.

Multispecific antigen-binding molecules according to the present disclosure can also be produced recombinantly, by expression from e.g. a nucleic acid construct encoding polypeptides for the antigen-binding molecules, for example as described in Antibody Engineering: Methods and Protocols, Second Edition (Humana Press, 2012), at Chapter 40: Production of Bispecific Antigen-binding molecules: Diabodies and Tandem scFv (Hornig and Färber-Schwarz), or French, How to make bispecific antigen-binding molecules, Methods Mol. Med. 2000; 40:333-339, the entire contents of both of which are hereby incorporated by reference.

For example, a DNA construct encoding the light and heavy chain variable domains for the two antigen-binding fragments (i.e. the light and heavy chain variable domains for the antigen-binding fragment capable of binding CNX, and the light and heavy chain variable domains for the antigen-binding fragment capable of binding to another target protein), and including sequences encoding a suitable linker or dimerization domain between the antigen-binding fragments can be prepared by molecular cloning techniques. Recombinant bispecific antibody can thereafter be produced by expression (e.g. in vitro) of the construct in a suitable host cell (e.g. a mammalian host cell), and expressed recombinant bispecific antibody can then optionally be purified.

### Fc Regions

In some embodiments, the antigen-binding molecules of the present disclosure comprise an Fc region.

An Fc region is composed of CH2 and CH3 regions from one polypeptide, and CH2 and CH3 regions from another polypeptide. The CH2 and CH3 regions from the two polypeptides together form the Fc region.

Fc-mediated functions include Fc receptor binding, antibody-dependent cellular cytotoxicity (ADCC), antibody-dependent cell-mediated phagocytosis (ADCP), complement-dependent cytotoxicity (CDC), formation of the membrane attack complex (MAC), cell degranulation, cytokine and/or chemokine production, and antigen processing and presentation. Modifications to antibody Fc regions that influence Fc-mediated functions are known in the art, such as those described e.g. in Wang et al., Protein Cell (2018) 9(1):63-73, which is hereby incorporated by reference in its entirety. Exemplary Fc region modifications known to influence antibody effector function are summarised in Table 1 of Wang et al., Protein Cell (2018) 9(1):63-73. In some embodiments, the antigen-binding molecule of the present disclosure comprises an Fc region comprising modification to increase or reduce an Fc-mediated function as compared to an antigen-binding molecule comprising the corresponding unmodified Fc region.

Where an Fc region/CH2/CH3 is described as comprising modification(s) ‘corresponding to’ reference substitution(s), equivalent substitution(s) in the homologous Fc/CH2/CH3 are contemplated. By way of illustration, L234A/L235A substitutions in human IgG1 (numbered according to the EU numbering system as described in Kabat et al., Sequences of Proteins of Immunological Interest, 5th Ed. Public Health Service, National Institutes of Health, Bethesda, MD, 1991) correspond to L to A substitutions at positions 117 and 118 of the mouse Ig gamma-2A chain C region (UniProtKB: P01863-1, v1).

Where an Fc region is described as comprising a modification, the modification may be present in one or both of the polypeptide chains which together form the Fc region.

In some embodiments, the antigen-binding molecule of the present disclosure comprises an Fc region comprising modification. In some embodiments, the antigen-binding molecule of the present disclosure comprises an Fc region comprising modification in one or more of the CH2 and/or CH3 regions.

In some embodiments, the Fc region comprises modification to increase an Fc-mediated function. In some embodiments, the Fc region comprises modification to increase ADCC. In some embodiments, the Fc region comprises modification to increase ADCP. In some embodiments, the Fc region comprises modification to increase CDC. An antigen-binding molecule comprising an Fc region comprising modification to increase an Fc-mediated function (e.g. ADCC, ADCP, CDC) induces an increased level of the relevant effector function as compared to an antigen-binding molecule comprising the corresponding unmodified Fc region.

In some embodiments, the Fc region comprises modification to increase binding to an Fc receptor. In some embodiments, the Fc region comprises modification to increase binding to an Fcγ receptor. In some embodiments, the Fc region comprises modification to increase binding to one or more of FcγRI, FcγRIIa, FcγRIIb, FcγRIIc, FcγRIIIa and FcγRIIIb. In some embodiments, the Fc region comprises modification to increase binding to FcγRIIIa. In some embodiments, the Fc region comprises modification to increase binding to FcγRIIa. In some embodiments, the Fc region comprises modification to increase binding to FcγRIIb. In some embodiments, the Fc region comprises modification to increase binding to FcRn. In some embodiments, the Fc region comprises modification to increase binding to a complement protein. In some embodiments, the Fc region comprises modification to increase binding to C1q. In some embodiments, the Fc region comprises modification to promote hexamerisation of the antigen-binding molecule. In some embodiments, the Fc region comprises modification to increase antigen-binding molecule half-life. In some embodiments, the Fc region comprises modification to increase co-engagement.

In some embodiments, the Fc region comprises modification corresponding to the combination of substitutions F243L/R292P/Y300L/V3051/P396L as described in Stavenhagen et al. Cancer Res. (2007) 67:8882-8890. In some embodiments, the Fc region comprises modification corresponding to the combination of substitutions S239D/1332E or S239D/1332E/A330L as described in Lazar et al., Proc Natl Acad Sci USA. (2006)103:4005-4010. In some embodiments, the Fc region comprises modification corresponding to the combination of substitutions S298A/E333A/K334A as described in Shields et al., J Biol Chem. (2001) 276:6591-6604. In some embodiments, the Fc region comprises modification to one of heavy chain polypeptides corresponding to the combination of substitutions L234Y/L235Q/G236W/S239M/H268D/D270E/S298A, and modification to the other heavy chain polypeptide corresponding to the combination of substitutions D270E/K326D/A330M/K334E, as described in Mimoto et al., MAbs. (2013): 5:229-236. In some embodiments, the Fc region comprises modification corresponding to the combination of substitutions G236A/S239D/1332E as described in Richards et al., Mol Cancer Ther. (2008) 7:2517-2527.

In some embodiments, the Fc region comprises modification corresponding to the combination of substitutions K326W/E333S as described in Idusogie et al. J Immunol. (2001) 166(4):2571-5. In some embodiments, the Fc region comprises modification corresponding to the combination of substitutions S267E/H268F/S324T as described in Moore et al. MAbs. (2010) 2(2):181-9. In some embodiments, the Fc region comprises modification corresponding to the combination of substitutions described in Natsume et al., Cancer Res. (2008) 68(10):3863-72. In some embodiments, the Fc region comprises modification corresponding to the combination of substitutions E345R/E430G/S440Y as described in Diebolder et al. Science (2014) 343(6176):1260-3.

In some embodiments, the Fc region comprises modification corresponding to the combination of substitutions M252Y/S254T/T256E as described in Dall'Acqua et al. J Immunol. (2002) 169:5171-5180.

In some embodiments, the Fc region comprises modification corresponding to the combination of substitutions M428L/N434S as described in Zalevsky et al. Nat Biotechnol. (2010) 28:157-159.

In some embodiments, the Fc region comprises modification corresponding to the combination of substitutions S267E/L328F as described in Chu et al., Mol Immunol. (2008) 45:3926-3933. In some embodiments, the Fc region comprises modification corresponding to the combination of substitutions N325S/L328F as described in Shang et al. Biol Chem. (2014) 289:15309-15318.

In some embodiments, the Fc region comprises modification to reduce/prevent an Fc-mediated function. In some embodiments, the Fc region comprises modification to reduce/prevent ADCC. In some embodiments, the Fc region comprises modification to reduce/prevent ADCP. In some embodiments, the Fc region comprises modification to reduce/prevent CDC. An antigen-binding molecule comprising an Fc region comprising modification to reduce/prevent an Fc-mediated function (e.g. ADCC, ADCP, CDC) induces an reduced level of the relevant effector function as compared to an antigen-binding molecule comprising the corresponding unmodified Fc region.

In some embodiments, the Fc region comprises modification to reduce/prevent binding to an Fc receptor. In some embodiments, the Fc region comprises modification to reduce/prevent binding to an Fcγ receptor. In some embodiments, the Fc region comprises modification to reduce/prevent binding to one or more of FcγRI, FcγRIIa, FcγRIIb, FcγRIIc, FcγRIIIa and FcγRIIIb. In some embodiments, the Fc region comprises modification to reduce/prevent binding to FcγRIIIa. In some embodiments, the Fc region comprises modification to reduce/prevent binding to FcγRIIa. In some embodiments, the Fc region comprises modification to reduce/prevent binding to FcγRIIb. In some embodiments, the Fc region comprises modification to reduce/prevent binding to a complement protein. In some embodiments, the Fc region comprises modification to reduce/prevent binding to C1q. In some embodiments, the Fc region comprises modification to reduce/prevent glycosylation of the amino acid residue corresponding to N297.

In some embodiments, the Fc region is not able to induce one or more Fc-mediated functions (i.e. lacks the ability to elicit the relevant Fc-mediated function(s)). Accordingly, antigen-binding molecules comprising such Fc regions also lack the ability to induce the relevant function(s). Such antigen-binding molecules may be described as being devoid of the relevant function(s).

In some embodiments, the Fc region is not able to induce ADCC. In some embodiments, the Fc region is not able to induce ADCP. In some embodiments, the Fc region is not able to induce CDC. In some embodiments, the Fc region is not able to induce ADCC and/or is not able to induce ADCP and/or is not able to induce CDC.

In some embodiments, the Fc region is not able to bind to an Fc receptor. In some embodiments, the Fc region is not able to bind to an Fcγ receptor. In some embodiments, the Fc region is not able to bind to one or more of FcγRI, FcγRIIa, FcγRIIb, FcγRIIc, FcγRIIIa and FcγRIIIb. In some embodiments, the Fc region is not able to bind to FcγRIIIa. In some embodiments, the Fc region is not able to bind to FcγRIIa.

In some embodiments, the Fc region is not able to bind to FcγRIIb. In some embodiments, the Fc region is not able to bind to FcRn. In some embodiments, the Fc region is not able to bind to a complement protein. In some embodiments, the Fc region is not able to bind to C1q. In some embodiments, the Fc region is not glycosylated at the amino acid residue corresponding to N297.

In some embodiments, the Fc region comprises modification corresponding to N297A or N297Q or N297G as described in Leabman et al., MAbs. (2013) 5:896-903. In some embodiments, the Fc region comprises modification corresponding to L235E as described in Alegre et al., J Immunol. (1992) 148:3461-3468. In some embodiments, the Fc region comprises modification corresponding to the combination of substitutions L234A/L235A or F234A/L235A as described in Xu et al., Cell Immunol. (2000) 200:16-26. In some embodiments, the Fc region comprises modification corresponding to P329A or P329G as described in Schlothauer et al., Protein Engineering, Design and Selection (2016), 29(10):457-466. In some embodiments, the Fc region comprises modification corresponding to the combination of substitutions L234A/L235A/P329G as described in Lo et al. J. Biol. Chem (2017) 292(9):3900-3908. In some embodiments, the Fc region comprises modification corresponding to the combination of substitutions described in Rother et al., Nat Biotechnol. (2007) 25:1256-1264. In some embodiments, the Fc region comprises modification corresponding to the combination of substitutions S228P/L235E as described in Newman et al., Clin. Immunol. (2001) 98:164-174. In some embodiments, the Fc region comprises modification corresponding to the combination of substitutions H268Q/V309L/A330S/P331S as described in An et al., MAbs. (2009) 1:572-579. In some embodiments, the Fc region comprises modification corresponding to the combination of substitutions V234A/G237A/P238S/H268A/V309L/A330S/P331S as described in Vafa et al., Methods. (2014) 65:114-126. In some embodiments, the Fc region comprises modification corresponding to the combination of substitutions L234A/L235E/G237A/A330S/P331S as described in US 2015/0044231 A1.

The combination of substitutions ‘L234A/L235A’ and corresponding substitutions (such as e.g. F234A/L235A in human IgG4) are known to disrupt binding of Fc to Fcγ receptors and inhibit ADCC, ADCP, and also to reduce C1q binding and thus CDC (Schlothauer et al., Protein Engineering, Design and Selection (2016), 29(10):457-466, hereby incorporated by reference in entirety). The substitutions ‘P329G’ and ‘P329A’ reduce C1q binding (and thereby CDC). Substitution of ‘N297’ with ‘A’, ‘G’ or ‘Q’ is known to eliminate glycosylation, and thereby reduce Fc binding to C1q and Fcγ receptors, and thus CDC and ADCC. Lo et al. J. Biol. Chem (2017) 292(9):3900-3908 (hereby incorporated by reference in its entirety) reports that the combination of substitutions L234A/L235A/P329G eliminated complement binding and fixation as well as Fc γ receptor dependent, antibody-dependent, cell-mediated cytotoxicity in both murine IgG2a and human IgG1.

The combination of substitutions L234A/L235E/G237A/A330S/P331S in IgG1 Fc is disclosed in US 2015/0044231 A1 to abolish induction of phagocytosis, ADCC and CDC.

In some embodiments, the Fc region comprises modification corresponding to the substitution S228P as described in Silva et al., J Biol Chem. (2015) 290(9):5462-5469. The substitution S228P in IgG4 Fc reduces Fab-arm exchange (Fab arm exchange can be undesirable).

In some embodiments, the Fc region comprises modification corresponding to corresponding to the combination of substitutions L234A/L235A. In some embodiments, the Fc region comprises modification corresponding to corresponding to the substitution P329G. In some embodiments, the Fc region comprises modification corresponding to corresponding to the substitution N297Q.

In some embodiments, the Fc region comprises modification corresponding to corresponding to the combination of substitutions L234A/L235A/P329G.

In some embodiments, the Fc region comprises modification corresponding to corresponding to the combination of substitutions L234A/L235A/P329G/N297Q.

In some embodiments, the Fc region comprises modification corresponding to corresponding to the combination of substitutions L234A/L235E/G237A/A330S/P331S.

In some embodiments, the Fc region comprises modification corresponding to corresponding to the substitution S228P, e.g. in IgG4.

In some embodiments—particularly embodiments in which the antigen-binding molecule is a multispecific (e.g. bispecific) antigen-binding molecule—the antigen-binding molecule comprises an Fc region comprising modification in one or more of the CH2 and CH3 regions promoting association of the Fc region. Recombinant co-expression of constituent polypeptides of an antigen-binding molecule and subsequent association leads to several possible combinations. To improve the yield of the desired combinations of polypeptides in antigen-binding molecules in recombinant production, it is advantageous to introduce in the Fc regions modification(s) promoting association of the desired combination of heavy chain polypeptides. Modifications may promote e.g. hydrophobic and/or electrostatic interaction between CH2 and/or CH3 regions of different polypeptide chains. Suitable modifications are described e.g. in Ha et al., Front. Immnol (2016) 7:394, which is hereby incorporated by reference in its entirety.

In some embodiments, the antigen antigen-binding molecule of the present disclosure comprises an Fc region comprising paired substitutions in the CH3 regions of the Fc region according to one of the following formats, as shown in Table 1 of Ha et al., Front. Immnol (2016) 7:394: KiH, KiHₛ₋ₛ, HA-TF, ZW1, 7.8.60, DD-KK, EW-RVT, EW-RVTₛ₋ₛ, SEED or A107.

### Polypeptides and Particular Exemplary Antigen-Binding Molecules

The present disclosure also provides polypeptide constituents of antigen-binding molecules. The polypeptides may be provided in isolated or substantially purified form.

The antigen-binding molecule of the present disclosure may be, or may comprise, a complex of polypeptides.

In the present specification where a polypeptide comprises more than one domain or region, it will be appreciated that the plural domains/regions are preferably present in the same polypeptide chain. That is, the polypeptide comprising more than one domain or region is a fusion polypeptide comprising the domains/regions.

In some embodiments a polypeptide according to the present disclosure comprises, or consists of, a VH as described herein. In some embodiments a polypeptide according to the present disclosure comprises, or consists of, a VL as described herein.

In some embodiments, the polypeptide additionally comprises one or more antibody heavy chain constant regions (CH). In some embodiments, the polypeptide additionally comprises one or more antibody light chain constant regions (CL). In some embodiments, the polypeptide comprises a CH1, CH2 region and/or a CH3 region of an immunoglobulin (Ig).

In some embodiments, the polypeptide comprises one or more regions of an immunoglobulin heavy chain constant sequence. In some embodiments, the polypeptide comprises a CH1 region as described herein.

In some embodiments, the polypeptide comprises a CH1-CH2 hinge region as described herein. In some embodiments, the polypeptide comprises a CH2 region as described herein. In some embodiments, the polypeptide comprises a CH3 region as described herein.

In some embodiments, the polypeptide comprises one or more regions of an immunoglobulin light chain constant sequence. In some embodiments, the polypeptide comprises a CL region as described herein.

In some embodiments, the polypeptide according to the present disclosure comprises a structure from N- to C-terminus according to one of the following:

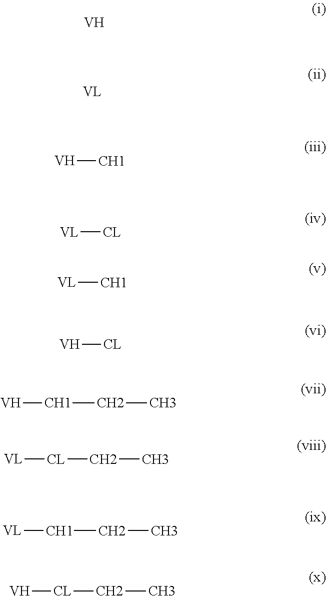

Also provided by the present disclosure are antigen-binding molecules composed of the polypeptides of the present disclosure. In some embodiments, the antigen-binding molecule of the present disclosure comprises one of the following combinations of polypeptides:

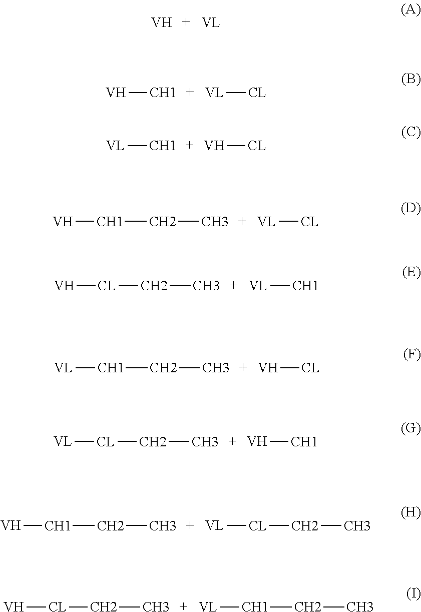

In some embodiments, the antigen-binding molecule comprises more than one polypeptide of the combinations shown in (A) to (I) above. By way of example, with reference to (D) above, in some embodiments, the antigen-binding molecule comprises two polypeptides comprising the structure VH-CH1-CH2-CH3, and two polypeptides comprising the structure VL-CL.

In some embodiments, the antigen-binding molecule of the present disclosure comprises one of the following combinations of polypeptides:

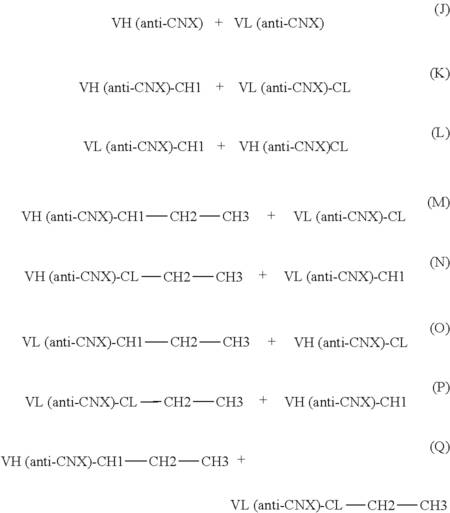

Wherein: ‘VH(anti-CNX)’ refers to the VH of an antigen-binding molecule capable of binding to CNX as described herein, e.g. as defined in one of (1) to (75); and ‘VL(anti-CNX)’ refers to the VL of an antigen-binding molecule capable of binding to CNX as described herein, e.g. as defined in one of (76) to (164).

In some embodiments, the antigen-binding molecule of the present disclosure comprises a polypeptide which comprises or consists of an amino acid sequence having at least 70%, preferably one of 80%, 85%, 90%, 91%, 92%, 93%, 94%, 95%, 96%, 97%, 98%, 99% or 100% amino acid sequence identity to SEQ ID NO:1, 17, 32, 47, 60, 82, 85, 94, 107, 121, 131, 145, 155, 165, 184, 198, 210, 221, or 242.

In some embodiments, the antigen-binding molecule of the present disclosure comprises a polypeptide which comprises or consists of an amino acid sequence having at least 70%, preferably one of 80%, 85%, 90%, 91%, 92%, 93%, 94%, 95%, 96%, 97%, 98%, 99% or 100% amino acid sequence identity to SEQ ID NO:9, 24, 40, 52, 67, 72, 77, 88, 100, 114, 124, 138, 152, 157, 170, 178, 191, 204, 215, 228, 234 or 247.

In some embodiments, the antigen-binding molecule of the present disclosure comprises a polypeptide which comprises or consists of an amino acid sequence having at least 70%, preferably one of 80%, 85%, 90%, 91%, 92%, 93%, 94%, 95%, 96%, 97%, 98%, 99% or 100% amino acid sequence identity to SEQ ID NO:273, 276, 279, 282, 285, 290, 292, 295, 298, 301, 304, 307, 310, 313, 317, 320, 323, 326, 330, 274, 277, 280, 283, 286, 291, 293, 296, 299, 302, 305, 308, 311, 314, 318, 321, 324, 327, or 331.

In some embodiments, the antigen-binding molecule of the present disclosure comprises a polypeptide which comprises or consists of an amino acid sequence having at least 70%, preferably one of 80%, 85%, 90%, 91%, 92%, 93%, 94%, 95%, 96%, 97%, 98%, 99% or 100% amino acid sequence identity to SEQ ID NO:275, 278, 281, 284, 287, 288, 289, 294, 297, 300, 303, 306, 309, 312, 315, 316, 319, 322, 325, 328, 329 or 332.

In some embodiments, the antigen-binding molecule of the present disclosure comprises a polypeptide or polypeptides comprising a VH region comprising the heavy chain CDRs, and a VL region comprising the light chain CDRs, of a clone selected from 1D3, 1D6, 1E1, 1E6, 2C6, 2H6, 3D1, 2G9, 2G12, 2H5, 3F8, 3F9, 4G9, 5A3, 5E8, C001, 0008, C010, C023, C025, C040, C046 and C117, as shown in Table A herein. That is, in some embodiments, the antigen-binding molecule comprises a polypeptide or polypeptides comprising: (i) a VH region comprising HC-CDR1, HC-CDR2 and HC-CDR3 as indicated in column A of Table A, and (ii) a VL region comprising LC-CDR1, LC-CDR2 and LC-CDR3 as indicated in column B of Table A, wherein the sequences of columns A and B are selected from the same row of Table A.

In some embodiments, the antigen-binding molecule of the present disclosure comprises a polypeptide or polypeptides comprising a VH region comprising the heavy chain FRs, and a VL region comprising the light chain FRs, of a clone selected from 1D3, 1D6, 1E1, 1E6, 2C6, 2H6, 3D1, 2G9, 2G12, 2H5, 3F8, 3F9, 4G9, 5A3, 5E8, 0001, 0008, C010, C023, C025, C040, C046 and C117, as shown in Table B herein. That is, in some embodiments, the antigen-binding molecule comprises a polypeptide or polypeptides comprising: (i) a VH region comprising HC-FR1, HC-FR2, HC-FR3 and HC-FR4 as indicated in column A of Table B, and (ii) a VL region comprising LC-FR1, LC-FR2, LC-FR3, and LC-FR4 as indicated in column B of Table B, wherein the sequences of columns A and B are selected from the same row of Table B.

In some embodiments, the antigen-binding molecule of the present disclosure comprises a polypeptide or polypeptides comprising: (i) an amino acid sequence having at least 70%, preferably one of 80%, 85%, 90%, 91%, 92%, 93%, 94%, 95%, 96%, 97%, 98%, 99% or 100% amino acid sequence identity to an amino acid sequence indicated in column A of Table C, and (ii) an amino acid sequence having at least 70%, preferably one of 80%, 85%, 90%, 91%, 92%, 93%, 94%, 95%, 96%, 97%, 98%, 99% or 100% amino acid sequence identity to an amino acid sequence indicated in column B of Table C, wherein the sequences of columns A and B are selected from the same row of Table C.

In some embodiments, the antigen-binding molecule of the present disclosure comprises a polypeptide or polypeptides comprising a VH region and a VL region of a clone selected from 1D3, 1D6, 1E1, 1E6, 2C6, 2H6, 3D1, 2G9, 2G12, 2H5, 3F8, 3F9, 4G9, 5A3, 5E8, C001, C008, C010, C023, C025, C040, C046 and C117, as shown in Table C herein. That is, in some embodiments, the antigen-binding molecule comprises a polypeptide or polypeptides comprising: (i) an amino acid sequence indicated in column A of Table C, and (ii) an amino acid sequence indicated in column B of Table C, wherein the sequences of columns A and B are selected from the same row of Table C.

In some embodiments, the antigen-binding molecule of the present disclosure comprises: (i) a polypeptide comprising or consisting of an amino acid sequence having at least 70%, preferably one of 80%, 85%, 90%, 91%, 92%, 93%, 94%, 95%, 96%, 97%, 98%, 99% or 100% amino acid sequence identity to an amino acid sequence indicated in column A of Table D, and (ii) a polypeptide comprising or consisting of an amino acid sequence having at least 70%, preferably one of 80%, 85%, 90%, 91%, 92%, 93%, 94%, 95%, 96%, 97%, 98%, 99% or 100% amino acid sequence identity to an amino acid sequence indicated in column B of Table D, wherein the sequences of columns A and B are selected from the same row of Table D.

In some embodiments, the antigen-binding molecule of the present disclosure comprises the polypeptides of an antigen-binding molecule according to any one of [1] to [46] as detailed in Table D herein. That is, in some embodiments, the antigen-binding molecule comprises: (i) a polypeptide comprising or consisting of an amino acid sequence indicated in column A of Table D, and (ii) a polypeptide comprising or consisting of an amino acid sequence indicated in column B of Table D, wherein the sequences of columns A and B are selected from the same row of Table D.

### Linkers and Additional Sequences

In some embodiments, the antigen-binding molecules and polypeptides of the present disclosure comprise one or more linker sequences between amino acid sequences. A linker sequence may be provided at one or both ends of one or more of a VH, VL, CH1-CH2 hinge region, CH2 region and a CH3 region of the antigen-binding molecule/polypeptide.

Linker sequences are known to the skilled person, and are described, for example in Chen et al., Adv Drug Deliv Rev (2013) 65(10): 1357-1369, which is hereby incorporated by reference in its entirety. In some embodiments, a linker sequence may be a flexible linker sequence. Flexible linker sequences allow for relative movement of the amino acid sequences which are linked by the linker sequence. Flexible linkers are known to the skilled person, and several are identified in Chen et al., Adv Drug Deliv Rev (2013) 65(10): 1357-1369. Flexible linker sequences often comprise high proportions of glycine and/or serine residues.

In some embodiments, the linker sequence comprises at least one glycine residue and/or at least one serine residue. In some embodiments, the linker sequence comprises or consists of glycine and serine residues. In some embodiments, the linker sequence has the structure: (GxS)n (SEQ ID NO; 410 and 411) or (GxS)nGm (SEQ ID NO:412 and 413); wherein G=glycine, S=serine, x=3 or 4, n=2, 3, 4, 5 or 6, and m=0, 1, 2 or 3. In some embodiments, the linker sequence comprises one or more (e.g. 1, 2, 3, 4, 5 or 6) copies (e.g. in tandem) of the sequence motif G₄S (SEQ ID NO:414). In some embodiments, the linker sequence comprises or consists of (G₄S)₄ (SEQ ID NO:415) or (G₄S)₆ (SEQ ID NO:416). In some embodiments, the linker sequence has a length of 1-2, 1-3, 1-4, 1-5, 1-10, 1-15, 1-20, 1-25, or 1-30 amino acids.

The antigen-binding molecules and polypeptides of the present disclosure may additionally comprise further amino acids or sequences of amino acids. For example, the antigen-binding molecules and polypeptides may comprise amino acid sequence(s) to facilitate expression, folding, trafficking, processing, purification, or detection of the antigen-binding molecule/polypeptide. For example, antigen-binding molecules and polypeptides of the present disclosure may additionally comprise a sequence of amino acids forming a detectable moiety, e.g. as described hereinbelow.

The antigen-binding molecules and polypeptides of the present disclosure may additionally comprise a signal peptide (also known as a leader sequence or signal sequence). Signal peptides normally consist of a sequence of 5-30 hydrophobic amino acids, which form a single alpha helix. Secreted proteins and proteins expressed at the cell surface often comprise signal peptides.

The signal peptide may be present at the N-terminus of the antigen-binding molecule/polypeptide, and may be present in the newly synthesised antigen-binding molecule/polypeptide. The signal peptide provides for efficient trafficking and secretion of the antigen-binding molecule/polypeptide. Signal peptides are often removed by cleavage, and thus are not comprised in the mature antigen-binding molecule/polypeptide secreted from the cell expressing the antigen-binding molecule/polypeptide.

Signal peptides are known for many proteins, and are recorded in databases such as GenBank, UniProt, Swiss-Prot, TrEMBL, Protein Information Resource, Protein Data Bank, Ensembl, and InterPro, and/or can be identified/predicted e.g. using amino acid sequence analysis tools such as SignalP (Petersen et al., 2011 Nature Methods 8: 785-786) or Signal-BLAST (Frank and Sippl, 2008 Bioinformatics 24: 2172-2176).

### Labels and Conjugates

In some embodiments, the antigen-binding molecules of the present disclosure additionally comprise a detectable moiety.

In some embodiments, the antigen-binding molecule comprises a detectable moiety, e.g. a fluorescent label, phosphorescent label, luminescent label, immuno-detectable label (e.g. an epitope tag), radiolabel, chemical, nucleic acid or enzymatic label. The antigen-binding molecule may be covalently or non-covalently labelled with the detectable moiety.

Fluorescent labels include e.g. fluorescein, rhodamine, allophycocyanin, eosine and NDB, green fluorescent protein (GFP), chelates of rare earths such as europium (Eu), terbium (Tb) and samarium (Sm), tetramethyl rhodamine, Texas Red, 4-methyl umbelliferone, 7-amino-4-methyl coumarin, Cy3, and Cy5. Radiolabels include radioisotopes such as Hydrogen³, Sulfur³⁵, Carbon¹⁴, Phosphorus³², Iodine¹²³, Iodine¹²⁵, Iodine¹²⁶, Iodine¹³¹, Iodine¹³³, Bromine⁷⁷, Technetium^{99m}, Indium¹¹¹, Indium^{113m}, Gallium⁶⁷, Gallium⁶⁸, Ruthenium⁹⁵, Ruthenium⁹⁷, Ruthenium¹⁰³, Ruthenium¹⁰⁵, Mercury²⁰⁷, Mercury²⁰³, Rhenium^{99m}, Rhenium¹⁰¹, Rhenium¹⁰⁵, Scandium⁴⁷, Tellurium^{121m}, Tellurium^{122m}, Tellurium^{125m}, Thulium¹⁶⁵, ThuliumI¹⁶⁷, Thulium¹⁶⁸, Copper⁶⁷, Fluorine¹⁸, Yttrium⁹⁰, Palladium¹⁰⁰, Bismuth²¹⁷ and Antimony²¹¹. Luminescent labels include as radioluminescent, chemiluminescent (e.g. acridinium ester, luminol, isoluminol) and bioluminescent labels. Immuno-detectable labels include haptens, peptides/polypeptides, antibodies, receptors and ligands such as biotin, avidin, streptavidin or digoxigenin. Nucleic acid labels include aptamers.

In some embodiments, the antigen-binding molecule/polypeptide comprises an epitope tag, e.g. a His, (e.g. 6×His), FLAG, c-Myc, StrepTag, haemagglutinin, calmodulin-binding protein (CBP), glutathione-s-transferase (GST), maltose-binding protein (MBP), thioredoxin, S-peptide, T7 peptide, SH2 domain, avidin, streptavidin, and haptens (e.g. biotin, digoxigenin, dinitrophenol), optionally at the N- or C-terminus of the antigen-binding molecule/polypeptide.

In some embodiments, the antigen-binding molecule/polypeptide comprises a moiety having a detectable activity, e.g. an enzymatic moiety. Enzymatic moieties include e.g. luciferases, glucose oxidases, galactosidases (e.g. beta-galactosidase), glucorinidases, phosphatases (e.g. alkaline phosphatase), peroxidases (e.g. horseradish peroxidase) and cholinesterases.

In some embodiments, the antigen-binding molecules of the present disclosure are conjugated to a chemical moiety. The chemical moiety may be a moiety for providing a therapeutic effect, i.e. a drug moiety. A drug moiety may be a small molecule (e.g. a low molecular weight (<1000 daltons, typically between ~300-700 daltons) organic compound). Drug moieties are described e.g. in Parslow et al., Biomedicines. 2016 September; 4(3):14 (hereby incorporated by reference in its entirety). In some embodiments, a drug moiety may be or comprise a cytotoxic agent. In some embodiments, a drug moiety may be or comprise a chemotherapeutic agent. Drug moieties include e.g. calicheamicin, DM1, DM4, monomethylauristatin E (MMAE), monomethylauristatin F (MMAF), SN-38, doxorubicin, duocarmycin, D6.5 and PBD.

Antigen-binding molecules according to the present disclosure also include antibody-derived molecules, e.g. molecules comprising an antigen-binding region/domain derived from an antibody. Antibody-derived antigen-binding molecules may comprise an antigen-binding region/domain that comprises, or consists of, the antigen-binding region of an antibody (e.g. an antigen-binding fragment of an antibody). In some embodiments, the antigen-binding region/domain of an antibody-derived antigen-binding molecule may be or comprise the Fv (e.g. provided as an scFv) or the Fab region of an antibody, or the whole antibody. For example, antigen-binding molecules according to the present disclosure include antibody-drug conjugates (ADCs) comprising a (cytotoxic) drug moiety. Antigen-binding molecules according to the present disclosure also include multispecific antigen-binding molecules such as immune cell engager molecules comprising a domain for recruiting (effector) immune cells (reviewed e.g. in Goebeler and Bargou, Nat. Rev. Clin. Oncol. (2020) 17: 418-434 and Ellerman, Methods (2019) 154:102-117, both of which are hereby incorporated by reference in their entirety), including BiTEs, BiKEs and TriKEs. Antigen-binding molecules according to the present disclosure also include chimeric antigen receptors (CARs), which are recombinant receptors providing both antigen-binding and T cell activating functions (CAR structure, function and engineering is reviewed e.g. in Dotti et al., Immunol Rev (2014) 257(1), which is hereby incorporated by reference in its entirety).

In some embodiments, an antigen-binding molecule according to the present disclosure comprises a drug moiety. The antigen-binding molecule may be conjugated to the drug moiety. Antibody-drug conjugates are reviewed e.g. in Parslow et al., Biomedicines. 2016 September; 4(3):14 (hereby incorporated by reference in its entirety). FDA approved ADCs currently on the market are described in Tong et al., Molecules. 2021 October; 26(19): 5847 (hereby incorporated by reference in its entirety).

In some embodiments the antibody-drug conjugate comprises an antigen binding molecule moiety, a drug moiety (or payload moiety), and a linker to join the drug moiety to the antibody. In some embodiments the antibody-drug conjugate consists of an antibody moiety, a drug moiety (or payload moiety), and a linker to join the drug moiety to the antibody.

The antigen binding molecule moiety may be a molecule that binds to a given target antigen. Antigen-binding molecules include antibodies (i.e. immunoglobulins (Igs)) and antigen-binding fragments thereof. As used herein, ‘antibodies’ include monoclonal antibodies, polyclonal antibodies, monospecific and multispecific (e.g., bispecific, trispecific, etc.) antibodies, and antibody-derived antigen-binding molecules such as scFv, scFab, diabodies, triabodies, scFv-Fc, minibodies, single domain antibodies (e.g. VhH), etc.). Antigen-binding fragments of antibodies include e.g. Fv, Fab, F(ab′)2 and F(ab′) fragments.

The linker may be cleavable or non-cleavable. The linker may be based on a chemical motifs such as disulfides, hydrazones or peptides (cleavable), or thioethers (non-cleavable). The type of linker, cleavable or noncleavable, lends specific properties to the cytotoxic drug. For example, a non-cleavable linker keeps the drug within the cell. As a result, the entire antibody, linker and cytotoxic (anti-cancer) agent enter the targeted cancer cell where the antibody is degraded into an amino acid. The resulting complex—amino acid, linker and cytotoxic agent—is considered to be the active drug. In contrast, cleavable linkers are detached by enzymes in the cancer cell.

The drug moiety (or payload) may be a small molecule or a nucleic acid drug. In some embodiments, the drug moiety (or payload), is or comprises a cytotoxic agent. In some embodiments, the drug moiety is or comprises a chemotherapeutic agent. In some embodiments, the drug moiety is or comprises an anti-arthritis drug. In some embodiments, the drug moiety is or comprises a steroid.

### Functional Properties of the Antigen-Binding Molecules

The antigen-binding molecules described herein may be characterised by reference to certain functional properties. In some embodiments, the antigen-binding molecule described herein may possess one or more of the following properties:
binds to CNX (e.g. human CNX and/or mouse CNX);
binds to CRT (e.g. human CRT);
binds cross-reactively to CNX (e.g. human CNX and/or mouse CNX) and CRT (e.g. human CRT);
reduces a function of CNX/CRT and/or a function of a complex comprising CNX/CRT;
reduces or inhibits extracellular matrix degradation (e.g. collagen and/or gelatin degradation);
reduces or inhibits extracellular matrix degradation activity of a cell characterised by CNX expression;
reduces or inhibits extracellular matrix degradation activity of a cancer cell;
reduces or inhibits extracellular matrix degradation activity of a fibroblast;
reduces or inhibits extracellular matrix degradation activity of a synovial fibroblast;
reduces or inhibits extracellular matrix degradation by a cell characterised by CNX expression;
reduces or inhibits extracellular matrix degradation by a cancer cell;
reduces or inhibits extracellular matrix degradation by a fibroblast;
reduces or inhibits extracellular matrix degradation by a synovial fibroblast;
reduces oxireductase activity;
reduces disulfide bond reductase activity;
reduces cartilage degradation;
increases killing of cells expressing CNX/CRT;
increases ADCC of cells expressing CNX/CRT;
inhibits tumor growth;
reduces or prevents metastasis of a cancer;
increases survival of subjects having a cancer; and/or
reduces the pathology of a disease/condition characterised by ECM degradation in a subject;
reduces the pathology of a disease/condition characterised by cartilage degradation (e.g. arthritis) in a subject.

It will be appreciated that a given antigen-binding molecule may display more than one of the properties recited in the preceding paragraph. A given antigen-binding molecule may be evaluated for the properties recited in the preceding paragraph using suitable assays. For example, the assays may be e.g. in vitro assays, optionally cell-based assays or cell-free assays. In some embodiments, the assays may be e.g. in vivo assays, i.e. performed in non-human animals. In some embodiments, the assays may be e.g. ex vivo assays, i.e. performed using cells/tissue/an organ obtained from a subject.

Where assays are cell-based assays, they may comprise treating cells with a given antigen-binding molecule in order to determine whether the antigen-binding molecule displays one or more of the recited properties. Assays may employ species labelled with detectable entities in order to facilitate their detection. Assays may comprise evaluating the recited properties following treatment of cells separately with a range of quantities/concentrations of a given antigen-binding molecule (e.g. a dilution series). It will be appreciated that the cells preferably express the target antigen for the antigen-binding molecule (i.e. CNX/CRT).

Analysis of the results of such assays may comprise determining the concentration at which 50% of the maximal level of the relevant activity is attained. The concentration of a given agent at which 50% of the maximal level of the relevant activity is attained may be referred to as the ‘half-maximal effective concentration’ of the agent in relation to the relevant activity, which may also be referred to as the ‘EC₅₀’. By way of illustration, the EC₅₀ of a given antigen-binding molecule for binding to human CNX may be the concentration of the antigen-binding molecule at which 50% of the maximal level of binding to human CNX is achieved.

Depending on the property, the EC₅₀ may also be referred to as the ‘half-maximal inhibitory concentration’ or ‘IC₅₀’, this being the concentration of the agent at which 50% of the maximal level of inhibition of a given property is observed.

The antigen-binding molecules described herein bind to CNX. In some embodiments, the antigen-binding molecules bind to CRT. The antigen-binding molecules and antigen-binding domains described herein preferably display specific binding to the relevant target antigen (e.g. CNX). As used herein, ‘specific binding’ refers to binding which is selective for the antigen, and which can be discriminated from non-specific binding to non-target antigen. An antigen-binding molecule/domain that specifically binds to a target molecule preferably binds the target with greater affinity, and/or with greater duration than it binds to other, non-target molecules.

The ability of a given polypeptide to bind specifically to a given molecule can be determined by analysis according to methods known in the art, such as by ELISA, Surface Plasmon Resonance (SPR; see e.g. Hearty et al., Methods Mol Biol (2012) 907:411-442), Bio-Layer Interferometry (see e.g. Lad et al., (2015) J Biomol Screen 20(4): 498-507), flow cytometry, or by a radiolabeled antigen-binding assay (RIA) enzyme-linked immunosorbent assay. Through such analysis binding to a given molecule can be measured and quantified. In some embodiments, the binding may be the response detected in a given assay.

In some embodiments, the extent of binding of the antigen-binding molecule to a non-target molecule is less than about 10% of the binding of the antibody to the target molecule as measured, e.g. by ELISA, SPR, Bio-Layer Interferometry or by RIA. Alternatively, binding specificity may be reflected in terms of binding affinity where the antigen-binding molecule binds with a dissociation constant (K_{D}) that is at least 0.1 order of magnitude (i.e. 0.1×10ⁿ, where n is an integer representing the order of magnitude) greater than the Ko of the antigen-binding molecule towards a non-target molecule. This may optionally be one of at least 0.2, 0.3, 0.4, 0.5, 0.6, 0.7, 0.8, 0.9, 1.0, 1.5, or 2.0.

The affinity of binding to a given target antigen for an antigen-binding molecule described herein may be determined by Bio-Layer Interferometry, e.g. as described in the Examples of the present disclosure.

In some embodiments, the antigen-binding molecule described herein binds to CNX with an affinity in the micromolar range, i.e. K_{D}=9.9×10⁻⁴ to 1×10⁻⁶ M. In some embodiments, the antigen-binding molecule described herein binds to CNX with sub-micromolar affinity, i.e. K_{D}<1×10⁻⁶ M. In some embodiments, the antigen-binding molecule described herein binds to CNX with an affinity in the nanomolar range, i.e. K_{D}=9.9×10⁻⁷ to 1×10⁻⁹ M. In some embodiments, the antigen-binding molecule described herein binds to CNX with sub-nanomolar affinity, i.e. K_{D}<1×10⁻⁹ M. In some embodiments, the antigen-binding molecule described herein binds to CNX with an affinity in the picomolar range, i.e. K_{D}=9.9×10⁻¹⁰ to 1×10⁻¹² M. In some embodiments, the antigen-binding molecule described herein binds to CNX with sub-picomolar affinity, i.e. K_{D}<1×10⁻¹² M.

In some embodiments, the antigen-binding molecule described herein binds to human CNX with a K_{D} of 10 μM or less, preferably one of ≤5 μM, ≤2 μM, ≤1 μM, ≤500 nM, ≤100 nM, ≤75 nM, ≤50 nM, ≤40 nM, ≤30 nM, ≤20 nM, ≤15 nM, ≤12.5 nM, ≤10 nM, ≤9 nM, ≤8 nM, ≤7 nM, ≤6 nM, ≤5 nM, ≤4 nM≤3 nM, ≤2 nM, ≤1 nM, ≤500 μM, ≤400 μM, ≤300 μM, ≤200 μM, ≤100 μM, ≤50 μM, ≤40 μM, ≤30 μM, ≤20 μM, ≤10 μM or ≤1 μM (e.g. as determined by analysis as described in Example 2 herein). In some embodiments, the antigen-binding molecule described herein binds to human CNX with a K_{D} of 100 nM or less, preferably one of ≤50 nM, ≤40 nM, ≤30 nM, ≤20 nM, ≤15 nM, ≤12.5 nM, ≤10 nM, ≤9 nM, ≤8 nM, ≤7 nM, ≤6 nM, ≤5 nM, ≤4 nM≤3 nM, ≤2 nM, ≤1 nM, ≤500 μM, ≤400 μM, ≤300 μM, ≤200 μM, ≤100 μM, ≤50 μM, ≤40 μM, ≤30 μM, ≤20 μM, ≤10 μM or ≤1 μM (e.g. as determined by analysis as described in Example 2 herein).

In some embodiments, the antigen-binding molecule described herein binds to human CNX with an EC₅₀ of 10 μM or less, preferably one of ≤5 μM, ≤2 μM, ≤1 μM, ≤500 nM, ≤100 nM, ≤75 nM, ≤50 nM, ≤40 nM, ≤30 nM, ≤20 nM, ≤15 nM, ≤12.5 nM, ≤10 nM, ≤9 nM, ≤8 nM, ≤7 nM, ≤6 nM, ≤5 nM, ≤4 nM≤3 nM, ≤2 nM, ≤1 nM, ≤500 μM, ≤400 μM, ≤300 μM, ≤200 μM, ≤100 μM, ≤50 μM, ≤40 μM, ≤30 μM, ≤20 μM, ≤10 μM or ≤1 μM (e.g. as determined by analysis as described in Example 2 herein).

In some embodiments, the antigen-binding molecule is cross-reactive for human CNX and a homologue thereof (e.g. mouse CNX). In some embodiments, the antigen-binding molecule is cross-reactive for CNX and CRT. As used herein, a ‘cross-reactive’ antigen-binding molecule/domain binds to the target antigens for which the antigen-binding molecule/domain is cross-reactive. For example, an antigen-binding molecule/domain/polypeptide which is cross-reactive for human CNX and mouse CNX binds to human CNX, and is also capable of binding to mouse CNX. Similarly, an antigen-binding molecule/domain/polypeptide which is cross-reactive for human CNX and human CRT binds to CNX, and is also capable of binding to CRT. Cross-reactive antigen-binding molecules/domains/polypeptides may display specific binding to each of the target antigens.

In some embodiments, the antigen-binding molecule binds to human CNX (e.g. isoform 1), and mouse CNX. In some embodiments, the antigen-binding molecule binds to human CNX (e.g. isoform 1) and human CRT.

The antigen-binding molecules of the present disclosure may bind to a particular region of interest of CNX. Antigen-binding molecules according to the present disclosure may bind to linear epitope of CNX, consisting of a contiguous sequence of amino acids (i.e. an amino acid primary sequence). In some embodiments, an antigen-binding molecules may bind to a conformational epitope of CNX, consisting of a discontinuous sequence of amino acids of the amino acid sequence.

The region of a given target molecule to which an antigen-binding molecule binds can be determined by the skilled person using various methods well known in the art, including X-ray co-crystallography analysis of antibody-antigen complexes, peptide scanning, mutagenesis mapping, hydrogen-deuterium exchange analysis by mass spectrometry, phage display, competition ELISA and proteolysis-based ‘protection’ methods. Such methods are described, for example, in Gershoni et al., BioDrugs, 2007, 21(3):145-156, which is hereby incorporated by reference in its entirety. In preferred embodiments, the region of a peptide/polypeptide to which an antigen-binding molecule binds is determined by hydrogen-deuterium exchange analysis by mass spectrometry, performed essentially as described in Example 2 herein.

In some embodiments, the antigen-binding molecule of the present disclosure binds to a domain of CNX described herein, e.g. the lumenal domain (e.g. lectin domain 1, P domain, lectin domain 2), transmembrane domain or cytoplasmic domain.

In some embodiments, the antigen-binding molecule of the present disclosure binds to the lumenal domain of CNX. In some embodiments, the antigen-binding molecule binds to the region of CNX shown in SEQ ID NO:337. In some embodiments, the antigen-binding molecule binds to a polypeptide comprising or consisting of the amino acid sequence shown in SEQ ID NO:337.

In some embodiments, the antigen-binding molecule of the present disclosure binds to the lectin domain of CNX. In some embodiments, the antigen-binding molecule binds to the region of CNX shown in SEQ ID NO:340. In some embodiments, the antigen-binding molecule binds to a polypeptide comprising or consisting of the amino acid sequence shown in SEQ ID NO:340.

In some embodiments, the antigen-binding molecule of the present disclosure binds to the P domain of CNX. In some embodiments, the antigen-binding molecule binds to the region of CNX shown in SEQ ID NO:341. In some embodiments, the antigen-binding molecule binds to a polypeptide comprising or consisting of the amino acid sequence shown in SEQ ID NO:341.

In some embodiments, the antigen-binding molecule binds to the region of CNX shown in SEQ ID NO:361. In some embodiments, the antigen-binding molecule contacts the region of CNX shown in SEQ ID NO:361. In some embodiments, the antigen-binding molecule binds to CNX via contact with one or more amino acids of the region shown in SEQ ID NO:361. In some embodiments, the epitope of the antigen-binding molecule comprises or consists of the amino acid sequence shown in SEQ ID NO:361. In some embodiments, the antigen-binding molecule binds to a polypeptide comprising or consisting of the amino acid sequence shown in SEQ ID NO:361.

In some embodiments, the antigen-binding molecule binds to the region of CNX shown in SEQ ID NO:362. In some embodiments, the antigen-binding molecule contacts the region of CNX shown in SEQ ID NO:362. In some embodiments, the antigen-binding molecule binds to CNX via contact with one or more amino acids of the region shown in SEQ ID NO:362. In some embodiments, the epitope of the antigen-binding molecule comprises or consists of the amino acid sequence shown in SEQ ID NO:362. In some embodiments, the antigen-binding molecule binds to a polypeptide comprising or consisting of the amino acid sequence shown in SEQ ID NO:362.

In some embodiments, the antigen-binding molecule binds to the region of CNX shown in SEQ ID NO:363. In some embodiments, the antigen-binding molecule contacts the region of CNX shown in SEQ ID NO:363. In some embodiments, the antigen-binding molecule binds to CNX via contact with one or more amino acids of the region shown in SEQ ID NO:363. In some embodiments, the epitope of the antigen-binding molecule comprises or consists of the amino acid sequence shown in SEQ ID NO:363. In some embodiments, the antigen-binding molecule binds to a polypeptide comprising or consisting of the amino acid sequence shown in SEQ ID NO:363.

In some embodiments, the antigen-binding molecule binds to the region of CNX shown in SEQ ID NO:364. In some embodiments, the antigen-binding molecule contacts the region of CNX shown in SEQ ID NO:364. In some embodiments, the antigen-binding molecule binds to CNX via contact with one or more amino acids of the region shown in SEQ ID NO:364. In some embodiments, the epitope of the antigen-binding molecule comprises or consists of the amino acid sequence shown in SEQ ID NO:364. In some embodiments, the antigen-binding molecule binds to a polypeptide comprising or consisting of the amino acid sequence shown in SEQ ID NO:364.

In some embodiments, the antigen-binding molecule binds to the region of CNX shown in SEQ ID NO:365. In some embodiments, the antigen-binding molecule contacts the region of CNX shown in SEQ ID NO:365. In some embodiments, the antigen-binding molecule binds to CNX via contact with one or more amino acids of the region shown in SEQ ID NO:365. In some embodiments, the epitope of the antigen-binding molecule comprises or consists of the amino acid sequence shown in SEQ ID NO:365. In some embodiments, the antigen-binding molecule binds to a polypeptide comprising or consisting of the amino acid sequence shown in SEQ ID NO:365.

In some embodiments, the antigen-binding molecule binds to the region of CNX shown in SEQ ID NO:366. In some embodiments, the antigen-binding molecule contacts the region of CNX shown in SEQ ID NO:366. In some embodiments, the antigen-binding molecule binds to CNX via contact with one or more amino acids of the region shown in SEQ ID NO:366. In some embodiments, the epitope of the antigen-binding molecule comprises or consists of the amino acid sequence shown in SEQ ID NO:366. In some embodiments, the antigen-binding molecule binds to a polypeptide comprising or consisting of the amino acid sequence shown in SEQ ID NO:366.

In some embodiments, the antigen-binding molecule binds to the region of CNX shown in SEQ ID NO:367. In some embodiments, the antigen-binding molecule contacts the region of CNX shown in SEQ ID NO:367. In some embodiments, the antigen-binding molecule binds to CNX via contact with one or more amino acids of the region shown in SEQ ID NO:367. In some embodiments, the epitope of the antigen-binding molecule comprises or consists of the amino acid sequence shown in SEQ ID NO:367. In some embodiments, the antigen-binding molecule binds to a polypeptide comprising or consisting of the amino acid sequence shown in SEQ ID NO:367.

In some embodiments, the antigen-binding molecule binds to the region of CNX shown in SEQ ID NO:368. In some embodiments, the antigen-binding molecule contacts the region of CNX shown in SEQ ID NO:368. In some embodiments, the antigen-binding molecule binds to CNX via contact with one or more amino acids of the region shown in SEQ ID NO:368. In some embodiments, the epitope of the antigen-binding molecule comprises or consists of the amino acid sequence shown in SEQ ID NO:368. In some embodiments, the antigen-binding molecule binds to a polypeptide comprising or consisting of the amino acid sequence shown in SEQ ID NO:368.

In some embodiments, the antigen-binding molecule binds to the region of CNX shown in SEQ ID NO:369. In some embodiments, the antigen-binding molecule contacts the region of CNX shown in SEQ ID NO:369. In some embodiments, the antigen-binding molecule binds to CNX via contact with one or more amino acids of the region shown in SEQ ID NO:369. In some embodiments, the epitope of the antigen-binding molecule comprises or consists of the amino acid sequence shown in SEQ ID NO:369. In some embodiments, the antigen-binding molecule binds to a polypeptide comprising or consisting of the amino acid sequence shown in SEQ ID NO:369.

In some embodiments, the antigen-binding molecule binds to the region of CNX shown in SEQ ID NO:370. In some embodiments, the antigen-binding molecule contacts the region of CNX shown in SEQ ID NO:370. In some embodiments, the antigen-binding molecule binds to CNX via contact with one or more amino acids of the region shown in SEQ ID NO:370. In some embodiments, the epitope of the antigen-binding molecule comprises or consists of the amino acid sequence shown in SEQ ID NO:370. In some embodiments, the antigen-binding molecule binds to a polypeptide comprising or consisting of the amino acid sequence shown in SEQ ID NO:370.

In some embodiments, the antigen-binding molecule binds to the region of CNX shown in SEQ ID NO:371. In some embodiments, the antigen-binding molecule contacts the region of CNX shown in SEQ ID NO:371. In some embodiments, the antigen-binding molecule binds to CNX via contact with one or more amino acids of the region shown in SEQ ID NO:371. In some embodiments, the epitope of the antigen-binding molecule comprises or consists of the amino acid sequence shown in SEQ ID NO:371. In some embodiments, the antigen-binding molecule binds to a polypeptide comprising or consisting of the amino acid sequence shown in SEQ ID NO:371.

In some embodiments, the antigen-binding molecule binds to the region of CNX shown in SEQ ID NO:372. In some embodiments, the antigen-binding molecule contacts the region of CNX shown in SEQ ID NO:372. In some embodiments, the antigen-binding molecule binds to CNX via contact with one or more amino acids of the region shown in SEQ ID NO:372. In some embodiments, the epitope of the antigen-binding molecule comprises or consists of the amino acid sequence shown in SEQ ID NO:372. In some embodiments, the antigen-binding molecule binds to a polypeptide comprising or consisting of the amino acid sequence shown in SEQ ID NO:372.

In some embodiments, the antigen-binding molecule binds to the region of CNX shown in SEQ ID NO:373. In some embodiments, the antigen-binding molecule contacts the region of CNX shown in SEQ ID NO:373. In some embodiments, the antigen-binding molecule binds to CNX via contact with one or more amino acids of the region shown in SEQ ID NO:373. In some embodiments, the epitope of the antigen-binding molecule comprises or consists of the amino acid sequence shown in SEQ ID NO:373. In some embodiments, the antigen-binding molecule binds to a polypeptide comprising or consisting of the amino acid sequence shown in SEQ ID NO:373.

The ability of an antigen-binding molecule to bind to a given peptide/polypeptide can be analysed by methods well known to the skilled person, including analysis by ELISA, immunoblot (e.g. western blot), immunoprecipitation, surface plasmon resonance and biolayer interferometry.

In some embodiments, the antigen-binding molecule is capable of binding the same region of CNX, or an overlapping region of CNX, to the region of CNX which is bound by an antibody comprising the VH and VL regions (see e.g. Table C) of one of clones 1D3, 1D6, 1E1, 1E6, 2C6, 2H6, 3D1, 2G9, 2G12, 2H5, 3F8, 3F9, 4G9, 5A3, 5E8, 0001, 0008, C010, C023, C025, C040, C046 and C117. In some embodiments, the antigen-binding molecule is capable of binding the same region of CNX, or an overlapping region of CNX, to the region of CNX which is bound by an antibody comprising the VH and VL regions of 0008 or 1E1.

Whether a test antigen-binding molecule binds to the same or an overlapping region of a given target as a reference antigen-binding molecule can be evaluated, for example, by analysis of (i) interaction between the test antigen-binding molecule and the target in the absence of the reference binding molecule, and (ii) interaction between the test antigen-binding molecule in the presence of the reference antigen-binding molecule, or following incubation of the target with the reference antigen-binding molecule. Determination of a reduced level of interaction between the test antigen-binding molecule and the target following analysis according to (ii) as compared to (i) might support an inference that the test and reference antigen-binding molecule bind to the same or an overlapping region of the target. Suitable assays for such analysis include e.g. competition ELISA assays and epitope binning assays.

In some embodiments, the antigen-binding molecule is an antagonist of CNX, CRT and/or an antagonist of a complex comprising CNX or CRT. In some embodiments, the antigen-binding molecule is capable of inhibiting a function or process mediated by CNX and/or CRT, or mediated by complexes comprising CNX/CRT. In some embodiments, the antigen-binding molecule is capable of inhibiting a function or process mediated by a polypeptide complex comprising CNX or CRT. Herein, ‘inhibition’ refers to a reduction, decrease or lessening relative to a control condition. Suitable assays for investigating the function of CNX and/or CRT, and of complexes comprising CNX/CRT are well known to the skilled person.

In some embodiments, a complex comprising CNX may be selected from: a CNX:ERp57 complex, a CNX:ERp29 complex and a CNX:CypB complex. In some embodiments, a complex comprising CNX may comprise CNX and a glycopolypeptide. In some embodiments, a complex comprising CRT may be selected from: a CRT:ERp57 complex, a CRT:ERp29 complex and a CRT:CypB complex. In some embodiments, a complex comprising CRT may comprise CRT and a glycopolypeptide.

In preferred embodiments, a complex comprising CNX is a CNX:ERp57 complex. In preferred embodiments, a complex comprising CRT is a CRT:ERp57 complex.

Assays for the identification of antigen-binding molecules capable of reducing/inhibiting a function of CNX/CRT and/or of complexes comprising CNX/CRT may comprise treating cells/tissue expressing CNX/CRT and/or a complex comprising CNX/CRT with a test antigen-binding molecule, and subsequently comparing the level of the relevant function to the level observed in an appropriate control condition (e.g. untreated/vehicle-treated/control-treated cells/tissue).

Antigen-binding molecules capable of reducing/inhibiting a function of CNX/CRT, and/or of a complex comprising CNX/CRT, may be identified using assays comprising detecting the level of a correlate of a function of CNX/CRT, and/or of a complex comprising CNX/CRT, (e.g. the gene and/or protein expression, and/or activity, of one or more proteins whose expression is directly/indirectly upregulated or downregulated as a consequence of a function of CNX/CRT and/or a complex comprising CNX/CRT). Such assays may comprise treating cells/tissue expressing CNX/CRT and/or a complex comprising CNX/CRT with the antigen-binding molecule, and subsequently (e.g. after an appropriate period of time, i.e. a period of time sufficient for the functional consequences of an activity of CNX/CRT and/or a complex comprising CNX/CRT to be observed) comparing the level of the correlate of a function of CNX/CRT, and/or of a complex comprising CNX/CRT, in such cells/tissue to the level of the correlate of the relevant function in an appropriate control condition (e.g. untreated/vehicle-treated/control-treated cells/tissue).

In some embodiments, the antigen-binding molecule of the present disclosure is capable of reducing/inhibiting a function of CNX/CRT, or of a complex comprising CNX/CRT to less than 1 times, e.g. ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or ≤0.01 times the level of the relevant function observed in the absence of the antigen-binding molecule (or in the presence of an appropriate control antigen-binding molecule), in a given assay.

In some embodiments, the antigen-binding molecule is able to inhibit a function of CNX/CRT, and/or of a complex comprising CNX/CRT, by a mechanism not requiring or involving Fc-mediated function. In some embodiments, the antigen-binding molecule is able to inhibit a function of CNX/CRT, and/or of a complex comprising CNX/CRT, independently of Fc-mediated function. That is, in some embodiments, the antigen-binding molecule is able to inhibit a function of CNX/CRT, and/or of a complex comprising CNX/CRT, in an Fc region-independent manner.

The ability of an antigen-binding molecule to inhibit a function of CNX/CRT, and/or of a complex comprising CNX/CRT, by a mechanism not requiring/involving Fc-mediated function can be evaluated e.g. by analysing the ability of the antigen-binding molecule provided in a format lacking a functional Fc region to inhibit a function of CNX/CRT and/or a complex comprising CNX/CRT. For example, the effect on a function of CNX/CRT, and/or of a complex comprising CNX/CRT, can be investigated using an antigen-binding molecule comprising a ‘silent’ Fc region (e.g. comprising LALA PG substitutions), or using an antigen-binding molecule provided in a format lacking an Fc region (e.g. scFv, Fab etc.).

In some embodiments, the antigen-binding molecule is able to inhibit a function of CNX/CRT, and/or of a complex comprising CNX/CRT, by a mechanism not involving ADCC. In some embodiments, the antigen-binding molecule is able to inhibit a function of CNX/CRT, and/or of a complex comprising CNX/CRT, by a mechanism not involving ADCP. In some embodiments, the antigen-binding molecule is able to inhibit a function of CNX/CRT, and/or of a complex comprising CNX/CRT, by a mechanism not involving CDC.

In some embodiments, the antigen-binding molecule is able to inhibit a function of CNX/CRT, and/or of a complex comprising CNX/CRT, by a mechanism not requiring binding of the antigen-binding molecule to an Fc receptor. In some embodiments, the antigen-binding molecule is able to inhibit a function of CNX/CRT, and/or of a complex comprising CNX/CRT, by a mechanism not requiring binding of the antigen-binding molecule to an Fcγ receptor. In some embodiments, the antigen-binding molecule is able to inhibit a function of CNX/CRT, and/or of a complex comprising CNX/CRT, by a mechanism not requiring binding of the antigen-binding molecule to one or more of FcγRI, FcγRIIa, FcγRIIb, FcγRIIc, FcγRIIIa and FcγRIIIb. In some embodiments, the antigen-binding molecule is able to inhibit a function of CNX/CRT, and/or of a complex comprising CNX/CRT, by a mechanism not requiring binding to FcγRIIIa. In some embodiments, the antigen-binding molecule is able to inhibit a function of CNX/CRT, and/or of a complex comprising CNX/CRT, by a mechanism not requiring binding to FcγRIIa. In some embodiments, the antigen-binding molecule is able to inhibit a function of CNX/CRT, and/or of a complex comprising CNX/CRT, by a mechanism not requiring binding to FcγRIIb. In some embodiments, the antigen-binding molecule is able to inhibit a function of CNX/CRT, and/or of a complex comprising CNX/CRT, by a mechanism not requiring binding to a complement protein. In some embodiments, the antigen-binding molecule is able to inhibit a function of CNX/CRT, and/or of a complex comprising CNX/CRT, by a mechanism not requiring binding to C1q. In some embodiments, the antigen-binding molecule is able to inhibit a function of CNX/CRT, and/or of a complex comprising CNX/CRT, by a mechanism not requiring N297 glycosylation.

It will be appreciated that in some embodiments, the antigen-binding molecule of the present disclosure achieves is functional effects via a mechanism not involving Fc-mediated function. In some embodiments, the antigen-binding molecule of the present disclosure achieves is functional effects via a mechanism not involving killing/depletion of cells expressing CNX/CRT, or of cells expressing complexes comprising CNX/CRT, e.g. Fc-mediated killing/depletion of such cells.

In some embodiments, a function of CNX/CRT, or a function of a complex comprising CNX/CRT, may be selected from: extracellular matrix (ECM) degradation, collagen degradation, gelatin degradation, oxireductase activity and disulfide bond reductase activity. A correlate of a function of CNX/CRT, or of a complex comprising CNX/CRT, may e.g. be a product of ECM/collagen/gelatin degradation, or oxireductase/disulfide bond reductase activity.

In some embodiments, the antigen-binding molecule reduces/inhibits extracellular matrix (ECM) degradation. In some embodiments, the antigen-binding molecule reduces/inhibits collagen degradation. In some embodiments, the antigen-binding molecule reduces/inhibits gelatin degradation. In some embodiments, the antigen-binding molecule reduces/inhibits oxireductase activity. In some embodiments, the antigen-binding molecule reduces/inhibits disulfide bond reductase activity. In some embodiments, the antigen-binding molecule reduces/inhibits ECM degradation mediated by CNX/CRT or a complex comprising CNX/CRT (e.g. a CNX/CRT:ERp57 complex). In some embodiments, the antigen-binding molecule reduces/inhibits collagen degradation mediated by CNX/CRT or a complex comprising CNX/CRT (e.g. a CNX/CRT:ERp57 complex). In some embodiments, the antigen-binding molecule reduces/inhibits gelatin degradation mediated by CNX/CRT or a complex comprising CNX/CRT (e.g. a CNX/CRT:ERp57 complex). In some embodiments, the antigen-binding molecule reduces/inhibits oxireductase activity mediated by CNX/CRT or a complex comprising CNX/CRT (e.g. a CNX/CRT:ERp57 complex). In some embodiments, the antigen-binding molecule reduces/inhibits disulfide bond reductase activity mediated by CNX/CRT or a complex comprising CNX/CRT (e.g. a CNX/CRT:ERp57 complex).

The ability of an antigen-binding molecule to inhibit ECM/collagen/gelatin degradation can be determined for example by analysis of ECM/collagen/gelatin degradation in the presence of, or following incubation with, the antigen-binding molecule. An antigen-binding molecule which is capable of inhibiting ECM/collagen/gelatin degradation is identified by the observation of a reduction/decrease in the level of ECM/collagen/gelatin degradation in the presence of—or following incubation with—the antigen-binding molecule, as compared to the level of ECM/collagen/gelatin degradation in the absence of the antigen-binding molecule (or in the presence of an appropriate control antigen-binding molecule).

Antigen-binding molecules capable of reducing/inhibiting ECM/collagen/gelatin degradation (e.g. by CNX/CRT and/or a complex comprising CNX/CRT) may be identified using assays comprising detecting the level of ECM/collagen/gelatin, or the level of a correlate of ECM/collagen/gelatin degradation (e.g. a product of degraded ECM/collagen/gelatin), e.g. using antibody/reporter-based methods. Collagen/gelatin degradation assays are described e.g. in Hollander, Methods Mol. Biol. (2010) 622:367-78 and Vandooren et al., World J. Biol. Chem. (2011) 2(1): 14-24. In preferred embodiments, ECM/collagen/gelatin degradation can be evaluated in an assay performed essentially as described in Example 4 herein.

For example, a commercial solution of gelatin (2%) can be labeled with 5-Carboxy-X-Rhodamine, Succinimidyl Ester. The labeled gelatin can then be transferred onto sterile coverslips to create a thin layer, and stabilised by glutaraldehyde fixation. A solution of rat tail collagen can be used to coat the coverslips, creating a thin layer of collagen on top of the gelatin. The coverslips can then be transferred in culture vessels and cells with the appropriate degradative activity (e.g. human hepatocellular carcinoma Huh7 cells) can be seeded on the coverslips in the presence of test antigen-binding molecules, and incubated for 48 h to allow degradation to occur. The coverslips can then be fixed, and subsequently stained with Hoescht to permit the counting of cells, and then analysed by confocal microscopy. The images acquired can be analysed using ImageJ to determine the surface of degraded gelatin and the total area per field. In parallel, the number of nuclei can be calculated and the final result can be normalised to the number of cells in each field.

For example, a mixture of rat tail collagen and quenched fluorescent DQ collagen type I can be coated and polymerised on the bottom of a 384 well optical grade plate. Cells of the 3t3-vSrc mouse cell line can be seeded on top of the collagen layer in the presence of test antigen-binding molecules, and incubated for 48 h to 72h. The fluorescent area of DQ signal from live cells can subsequently be evaluated by high content imaging, and normalised by nucleus count to determine the degraded area/cell.

In some embodiments, the antigen-binding molecule of the present disclosure is capable of reducing/inhibiting ECM degradation, collagen degradation or gelatin degradation to less than 1 times, e.g. ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or ≤0.01 times the level of ECM degradation/collagen degradation/gelatin degradation observed in the absence of the antigen-binding molecule (or in the presence of an appropriate control antigen-binding molecule), in a given assay.

The ability of an antigen-binding molecule to inhibit oxireductase activity can be determined for example by analysis of oxireductase activity in the presence of, or following incubation with, the antigen-binding molecule. An antigen-binding molecule which is capable of inhibiting oxireductase activity is identified by the observation of a reduction/decrease in the level oxireductase activity in the presence of—or following incubation with—the antigen-binding molecule, as compared to the level of oxireductase activity in the absence of the antigen-binding molecule (or in the presence of an appropriate control antigen-binding molecule).

Oxireductase activity can be evaluated using any one of a number of methods known to the person skilled in the art. For example, oxireductase activity can be evaluated in an insulin reduction assay, e.g. as described in Hirano et al., Eur J Biochem. (1995) 234(1):336-42.

In some embodiments, the antigen-binding molecule of the present disclosure is capable of reducing/inhibiting oxireductase activity to less than 1 times, e.g. ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or ≤0.01 times the level of oxireductase activity observed in the absence of the antigen-binding molecule (or in the presence of an appropriate control antigen-binding molecule), in a given assay.

The ability of an antigen-binding molecule to inhibit disulfide bond reductase activity can be determined for example by analysis of disulfide bond reductase activity in the presence of, or following incubation with, the antigen-binding molecule. An antigen-binding molecule which is capable of inhibiting disulfide bond reductase activity is identified by the observation of a reduction/decrease in the level disulfide bond reductase activity in the presence of—or following incubation with—the antigen-binding molecule, as compared to the level of disulfide bond reductase activity in the absence of the antigen-binding molecule (or in the presence of an appropriate control antigen-binding molecule).

Disulfide bond reductase activity can be evaluated using any one of a number of methods known to the person skilled in the art. For example, disulfide bond reductase assays may employ antibodies for detecting reduced disulfide bonds in proteins, e.g. antibody clone OX133, which recognizes polypeptide resident, N-ethylmaleimide (NEM)-modified cysteine residues (see Holbrook et al., Mabs (2016) 8(4): 672-677).

In some embodiments, the antigen-binding molecule of the present disclosure is capable of reducing/inhibiting disulfide bond reductase activity to less than 1 times, e.g. ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or ≤0.01 times the level of disulfide bond reductase activity observed in the absence of the antigen-binding molecule (or in the presence of an appropriate control antigen-binding molecule), in a given assay.

In some embodiments, an antigen-binding molecule according to the present disclosure reduces/inhibits cartilage degradation. Antigen-binding molecules capable of reducing/inhibiting cartilage degradation (e.g. by CNX/CRT and/or a complex comprising CNX/CRT) may be identified using assays comprising detecting the level of cartilage, or the level of a correlate of cartilage degradation (e.g. a product of degraded cartilage), e.g. using antibody/reporter-based methods. Cartilage degradation can be evaluated essentially as described in Example 6 herein. An ex vivo assay of cartilage degradation is also described e.g. in Neidlin et al., PLoS One (2019) 14(10):e0224231.

In some embodiments, the antigen-binding molecule of the present disclosure is capable of reducing/inhibiting cartilage degradation to less than 1 times, e.g. ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or ≤0.01 times the level of cartilage degradation observed in the absence of the antigen-binding molecule (or in the presence of an appropriate control antigen-binding molecule), in a given assay.

In some embodiments, an antigen-binding molecule according to the present disclosure may potentiate (i.e. upregulate, enhance) cell killing of cells comprising/expressing CNX/CRT, or a complex comprising CNX/CRT. In some embodiments, an antigen-binding molecule according to the present disclosure may inhibit growth or reduce metastasis of a cancer comprising cells comprising/expressing CNX/CRT, or a complex comprising CNX/CRT. In some embodiments, an antigen-binding molecule according to the present disclosure may potentiate (i.e. upregulate, enhance) cell killing of cells comprising/expressing CNX/CRT, or a complex comprising CNX/CRT. In some embodiments, an antigen-binding molecule according to the present disclosure may inhibit growth or reduce metastasis of a cancer comprising cells comprising/expressing CNX/CRT, or a complex comprising CNX/CRT.

Cell killing can be investigated, for example, using any of the methods reviewed in Zaritskaya et al., Expert Rev Vaccines (2011), 9(6):601-616, hereby incorporated by reference in its entirety. Examples of in vitro assays of cytotoxicity/cell killing assays include release assays such as the ⁵¹Cr release assay, the lactate dehydrogenase (LDH) release assay, the 3-(4,5-dimethylthiazol-2-yl)-2,5-diphenyl tetrazolium bromide (MTT) release assay, and the calcein-acetoxymethyl (calcein-AM) release assay. These assays measure cell killing based on the detection of factors released from lysed cells. Cell killing of a given test cell type by a given effector immune cell type can be analysed e.g. by co-culturing the test cells with the effector immune cells, and measuring the number/proportion of viable/dead (e.g. lysed) test cells after a suitable period of time. Other suitable assays include the xCELLigence real-time cytolytic in vitro potency assay described in Cerignoli et al., PLoS One. (2018) 13(3): e0193498 (hereby incorporated by reference in its entirety). An increase in resistance to cell killing by granzyme B-expressing cells (e.g. effector immune cells), and/or a reduction in susceptibility to cell killing by such cells, relative to a reference level of cell killing (e.g. for that cell type) can be determined by detection of a reduction in the number/proportion of dead (e.g. lysed) test cells, and/or an increase in the number/proportion of live (e.g. viable, non-lysed) test cells, after a given period of time.

In some embodiments an antigen-binding molecule according to the present disclosure is capable of reducing the number/proportion of cells expressing CNX/CRT, or a complex comprising CNX/CRT. In some embodiments an antigen-binding molecule according to the present disclosure is capable of reducing the number/proportion of cells expressing CNX/CRT, or a complex comprising CNX/CRT. In some embodiments, an antigen-binding molecule according to the present disclosure is capable of depleting/enhancing depletion of such cells.

Antigen-binding molecules according to the present disclosure may comprise one or more moieties for potentiating a reduction in the number/proportion of cells expressing CNX/CRT, or a complex comprising CNX/CRT. For example, an antigen-binding molecule according to the present disclosure may e.g. comprise an Fc region and/or a drug moiety.

Fc regions provide for interaction with Fc receptors and other molecules of the immune system to bring about functional effects. IgG Fc-mediated effector functions are reviewed e.g. in Jefferis et al., Immunol Rev 1998 163:59-76 (hereby incorporated by reference in its entirety), and are brought about through Fc-mediated recruitment and activation of immune cells (e.g. macrophages, dendritic cells, neutrophils, basophils, eosinophils, platelets, mast cells, NK cells and T cells) through interaction between the Fc region and Fc receptors expressed by the immune cells, recruitment of complement pathway components through binding of the Fc region to complement protein C1q, and consequent activation of the complement cascade. Fc-mediated functions include Fc receptor binding, antibody-dependent cellular cytotoxicity (ADCC), antibody-dependent cell-mediated phagocytosis (ADCP), complement-dependent cytotoxicity (CDC), formation of the membrane attack complex (MAC), cell degranulation, cytokine and/or chemokine production, and antigen processing and presentation.

In some embodiments, an antigen-binding molecule according to the present disclosure comprises an Fc region capable of potentiating/directing one or more of ADCC, ADCP, CDC against, and/or potentiating formation of a MAC on or cell degranulation of, a cell expressing CNX/CRT, or a complex comprising CNX/CRT (e.g. a cell expressing CNX/CRT, or a complex comprising CNX/CRT at the cell surface).

In some embodiments, an antigen-binding molecule according to the present disclosure is capable of potentiating/directing ADCC against a cell expressing CNX/CRT, or a complex comprising CNX/CRT.

The ability of, and extent to which, a given antigen-binding molecule is able to induce ADCC of a given target cell type can be analysed e.g. according to the method described in Yamashita et al., Scientific Reports (2016) 6:19772 (hereby incorporated by reference in its entirety), or by ⁵¹Cr release assay as described e.g. in Jedema et al., Blood (2004) 103: 2677-82 (hereby incorporated by reference in its entirety). The ability of, and extent to which, a given antigen-binding molecule is able to induce ADCP can be analysed e.g. according to the method described in Kamen et al., J Immunol (2017) 198 (1 Supplement) 157.17 (hereby incorporated by reference in its entirety). The ability of, and extent to which, a given antigen-binding molecule is able to induce CDC can be analysed e.g. using a C1q binding assay, e.g. as described in Schlothauer et al., Protein Engineering, Design and Selection (2016), 29(10):457-466 (hereby incorporated by reference in its entirety).

In some embodiments, the antigen-binding molecule of the present disclosure does not induce ADCC of cells expressing CNX/CRT, or complexes comprising CNX/CRT, at the cell surface. In some embodiments, the antigen-binding molecule does not induce ADCP of cells expressing CNX/CRT, or complexes comprising CNX/CRT, at the cell surface. In some embodiments, the antigen-binding molecule does not induce CDC of cells expressing CNX/CRT, or complexes comprising CNX/CRT, at the cell surface. In some embodiments, the antigen-binding molecule does not induce ADCC, ADCP or CDC of cells expressing CNX/CRT, or complexes comprising CNX/CRT, at the cell surface.

Antigen-binding molecules which do not induce (i.e. are not able to induce) ADCC/ADCP/CDC elicit substantially no ADCC/ADCP/CDC activity against the relevant cell type, e.g. as determined by analysis in an appropriate assay for the relevant activity. “Substantially no ADCC/ADCP/CDC activity” refers to a level of ADCC/ADCP/CDC that is not significantly greater than ADCC/ADCP/CDC determined for an appropriate negative control molecule in a given assay (e.g. an antigen-binding molecule lacking an Fc region, or an antigen-binding molecule comprising a ‘silent’ Fc region (e.g. as described in Schlothauer et al., Protein Engineering, Design and Selection (2016), 29(10):457-466, which is incorporated by reference hereinabove)). “Substantially no activity” may be a level of the relevant activity which is ≤5 times, e.g. ≤4 times, ≤3 times, ≤2.5 times, ≤2 times or ≤1.5 times the level of activity determined for an appropriate negative control molecule in a given assay.

In some embodiments, an antigen-binding molecule according to the present disclosure comprises a drug moiety. The antigen-binding molecule may be conjugated to the drug moiety. Antibody-drug conjugates are reviewed e.g. in Parslow et al., Biomedicines. 2016 September; 4(3):14 (hereby incorporated by reference in its entirety). In some embodiments, the drug moiety is or comprises a cytotoxic agent, such that the antigen-binding molecule displays cytotoxicity to a cell expressing CNX/CRT, or a complex comprising CNX/CRT (e.g. a cell expressing CNX/CRT, or a complex comprising CNX/CRT at the cell surface). In some embodiments, the drug moiety is or comprises a chemotherapeutic agent.

In some embodiments, an antigen-binding molecule according to the present disclosure comprises an immune cell-engaging moiety. In some embodiments, the antigen-binding molecule comprises a CD3 polypeptide-binding moiety (e.g. an antigen-binding domain capable of binding to a CD3 polypeptide).

In some embodiments, an antigen-binding molecule according to the present disclosure is capable of potentiating/directing T cell-mediated cytolytic activity against a cell expressing CNX/CRT, or a complex comprising CNX/CRT.

In some embodiments, the antigen-binding molecule of the present disclosure displays anticancer activity. In some embodiments, the antigen-binding molecule of the present disclosure increases killing of cancer cells. In some embodiments, the antigen-binding molecule of the present disclosure causes a reduction in the number of cancer cells in vivo, e.g. as compared to an appropriate control condition. The cancer may be a cancer expressing CNX/CRT, or a complex comprising CNX/CRT.

In some embodiments, an antigen-binding molecule according to the present disclosure reduces/inhibits growth of a cancer and/or of a tumor of a cancer. In some embodiments, an antigen-binding molecule reduces tissue invasion by cells of a cancer. In some embodiments, an antigen-binding molecule reduces metastasis of a cancer. In some embodiments, the antigen-binding molecule displays anticancer activity. In some embodiments, the antigen-binding molecule reduces the growth/proliferation of cancer cells. In some embodiments, the antigen-binding molecule reduces the survival of cancer cells. In some embodiments, the antigen-binding molecule increases the killing of cancer cells. In some embodiments, the antigen-binding molecule of the present disclosure causes a reduction in the number of cancer cells e.g. in vivo. The cancer may be a cancer comprising cells expressing CNX and/or CRT.

The antigen-binding molecule of the present disclosure may be analysed for the properties described in the preceding paragraph in appropriate assays. Such assays include e.g. in vivo models, e.g. performed essentially as described in Example 5 herein.

In some embodiments, administration of an antigen-binding molecule according to the present disclosure may cause one or more of: inhibition of the development/progression of the cancer, a delay to/prevention of onset of the cancer, a reduction in/delay to/prevention of tumor growth, a reduction in/delay to/prevention of tissue invasion, a reduction in/delay to/prevention of metastasis, a reduction in the severity of the symptoms of the cancer, a reduction in the number of cancer cells, a reduction in tumour size/volume, and/or an increase in survival (e.g. progression free survival or overall survival), e.g. as determined in an appropriate model.

In some embodiments, the antigen-binding molecule of the present disclosure is capable of reducing/inhibiting tumor growth (e.g. in an in vivo model, e.g. of liver cancer) to less than 1 times, e.g. ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or ≤0.01 times the tumor growth observed in the absence of treatment with the antigen-binding molecule (or following treatment with an appropriate control antigen-binding molecule known not to influence tumor growth), in a given assay.

In some embodiments, the antigen-binding molecule of the present disclosure is capable of reducing/inhibiting metastasis (e.g. in an in vivo model, e.g. of metastasis of breast cancer to the lung) to less than 1 times, e.g. ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or ≤0.01 times the level of metastasis observed in the absence of treatment with the antigen-binding molecule (or following treatment with an appropriate control antigen-binding molecule known not to influence metastasis), in a given assay.

In some embodiments, the antigen-binding molecule of the present disclosure is capable of increasing survival of subjects having a cancer (e.g. in an in vivo model, e.g. of liver cancer or breast cancer) to more than 1 times, e.g. one of 1.01 times, ≥1.02 times, ≥1.03 times, ≥1.04 times, 1.05 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, ≥3 times, ≥4 times, ≥5 times, ≥6 times, ≥7 times, ≥8 times, ≥9 times or 10 times the level of survival observed in the absence of treatment with the antigen-binding molecule (or following treatment with an appropriate control antigen-binding molecule known not to influence survival), in a given assay.

In some embodiments, an antigen-binding molecule according to the present disclosure reduces/inhibits pathology of a disease/condition characterised by ECM degradation in a subject.

In some embodiments, an antigen-binding molecule according to the present disclosure reduces/inhibits pathology of a disease/condition characterised by cartilage degradation (e.g. arthritis) in a subject. In some embodiments, an antigen-binding molecule according to the present disclosure reduces arthritis score in a subject having arthritis. Arthritis pathology may be evaluated in assays performed in appropriate in vivo models, which are well known to the skilled person. Such models include the mouse collagen antibody-induced arthritis (CAIA) model described e.g. in Khachigian, Nat Protoc. (2006) 1(5):2512-6, and such assays may be performed essentially as described in Example 5 or Example 6 herein. In some embodiments, subjects treated with an antigen-binding molecule according to the present disclosure are determined to have a lower arthritis score (e.g. on day 7, 8, 9 or 10) compared to subjects not treated with antigen-binding molecule (or compared to subjects treated with an appropriate control antigen-binding molecule known not to influence arthritis pathology).

In some embodiments, the antigen-binding molecule of the present disclosure is capable of reducing/inhibiting pathology of a disease/condition characterised by ECM degradation or cartilage degradation (e.g. arthritis) in a subject (e.g. in a CAIA model, e.g. as determined by arthritis score) to less than 1 times, e.g. ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or ≤0.01 times the level of observed in the absence of treatment with the antigen-binding molecule (or following treatment with an appropriate control antigen-binding molecule known not to influence pathology of the disease/condition), in a given assay.

The antigen-binding molecules of the present disclosure preferably possess novel and/or improved properties compared to known antigen-binding molecules that bind to CNX. Known antibodies to CNX include monoclonal antibody clone AF18 (Invitrogen Cat. No. MA3-027), clone AF8 (Merck Cat. No. MABF2067), clone TO-5 (Merck Cat. No. C7617), clone 3H4A7 (Invitrogen Cat. No. MA5-15389), clone ARC0648 (Invitrogen Cat. No. MA5-35588), clone GT1563 (GeneTex Cat. No. GTX629976), clone CANX/1541 (GeneTex Cat. No. GTX34446), clone IE2.1C12 (Novus Biologicals Cat No. NBP2-36571), clone 1C2.2D11 (Novus Biologicals Cat No. NBP2-36570SS), clone 2A2C6 (Proteintech Cat. No. 66903-1-Ig) clone C5C9 (Cell Signaling Technology, Inc Cat. No. 2679), clone E-10 (Santa Cruz Biotechnology Cat No. sc-46669), polyclonal antibodies ab10286 and ab22595 (Abcam), and anti-CNX antibodies disclosed in CN 101659702 A (e.g. the antibody produced by hybridoma CGMCC No. 3240). In some embodiments, a known antibody to CNX is polyclonal antibody ab10286.

In some embodiments, an antigen-binding molecule according to the present disclosure:
binds to CNX (e.g. human CNX and/or mouse CNX) with greater affinity than a known antibody to CNX;
binds to CRT (e.g. human CRT) with greater affinity than a known antibody to CNX;
reduces a function of CNX/CRT and/or a function of a complex comprising CNX/CRT with greater potency/to a greater extent than a known antibody to CNX;
reduces extracellular matrix degradation (e.g. collagen and/or gelatin degradation) with greater potency/to a greater extent than a known antibody to CNX;
reduces an oxireductase activity with greater potency/to a greater extent than a known antibody to CNX;
reduces a disulfide bond reductase activity with greater potency/to a greater extent than a known antibody to CNX;
reduces cartilage degradation with greater potency/to a greater extent than a known antibody to CNX;
increases killing of cells expressing CNX/CRT with greater potency/to a greater extent than a known antibody to CNX;
increases ADCC of cells expressing CNX/CRT with greater potency/to a greater extent than a known antibody to CNX;
inhibits tumor growth with greater potency/to a greater extent than a known antibody to CNX;
reduces metastasis of a cancer with greater potency/to a greater extent than a known antibody to CNX;
increases survival of subjects having a cancer to a greater extent than a known antibody to CNX; and/or
reduces the pathology of a disease/condition characterised by ECM degradation in a subject to a greater extent than a known antibody to CNX.
reduces the pathology of a disease/condition characterised by cartilage degradation (e.g. arthritis) in a subject to a greater extent than a known antibody to CNX.

In some embodiments, an antigen-binding molecule according to the present disclosure binds to CNX (e.g. human CNX and/or mouse CNX) with an EC₅₀ which is less than 1 times, e.g. one of ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or ≤0.01 the EC₅₀ of a known antibody to CNX, as determined in a given assay.

In some embodiments, an antigen-binding molecule according to the present disclosure binds to CNX (e.g. human CNX and/or mouse CNX) with a Ko which is less than 1 times, e.g. one of ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or ≤0.01 the Ko of a known antibody to CNX, as determined in a given assay.

In some embodiments, an antigen-binding molecule according to the present disclosure binds to CRT (e.g. human CRT) with an EC₅₀ which is less than 1 times, e.g. one of ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or ≤0.01 the EC₅₀ of a known antibody to CNX, as determined in a given assay.

In some embodiments, an antigen-binding molecule according to the present disclosure binds to CRT (e.g. human CRT) with a Ko which is less than 1 times, e.g. one of ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or ≤0.01 the Ko of a known antibody to CNX, as determined in a given assay.

In some embodiments, an antigen-binding molecule according to the present disclosure reduces a function of CNX/CRT and/or a function of a complex comprising CNX/CRT with an IC₅₀ which is less than 1 times, e.g. one of ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or ≤0.01 the IC₅₀ of a known antibody to CNX, as determined in a given assay.

In some embodiments, an antigen-binding molecule according to the present disclosure reduces a function of CNX/CRT and/or a function of a complex comprising CNX/CRT to less than 1 times, e.g. one of ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or ≤0.01 the level to which the function is reduced by a comparable concentration of a known antibody to CNX, in a given assay.

In some embodiments, an antigen-binding molecule according to the present disclosure reduces extracellular matrix degradation, collagen degradation and/or gelatin degradation with an IC₅₀ which is less than 1 times, e.g. one of ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or ≤0.01 the IC₅₀ of a known antibody to CNX, as determined in a given assay.

In some embodiments, an antigen-binding molecule according to the present disclosure reduces extracellular matrix degradation, collagen degradation and/or gelatin degradation to less than 1 times, e.g. one of ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or ≤0.01 the level to which the ECM/collagen/gelatin degradation is reduced by a comparable concentration of a known antibody to CNX, in a given assay.

In some embodiments, an antigen-binding molecule according to the present disclosure reduces oxireductase activity with an IC₅₀ which is less than 1 times, e.g. one of ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or ≤0.01 the IC₅₀ of a known antibody to CNX, as determined in a given assay.

In some embodiments, an antigen-binding molecule according to the present disclosure reduces oxireductase activity to less than 1 times, e.g. one of ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or ≤0.01 the level to which oxireductase activity is reduced by a comparable concentration of a known antibody to CNX, in a given assay.

In some embodiments, an antigen-binding molecule according to the present disclosure reduces disulfide bond reductase activity with an IC₅₀ which is less than 1 times, e.g. one of ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or ≤0.01 the IC₅₀ of a known antibody to CNX, as determined in a given assay.

In some embodiments, an antigen-binding molecule according to the present disclosure reduces disulfide bond reductase activity to less than 1 times, e.g. one of ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or ≤0.01 the level to which disulfide bond reductase activity is reduced by a comparable concentration of a known antibody to CNX, in a given assay.

In some embodiments, an antigen-binding molecule according to the present disclosure reduces cartilage degradation with an IC₅₀ which is less than 1 times, e.g. one of ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or ≤0.01 the IC₅₀ of a known antibody to CNX, as determined in a given assay.

In some embodiments, an antigen-binding molecule according to the present disclosure reduces cartilage degradation to less than 1 times, e.g. one of ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or ≤0.01 the level to which the cartilage degradation is reduced by a comparable concentration of a known antibody to CNX, in a given assay.

In some embodiments, an antigen-binding molecule according to the present increases killing or ADCC of cells expressing CNX/CRT, and/or a complex comprising CNX/CRT, to more than 1 times, e.g. one of 1.01 times, ≥1.02 times, ≥1.03 times, 1.04 times, ≥1.05 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, ≥3 times, ≥4 times, ≥≤5 times, ≥6 times, ≥7 times, ≥8 times, ≥9 times or ≥10 times the level of killing/ADCC achieved by treatment with a comparable concentration of a known antibody to CNX, in a given assay.

In some embodiments, an antigen-binding molecule according to the present disclosure reduces inhibits tumor growth (e.g. in an in vivo model, e.g. of liver cancer) to less than 1 times, e.g. one of ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or ≤0.01 the level to which tumor growth is inhibited by treatment with a comparable concentration of a known antibody to CNX, in a given assay.

In some embodiments, an antigen-binding molecule according to the present disclosure reduces metastasis (e.g. in an in vivo model, e.g. of metastasis of breast cancer to the lung) to less than 1 times, e.g. one of ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or ≤0.01 the level to which metastasis is reduced by treatment with a comparable concentration of a known antibody to CNX, in a given assay.

In some embodiments, an antigen-binding molecule according to the present increases survival of subjects having a cancer to more than 1 times, e.g. one of 1.01 times, ≥1.02 times, ≥1.03 times, ≥1.04 times, ≥1.05 times, ≥1.1 times, ≥1.2 times, ≥1.3 times, ≥1.4 times, ≥1.5 times, ≥1.6 times, ≥1.7 times, ≥1.8 times, 1.9 times, ≥2 times, ≥3 times, ≥4 times, ≥≤5 times, ≥6 times, ≥7 times, ≥8 times, ≥9 times or ≥10 times the level of survival achieved by treatment with a comparable concentration of a known antibody to CNX, in a given assay.

In some embodiments, an antigen-binding molecule according to the present disclosure reduces pathology of a disease/condition characterised by ECM degradation or cartilage degradation (e.g. arthritis) in a subject (e.g. in a CAIA model, e.g. as determined by arthritis score) to less than 1 times, e.g. one of ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or ≤0.01 the level to which pathology is inhibited by treatment with a comparable concentration of a known antibody to CNX, in a given assay.

### Chimeric Antigen Receptors (CARs)

The present disclosure also provides Chimeric Antigen Receptors (CARs) comprising the antigen-binding polypeptides or polypeptides of the present disclosure.

CARs are recombinant receptors that provide both antigen-binding and T cell activating functions. CAR structure and engineering is reviewed, for example, in Dotti et al., Immunol Rev (2014) 257(1), hereby incorporated by reference in its entirety. CARs comprise an antigen-binding region linked to a cell membrane anchor region and a signalling region. An optional hinge region may provide separation between the antigen-binding region and cell membrane anchor region, and may act as a flexible linker.

The CAR of the present disclosure comprises an antigen-binding region which comprises or consists of the antigen-binding molecule of the present disclosure, or which comprises or consists of a polypeptide according to the present disclosure.

The cell membrane anchor region is provided between the antigen-binding region and the signalling region of the CAR and provides for anchoring the CAR to the cell membrane of a cell expressing a CAR, with the antigen-binding region in the extracellular space, and signalling region inside the cell. In some embodiments, the CAR comprises a cell membrane anchor region comprising or consisting of an amino acid sequence which comprises, consists of, or is derived from, the transmembrane region amino acid sequence for one of CD3-ζ, CD4, CD8 or CD28. As used herein, a region which is ‘derived from’ a reference amino acid sequence comprises an amino acid sequence having at least 60%, e.g. one of at least 65%, 70%, 75%, 80%, 85%, 90%, 91%, 92%, 93%, 94%, 95%, 96%, 97%, 98%, 99% or 100% sequence identity to the reference sequence.

The signalling region of a CAR allows for activation of the T cell. The CAR signalling regions may comprise the amino acid sequence of the intracellular domain of CD3-ζ, which provides immunoreceptor tyrosine-based activation motifs (ITAMs) for phosphorylation and activation of the CAR-expressing T cell. Signalling regions comprising sequences of other ITAM-containing proteins such as FcγRI have also been employed in CARs (Haynes et al., 2001 J Immunol 166(1):182-187). Signalling regions of CARs may also comprise co-stimulatory sequences derived from the signalling region of co-stimulatory molecules, to facilitate activation of CAR-expressing T cells upon binding to the target protein. Suitable co-stimulatory molecules include CD28, OX40, 4-1BB, ICOS and CD27. In some cases CARs are engineered to provide for co-stimulation of different intracellular signalling pathways. For example, signalling associated with CD28 costimulation preferentially activates the phosphatidylinositol 3-kinase (PI3K) pathway, whereas the 4-1 BB-mediated signalling is through TNF receptor associated factor (TRAF) adaptor proteins. Signalling regions of CARs therefore sometimes contain co-stimulatory sequences derived from signalling regions of more than one co-stimulatory molecule. In some embodiments, the CAR of the present disclosure comprises one or more co-stimulatory sequences comprising or consisting of an amino acid sequence which comprises, consists of, or is derived from, the amino acid sequence of the intracellular domain of one or more of CD28, OX40, 4-1 BB, ICOS and CD27. An optional hinge region may provide separation between the antigen-binding domain and the transmembrane domain, and may act as a flexible linker. Hinge regions may be derived from IgG1. In some embodiments, the CAR of the present disclosure comprises a hinge region comprising or consisting of an amino acid sequence which comprises, consists of, or is derived from, the amino acid sequence of the hinge region of IgG1.

Also provided is a cell comprising a CAR according to the present disclosure. The CAR according to the present disclosure may be used to generate CAR-expressing immune cells, e.g. CAR-T or CAR-NK cells. Engineering of CARs into immune cells may be performed during culture, in vitro.

The antigen-binding region of the CAR of the present disclosure may be provided with any suitable format, e.g. scFv, scFab, etc.

### Nucleic Acids and Vectors

The present disclosure provides a nucleic acid, or a plurality of nucleic acids, encoding an antigen-binding molecule, polypeptide or CAR according to the present disclosure. In some embodiments, the nucleic acid(s) comprise or consist of DNA and/or RNA.

In some embodiments, the nucleic acid(s) may be, or may be comprised in, a vector, or a plurality of vectors. That is, the nucleotide sequence(s) of the nucleic acid(s) may be contained in vector(s). The antigen-binding molecule, polypeptide or CAR according to the present disclosure may be produced within a cell by transcription from a vector encoding the antigen-binding molecule, polypeptide or CAR, and subsequent translation of the transcribed RNA.

Accordingly, the present disclosure also provides a vector, or plurality of vectors, comprising the nucleic acid or plurality of nucleic acids according to the present disclosure. The vector may facilitate delivery of the nucleic acid(s) encoding an antigen-binding molecule, polypeptide or CAR according to the present disclosure. The vector may be an expression vector comprising elements required for expressing nucleic acid(s) comprising/encoding an antigen-binding molecule, polypeptide or CAR according to the present disclosure.

Nucleic acids and vectors according to the present disclosure may be provided in purified or isolated form, i.e. from other nucleic acid, or naturally-occurring biological material.

The nucleotide sequence may be contained in a vector, e.g. an expression vector. A ‘vector’ as used herein is a nucleic acid molecule used as a vehicle to transfer exogenous nucleic acid into a cell. The vector may be a vector for expression of the nucleic acid in the cell. Such vectors may include a promoter sequence operably linked to the nucleotide sequence encoding the sequence to be expressed. A vector may also include a termination codon and expression enhancers. Any suitable vectors, promoters, enhancers and termination codons known in the art may be used to express a peptide or polypeptide from a vector according to the present disclosure.

The term ‘operably linked’ may include the situation where a selected nucleic acid sequence and regulatory nucleic acid sequence (e.g. promoter and/or enhancer) are covalently linked in such a way as to place the expression of nucleic acid sequence under the influence or control of the regulatory sequence (thereby forming an expression cassette). Thus a regulatory sequence is operably linked to the selected nucleic acid sequence if the regulatory sequence is capable of effecting transcription of the nucleic acid sequence. The resulting transcript(s) may then be translated into a desired peptide(s)/polypeptide(s).

Suitable vectors include plasmids, binary vectors, DNA vectors, mRNA vectors, viral vectors (e.g. retroviral vectors, e.g. gammaretroviral vectors (e.g. murine Leukemia virus (MLV)-derived vectors, e.g. SFG vector), lentiviral vectors, adenovirus vectors, adeno-associated virus vectors, vaccinia virus vectors and herpesvirus vectors), transposon-based vectors, and artificial chromosomes (e.g. yeast artificial chromosomes), e.g. as described in Maus et al., Annu Rev Immunol (2014) 32:189-225 or Morgan and Boyerinas, Biomedicines (2016) 4:9, which are both hereby incorporated by reference in their entirety.

In some embodiments, the vector may be a eukaryotic vector, e.g. a vector comprising the elements necessary for expression of protein from the vector in a eukaryotic cell. In some embodiments, the vector may be a mammalian vector, e.g. comprising a cytomegalovirus (CMV) or SV40 promoter to drive protein expression.

Constituent polypeptides of an antigen-binding molecule according to the present disclosure may be encoded by different nucleic acids of the plurality of nucleic acids, or by different vectors of the plurality of vectors.

### Cells Comprising/Expressing the Antigen-Binding Molecules and Polypeptides

The present disclosure also provides a cell comprising or expressing an antigen-binding molecule, polypeptide or CAR according to the present disclosure. Also provided is a cell comprising or expressing a nucleic acid, a plurality of nucleic acids, a vector or a plurality of vectors according to the present disclosure.

The cell may be a eukaryotic cell, e.g. a mammalian cell. The mammal may be a primate (rhesus, cynomolgous, non-human primate or human) or a non-human mammal (e.g. rabbit, guinea pig, rat, mouse or other rodent (including any animal in the order Rodentia), cat, dog, pig, sheep, goat, cattle (including cows, e.g. dairy cows, or any animal in the order Bos), horse (including any animal in the order Equidae), donkey, and non-human primate).

In some embodiments, the cell is, or is derived from, a cell type commonly used for the expression of polypeptides for use in therapy in humans. Exemplary cells are described e.g. in Kunert and Reinhart, Appl Microbiol Biotechnol. (2016) 100:3451-3461 (hereby incorporated by reference in its entirety), and include e.g. CHO, HEK 293, PER.C6, NS0 and BHK cells. In preferred embodiments, the cell is, or is derived from, a CHO cell.

The present disclosure also provides a method for producing a cell comprising a nucleic acid(s) or vector(s) according to the present disclosure, comprising introducing a nucleic acid, a plurality of nucleic acids, a vector or a plurality of vectors according to the present disclosure into a cell. In some embodiments, introducing an isolated nucleic acid(s) or vector(s) according to the present disclosure into a cell comprises transformation, transfection, electroporation or transduction (e.g. retroviral transduction).

The present disclosure also provides a method for producing a cell expressing/comprising an antigen-binding molecule, polypeptide or CAR according to the present disclosure, comprising introducing a nucleic acid, a plurality of nucleic acids, a vector or a plurality of vectors according to the present disclosure in a cell. In some embodiments, the methods additionally comprise culturing the cell under conditions suitable for expression of the nucleic acid(s) or vector(s) by the cell. In some embodiments, the methods are performed in vitro.

The present disclosure also provides cells obtained or obtainable by the methods according to the present disclosure.

### Producing the Antigen-Binding Molecules and Polypeptides

Antigen-binding molecules and polypeptides according to the present disclosure may be prepared according to methods for the production of polypeptides known to the skilled person.

Polypeptides may be prepared by chemical synthesis, e.g. liquid or solid phase synthesis. For example, peptides/polypeptides can be synthesised using the methods described in, for example, Chandrudu et al., Molecules (2013), 18: 4373-4388, which is hereby incorporated by reference in its entirety.

Alternatively, antigen-binding molecules and polypeptides may be produced by recombinant expression. Molecular biology techniques suitable for recombinant production of polypeptides are well known in the art, such as those set out in Green and Sambrook, Molecular Cloning: A Laboratory Manual (4th Edition), Cold Spring Harbor Press, 2012, and in Nat Methods. (2008); 5(2): 135-146 both of which are hereby incorporated by reference in their entirety. Methods for the recombinant production of antigen-binding molecules are also described in Frenzel et al., Front Immunol. (2013); 4: 217 and Kunert and Reinhart, Appl Microbiol Biotechnol. (2016) 100: 3451-3461, both of which are hereby incorporated by reference in their entirety.

In some cases, the antigen-binding molecules of the present disclosure are comprised of more than one polypeptide chain. In such cases, production of the antigen-binding molecules may comprise transcription and translation of more than one polypeptide, and subsequent association of the polypeptide chains to form the antigen-binding molecule.

For recombinant production according to the present disclosure, any cell suitable for the expression of polypeptides may be used. The cell may be a prokaryote or eukaryote. In some embodiments, the cell is a prokaryotic cell, such as a cell of archaea or bacteria. In some embodiments, the bacteria may be Gram-negative bacteria such as bacteria of the family Enterobacteriaceae, for example Escherichia coli. In some embodiments, the cell is a eukaryotic cell such as a yeast cell, a plant cell, insect cell or a mammalian cell, e.g. a cell described hereinabove.

In some cases, the cell is not a prokaryotic cell because some prokaryotic cells do not allow for the same folding or post-translational modifications as eukaryotic cells. In addition, very high expression levels are possible in eukaryotes and proteins can be easier to purify from eukaryotes using appropriate tags. Specific plasmids may also be utilised which enhance secretion of the protein into the media.

In some embodiments polypeptides may be prepared by cell-free-protein synthesis (CFPS), e.g. according to a system described in Zemella et al. Chembiochem (2015) 16(17): 2420-2431, which is hereby incorporated by reference in its entirety.

Production may involve culture or fermentation of a eukaryotic cell modified to express the polypeptide(s) of interest. The culture or fermentation may be performed in a bioreactor provided with an appropriate supply of nutrients, air/oxygen and/or growth factors. Secreted proteins can be collected by partitioning culture media/fermentation broth from the cells, extracting the protein content, and separating individual proteins to isolate secreted polypeptide(s). Culture, fermentation and separation techniques are well known to those of skill in the art, and are described, for example, in Green and Sambrook, Molecular Cloning: A Laboratory Manual (4th Edition; incorporated by reference herein above).

Bioreactors include one or more vessels in which cells may be cultured. Culture in the bioreactor may occur continuously, with a continuous flow of reactants into, and a continuous flow of cultured cells from, the reactor. Alternatively, the culture may occur in batches. The bioreactor monitors and controls environmental conditions such as pH, oxygen, flow rates into and out of, and agitation within the vessel such that optimum conditions are provided for the cells being cultured.

Following culturing the cells that express the antigen-binding molecule/polypeptide(s), the polypeptide(s) of interest may be isolated. Any suitable method for separating proteins from cells known in the art may be used. In order to isolate the polypeptide, it may be necessary to separate the cells from nutrient medium. If the polypeptide(s) are secreted from the cells, the cells may be separated by centrifugation from the culture media that contains the secreted polypeptide(s) of interest. If the polypeptide(s) of interest collect within the cell, protein isolation may comprise centrifugation to separate cells from cell culture medium, treatment of the cell pellet with a lysis buffer, and cell disruption e.g. by sonification, rapid freeze-thaw or osmotic lysis.

It may then be desirable to isolate the polypeptide(s) of interest from the supernatant or culture medium, which may contain other protein and non-protein components. A common approach to separating protein components from a supernatant or culture medium is by precipitation. Proteins of different solubilities are precipitated at different concentrations of precipitating agent such as ammonium sulfate. For example, at low concentrations of precipitating agent, water soluble proteins are extracted. Thus, by adding different increasing concentrations of precipitating agent, proteins of different solubilities may be distinguished. Dialysis may be subsequently used to remove ammonium sulfate from the separated proteins.

Other methods for distinguishing different proteins are known in the art, for example ion exchange chromatography and size chromatography. These may be used as an alternative to precipitation or may be performed subsequently to precipitation.

Once the polypeptide(s) of interest have been isolated from culture it may be desired or necessary to concentrate the polypeptide(s). A number of methods for concentrating proteins are known in the art, such as ultrafiltration or lyophilisation.

### Compositions

The present disclosure also provides compositions comprising the antigen-binding molecules, polypeptides, CARs, nucleic acids, expression vectors and cells described herein.

The antigen-binding molecules, polypeptides, CARs, nucleic acids, expression vectors and cells described herein may be formulated as pharmaceutical compositions or medicaments for clinical use and may comprise a pharmaceutically acceptable carrier, diluent, excipient or adjuvant.

The compositions of the present disclosure may comprise one or more pharmaceutically-acceptable carriers (e.g. liposomes, micelles, microspheres, nanoparticles), diluents/excipients (e.g. starch, cellulose, a cellulose derivative, a polyol, dextrose, maltodextrin, magnesium stearate), adjuvants, fillers, buffers, preservatives (e.g. vitamin A, vitamin E, vitamin C, retinyl palmitate, selenium, cysteine, methionine, citric acid, sodium citrate, methyl paraben, propyl paraben), anti-oxidants (e.g. vitamin A, vitamin E, vitamin C, retinyl palmitate, selenium), lubricants (e.g. magnesium stearate, talc, silica, stearic acid, vegetable stearin), binders (e.g. sucrose, lactose, starch, cellulose, gelatin, polyethylene glycol (PEG), polyvinylpyrrolidone (PVP), xylitol, sorbitol, mannitol), stabilisers, solubilisers, surfactants (e.g., wetting agents), masking agents or colouring agents (e.g. titanium oxide).

The term ‘pharmaceutically-acceptable’ as used herein pertains to compounds, ingredients, materials, compositions, dosage forms, etc., which are, within the scope of sound medical judgment, suitable for use in contact with the tissues of the subject in question (e.g. a human subject) without excessive toxicity, irritation, allergic response, or other problem or complication, commensurate with a reasonable benefit/risk ratio. Each carrier, diluent, excipient, adjuvant, filler, buffer, preservative, anti-oxidant, lubricant, binder, stabiliser, solubiliser, surfactant, masking agent, colouring agent, flavouring agent or sweetening agent of a composition according to the present disclosure must also be ‘acceptable’ in the sense of being compatible with the other ingredients of the formulation. Suitable carriers, diluents, excipients, adjuvants, fillers, buffers, preservatives, anti-oxidants, lubricants, binders, stabilisers, solubilisers, surfactants, masking agents, colouring agents, flavouring agents or sweetening agents can be found in standard pharmaceutical texts, for example, Remington's ‘The Science and Practice of Pharmacy’ (Ed. A. Adejare), 23rd Edition (2020), Academic Press.

Compositions may be formulated for topical, parenteral, systemic, intracavitary, intravenous, intra-arterial, intramuscular, intrathecal, intraocular, intraconjunctival, intratumoral, subcutaneous, intradermal, intrathecal, oral ortransdermal routes of administration. In some embodiments, a pharmaceutical composition/medicament may be formulated for administration by injection or infusion, or administration by ingestion.

Suitable formulations may comprise the relevant article in a sterile or isotonic medium. Medicaments and pharmaceutical compositions may be formulated in fluid, including gel, form. Fluid formulations may be formulated for administration by injection or infusion (e.g. via catheter) to a selected region of the human or animal body.

In some embodiments, the composition is formulated for injection or infusion, e.g. into a blood vessel, tissue/organ of interest, or tumor.

The present disclosure also provides methods for the production of pharmaceutically useful compositions, such methods of production may comprise one or more steps selected from: producing an antigen-binding molecule, polypeptide, CAR, nucleic acid (or plurality thereof), expression vector (or plurality thereof) or cell described herein; isolating an antigen-binding molecule, polypeptide, CAR, nucleic acid (or plurality thereof), expression vector (or plurality thereof) or cell described herein; and/or mixing an antigen-binding molecule, polypeptide, CAR, nucleic acid (or plurality thereof), expression vector (or plurality thereof) or cell described herein with a pharmaceutically acceptable carrier, adjuvant, excipient or diluent.

For example, a further aspect the present disclosure relates to a method of formulating or producing a medicament or pharmaceutical composition for use in the treatment of a disease/condition (e.g. a cancer), the method comprising formulating a pharmaceutical composition or medicament by mixing an antigen-binding molecule, polypeptide, CAR, nucleic acid (or plurality thereof), expression vector (or plurality thereof) or cell described herein with a pharmaceutically acceptable carrier, adjuvant, excipient or diluent.

### Therapeutic and Prophylactic Applications

The antigen-binding molecules, polypeptides, CARs, nucleic acids, expression vectors, cells and compositions described herein find use in therapeutic and prophylactic methods.

The present disclosure provides an antigen-binding molecule, polypeptide, CAR, nucleic acid (or plurality thereof), expression vector (or plurality thereof), cell or composition described herein for use in a method of medical treatment or prophylaxis. Also provided is an antigen-binding molecule, polypeptide, CAR, nucleic acid (or plurality thereof), expression vector (or plurality thereof), cell or composition described herein for use in a method of treating or preventing a disease or condition described herein. Also provided is the use of an antigen-binding molecule, polypeptide, CAR, nucleic acid (or plurality thereof), expression vector (or plurality thereof), cell or composition described herein in the manufacture of a medicament for treating or preventing a disease or condition described herein. Also provided is a method of treating or preventing a disease or condition described herein, comprising administering to a subject a therapeutically or prophylactically effective amount of an antigen-binding molecule, polypeptide, CAR, nucleic acid (or plurality thereof), expression vector (or plurality thereof), cell or composition described herein.

The methods may be effective to reduce the development or progression of a disease/condition, alleviation of the symptoms of a disease/condition or reduction in the pathology of a disease/condition. The methods may be effective to prevent progression of the disease/condition, e.g. to prevent worsening of, or to slow the rate of development of, the disease/condition. In some embodiments, the methods may lead to an improvement in the disease/condition, e.g. a reduction in the symptoms of the disease/condition or reduction in some other correlate of the severity/activity of the disease/condition. In some embodiments, the methods may prevent development of the disease/condition a later stage (e.g. a chronic stage or metastasis).

It will be appreciated that the articles of the present disclosure may be used for the treatment/prevention of any disease/condition that would derive therapeutic or prophylactic benefit from a reduction in the level/activity of CNX, CRT, complexes comprising CNX/CRT, or a reduction in the number or activity of cells comprising/expressing CNX, CRT, or complexes comprising CNX/CRT.

For example, the disease/condition may be a disease/condition in which CNX, CRT, complexes comprising CNX/CRT, or cells expressing/expressing the same are pathologically-implicated, e.g. a disease/condition in which an increased level/activity of CNX, CRT, complexes comprising CNX/CRT, of an increase in the number/proportion of cells comprising/expressing CNX, CRT or complexes comprising CNX/CRT is positively associated with the onset, development or progression of the disease/condition, and/or severity of one or more symptoms of the disease/condition. In some embodiments, an increased level/activity of CNX, CRT, complexes comprising CNX/CRT, of an increase in the number/proportion of cells comprising/expressing CNX, CRT or complexes comprising CNX/CRT may be a risk factor for the onset, development or progression of the disease/condition.

In some embodiments, the disease/condition to be treated/prevented in accordance with the present disclosure is a disease/condition characterised by an increase in the level of expression or activity of CNX, CRT or complexes comprising CNX or CRT, e.g. as compared to the level of expression/activity in the absence of the disease/condition. In some embodiments, the disease/condition to be treated/prevented is a disease/condition characterised by an increase in the number/proportion/activity of cells expressing CNX, CRT or complexes comprising CNX or CRT, e.g. as compared to the level/number/proportion/activity in the absence of the disease/condition.

In some embodiments, the disease/condition to be treated/prevented in accordance with the present disclosure is a disease/condition described in WO 2020/159445 A1 (hereby incorporated by reference in its entirety). In some embodiments, the disease/condition to be treated/prevented in accordance with the present disclosure is a disease/condition described in PCT/EP2022/051297 (hereby incorporated by reference in its entirety).

Treatment in accordance with the methods of the present disclosure may achieve one or more of the following in a subject (compared to an equivalent untreated subject, or subject treated with an appropriate control): a reduction in the level of CNX, CRT or complexes comprising CNX or CRT; a reduction in the activity of CNX, CRT or complexes comprising CNX or CRT; and/or a reduction in the number/proportion of cells comprising/expressing CNX, CRT or complexes comprising CNX or CRT.

In some embodiments, the disease/condition to be treated/prevented in accordance with the present disclosure is characterised by elevated O-glycosylation activity. For example, where the disease/condition is a cancer, the cancer may comprise cells having elevated O-glycosylation activity. As used herein ‘O-glycosylation activity’ refers to addition of O-linked glycan to the hydroxyl group of the side chain of e.g. a serine, threonine, tyrosine, hydroxylysine, or hydroxyproline residue of a protein. An ‘elevated’ level of O-glycosylation activity may refer to a level of O-glycosylation activity which is greater than the level of O-glycosylation activity in the absence of the disease/condition (e.g. in a healthy subject, or in equivalent non-diseased tissue). Where the disease/condition is a cancer, the level of O-glycosylation activity may be greater than the level of O-glycosylation activity in equivalent non-cancerous cells/non-tumor tissue. A cancer/cell thereof may comprise one or more mutations (e.g. relative to equivalent non-cancerous cells/non-tumor tissue) causing upregulation of O-glycosylation activity.

In some embodiments, the disease/condition to be treated/prevented in accordance with the present disclosure is characterised by elevated Src activity. For example, where the disease/condition is a cancer, the cancer may comprise cells having elevated Src activity. As used herein ‘Src activity’ refers to Src-mediated phosphorylation of tyrosine residues. An ‘elevated’ level of Src activity may refer to a level of Src activity which is greater than the level of Src activity in the absence of the disease/condition (e.g. in a healthy subject, or in equivalent non-diseased tissue). Where the disease/condition is a cancer, the level of Src activity may be greater than the level of Src activity in equivalent non-cancerous cells/non-tumor tissue. A cancer/cell thereof may comprise one or more mutations (e.g. relative to equivalent non-cancerous cells/non-tumor tissue) causing upregulation of Src activity.

In some embodiments, the disease/condition to be treated/prevented in accordance with the present disclosure is characterised by elevated GalNAc-transferase (GALNT) activity. For example, where the disease/condition is a cancer, the cancer may comprise cells having elevated GALNT activity. As used herein ‘GALNT activity’ refers to GALNT-mediated transfer of N-acetylgalactosamine (GalNAc) from UDP-GalNAc to the hydroxyl group of the side chain of e.g. a serine or threonine residue. An ‘elevated’ level of GALNT activity may refer to a level of GALNT activity which is greater than the level of GALNT activity in the absence of the disease/condition (e.g. in a healthy subject, or in equivalent non-diseased tissue). Where the disease/condition is a cancer, the level of GALNT activity may be greater than the level of GALNT activity in equivalent non-cancerous cells/non-tumor tissue. A cancer/cell thereof may comprise one or more mutations (e.g. relative to equivalent non-cancerous cells/non-tumor tissue) causing upregulation of GALNT activity.

In some embodiments, the disease/condition to be treated/prevented in accordance with the present disclosure is characterised by elevated O-glycosylation. For example, where the disease/condition is a cancer, the cancer may comprise cells having an elevated level of O-glycosylation of a protein expressed by the cells. An ‘elevated’ level of O-glycosylation may refer to a level of O-glycosylation which is greater than the level of O-glycosylation in the absence of the disease/condition (e.g. in a healthy subject, or in equivalent non-diseased tissue). Where the disease/condition is a cancer, the level of O-glycosylation may be greater than the level of O-glycosylation in equivalent non-cancerous cells/non-tumor tissue. A cancer/cell thereof may comprise one or more mutations (e.g. relative to equivalent non-cancerous cells/non-tumor tissue) causing upregulation of O-glycosylation.

In some embodiments, the disease/condition to be treated/prevented in accordance with the present disclosure is characterised by elevated Tn glycosylation. For example, where the disease/condition is a cancer, the cancer may comprise cells having Tn glycosylation of a protein expressed by the cells. As used herein ‘Tn glycosylation’ refers to the presence of N-acetylgalactosamine (GalNAc) linked to the hydroxyl group of the side chain of a serine or threonine residue of a protein by a glycosidic bond. A ‘Tn glycosylated’ protein comprises at least one Tn glycan, which may also be referred to as Tn antigen.

In some embodiments, the disease/condition to be treated/prevented in accordance with the present disclosure is characterised by elevated Tn glycosylation. For example, where the disease/condition is a cancer, the cancer may comprise cells having an elevated level of Tn glycosylation of a protein expressed by the cells. An ‘elevated’ level of Tn glycosylation may refer to a level of Tn glycosylation which is greater than the level of Tn glycosylation in the absence of the disease/condition (e.g. in a healthy subject, or in equivalent non-diseased tissue). Where the disease/condition is a cancer, the level of Tn glycosylation may be greater than the level of Tn glycosylation in equivalent non-cancerous cells/non-tumor tissue. A cancer/cell thereof may comprise one or more mutations (e.g. relative to equivalent non-cancerous cells/non-tumor tissue) causing upregulation of Tn glycosylation. A protein having an ‘elevated’ level of Tn glycosylation as compared to a reference protein may possess more Tn glycans than the reference protein.

The anti-CNX antibodies of the present disclosure are demonstrated to be useful to inhibit ECM degradation. Accordingly, in some embodiments, the disease/condition to be treated/prevented in accordance with the present disclosure is a disease/condition characterised by extracellular matrix (ECM) degradation. A disease/condition which is ‘characterised by ECM degradation’ may be a disease/condition in which ECM degradation is a symptom of the disease/condition.

The disease/condition to be treated/prevented in accordance with the present disclosure may be a disease/condition in which ECM degradation is pathologically-implicated. For example, the disease/condition may be a disease/condition in which ECM degradation, and/or an increased level of ECM degradation, is implicated in the pathology of the disease/condition.

Diseases/conditions characterised by ECM degradation include e.g. cancers, and diseases/conditions characterised by cartilage degradation.

The involvement of ECM degradation in the development and progression of cancers is well known, and is reviewed e.g. in Walker et al., Int. J. Mol. Sci. (2018) 19(10): 3028, Najafi et al., J. Cell Biochem. (2019) 120(3):2782-2790 and Winkler et al., Nat. Commun. (2020) 11(1):5120, all of which are hereby incorporated by reference in their entirety.

In some embodiments the disease/condition to be treated/prevented is a cancer. Cancer may refer to any unwanted cell proliferation (or any disease manifesting itself by unwanted cell proliferation), neoplasm or tumor. The cancer may be benign or malignant and may be primary or secondary (metastatic). A neoplasm or tumor may be any abnormal growth or proliferation of cells and may be located in any tissue. The cancer may be of tissues/cells derived from e.g. the adrenal gland, adrenal medulla, anus, appendix, bladder, blood, bone, bone marrow, brain, breast, cecum, central nervous system (including or excluding the brain) cerebellum, cervix, colon, duodenum, endometrium, epithelial cells (e.g. renal epithelia), gallbladder, oesophagus, glial cells, heart, ileum, jejunum, kidney, lacrimal glad, larynx, liver, lung, lymph, lymph node, lymphoblast, maxilla, mediastinum, mesentery, myometrium, nasopharynx, omentum, oral cavity, ovary, pancreas, parotid gland, peripheral nervous system, peritoneum, pleura, prostate, salivary gland, sigmoid colon, skin, small intestine, soft tissues, spleen, stomach, testis, thymus, thyroid gland, tongue, tonsil, trachea, uterus, vulva, and/or white blood cells.

Tumors may be nervous or non-nervous system tumors. Nervous system tumors may originate either in the central or peripheral nervous system, e.g. glioma, medulloblastoma, meningioma, neurofibroma, ependymoma, Schwannoma, neurofibrosarcoma, astrocytoma and oligodendroglioma. Non-nervous system cancers/tumors may originate in any other non-nervous tissue, examples include melanoma, mesothelioma, lymphoma, myeloma, leukemia, Non-Hodgkin's lymphoma (NHL), Hodgkin's lymphoma, chronic myelogenous leukemia (CML), acute myeloid leukemia (AML), myelodysplastic syndrome (MDS), cutaneous T cell lymphoma (CTCL), chronic lymphocytic leukemia (CLL), hepatoma, epidermoid carcinoma, prostate carcinoma, breast cancer, lung cancer, colon cancer, ovarian cancer, pancreatic cancer, thymic carcinoma, NSCLC, hematologic cancer and sarcoma.

Anti-CNX antibodies are shown herein and also e.g. in Ros et al. Nat. Cell Biol. (2020) 22(11):1371-1381 and WO 2020/159445 A1 to be useful to inhibit tumor growth and metastasis, including of breast and liver cancers. Ryan et al., J. Transl. Med. (2016) 14:196 proposes CNX as a therapeutic target in colorectal cancer.

Chen et al., Cancer Immunol. Res. (2019) 7(1):123-135 demonstrate that expression of CNX is upregulated in oral squamous cell carcinoma, and that knockdown of CNX improved control of tumor growth in a model of melanoma. The authors also demonstrated that CNX inhibits the proliferation and effector function of CD4+ and CD8+ T cells, by a mechanism involving upregulation of the expression of immune checkpoint molecule PD-1. Thus, Chen et al. suggests that intervention targeting CNX could be useful for the treatment/prevention of a wide range of cancers, through indirect antagonism of PD-1/PD-L1-mediated suppression of anticancer responses.

In some embodiments, the cancer is liver cancer, breast cancer, oral cancer (e.g. oral squamous cell carcinoma) sarcoma, lung cancer, prostate cancer, bladder cancer, renal cancer, melanoma, pancreatic cancer, endometrial cancer, colorectal cancer and thyroid cancer.

In some embodiments, the liver cancer is a primary liver cancer. In some embodiments, the liver cancer is hepatocellular carcinoma (HCC), fibrolamellar carcinoma, bile duct cancer (cholangiocarcinoma), angiosarcoma or hepatoblastoma.

In some embodiments, the breast cancer is a primary breast cancer. In some embodiments, the breast cancer is ductal carcinoma, lobular carcinoma, in situ breast cancer (e.g. ductal carcinoma in situ (DCIS) invasive carcinoma (e.g. invasive ductal carcinoma (IDC), invasive lobular carcinoma (ILC), triple negative breast cancer or inflammatory breast cancer), Paget disease, angiosarcoma or Phyllodes tumor.

In some embodiments, the cancer is a cancer that would derive therapeutic or prophylactic benefit from a reduction in the expression or activity of CNX, CRT or complexes comprising CNX or CRT. In some embodiments, the cancer is a cancer which is caused or exacerbated by expression/overexpression or activity of CNX, CRT or complexes comprising CNX or CRT. In some embodiments, the cancer is a cancer for which expression/overexpression or activity of CNX, CRT or complexes comprising CNX or CRT is a risk factor for the development or progression of the cancer. In some embodiments the cancer is a cancer for which expression/overexpression or activity of CNX, CRT or complexes comprising CNX or CRT is positively associated with onset, development, progression, severity or metastasis.

As used herein, overexpression of a given protein/protein complex (e.g. CNX, CRT, complexes comprising the same) refers to a level of gene or protein expression of the relevant protein/protein complex which is greater than the level of expression by equivalent non-cancerous cells/non-tumor tissue.

In some embodiments, the cancer may be a cancer characterised by expression/overexpression of CNX or CRT (i.e. ‘CNX-positive’ cancers and ‘CRT-positive’ cancers, respectively), or of polypeptide complexes comprising CNX/CRT. The cancer may comprise cells expressing/overexpressing CNX, CRT, or polypeptide complexes comprising CNX/CRT.

CNX/CRT expression may be determined by any suitable means. Expression may be gene expression or protein expression. Gene expression can be determined e.g. by detection of mRNA encoding CNX/CRT, for example by quantitative real-time PCR (qRT-PCR). Protein expression can be determined e.g. by detection of CNX/CRT, for example by antibody-based methods, for example by western blot, immunohistochemistry, immunocytochemistry, flow cytometry, or ELISA.

In some embodiments, the cancer may be a cancer characterised by surface expression of CNX/CRT. In some embodiments, the cancer may comprise cells expressing CNX/CRT at the cell surface. CNX/CRT may be present in or at the cell membrane of cells of the cancer.

In some embodiments, the cancer may be a cancer characterised by expression/overexpression of 0-glycosylated CNX/CRT. The cancer may comprise cells expressing/overexpressing O-glycosylated CNX/CRT. In some embodiments, the cancer may be a cancer characterised by expression of CNX/CRT having an elevated level of O-glycosylation. The cancer may comprise cells expressing CNX/CRT having an elevated level of O-glycosylation.

In some embodiments, the cancer may be a cancer characterised by expression/overexpression of Tn glycosylated CNX/CRT. The cancer may comprise cells expressing/overexpressing Tn glycosylated CNX/CRT. In some embodiments, the cancer may be a cancer characterised by expression of CNX/CRT having an elevated level of Tn glycosylation. The cancer may comprise cells expressing CNX/CRT having an elevated level of Tn glycosylation.

Treatment of a subject with an antigen-binding molecule in accordance with the present disclosure may: delay/prevent the onset of one or more symptoms of the cancer; reduce the severity of one or more symptoms of the cancer; increase survival of the subject; reduce/inhibit survival of cells of the cancer; reduce the number of cells of the cancer in the subject; reduce tumor size/volume; reduce cancer/tumor burden in the subject; reduce/inhibit growth of cells of the cancer; reduce/inhibit tumor growth; reduce/inhibit invasion by cells of the cancer; and/or reduce/inhibit metastasis of the cancer.

In some embodiments, the disease/condition to be treated/prevented in accordance with the present disclosure is cartilage degradation, or a disease/condition characterised by cartilage degradation.

As used herein, ‘cartilage degradation’ refers to the degradation/degeneration/loss/destruction of cartilage tissue. Cartilage tissue is formed of chondrocytes, and extracellular matrix rich in glycosaminoglycans, proteoglycans, collagen and in some instances also elastin.

Cartilage is an avascular, aneural, alymphatic connective tissue found in the synovial joints, spine, ribs, external ears, nose, and airways, and in the growth plates of children and adolescents. There are three major types of cartilage found in humans: hyaline, fibrous and elastic (Wachsmuth et al., Histol Histopathol. 2006 May; 21(5):477-85). Hyaline cartilage is the most widespread type of cartilage and is the type that makes up the embryonic skeleton. It persists in human adults at the ends of bones in free-moving joints as articular cartilage, at the ends of the ribs, and in the nose, larynx, trachea, and bronchi. Fibrocartilage is tough, strong tissue found predominantly in the intervertebral disks and at the insertions of ligaments and tendons; it is similar to other fibrous tissues but contains cartilage ground substance and chondrocytes. Elastic cartilage, is more pliable than the other two forms because it contains elastin fibres in addition to collagen. In humans it makes up the external ear, the auditory tube of the middle ear, and the epiglottis.

In some embodiments, the cartilage degradation may be of hyaline cartilage, fibrous cartilage and/or elastic cartilage.

In most tissues, fibroblasts are the key cell type involved in producing extracellular matrices. However, fibroblasts can also degrade the matrix, allowing the turn-over of this essential component of tissues. How fibroblasts regulate these two opposite activities remains unclear. Synovial fibroblasts (SF) also called synoviocytes are the prototypical Janus-faced cells. In healthy individuals, SFs contribute to the viscosity of the synovial fluid by secreting proteins such as hyaluronic acid and lubricin (Jay et al., J. Rheumatol. 27, 594-600, 2000). In arthritic diseases, SFs adhere and degrade the cartilage, specifically the extracellular matrix (ECM) of the cartilage. Understanding this change in activity during arthritis has been a major focus of research in recent years (Ospelt. RMD Open. 3, e000471, 2017). The GALNTs Activation pathway (GALA) regulates ECM degradation in cancer cells through glycosylation of MMP14 and CNX. The inventors demonstrate herein that cartilage degradation, disorders associated with cartilage degradation, and joint disorders are also associated with increased levels of GALA and O-glycosylation.

GALA induces matrix degradation through at least two mechanisms. First, it stimulates glycosylation of MMP14, which is required for its proteolytic activity (Nguyen et al., Cancer Cell. 32, 639-653.e6, 2017). Second, GALA induces the glycosylation of the ER-resident protein CNX, which forms a complex with ERp57 (Ros et al., Nat. Cell Biol. 22, 1371-1381. 2020). Following GALA-glycosylation, a fraction of the CNX:ERp57 complex is translocated to the surface of cancer cells. The CNX:ERp57 complex accumulates in invadosomes and reduces disulfide bridges in the ECM (Ros et al., Nat. Cell Biol. 22, 1371-1381. 2020). This reduction of disulfide bridges is essential for the effective degradation of ECM (Ros et al., Nat. Cell Biol. 22, 1371-1381. 2020).

The inventors demonstrate herein the treatment of cartilage degradation using anti-CNX antibodies. Anti-CNX antibodies are also shown to inhibit ECM degradation, which is a key factor in cartilage degeneration, cartilage degradation and diseases/conditions characterised by cartilage degradation. Anti-CNX antibodies are furthermore shown herein to reduce the pathology of arthritis in vivo, which is a disease characterised by cartilage degradation.

Aspects and embodiments of the present disclosure relate to the treatment/prevention of diseases/conditions characterised by cartilage degradation. A disease/condition which is ‘characterised by cartilage degradation’ is a disease/condition in which cartilage degradation is a symptom of the disease/condition. The disease/condition to be treated/prevented in accordance with the present disclosure may be a disease/condition in which cartilage degradation is pathologically-implicated. For example, the disease/condition may be a disease/condition in which cartilage degradation, and/or an increased level of cartilage degradation, is implicated in the pathology of the disease/condition.

Cartilage degradation can occur through, and/or lead to the development, progression or worsening of, disorders such as osteoarthritis, psoriasis arthritis, rheumatoid arthritis, juvenile arthritis, post-traumatic arthritis, bursitis, gout, chondrocalcinosis, fibromyalgia, costochondritis, osteochondritis dissecans, cartilage damage, and polychondritis. Cartilage degradation can also occur as a consequence of physical trauma/mechanical damage, e.g. through sports injury (e.g. as a consequence of collision, or hyperextension) or surgery. Subjects having with cartilage degradation commonly experience joint pain, stiffness, and inflammation, which can impact quality of life.

In some embodiments, a disease/condition characterised by cartilage degradation in accordance with the present disclosure may be selected from: a joint disorder, arthritis, osteoarthritis, psoriasis arthritis, rheumatoid arthritis, juvenile arthritis, post-traumatic arthritis, gout, chondrocalcinosis, fibromyalgia, costochondritis, osteochondritis dissecans, cartilage damage and polychondritis.

Arthritis is a group of diseases affecting joints (Barbour et al., Morbidity and Mortality Weekly Report. 65. 2016, pp. 1052-1056). Rheumatoid arthritis (RA) and Osteoarthritis (OA) are two of the most common types (Murphy and Nagase. Nat. Clin. Pract. Rheumatol. 4, 128-135. 2008). It is thought that mechanical damage of the cartilage leads to a low grade inflammatory condition that mediates progressive cartilage loss in arthritis (Kapoor et al., Nat. Rev. Rheumatol. 7, 33-42. 2011; Pap and Korb-Pap. Rheumatol. 11, 606-615. 2015). Post-traumatic arthritis (PTA) develops after an acute direct trauma to the joints. PTA causes about 12% of all osteoarthritis cases, and a history of physical trauma may also be found in patients with chronic inflammatory arthritis.

In healthy synovial joints, the synovial membrane surrounds and isolates the joint cavity, secreting extracellular matrix proteins in the synovial fluid. Synovial fibroblasts are the main stromal cells of the synovial membrane, interspaced with resident macrophages (Barbour et al., Morbidity and Mortality Weekly Report. 65. 2016, pp. 1052-1056). During the active phases of RA, SFs become activated, expressing the Fibroblast Activation Protein alpha and proliferate. SF cells, as other stromal cells, express innate immune receptors such as Toll-Like Receptors. They can detect local pathogens and molecular damage, secreting cytokines that activate immune cells (Ospelt et al., Arthritis Rheum. 58, 3684-3692. 2008). During inflammation, SF proliferate, forming, together with infiltrating immune cells, an enlarged synovial membrane called a pannus (Choy. Rheumatology. 51 Suppl 5, v3-11. 2012). The pannus invades the joint cavity and degrades cartilage (Pap and Korb-Pap. Rheumatol. 11, 606-615. 2015). In particular, SF in the synovial lining layer have been shown to mediate cartilage degradation, while SF in the sub-lining tend to mediate inflammation (Croft et al., Nature. 570, 246-251. 2019). The ECM degrading activity is due to increased production of matrix metalloproteinases (MMPs), A Disintegrin And Metalloproteinase with Thrombospondin motifs (ADAMTs) and cathepsins (Rengel and Ospelt. Arthritis Res. Ther. 9, 221 2007). Arthritic synovial fibroblasts express both secreted (Jay et al., J. Rheumatol. 27, 594-600, 2000; Barbour et al., Morbidity and Mortality Weekly Report. 65. 2016, pp. 1052-1056; Smolen et al., Nature Reviews Disease Primers. 4. 2018. doi:10.1038/nrdp.2018.1) and cell surface MMPs (Lange-Brokaar et al., Osteoarthritis Cartilage. 20, 1484-1499. 2012; Nygaard and Firestein. Nat. Rev. Rheumatol. 16, 316-333. 2020; Bauer et al., Arthritis Res. Ther. 8, R171; 2006) MMPs. MMP14 (MT1-MMP) in particular is essential for the invasive properties of SFs.

The acquisition of aberrant matrix degradation is also characteristic of SFs in OA (Fuchs et al., Osteoarthritis Cartilage. 12, 409-418. 2004). While the OA synovial membrane typically has less immune cells than in RA, it drives cartilage degradation as in RA. What controls the switch to ECM-degradation mode of SFs is not well understood. Changes in gene expressions are obviously suspected and similar transcriptional signatures have been detected in both diseases (Cai et al., J Immunol Res. 2019, 4080735. 2019). Epigenetic changes have been detected and proposed to drive the phenotype of arthritic SFs (Nakano et al., Ann. Rheum. Dis. 72, 110-117. 2013). Whether these alterations are sufficient remains unclear.

The phenotype of SFs during arthritis has been compared to that of malignant cancer cells. Indeed, cancer growth requires a profound remodelling of the ECM in the tissue of origin, with degradation of the original tissue ECM (Hotary et al., Cell. 114, 33-45. 2003). MMPs and other matrix degradation enzymes are particularly active in malignant cells (Castro-Castro et al., Cell Dev. Biol. 32, 555-576. 2016).

Gout is an inflammatory type of arthritis, also known as gouty arthritis. Gout is the most common inflammatory arthritis with a prevalence of 2.5% in the UK. Although it has the potential to be cured, its treatment remains suboptimal (Abhishek et al., Clin Med (Lond). 2017 February; 17(1): 54-59). The ultrasonographic findings of gout include double contour sign (MSU crystal deposition on surface of hyaline articular cartilage). Normal adult articular cartilage is made up of an abundant ECM composed mainly of type II collagen fibrils interspersed with types IX and XI collagens. Cartilage loss tends to be a late feature of gouty arthropathy and, similar to bone erosion, is localized rather than diffuse. Cartilage damage is often associated with erosion and has been described as occurring in regions of biomechanical stress.

Chondrocalcinosis, or cartilage calcification, is calcification (accumulation of calcium salts) in hyaline cartilage and/or fibrocartilage. Build-up of calcium phosphate in the ankle joints has been found in about 50% of the general population, and may be associated with osteoarthritis (Hubert et al., BMC Musculoskelet Disord. 2018; 19: 169). It is often found in weight bearing joints such as the hip, ankle and knee. The molecular structure of calcium pyrophosphate has the potential of triggering inflammatory responses. The presence of chondrocalcinosis has associations with the degradation of cartilage menisci and synovial tissue. It has been reported that presence of calcium-containing crystals, which are associated with chondrocalcinosis, was associated with higher prevalence of cartilage and meniscal damage (Gersing et al., Eur Radiol. 2017 June; 27(6):2497-2506. doi: 10.1007/s00330-016-4608-8. Epub 2016 Oct. 4).

Fibromyalgia (FM) is a medical condition characterized by chronic widespread pain and a heightened pain response to pressure. FM is common in rheumatoid arthritis, axial spondyloarthritis and psoriatic arthritis, and could therefore influence management of these rheumatic conditions. FM is also associated with costochondritis.

Costochondritis is an inflammation of the cartilage in the rib cage. The condition usually affects the cartilage where the upper ribs attach to the breastbone, or sternum, an area known as the costosternal joint or costosternal junction. Costochondritis can be caused by mechanical stress, leading to cartilage loss and/or ECM degradation.

Osteochondritis dissecans (OCD or OD) is a disorder in which cracks form in the articular cartilage and the underlying subchondral bone. OCD usually causes pain during and after sports. In later stages of the disorder there will be swelling of the affected joint which catches and locks during movement. Physical examination in the early stages can identify pain as symptom, in later stages there could be an effusion, tenderness, and a crackling sound with joint movement. Treatment to prevent, reduce, or reverse of ECM degradation and/or cartilage loss would benefit patients with osteochondritis dissecans. In some cases, the osteochondritis dissecans to be treated is associated with ECM degradation and/or cartilage loss. Polychondritis, or relapsing polychondritis (RP), is an immune-mediated systemic disease characterized by recurrent inflammatory episodes of cartilaginous and proteoglycan-rich tissues, including the elastic cartilage of the ear and nose, the hyaline cartilage of peripheral joints, the fibrocartilage at axial sites and the cartilage of the tracheobronchial tree, which result in progressive anatomical deformation and functional impairment of the involved structures (Borgio et al., Biomedicines. 2018 September; 6(3): 84). Mono- or, more frequently, bilateral auricular chondritis is the most common feature of RP, which is observed in up to 90% of patients during the course of the disease, and is the inaugural symptom in 20% of cases. The onset is abrupt, with painful red to violaceous erythema and edema confined to the cartilaginous part of the ear, typically sparing the lobe, which lacks cartilage. Acute inflammatory episodes tend to resolve spontaneously within few days or weeks, with recurrence at variable intervals. As a long-term consequence of repeated flares, the cartilage matrix is severely damaged and replaced by fibrous connective tissue (Borgio et al., Biomedicines. 2018 September; 6(3): 84).

In some embodiments, the disease/condition to be treated in accordance with the present disclosure is a joint disorder. A joint is defined as a connection between two bones in the skeletal system. Joints can be classified by the type of the tissue present (fibrous, cartilaginous or synovial), or by the degree of movement permitted (synarthrosis, amphiarthrosis or diarthrosis). Therefore, a joint disorder is defined as a condition which affects a connection between two bones in the skeletal system. Definitions of specific joints and related aspects of anatomy can be found in “Netter, F. H. (2006). Atlas of human anatomy. Philadelphia, PA: Saunders/Elsevier”, which is incorporated by reference in its entirety. The joint disorder may affect a fibrous, cartilaginous or synovial joint.

Fibrous joints are connected by dense connective tissue consisting mainly of collagen. These joints are also called fixed or immovable joints because they do not move. Fibrous joints have no joint cavity and are connected via fibrous connective tissue. The skull bones are connected by fibrous joints called sutures.

Cartilaginous joints are a type of joint where the bones are entirely joined by cartilage, either hyaline cartilage or fibrocartilage. These joints generally allow more movement than fibrous joints but less movement than synovial joints.

A synovial joint is characterised by the presence of a fluid-filled joint cavity contained within a fibrous capsule. It is the most common type of joint found in the human body and contains several structures which are not seen in fibrous or cartilaginous joints. The three main features of a synovial joint are: (i) articular capsule, (ii) articular cartilage, (iii) synovial fluid. The articular capsule surrounds the joint and is continuous with the periosteum of articulating bones. The articulating surfaces of a synovial joint (i.e. the surfaces that directly contact each other as the bones move) are covered by a thin layer of hyaline cartilage. The articular cartilage has two main roles: (i) minimising friction upon joint movement, and (ii) absorbing shock. The synovial fluid is located within the joint cavity of a synovial joint. It has three primary functions. Synovial joints can include accessory structures such as tendons, ligaments, bursae, and vasculature. There are numerous types of synovial joints. In some cases, the joint disorder is of a gliding joint, a hinge joint, a pivot joint, an ellipsoid joint, saddle joint, or a ball and socket joint. A gliding joint, also known as a plane joint or planar joint, is a common type of synovial joint formed between bones that meet at flat or nearly flat articular surfaces. Gliding joints allow the bones to glide past one another in any direction along the plane of the joint—up and down, left and right, and diagonally. Slight rotations can also occur at these joints, but are limited by the shape of the bones and the elasticity of the joint capsule surrounding them. A hinge joint (ginglymus) is a bone joint in which the articular surfaces are molded to each other in such a manner as to permit motion only in one plane. According to one classification system they are said to be uniaxial (having one degree of freedom) (Platzer, Werner (2008) Color Atlas of Human Anatomy, Volume 1). The direction which the distal bone takes in this motion is seldom in the same plane as that of the axis of the proximal bone; there is usually a certain amount of deviation from the straight line during flexion. The articular surfaces of the bones are connected by strong collateral ligaments. The best examples of ginglymoid joints are the Interphalangeal joints of the hand and those of the foot and the joint between the humerus and ulna. The knee joints and ankle joints are less typical, as they allow a slight degree of rotation or of side-to-side movement in certain positions of the limb. The knee is the largest hinge joint in the human body. A pivot joint (trochoid joint, rotary joint or lateral ginglymus) is a type of synovial joint whose movement axis is parallel to the long axis of the proximal bone, which typically has a convex articular surface. According to one classification system, a pivot joint has one degree of freedom (Platzer, Werner (2008) Color Atlas of Human Anatomy, Volume 1). An ellipsoid joint (also called a condyloid joint) is an ovoid articular surface, or condyle that is received into an elliptical cavity. This permits movement in two planes, allowing flexion, extension, adduction, abduction, and circumduction, as seen in the wrist joint. A saddle joint is a type of synovial joint in which the opposing surfaces are reciprocally concave and convex. It is found in the thumb, the thorax, and the middle ear, and the heel. A ball and socket joint (or spheroid joint) is a type of synovial joint in which the ball-shaped surface of one rounded bone fits into the cup-like depression of another bone. The distal bone is capable of motion around an indefinite number of axes, which have one common centre. This enables the joint to move in many directions.

The joint disorder may affect a synarthrosis, amphiarthrosis or diarthrosis joint. The hip and shoulder are ball and socket joints. A synarthrosis is a type of joint which allows no movement under normal conditions. Sutures and gomphoses are both synarthroses. An amphiarthrosis is a joint that has limited mobility. An example of this type of joint is the cartilaginous joint that unites the bodies of adjacent vertebrae. A diarthrosis joint is a freely moveable joint. Sometimes the terms diarthrosis joints and synovial joints are used interchangeably.

The joint disorder may affect any joint. In some cases, the joint disorder affects the hip, knee, ankle, foot, toe, shoulder, elbow, wrist, hand, finger, neck, spine, ribs, or sacroiliac joint.

In some embodiments, a joint disorder is selected from: osteoarthritis, psoriasis arthritis, rheumatoid arthritis, juvenile arthritis, post-trauma arthritis, bursitis, gout, chondrocalcinosis, fibromyalgia, costochondritis, osteochondritis dissecans, polychondritis, cartilage damage, tendon damage, or ligament damage.

Bursitis is inflammation of a bursa, a small fluid-filled sac that acts as a cushion between bone and muscle, skin or tendon. The type of bursitis depends on where the affected bursa is located. This soft tissue condition commonly affects the shoulder, elbow, hip, buttocks, knees and calf. Athletes, the elderly and people who do repetitive movements like manual laborers and musicians are more likely to get bursitis. Bursitis is sometimes mistaken for arthritis because the pain can occur in a joint. Tendon damage, or tendinopathy, can be caused in a number of ways, for example from overuse, aging, wear and tear, or a mechanical injury. Tendon damage may be tendinitis or tendinosis. Tendinitis refers to inflammation of a tendon, and tendinosis relates to tears in the tissue in and around the tendon. The tendon damage may be a strained tendon, a sprained tendon, a torn tendon, a partially ruptured tendon or a completely ruptured tendon.

Ligament damage can be caused in a number of ways for example from overuse, aging, wear and tear, or a mechanical injury. The ligament damage may be a strained ligament, a sprained ligament, a torn ligament, a partially ruptured ligament or a completely ruptured ligament.

Administration of the articles of the present disclosure is preferably in a ‘therapeutically-effective’ or ‘prophylactically-effective’ amount, this being sufficient to show therapeutic or prophylactic benefit to the subject. The actual amount administered, and rate and time-course of administration, will depend on the nature and severity of the disease/condition and the particular article administered. Prescription of treatment, e.g. decisions on dosage etc., is within the responsibility of general practitioners and other medical doctors, and typically takes account of the disease/disorder to be treated, the condition of the individual subject, the site of delivery, the method of administration and other factors known to practitioners. Examples of the techniques and protocols mentioned above can be found in Remington's ‘The Science and Practice of Pharmacy’ (ed. A. Adejare), 23rd Edition (2020), Academic Press.

Administration of the articles of the present disclosure may be topical, parenteral, systemic, intracavitary, intravenous, intra-arterial, intramuscular, intrathecal, intraocular, intravitreal, intraconjunctival, subretinal, suprachoroidal, subcutaneous, intradermal, intrathecal, oral, nasal ortransdermal. Administration may be by injection or infusion. Administration of the articles of the present disclosure may be intratumoral.

In some aspects and embodiments in accordance with the present disclosure there may be targeted delivery of articles of the present disclosure, i.e. wherein the concentration of the relevant agent in the subject is increased in some parts of the body relative to other parts of the body. In some embodiments, the methods comprise intravenous, intra-arterial, intramuscular or subcutaneous administration and wherein the relevant article is formulated in a targeted agent delivery system. Suitable targeted delivery systems include, for example, nanoparticles, liposomes, micelles, beads, polymers, metal particles, dendrimers, antibodies, aptamers, nanotubes or micro-sized silica rods. Such systems may comprise a magnetic element to direct the agent to the desired organ or tissue. Suitable nanocarriers and delivery systems will be apparent to one skilled in the art.

In some cases, the articles of the present disclosure are formulated for targeted delivery to specific cells, a tissue, an organ and/or a tumor.

Administration may be alone or in combination with other treatments, either simultaneously or sequentially dependent upon the condition to be treated. The antigen-binding molecule or composition described herein and a therapeutic agent may be administered simultaneously or sequentially.

In some embodiments, the methods comprise additional therapeutic or prophylactic intervention, e.g. for the treatment/prevention of a cancer. In some embodiments, the therapeutic or prophylactic intervention is selected from chemotherapy, immunotherapy, radiotherapy, surgery, vaccination and/or hormone therapy. In some embodiments, the therapeutic or prophylactic intervention comprises leukapheresis. In some embodiments, the therapeutic or prophylactic intervention comprises a stem cell transplant.

Simultaneous administration refers to administration of the antigen-binding molecule, polypeptide, CAR, nucleic acid (or plurality thereof), expression vector (or plurality thereof), cell or composition and therapeutic agent together, for example as a pharmaceutical composition containing both agents (combined preparation), or immediately after each other and optionally via the same route of administration, e.g. to the same artery, vein or other blood vessel. Sequential administration refers to administration of one of the antigen-binding molecule/composition or therapeutic agent followed after a given time interval by separate administration of the other agent. It is not required that the two agents are administered by the same route, although this is the case in some embodiments. The time interval may be any time interval.

Chemotherapy and radiotherapy respectively refer to treatment of a cancer with a drug or with ionising radiation (e.g. radiotherapy using X-rays or γ-rays). The drug may be a chemical entity, e.g. small molecule pharmaceutical, antibiotic, DNA intercalator, protein inhibitor (e.g. kinase inhibitor), or a biological agent, e.g. antibody, antibody fragment, aptamer, nucleic acid (e.g. DNA, RNA), peptide, polypeptide, or protein.

The chemotherapy may be administered according to a treatment regime. The treatment regime may be a pre-determined timetable, plan, scheme or schedule of chemotherapy administration which may be prepared by a physician or medical practitioner and may be tailored to suit the patient requiring treatment. The treatment regime may indicate one or more of: the type of chemotherapy to administer to the patient; the dose of each drug or radiation; the time interval between administrations; the length of each treatment; the number and nature of any treatment holidays, if any etc. For a co-therapy a single treatment regime may be provided which indicates how each drug is to be administered.

Chemotherapeutic drugs may be selected from: Abemaciclib, Abiraterone Acetate, Abitrexate (Methotrexate), Abraxane (Paclitaxel Albumin-stabilized Nanoparticle Formulation), ABVD, ABVE, ABVE-PC, AC, Acalabrutinib, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ambochlorin (Chlorambucil), Amboclorin (Chlorambucil), Amifostine, Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Atezolizumab, Avastin (Bevacizumab), Avelumab, Axicabtagene Ciloleucel, Axitinib, Azacitidine, Bavencio (Avelumab), BEACOPP, Becenum (Carmustine), Beleodaq (Belinostat), Belinostat, Bendamustine Hydrochloride, BEP, Besponsa (Inotuzumab Ozogamicin), Bevacizumab, Bexarotene, Bexxar (Tositumomab and Iodine I 131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Brigatinib, BuMel, Busulfan, Busulfex (Busulfan), Cabazitaxel, Cabometyx (Cabozantinib-S-Malate), Cabozantinib-S-Malate, CAF, Calquence (Acalabrutinib), Campath (Alemtuzumab), Camptosar (Irinotecan Hydrochloride), Capecitabine, CAPOX, Carac (Fluorouracil—Topical), Carboplatin, CARBOPLATIN-TAXOL, Carfilzomib, Carmubris (Carmustine), Carmustine, Carmustine Implant, Casodex (Bicalutamide), CEM, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), Copanlisib Hydrochloride, COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Durvalumab, Efudex (Fluorouracil—Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enasidenib Mesylate, Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista (Raloxifene Hydrochloride), Evomela (Melphalan Hydrochloride), Exemestane, 5-FU (Fluorouracil Injection), 5-FU (Fluorouracil—Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil—Topical), Fluorouracil Injection, Fluorouracil—Topical, Flutamide, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Idhifa (Enasidenib Mesylate), Ifex (Ifosfamide), Ifosfamide, Ifosfamidum (Ifosfamide), IL-2 (Aldesleukin), Imatinib Mesylate, Imbruvica (Ibrutinib), Imfinzi (Durvalumab), Imiquimod, Imlygic (Talimogene Laherparepvec), Inlyta (Axitinib), Inotuzumab Ozogamicin, Interferon Alfa-2b, Recombinant, Interleukin-2 (Aldesleukin), Intron A (Recombinant Interferon Alfa-2b), Iodine I 131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and Hyaluronidase Human, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq (Atezolizumab), Temodar (Temozolomide), Temozolomide, Temsirolimus, Thalidomide, Thalomid (Thalidomide), Thioguanine, Thiotepa, Tisagenlecleucel, Tolak (Fluorouracil—Topical), Topotecan Hydrochloride, Toremifene, Torisel (Temsirolimus), Tositumomab and Iodine I 131 Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Valrubicin, Valstar (Valrubicin), Vandetanib, VAMP, Varubi (Rolapitant Hydrochloride), Vectibix (Panitumumab), VeIP, Velban (Vinblastine Sulfate), Velcade (Bortezomib), Velsar (Vinblastine Sulfate), Vemurafenib, Venclexta (Venetoclax), Venetoclax, Verzenio (Abemaciclib), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Vyxeos (Daunorubicin Hydrochloride and Cytarabine Liposome), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yescarta (Axicabtagene Ciloleucel), Yondelis (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib) and Zytiga (Abiraterone Acetate).

In some embodiments, the treatment may comprise administration of a corticosteroid, e.g. dexamethasone and/or prednisone.

In some embodiments, the methods comprise additional therapeutic or prophylactic intervention, e.g. for the treatment/prevention of cartilage degradation/a disease/condition characterised by cartilage degradation. Such intervention includes palliation (e.g., chondroplasty and debridement), repair (e.g., drilling and microfracture [MF]), and restoration (e.g., autologous chondrocyte implantation [ACI], osteochondral autograft [OAT], and osteochondral allograft [OCA]) (Richter et al., Sports Health. March-April 2016; 8(2):153-60. doi: 10.1177/1941738115611350. Epub 2015 Oct. 12).

Multiple doses of the antigen-binding molecule, polypeptide, CAR, nucleic acid (or plurality thereof), expression vector (or plurality thereof), cell or composition may be provided. One or more, or each, of the doses may be accompanied by simultaneous or sequential administration of another therapeutic agent.

Multiple doses may be separated by a predetermined time interval, which may be selected to be one of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or31 days, or 1, 2, 3, 4, 5, or 6 months. By way of example, doses may be given once every 7, 14, 21 or 28 days (plus or minus 3, 2, or 1 days).

In accordance with various aspects of the present disclosure, a method of treating and/or preventing a disease/condition may comprise one or more of the following: reducing a function of CNX/CRT and/or a function of a complex comprising CNX/CRT; reducing extracellular matrix degradation (e.g. collagen and/or gelatin degradation); reducing oxireductase activity; reducing disulfide bond reductase activity; reducing cartilage degradation; inhibiting tumor growth; reducing metastasis of a cancer; increasing survival of a subject having a cancer; reducing the pathology of a disease/condition characterised by ECM degradation in a subject; and/or reducing the pathology of a disease/condition characterised by cartilage degradation (e.g. arthritis) in a subject.

### Methods of Detection

The present disclosure also provides the articles of the present disclosure for use in methods for detecting, localizing or imaging CNX/CRT, or cells expressing CNX/CRT.

The antigen-binding molecules described herein may be used in methods that involve detecting binding of the antigen-binding molecule to CNX/CRT. Such methods may involve detection of the bound complex of the antigen-binding molecule and CNX/CRT.

As such, a method is provided, comprising contacting a sample containing, or suspected to contain, CNX, and detecting the formation of a complex of the antigen-binding molecule and CNX. Also provided is a method comprising contacting a sample containing, or suspected to contain, a cell expressing CNX, and detecting the formation of a complex of the antigen-binding molecule and a cell expressing CNX.

Suitable method formats are well known in the art, including immunoassays such as sandwich assays, e.g. ELISA. The methods may involve labelling the antigen-binding molecule, or target(s), or both, with a detectable moiety, e.g. a fluorescent label, phosphorescent label, luminescent label, immuno-detectable label, radiolabel, chemical, nucleic acid or enzymatic label as described herein. Detection techniques are well known to those of skill in the art and can be selected to correspond with the labelling agent.

Methods comprising detecting CNX/CRT, or cells expressing CNX/CRT, include methods for diagnosing/prognosing a disease/condition described herein.

Methods of this kind may be performed in vitro on a patient sample, or following processing of a patient sample. Once the sample is collected, the patient is not required to be present for the in vitro method to be performed, and therefore the method may be one which is not practised on the human or animal body. In some embodiments, the method is performed in vivo.

Such methods may involve detecting or quantifying CNX/CRT and/or cells expressing CNX/CRT, e.g. in a patient sample. Where the method comprises quantifying the relevant factor, the method may further comprise comparing the determined amount against a standard or reference value as part of the diagnostic or prognostic evaluation. Other diagnostic/prognostic tests may be used in conjunction with those described herein to enhance the accuracy of the diagnosis or prognosis or to confirm a result obtained by using the tests described herein.

Detection in a sample may be used for the purpose of diagnosis of a disease/condition (e.g. a cancer), predisposition to a disease/condition, or for providing a prognosis (prognosticating) for a disease/condition, e.g. a disease/condition described herein. The diagnosis or prognosis may relate to an existing (previously diagnosed) disease/condition.

A sample may be taken from any tissue or bodily fluid. The sample may comprise or may be derived from: a quantity of blood; a quantity of serum derived from the individual's blood which may comprise the fluid portion of the blood obtained after removal of the fibrin clot and blood cells; a tissue sample or biopsy; pleural fluid; cerebrospinal fluid (CSF); or cells isolated from said individual. In some embodiments, the sample may be obtained or derived from a tissue or tissues which are affected by the disease/condition (e.g. tissue or tissues in which symptoms of the disease manifest, or which are involved in the pathogenesis of the disease/condition).

A subject may be selected for diagnostic/prognostic evaluation based on the presence of symptoms indicative of a disease/condition described herein, or based on the subject being considered to be at risk of developing a disease/condition described herein.

The present disclosure also provides methods for selecting/stratifying a subject for treatment with a CNX/CRT-targeted agent. In some embodiments a subject is selected for treatment/prevention in accordance with the methods of the present disclosure, or is identified as a subject which would benefit from such treatment/prevention, based on detection/quantification of CNX/CRT, or cells expressing CNX/CRT, e.g. in a sample obtained from the individual.

### Subjects

A subject in accordance with the various aspects of the present disclosure may be any animal or human. Therapeutic and prophylactic applications may be in human or animals (veterinary use).

The subject to be administered with an article of the present disclosure (e.g. in accordance with therapeutic or prophylactic intervention) may be a subject in need of such intervention. The subject is preferably mammalian, more preferably human. The subject may be a non-human mammal, but is more preferably human. The subject may be male or female. The subject may be a patient.

A subject may have (e.g. may have been diagnosed with) a disease or condition described herein, may be suspected of having such a disease/condition, or may be at risk of developing/contracting such a disease/condition. In embodiments according to the present disclosure, a subject may be selected for treatment according to the methods based on characterisation for one or more markers of such a disease/condition.

In some embodiments, a subject may be selected for therapeutic or prophylactic intervention as described herein based on the detection of cells/tissue expressing CNX/CRT, or of cells/tissue overexpressing CNX/CRT, e.g. in a sample obtained from the subject.

### Kits

In some aspects of the present disclosure a kit of parts is provided. In some embodiments, the kit may have at least one container having a predetermined quantity of an antigen-binding molecule, polypeptide, CAR, nucleic acid (or plurality thereof), expression vector (or plurality thereof), cell or composition described herein.

In some embodiments, the kit may comprise materials for producing an antigen-binding molecule, polypeptide, CAR, nucleic acid (or plurality thereof), expression vector (or plurality thereof), cell or composition described herein.

The kit may provide the antigen-binding molecule, polypeptide, CAR, nucleic acid (or plurality thereof), expression vector (or plurality thereof), cell or composition together with instructions for administration to a patient in order to treat a specified disease/condition.

In some embodiments the kit may further comprise at least one container having a predetermined quantity of another therapeutic agent (e.g. as described herein). In such embodiments, the kit may also comprise a second medicament or pharmaceutical composition such that the two medicaments or pharmaceutical compositions may be administered simultaneously or separately such that they provide a combined treatment for the specific disease or condition.

Kits according to the present disclosure may include instructions for use, e.g. in the form of an instruction booklet or leaflet. The instructions may include a protocol for performing any one or more of the methods described herein.

### Sequence Identity

As used herein, ‘sequence identity’ refers to the percent of nucleotides/amino acid residues in a subject sequence that are identical to nucleotides/amino acid residues in a reference sequence, after aligning the sequences and, if necessary, introducing gaps, to achieve the maximum percent sequence identity between the sequences. Pairwise and multiple sequence alignment for the purposes of determining percent sequence identity between two or more amino acid or nucleic acid sequences can be achieved in various ways known to a person of skill in the art, for instance, using publicly available computer software such as ClustalOmega (Söding, J. 2005, Bioinformatics 21, 951-960), T-coffee (Notredame et al. 2000, J. Mol. Biol. (2000) 302, 205-217), Kalign (Lassmann and Sonnhammer 2005, BMC Bioinformatics, 6(298)) and MAFFT (Katoh and Standley 2013, Molecular Biology and Evolution, 30(4) 772-780) software. When using such software, the default parameters, e.g. for gap penalty and extension penalty, are preferably used.

Sequences

| SEQ | | |
|---|---|---|
| ID | | |
| NO: | DESCRIPTION | SEQUENCE |
| 1 | 1D3 VH | EVQLVESGGGVVQPGRSLRLSCAASGFTFSSYGMHWVRQAPGKGLEWVAVISYDGSNKYYADSV |
| | | KGRFTISRDNSKNTLYLQMNSLRAEDTAVYYCARGGRAFDIWGQGTMVTVSS |
| 2 | 1D3, 2G9, 3F9 | GFTFSSYG |
| | HC-CDR1 | |
| 3 | 1D3, 1E6, 2G9, | ISYDGSNK |
| | 2G12, 3F9, 5E8 | |
| | HC-CDR2 | |
| 4 | 1D3 HC-CDR3 | ARGGRAFDI |
| 5 | 1D3 HC-FR1 | EVQLVESGGGVVQPGRSLRLSCAAS |
| 6 | 1D3, 1E6, 2G9, | MHWVRQAPGKGLEWVAV |
| | 2G12, 3F9, 5E8 | |
| | HC-FR2 | |
| 7 | 1D3, 1E6, 2G9, | YYADSVKGRFTISRDNSKNTLYLQMNSLRAEDTAVYYC |
| | 2G12, 3F9, 5E8, | |
| | C001, C008, | |
| | C025 HC-FR3 | |
| 8 | 1D3, 1D6, C010, | WGQGTMVTVSS |
| | C023, C040, | |
| | C046 HC-FR4 | |
| 9 | 1D3 VL | DVVMTQSPLSLPVTLGQPASISCKSSQSLVNSGGDTFLNWFQQRPGQSPRRLIYKVSNRDSGVPD |
| | | RFSGSGSGTDFTLEISRVEAEDVGVYYCMQGTHWPRTFGQGTKVEIK |
| 10 | 1D3 LC-CDR1 | QSLVNSGGDTF |
| 11 | 1D3, 2G12 LC- | KVS |
| | CDR2 | |
| 12 | 1D3 LC-CDR3 | MQGTHWPRT |
| 13 | 1D3 LC-FR1 | DVVMTQSPLSLPVTLGQPASISCKSS |
| 14 | 1D3 LC-FR2 | LNWFQQRPGQSPRRLIY |
| 15 | 1D3 LC-FR3 | NRDSGVPDRFSGSGSGTDFTLEISRVEAEDVGVYYC |
| 16 | 1D3, 1E1, 2G12, | FGQGTKVEIK |
| | 3F9 LC-FR4 | |
| 17 | 1D6 VH | QVQLQESGAGLLKPSETLSLTCAVYGGSFSGYYWSWIRQPPGKGLEWIGRIYTSGSTNYNPSLKSR |
| | | VTISVDTSKNQFSLKLSSVTAADTAVYYCARVGAGAFDIWGQGTMVTVSS |
| 18 | 1D6 HC-CDR1 | GGSFSGYY |
| 19 | 1D6 HC-CDR2 | IYTSGST |
| 20 | 1D6 HC-CDR3 | ARVGAGAFDI |
| 21 | 1D6 HC-FR1 | QVQLQESGAGLLKPSETLSLTCAVY |
| 22 | 1D6 HC-FR2 | WSWIRQPPGKGLEWIGR |
| 23 | 1D6 HC-FR3 | NYNPSLKSRVTISVDTSKNQFSLKLSSVTAADTAVYYC |
| 24 | 1D6 VL | QAVLTQPSSLSASPGASASLTCTLRSDINVGAYRIYWYQQKPGSPPRYLLRYKSDSDKQQGSGVSN |
| | | RFSASKDASANAGILHISGLHSEDEADYYCLIWHNSVWVFGGGTKLTVL |
| 25 | 1D6 LC-CDR1 | SDINVGAYR |
| 26 | 1D6, 2C6, 2H6, | YKSDSDK |
| | 2G9, C010 LC- | |
| | CDR2 | |
| 27 | 1D6 LC-CDR3 | LIWHNSVWV |
| 28 | 1D6, 2C6, 2H6, | QAVLTQPSSLSASPGASASLTCTLR |
| | 2G9, 3D1, C010 | |
| | LC-FR1 | |
| 29 | 1D6 LC-FR2 | IYWYQQKPGSPPRYLLR |
| 30 | 1D6 LC-FR3 | QQGSGVSNRFSASKDASANAGILHISGLHSEDEADYYC |
| 31 | 1D6, 2C6, 2H6, | FGGGTKLTVL |
| | 2G9, 3D1, 2H5, | |
| | 3F8, C025, C040 | |
| | LC-FR4 | |
| 32 | 1E1 VH | QVQLQESGGGVVRPGGSLRLSCAASGFTFDDYGMSWVRQAPGKGLEWVSGINWNGGSTGYADS |
| | | VKGRFTISRDNAKNSLYLQMNSLRAEDTAVYYCARGIAVDYWGQGTLVTVSS |
| 33 | 1E1 HC-CDR1 | GFTFDDYG |
| 34 | 1E1 HC-CDR2 | INWNGGST |
| 35 | 1E1 HC-CDR3 | ARGIAVDY |
| 36 | 1E1 HC-FR1 | QVQLQESGGGVVRPGGSLRLSCAAS |
| 37 | 1E1 HC-FR2 | MSWVRQAPGKGLEWVSG |
| 38 | 1E1 HC-FR3 | GYADSVKGRFTISRDNAKNSLYLQMNSLRAEDTAVYYC |
| 39 | 1E1, 2C6, 2H6, | WGQGTLVTVSS |
| | 3D1, 2G9, 2H5, | |
| | 4G9, 5A3, 5E8, | |
| | C001, C008, | |
| | C025, C117 HC- | |
| | FR4 | |
| 40 | 1E1 VL | DVVMTQSPLSLPVTPGEPASISCRSSQSLLHSNGYNYLDWYLQKPGQSPQLLIYLGSNRASGVPDR |
| | | FSGSGSGTDFTLKISRVEAEDVGVYYCMQALQTPWTFGQGTKVEIK |
| 41 | 1E1, 5A3 LC- | QSLLHSNGYNY |
| | CDR1 | |
| 42 | 1E1, 5A3, C023 | LGS |
| | LC-CDR2 | |
| 43 | 1E1 LC-CDR3 | MQALQTPWT |
| 44 | 1E1, 5A3, C023 | DVVMTQSPLSLPVTPGEPASISCRSS |
| | LC-FR1 | |
| 45 | 1E1, 5A3 LC-FR2 | LDWYLQKPGQSPQLLIY |
| 46 | 1E1, 5A3 LC-FR3 | NRASGVPDRFSGSGSGTDFTLKISRVEAEDVGVYYC |
| 47 | 1E6 VH | QLQLQESGGGVVQPGRSLRLSCAASGFTFSSYAMHWVRQAPGKGLEWVAVISYDGSNKYYADSV |
| | | KGRFTISRDNSKNTLYLQMNSLRAEDTAVYYCARDIPYGGYAHYYYYGMDVWGQGTTVTVSS |
| 48 | 1E6, 2G12, 5E8, | GFTFSSYA |
| | C023, C046, | |
| | C025 HC-CDR1 | |
| 49 | 1E6 HC-CDR3 | ARDIPYGGYAHYYYYGMDV |
| 50 | 1E6 HC-FR1 | QLQLQESGGGVVQPGRSLRLSCAAS |
| 51 | 1E6, 2G12, 3F8, | WGQGTTVTVSS |
| | 3F9 HC-FR4 | |
| 52 | 1E6 VL | QTVVTQEPSLTVSPGGTVTLTCTSSTGAVTSASYPNWFQQKPGQAPRKLIYDTNNRHSWTPARFS |
| | | GSLLGGKAALTLSGAEPEDEAVYYCFLSYSGAYWIFGGGTHLTVL |
| 53 | 1E6 LC-CDR1 | TGAVTSASY |
| 54 | 1E6 LC-CDR2 | DTN |
| 55 | 1E6 LC-CDR3 | FLSYSGAYWI |
| 56 | 1E6 LC-FR1 | QTVVTQEPSLTVSPGGTVTLTCTSS |
| 57 | 1E6 LC-FR2 | PNWFQQKPGQAPRKLIY |
| 58 | 1E6 LC-FR3 | NRHSWTPARFSGSLLGGKAALTLSGAEPEDEAVYYC |
| 59 | 1E6 LC-FR4 | FGGGTHLTVL |
| 60 | 2C6, 2H6, 3D1 | QVQLVQSGAEVKKPGSSVKVSCKASGGTFSSYAISWVRQAPGQGLEWMGRIIPILGIANYAQKFQG |
| | VH | RVTITADKSTSTAYMELSSLRSEDTAVYYCAREERGSYFRAPFDYWGQGTLVTVSS |
| 61 | 2C6, 2H6, 3D1, | GGTFSSYA |
| | 2H5 HC-CDR1 | |
| 62 | 2C6, 2H6, 3D1 | IIPILGIA |
| | HC-CDR2 | |
| 63 | 2C6, 2H6, 3D1 | AREERGSYFRAPFDY |
| | HC-CDR3 | |
| 64 | 2C6, 2H6, 3D1 | QVQLVQSGAEVKKPGSSVKVSCKAS |
| | HC-FR1 | |
| 65 | 2C6, 2H6, 3D1 | ISWVRQAPGQGLEWMGR |
| | HC-FR2 | |
| 66 | 2C6, 2H6, 3D1, | NYAQKFQGRVTITADKSTSTAYMELSSLRSEDTAVYYC |
| | 2H5 HC-FR3 | |
| 67 | 2C6 VL | QAVLTQPSSLSASPGASASLTCTLRSDISVGTYRIYWYQQKPGSPPHYLLRYKSDSDKQQGSGVPS |
| | | RFSGFKDASANAGILLISGLQSEDEADYYCLIWHSNTWVFGGGTKLTVL |
| 68 | 2C6 LC-CDR1 | SDISVGTYR |
| 69 | 2C6 LC-CDR3 | LIWHSNTWV |
| 70 | 2C6 LC-FR2 | IYWYQQKPGSPPHYLLR |
| 71 | 2C6 LC-FR3 | QQGSGVPSRFSGFKDASANAGILLISGLQSEDEADYYC |
| 72 | 2H6, 2G9 VL | QAVLTQPSSLSASPGASASLTCTLRSGINVGTYRIYWYQQKPGSPPQYLLRYKSDSDKQQGSGVPS |
| | | RFSGSKDASANAGILLISGLQSEDEADYYCMIWHSSAWVFGGGTKLTVL |
| 73 | 2H6, 2G9, C010 | SGINVGTYR |
| | LC-CDR1 | |
| 74 | 2H6, 2G9 LC- | MIWHSSAWV |
| | CDR3 | |
| 75 | 2H6, 2G9, C010 | IYWYQQKPGSPPQYLLR |
| | LC-FR2 | |
| 76 | 2H6, 2G9, 3D1 | QQGSGVPSRFSGSKDASANAGILLISGLQSEDEADYYC |
| | LC-FR3 | |
| 77 | 3D1 VL | QAVLTQPSSLSASPGASASLTCTLRSGIHVGTYRIYWYQQKPGSPPQFLLKYKTDSDKQQGSGVPS |
| | | RFSGSKDASANAGILLISGLQSEDEADYYCMIWHNNAWVFGGGTKLTVL |
| 78 | 3D1 LC-CDR1 | SGIHVGTYR |
| 79 | 3D1 LC-CDR2 | YKTDSDK |
| 80 | 3D1, 4G9 LC- | MIWHNNAWV |
| | CDR3 | |
| 81 | 3D1 LC-FR2 | IYWYQQKPGSPPQFLLK |
| 82 | 2G9 VH | QVTLKESGGGVVQPGRSLRLSCAASGFTFSSYGMHWVRQAPGKGLEWVAVISYDGSNKYYADSV |
| | | KGRFTISRDNSKNTLYLQMNSLRAEDTAVYYCTQGRDYWGQGTLVTVSS |
| 83 | 2G9 HC-CDR3 | TQGRDY |
| 84 | 2G9 HC-FR1 | QVTLKESGGGVVQPGRSLRLSCAAS |
| 85 | 2G12 VH | QVQLVQSGGGVVQPGRSLRLSCAASGFTFSSYAMHWVRQAPGKGLEWVAVISYDGSNKYYADSV |
| | | KGRFTISRDNSKNTLYLQMNSLRAEDTAVYYCARASRGMDVWGQGTTVTVSS |
| 86 | 2G12 HC-CDR3 | ARASRGMDV |
| 87 | 2G12, 5E8, C001, | QVQLVQSGGGVVQPGRSLRLSCAAS |
| | C008 HC-FR1 | |
| 88 | 2G12 VL | DVVMTQSPLSLPVTPGQSASISCRSSQSLVDRDGNTYLTWFHQRPGQSPRRLIFKVSNRDSGVPD |
| | | RFSGSGSGTDFTLRISRVEAEDVGVYYCMQVTHWPRTFGQGTKVEIK |
| 89 | 2G12 LC-CDR1 | QSLVDRDGNTY |
| 90 | 2G12 LC-CDR3 | MQVTHWPRT |
| 91 | 2G12 LC-FR1 | DVVMTQSPLSLPVTPGQSASISCRSS |
| 92 | 2G12 LC-FR2 | LTWFHQRPGQSPRRLIF |
| 93 | 2G12 LC-FR3 | NRDSGVPDRFSGSGSGTDFTLRISRVEAEDVGVYYC |
| 94 | 2H5 VH | QVQLQQSGAEVKKPGSSVKVSCKASGGTFSSYAISWVRQAPGQGLEWMGGIIPIFGTANYAQKFQ |
| | | GRVTITADKSTSTAYMELSSLRSEDTAVYYCARARSGSYSLHYWGQGTLVTVSS |
| 95 | 2H5 HC-CDR2 | IIPIFGTA |
| 96 | 2H5 HC-CDR3 | ARARSGSYSLHY |
| 97 | 2H5 HC-FR1 | QVQLQQSGAEVKKPGSSVKVSCKAS |
| 98 | 2H5 HC-FR2 | ISWVRQAPGQGLEWMGG |
| 99 | 000 | 000 |
| 100 | 2H5 VL | NFMLTQPHSVSESPGKTVTISCTGSSGSIDSNYVQWYQQRPGSAPTTVIFEDSQRPSGVPDRFSGS |
| | | TDRSSNSASLTISGLKTEDEADYYCQSYDSSNNWVFGGGTKLTVL |
| 101 | 2H5 LC-CDR1 | SGSIDSNY |
| 102 | 2H5 LC-CDR2 | EDS |
| 103 | 2H5 LC-CDR3 | QSYDSSNNWV |
| 104 | 2H5 LC-FR1 | NFMLTQPHSVSESPGKTVTISCTGS |
| 105 | 2H5 LC-FR2 | VQWYQQRPGSAPTTVIF |
| 106 | 2H5 LC-FR3 | QRPSGVPDRFSGSTDRSSNSASLTISGLKTEDEADYYC |
| 107 | 3F8 VH | EVQLVQSGAEVKKPGASVKVSCKASGYTFTSYAMHWVRQAPGQRLEWMGWINAGNGNTKYSQK |
| | | FQGRVTITRDTSASTAYMELSSLRSEDTAVYYCARVLHVLRYFDWPGAFDIWGQGTTVTVSS |
| 108 | 3F8 HC-CDR1 | GYTFTSYA |
| 109 | 3F8 HC-CDR2 | INAGNGNT |
| 110 | 3F8 HC-CDR3 | ARVLHVLRYFDWPGAFDI |
| 111 | 3F8 HC-FR1 | EVQLVQSGAEVKKPGASVKVSCKAS |
| 112 | 3F8 HC-FR2 | MHWVRQAPGQRLEWMGW |
| 113 | 3F8 HC-FR3 | KYSQKFQGRVTITRDTSASTAYMELSSLRSEDTAVYYC |
| 114 | 3F8 VL | QSVLTQPPSASGTPGQRVTISCSGSSSNIGTNYVYWYQQLPGTAPKLLIYINDQRPSGVPDRFSGS |
| | | KSGTSASLALSGLRSEDEADYYCAAWDDSLSGLVFGGGTKLTVL |
| 115 | 3F8 LC-CDR1 | SSNIGTNY |
| 116 | 3F8 LC-CDR2 | IND |
| 117 | 3F8 LC-CDR3 | AAWDDSLSGLV |
| 118 | 3F8 LC-FR1 | QSVLTQPPSASGTPGQRVTISCSGS |
| 119 | 3F8 LC-FR2 | VYWYQQLPGTAPKLLIY |
| 120 | 3F8 LC-FR3 | QRPSGVPDRFSGSKSGTSASLALSGLRSEDEADYYC |
| 121 | 3F9 VH | QVQLVQSGGGVVQPGRSLRLSCATSGFTFSSYGMHWVRQAPGKGLEWVAVISYDGSNKYYADSV |
| | | KGRFTISRDNSKNTLYLQMNSLRAEDTAVYYCARDLVDSSGWYGYYYYYGMDVWGQGTTVTVSS |
| 122 | 3F9 HC-CDR3 | ARDLVDSSGWYGYYYYYGMDV |
| 123 | 3F9 HC-FR1 | QVQLVQSGGGVVQPGRSLRLSCATS |
| 124 | 3F9 VL | EIVLTQSPATLSLSPGERATLSCRASQSVSSYLAWYQQKPGQAPRLLIYDASNRATGIPARFSGSGS |
| | | GTDFTLTISSLEPEDFAVYFCQQRSNWPGTFGQGTKVEIK |
| 125 | 3F9 LC-CDR1 | QSVSSY |
| 126 | 3F9 LC-CDR2 | DAS |
| 127 | 3F9 LC-CDR3 | QQRSNWPGT |
| 128 | 3F9 LC-FR1 | EIVLTQSPATLSLSPGERATLSCRAS |
| 129 | 3F9 LC-FR2 | LAWYQQKPGQAPRLLIY |
| 130 | 3F9 LC-FR3 | NRATGIPARFSGSGSGTDFTLTISSLEPEDFAVYFC |
| 131 | 4G9 VH | EVQLVQSGAEVKKPGDSVKVSCKASGYTFTGYYMHWVRQAPGQGLEWMGRINPNSGGTNYAQK |
| | | FQGRVTMTTDTSTSTAYMELRSLRSDDTAVYYCARDLGGATVYWGQGTLVTVSS |
| 132 | 4G9 HC-CDR1 | GYTFTGYY |
| 133 | 4G9 HC-CDR2 | INPNSGGT |
| 134 | 4G9 HC-CDR3 | ARDLGGATVY |
| 135 | 4G9 HC-FR1 | EVQLVQSGAEVKKPGDSVKVSCKAS |
| 136 | 4G9 HC-FR2 | MHWVRQAPGQGLEWMGR |
| 137 | 4G9 HC-FR3 | NYAQKFQGRVTMTTDTSTSTAYMELRSLRSDDTAVYYC |
| 138 | 4G9 VL | QAVLTQPSSLSASPGASASLTCTLHSGVNVGTQRIYWYQQKPGSPPRYLVRYKSDSNKQQGSGVP |
| | | SRFSGSKDASANAGILIISGLQSDDEADYYCMIWHNNAWVFGGGTTLTVL |
| 139 | 4G9 LC-CDR1 | SGVNVGTQR |
| 140 | 4G9 LC-CDR2 | YKSDSNK |
| 141 | 4G9 LC-FR1 | QAVLTQPSSLSASPGASASLTCTLH |
| 142 | 4G9 LC-FR2 | IYWYQQKPGSPPRYLVR |
| 143 | 4G9 LC-FR3 | QQGSGVPSRFSGSKDASANAGILIISGLQSDDEADYYC |
| 144 | 4G9 LC-FR4 | FGGGTTLTVL |
| 145 | 5A3 VH | EVQLVESGGGLVKPGGSLRLSCAASGFTFSSYSMNWVRQAPGKGLEWVSSISSSSSYIYYADSVK |
| | | GRFTISRDNAKNSLYLQMNSLRAEDTAVYYCARNGAVAGKYFDYWGQGTLVTVSS |
| 146 | 5A3 HC-CDR1 | GFTFSSYS |
| 147 | 5A3 HC-CDR2 | ISSSSSYI |
| 148 | 5A3 HC-CDR3 | ARNGAVAGKYFDY |
| 149 | 5A3 HC-FR1 | EVQLVESGGGLVKPGGSLRLSCAAS |
| 150 | 5A3 HC-FR2 | MNWVRQAPGKGLEWVSS |
| 151 | 5A3 HC-FR3 | YYADSVKGRFTISRDNAKNSLYLQMNSLRAEDTAVYYC |
| 152 | 5A3 VL | DVVMTQSPLSLPVTPGEPASISCRSSQSLLHSNGYNYLDWYLQKPGQSPQLLIYLGSNRASGVPDR |
| | | FSGSGSGTDFTLKISRVEAEDVGVYYCMQALQTPLTFGGGTKVEIK |
| 153 | 5A3 LC-CDR3 | MQALQTPLT |
| 154 | 5A3 LC-FR4 | FGGGTKVEIK |
| 155 | 5E8 VH | QVQLVQSGGGVVQPGRSLRLSCAASGFTFSSYAMHWVRQAPGKGLEWVAVISYDGSNKYYADSV |
| | | KGRFTISRDNSKNTLYLQMNSLRAEDTAVYYCARDFYPFIVGATSFFDYWGQGTLVTVSS |
| 156 | 5E8 HC-CDR3 | ARDFYPFIVGATSFFDY |
| 157 | 5E8 VL | DIQLTQSPSSLSASVGDRVTITCRASQSISTYINWYQQKPGKAPKLLIYFASSLQSGVPSRFSGGGS |
| | | GTDFTLTISSLQPEDFATYYCQQSYNIPVTFGQGTKVEVT |
| 158 | 5E8 LC-CDR1 | QSISTY |
| 159 | 5E8 LC-CDR2 | FAS |
| 160 | 5E8 LC-CDR3 | QQSYNIPVT |
| 161 | 5E8 LC-FR1 | DIQLTQSPSSLSASVGDRVTITCRAS |
| 162 | 5E8 LC-FR2 | INWYQQKPGKAPKLLIY |
| 163 | 5E8 LC-FR3 | SLQSGVPSRFSGGGSGTDFTLTISSLQPEDFATYYC |
| 164 | 5E8 LC-FR4 | QQSYNIPVTFGQGTKVEVT |
| 165 | C001, C008 VH | QVQLVQSGGGVVQPGRSLRLSCAASGFTVSSNYMSWVRQAPGKGLEWVSVIYSGGSTYYADSVK |
| | | GRFTISRDNSKNTLYLQMNSLRAEDTAVYYCARGSRAVAGAFDYWGQGTLVTVSS |
| 166 | C001, C008 HC- | GFTVSSNY |
| | CDR1 | |
| 167 | C001, C008 HC- | IYSGGST |
| | CDR2 | |
| 168 | C001, C008 HC- | ARGSRAVAGAFDY |
| | CDR3 | |
| 169 | C001, C008 HC- | MSWVRQAPGKGLEWVSV |
| | FR2 | |
| 170 | C001 VL | SSELTQDPAVSVALGQTVSITCQGDSLRNYFAAWYQQKPGQAPLLVIYGKNNRPSGIPDRFSGSSS |
| | | GNTASLTITGAEAEDEADYYCGSRDSSGNHPVFGGGTKVTVL |
| 171 | C001 LC-CDR1 | SLRNYF |
| 172 | C001, C025, | GKN |
| | C040 LC-CDR2 | |
| 173 | C001, C008 LC- | GSRDSSGNHPV |
| | CDR3 | |
| 174 | C001 LC-FR1 | SSELTQDPAVSVALGQTVSITCQGD |
| 175 | C001 LC-FR2 | AAWYQQKPGQAPLLVIY |
| 176 | C001 LC-FR3 | NRPSGIPDRFSGSSSGNTASLTITGAEAEDEADYYC |
| 177 | C001, C008, | FGGGTKVTVL |
| | C010 LC-FR4 | |
| 178 | C008 VL | YSELTQDPAVSVALGQTVRITCQGDSLRTYYANWYQQKPGQAPVLVMYAKNHRPSGIPDRFSGSS |
| | | SGNTASLTITGAQAEDEADYYCGSRDSSGNHPVFGGGTKVTVL |
| 179 | C008 LC-CDR1 | SLRTYY |
| 180 | C008 LC-CDR2 | AKN |
| 181 | C008 LC-FR1 | YSELTQDPAVSVALGQTVRITCQGD |
| 182 | C008 LC-FR2 | ANWYQQKPGQAPVLVMY |
| 183 | C008 LC-FR3 | HRPSGIPDRFSGSSSGNTASLTITGAQAEDEADYYC |
| 184 | C010 VH | QVQLQQSGPGLVKPSQTLSVTCAISGDSVSSNSAAWNWFRQSPSRGLEWLGRTYYRSKWYSDYA |
| | | VSVKSRITINPDTSKNQFSLQLNSVTPEDTAVYYCARDVRAARAFDIWGQGTMVTVSS |
| 185 | C010, C117 HC- | SVSSNSAAWN |
| | CDR1 | |
| 186 | C010 HC-CDR2 | YRSKWYS |
| 187 | C010 HC-CDR3 | ARDVRAARAFDI |
| 188 | C010 HC-FR1 | QVQLQQSGPGLVKPSQTLSVTCAISGD |
| 189 | C010 HC-FR2 | WFRQSPSRGLEWLGRTY |
| 190 | C010, C117 HC- | DYAVSVKSRITINPDTSKNQFSLQLNSVTPEDTAVYYC |
| | FR3 | |
| 191 | C010 VL | QAVLTQPSSLSASPGASASLTCTLRSGINVGTYRIYWYQQKPGSPPQYLLRYKSDSDKQQGSGVPS |
| | | RFSGSKDASANAGILVISGLQSEDEADYYCLIWHNRGWVFGGGTKVTVL |
| 192 | 000 | 000 |
| 193 | 000 | 000 |
| 194 | C010 LC-CDR3 | LIWHNRGWV |
| 195 | 000 | 000 |
| 196 | 000 | 000 |
| 197 | C010 LC-FR3 | QQGSGVPSRFSGSKDASANAGILVISGLQSEDEADYYC |
| 198 | C023, C046 VH | EVQLVESGGGLVQPGRPLRLSCAASGFTFSSYAMSWVRQAPGKGLEWVSSISSGGSTYYADSVR |
| | | GRFTISRDDSKDTLYLQMNSLRAEDTAVYYCARTGVGGAFDIWGQGTMVTVSS |
| 199 | C023, C046 HC- | ISSGGST |
| | CDR2 | |
| 200 | C023, C046 HC- | ARTGVGGAFDI |
| | CDR3 | |
| 201 | C023, C046 HC- | EVQLVESGGGLVQPGRPLRLSCAAS |
| | FR1 | |
| 202 | C023, C046 HC- | MSWVRQAPGKGLEWVSS |
| | FR2 | |
| 203 | C023, C046 HC- | YYADSVRGRFTISRDDSKDTLYLQMNSLRAEDTAVYYC |
| | FR3 | |
| 204 | C023 VL | DVVMTQSPLSLPVTPGEPASISCRSSHSLLYDNRYNYLDWYLQRPGQSPQLLIYLGSTRASGVPDR |
| | | FSGSGSGTDFTLKISRVEAEDIGVYYCMQAVQTPIIFGQGTRLEIK |
| 205 | C023 LC-CDR1 | HSLLYDNRYNY |
| 206 | C023 LC-CDR3 | MQAVQTPI |
| 207 | C023 LC-FR2 | LDWYLQRPGQSPQLLIY |
| 208 | C023 LC-FR3 | TRASGVPDRFSGSGSGTDFTLKISRVEAEDIGVYYC |
| 209 | C023 LC-FR4 | FGQGTRLEIK |
| 210 | C025 VH | EVQLLESGGGLVQPGGSLRLSCAASGFTFSSYAMSWVRQAPGKGLEWVSTIYYAGSNTYYADSVK |
| | | GRFTISRDNSKNTLYLQMNSLRAEDTAVYYCAKGYYTFDYWGQGTLVTVSS |
| 211 | C025 HC-CDR2 | IYYAGSNT |
| 212 | C025 HC-CDR3 | AKGYYTFDY |
| 213 | C025 HC-FR1 | EVQLLESGGGLVQPGGSLRLSCAAS |
| 214 | C025 HC-FR2 | MSWVRQAPGKGLEWVST |
| 215 | C025 VL | QDPDVSVALGQTVRITCHGDSLRSSYASWYQQKPGQAPVLVFFGKNNRPSGIPDRFSGSSSGDTA |
| | | SLTITGAQAEDEADYYCHSRDTSDNHLRVFGGGTKLTVL |
| 216 | C025 LC-CDR1 | SLRSSY |
| 217 | C025 LC-CDR3 | HSRDTSDNHLRV |
| 218 | C025 LC-FR1 | QDPDVSVALGQTVRITCHGD |
| 219 | C025 LC-FR2 | ASWYQQKPGQAPVLVFF |
| 220 | C025 LC-FR3 | NRPSGIPDRFSGSSSGDTASLTITGAQAEDEADYYC |
| 221 | C040 VH | EVQLVESGGGLVRPGRSLRLSCRASGLTLSDYGMHWVRQAPGKGLEWVSGISGSGSNTYYTDSV |
| | | KGRFTISRDNAKNSLYLQMNSLRAEDTAVYFCARSVRGAFDNWGQGTMVTVSS |
| 222 | C040 HC-CDR1 | GLTLSDYG |
| 223 | C040 HC-CDR2 | ISGSGSNT |
| 224 | C040 HC-CDR3 | ARSVRGAFDN |
| 225 | C040 HC-FR1 | EVQLVESGGGLVRPGRSLRLSCRAS |
| 226 | C040 HC-FR2 | MHWVRQAPGKGLEWVSG |
| 227 | C040 HC-FR3 | YYTDSVKGRFTISRDNAKNSLYLQMNSLRAEDTAVYFC |
| 228 | C040 VL | SSELTQDPAVSVALGQTVRITCQGDSLRRFFASWYQQRPGQAPILVIYGKNNRPSGIPDRFSGSRS |
| | | GNTASLTIVGAQADDEADYYCSSRDSSRDRLVFGGGTKLTVL |
| 229 | C040 LC-CDR1 | SLRRFF |
| 230 | C040 LC-CDR3 | SSRDSSRDRLV |
| 231 | C040, C117 LC- | SSELTQDPAVSVALGQTVRITCQGD |
| | FR1 | |
| 232 | C040 LC-FR2 | ASWYQQRPGQAPILVIY |
| 233 | C040 LC-FR3 | NRPSGIPDRFSGSRSGNTASLTIVGAQADDEADYYC |
| 234 | C046 VL | QSVLTQPPSVSAAPGQKVTISCSGSSSNIGDNYVSWYQQLPGTAPKVLIYGNNNRPSGIPDRFSGS |
| | | KSGTSASLAISGLQSEDEADYYCATWDDSLKGYVFGTGTQLTVL |
| 235 | C046 LC-CDR1 | SSNIGDNY |
| 236 | C046 LC-CDR2 | GNN |
| 237 | C046 LC-CDR3 | ATWDDSLKGYV |
| 238 | C046 LC-FR1 | QSVLTQPPSVSAAPGQKVTISCSGS |
| 239 | C046 LC-FR2 | VSWYQQLPGTAPKVLIY |
| 240 | C046 LC-FR3 | NRPSGIPDRFSGSKSGTSASLAISGLQSEDEADYYC |
| 241 | C046 LC-FR4 | FGTGTQLTVL |
| 242 | C117 VH | QVQLQQSGPGLVKPSQTLSLTCAISGDSVSSNSAAWNWIRQSPSRGLEWLGRTYYRSKWYNDYA |
| | | VSVKSRITINPDTSKNQFSLQLNSVTPEDTAVYYCARGIVGAFDYWGQGTLVTVSS |
| 243 | C117 HC-CDR2 | YRSKWYN |
| 244 | C117 HC-CDR3 | ARGIVGAFDY |
| 245 | C117 HC-FR1 | QVQLQQSGPGLVKPSQTLSLTCAISGD |
| 246 | C117 HC-FR2 | WIRQSPSRGLEWLGRTY |
| 247 | C117 VL | SSELTQDPAVSVALGQTVRITCQGDSLRSYHASWYQQKPGQAPVLVIYGRNNRPSGIPDRFSGSTS |
| | | GNTASLTITGAQAEDEADYYCKSRDSSGSLSYVFGTGTKVTVL |
| 248 | C117 LC-CDR1 | SLRSYH |
| 249 | C117 LC-CDR2 | GRN |
| 250 | C117 LC-CDR3 | KSRDSSGSLSYV |
| 251 | C117 LC-FR2 | ASWYQQKPGQAPVLVIY |
| 252 | C117 LC-FR3 | NRPSGIPDRFSGSTSGNTASLTITGAQAEDEADYYC |
| 253 | C117 LC-FR4 | FGTGTKVTVL |
| 254 | Human IgG1 | ASTKGPSVFPLAPSSKSTSGGTAALGCLVKDYFPEPVTVSWNSGALTSGVHTFPAVLQSSGLYSLS |
| | constant region | SVVTVPSSSLGTQTYICNVNHKPSNTKVDKKVEPKSCDKTHTCPPCPAPELLGGPSVFLFPPKPKDT |
| | (IGHG1; | LMISRTPEVTCVVVDVSHEDPEVKFNWYVDGVEVHNAKTKPREEQYNSTYRVVSVLTVLHQDWLN |
| | UniProt: P01857-1, | GKEYKCKVSNKALPAPIEKTISKAKGQPREPQVYTLPPSRDELTKNQVSLTCLVKGFYPSDIAVEWE |
| | v1) | SNGQPENNYKTTPPVLDSDGSFFLYSKLTVDKSRWQQGNVFSCSVMHEALHNHYTQKSLSLSPGK |
| 255 | CH1 IgG1 | ASTKGPSVFPLAPSSKSTSGGTAALGCLVKDYFPEPVTVSWNSGALTSGVHTFPAVLQSSGLYSLS |
| | (positions 1-98 of | SVVTVPSSSLGTQTYICNVNHKPSNTKVDKKV |
| | P01857-1, v1) | |
| 256 | Hinge IgG1 | EPKSCDKTHTCP |
| | (positions 99-110 | |
| | of P01857-1, v1) | |
| 257 | CH2 IgG1 | PCPAPELLGGPSVFLFPPKPKDTLMISRTPEVTCVVVDVSHEDPEVKFNWYVDGVEVHNAKTKPRE |
| | (positions 111-223 | EQYNSTYRVVSVLTVLHQDWLNGKEYKCKVSNKALPAPIEKTISKAK |
| | of P01857-1, v1) | |
| 258 | CH3 IgG1 | GQPREPQVYTLPPSRDELTKNQVSLTCLVKGFYPSDIAVEWESNGQPENNYKTTPPVLDSDGSFFL |
| | (positions 224-330 | YSKLTVDKSRWQQGNVFSCSVMHEALHNHYTQKSLSLSPGK |
| | of P01857-1, v1) | |
| 259 | Human IgG1 | ASTKGPSVFPLAPSSKSTSGGTAALGCLVKDYFPEPVTVSWNSGALTSGVHTFPAVLQSSGLYSLS |
| | constant region | SVVTVPSSSLGTQTYICNVNHKPSNTKVDKKVEPKSCDKTHTCPPCPAPELLGGPSVFLFPPKPKDT |
| | encoded by | LMISRTPEVTCVVVDVSHEDPEVKFNWYVDGVEVHNAKTKPREEQYNSTYRVVSVLTVLHQDWLN |
| | pFUSEss-CHIg- | GKEYKCKVSNKALPAPIEKTISKAKGQPREPQVYTLPPSREEMTKNQVSLTCLVKGFYPSDIAVEWE |
| | hG1 (D356E and | SNGQPENNYKTTPPVLDSDGSFFLYSKLTVDKSRWQQGNVFSCSVMHEALHNHYTQKSLSLSPGK |
| | L358M (EU | |
| | numbering) | |
| | relative to | |
| | P01857-1) | |
| 260 | Human IgG1 | ASTKGPSVFPLAPSSKSTSGGTAALGCLVKDYFPEPVTVSWNSGALTSGVHTFPAVLQSSGLYSLS |
| | constant region | SVVTVPSSSLGTQTYICNVNHKPSNTKVDKRVEPKSCDKTHTCPPCPAPELLGGPSVFLFPPKPKD |
| | G1m3 allotype | TLMISRTPEVTCVVVDVSHEDPEVKFNWYVDGVEVHNAKTKPREEQYNSTYRVVSVLTVLHQDWL |
| | (K214R, D356E | NGKEYKCKVSNKALPAPIEKTISKAKGQPREPQVYTLPPSREEMTKNQVSLTCLVKGFYPSDIAVEW |
| | and L358M (EU | ESNGQPENNYKTTPPVLDSDGSFFLYSKLTVDKSRWQQGNVFSCSVMHEALHNHYTQKSLSLSPG |
| | numbering) | K |
| | relative to | |
| | P01857-1) | |
| 261 | CH1 IgG1 G1m3 | ASTKGPSVFPLAPSSKSTSGGTAALGCLVKDYFPEPVTVSWNSGALTSGVHTFPAVLQSSGLYSLS |
| | allotype | SVVTVPSSSLGTQTYICNVNHKPSNTKVDKRV |
| 262 | CH3 igG1 G1m3 | GQPREPQVYTLPPSREEMTKNQVSLTCLVKGFYPSDIAVEWESNGQPENNYKTTPPVLDSDGSFF |
| | allotype | LYSKLTVDKSRWQQGNVFSCSVMHEALHNHYTQKSLSLSPGK |
| 263 | Human IgG4 | ASTKGPSVFPLAPCSRSTSESTAALGCLVKDYFPEPVTVSWNSGALTSGVHTFPAVLQSSGLYSLS |
| | constant region | SVVTVPSSSLGTKTYTCNVDHKPSNTKVDKRVESKYGPPCPSCPAPEFLGGPSVFLFPPKPKDTLM |
| | (IGHG4; | ISRTPEVTCVVVDVSQEDPEVQFNWYVDGVEVHNAKTKPREEQFNSTYRVVSVLTVLHQDWLNGK |
| | UniProt:P01861-1, | EYKCKVSNKGLPSSIEKTISKAKGQPREPQVYTLPPSQEEMTKNQVSLTCLVKGFYPSDIAVEWESN |
| | v1) | GQPENNYKTTPPVLDSDGSFFLYSRLTVDKSRWQEGNVFSCSVMHEALHNHYTQKSLSLSLGK |
| 264 | Human IgG1 | PCPAPELLGGPSVFLFPPKPKDTLMISRTPEVTCVVVDVSHEDPEVKFNWYVDGVEVHNAKTKPRE |
| | CH2-CH3 region | EQYNSTYRVVSVLTVLHQDWLNGKEYKCKVSNKALPAPIEKTISKAKGQPREPQVYTLPPSRDELTK |
| | | NQVSLTCLVKGFYPSDIAVEWESNGQPENNYKTTPPVLDSDGSFFLYSKLTVDKSRWQQGNVFSC |
| | | SVMHEALHNHYTQKSLSLSPGK |
| 265 | Human IgG1 | PCPAPELLGGPSVFLFPPKPKDTLMISRTPEVTCVVVDVSHEDPEVKFNWYVDGVEVHNAKTKPRE |
| | G1m3 allotype | EQYNSTYRVVSVLTVLHQDWLNGKEYKCKVSNKALPAPIEKTISKAKGQPREPQVYTLPPSREEMT |
| | CH2-CH3 region | KNQVSLTCLVKGFYPSDIAVEWESNGQPENNYKTTPPVLDSDGSFFLYSKLTVDKSRWQQGNVFS |
| | | CSVMHEALHNHYTQKSLSLSPG |
| 266 | Human IgG4 | APEFLGGPSVFLFPPKPKDTLMISRTPEVTCVVVDVSQEDPEVQFNWYVDGVEVHNAKTKPREEQF |
| | CH2-CH3 region | NSTYRVVSVLTVLHQDWLNGKEYKCKVSNKGLPSSIEKTISKAKGQPREPQVYTLPPSQEEMTKNQ |
| | | VSLTCLVKGFYPSDIAVEWESNGQPENNYKTTPPVLDSDGSFFLYSRLTVDKSRWQEGNVFSCSV |
| | | MHEALHNHYTQKSLSLSLGK |
| 267 | Cκ CL (IGKC; | RTVAAPSVFIFPPSDEQLKSGTASVVCLLNNFYPREAKVQWKVDNALQSGNSQESVTEQDSKDSTY |
| | UniProt: P01834- | SLSSTLTLSKADYEKHKVYACEVTHQGLSSPVTKSFNRGEC |
| | 1, v2) | |
| 268 | Cλ CL1 (IGLC1; | GQPKANPTVTLFPPSSEELQANKATLVCLISDFYPGAVTVAWKADGSPVKAGVETTKPSKQSNNKY |
| | UniProt: P0CG04- | AASSYLSLTPEQWKSHRSYSCQVTHEGSTVEKTVAPTECS |
| | 1, v1) | |
| 269 | Cλ CL2 (IGLC2; | GQPKAAPSVTLFPPSSEELQANKATLVCLISDFYPGAVTVAWKADSSPVKAGVETTTPSKQSNNKY |
| | UniProt: P0DOY2- | AASSYLSLTPEQWKSHRSYSCQVTHEGSTVEKTVAPTECS |
| | 1, v1) | |
| 270 | Cλ CL3 (IGLC3; | GQPKAAPSVTLFPPSSEELQANKATLVCLISDFYPGAVTVAWKADSSPVKAGVETTTPSKQSNNKY |
| | UniProt: P0DOY3- | AASSYLSLTPEQWKSHKSYSCQVTHEGSTVEKTVAPTECS |
| | 1, v1) | |
| 271 | Cλ CL6 (IGLC6; | GQPKAAPSVTLFPPSSEELQANKATLVCLISDFYPGAVKVAWKADGSPVNTGVETTTPSKQSNNKY |
| | UniProt: P0CF74- | AASSYLSLTPEQWKSHRSYSCQVTHEGSTVEKTVAPAECS |
| | 1, v1) | |
| 272 | Cλ CL7 (IGLC7; | GQPKAAPSVTLFPPSSEELQANKATLVCLVSDFNPGAVTVAWKADGSPVKVGVETTKPSKQSNNK |
| | UniProt: A0M8Q6- | YAASSYLSLTPEQWKSHRSYSCRVTHEGSTVEKTVAPAECS |
| | 1, v3) | |
| 273 | 1D3 IgG1 HC | EVQLVESGGGVVQPGRSLRLSCAASGFTFSSYGMHWVRQAPGKGLEWVAVISYDGSNKYYADSV |
| | | KGRFTISRDNSKNTLYLQMNSLRAEDTAVYYCARGGRAFDIWGQGTMVTVSSASTKGPSVFPLAPS |
| | | SKSTSGGTAALGCLVKDYFPEPVTVSWNSGALTSGVHTFPAVLQSSGLYSLSSVVTVPSSSLGTQT |
| | | YICNVNHKPSNTKVDKKVEPKSCDKTHTCPPCPAPELLGGPSVFLFPPKPKDTLMISRTPEVTCVVV |
| | | DVSHEDPEVKFNWYVDGVEVHNAKTKPREEQYNSTYRVVSVLTVLHQDWLNGKEYKCKVSNKAL |
| | | PAPIEKTISKAKGQPREPQVYTLPPSRDELTKNQVSLTCLVKGFYPSDIAVEWESNGQPENNYKTTP |
| | | PVLDSDGSFFLYSKLTVDKSRWQQGNVFSCSVMHEALHNHYTQKSLSLSPGK |
| 274 | 1D3 IgG4 HC | EVQLVESGGGVVQPGRSLRLSCAASGFTFSSYGMHWVRQAPGKGLEWVAVISYDGSNKYYADSV |
| | | KGRFTISRDNSKNTLYLQMNSLRAEDTAVYYCARGGRAFDIWGQGTMVTVSSASTKGPSVFPLAP |
| | | CSRSTSESTAALGCLVKDYFPEPVTVSWNSGALTSGVHTFPAVLQSSGLYSLSSVVTVPSSSLGTK |
| | | TYTCNVDHKPSNTKVDKRVESKYGPPCPPCPAPEFLGGPSVFLFPPKPKDTLMISRTPEVTCVVVD |
| | | VSQEDPEVQFNWYVDGVEVHNAKTKPREEQFNSTYRVVSVLTVLHQDWLNGKEYKCKVSNKGLP |
| | | SSIEKTISKAKGQPREPQVYTLPPSQEEMTKNQVSLTCLVKGFYPSDIAVEWESNGQPENNYKTTP |
| | | PVLDSDGSFFLYSRLTVDKSRWQEGNVFSCSVMHEALHNHYTQKSLSLSLGK |
| 275 | 1D3 LC | DVVMTQSPLSLPVTLGQPASISCKSSQSLVNSGGDTFLNWFQQRPGQSPRRLIYKVSNRDSGVPD |
| | | RFSGSGSGTDFTLEISRVEAEDVGVYYCMQGTHWPRTFGQGTKVEIKRTVAAPSVFIFSPSDEQLK |
| | | SGTASVVCLLNNFYPREAKVQWKVDNALQSGNSQESVTEQDSKDSTYSLSSTLTLSKADYEKHKLY |
| | | ACEVTHQGLSSPVTKSFNRGEC |
| 276 | 1D6 IgG1 HC | QVQLQESGAGLLKPSETLSLTCAVYGGSFSGYYWSWIRQPPGKGLEWIGRIYTSGSTNYNPSLKSR |
| | | VTISVDTSKNQFSLKLSSVTAADTAVYYCARVGAGAFDIWGQGTMVTVSSASTKGPSVFPLAPSSK |
| | | STSGGTAALGCLVKDYFPEPVTVSWNSGALTSGVHTFPAVLQSSGLYSLSSVVTVPSSSLGTQTYI |
| | | CNVNHKPSNTKVDKKVEPKSCDKTHTCPPCPAPELLGGPSVFLFPPKPKDTLMISRTPEVTCVVVD |
| | | VSHEDPEVKFNWYVDGVEVHNAKTKPREEQYNSTYRVVSVLTVLHQDWLNGKEYKCKVSNKALPA |
| | | PIEKTISKAKGQPREPQVYTLPPSRDELTKNQVSLTCLVKGFYPSDIAVEWESNGQPENNYKTTPPV |
| | | LDSDGSFFLYSKLTVDKSRWQQGNVFSCSVMHEALHNHYTQKSLSLSPGK |
| 277 | 1D6 IgG4 HC | QVQLQESGAGLLKPSETLSLTCAVYGGSFSGYYWSWIRQPPGKGLEWIGRIYTSGSTNYNPSLKSR |
| | | VTISVDTSKNQFSLKLSSVTAADTAVYYCARVGAGAFDIWGQGTMVTVSSASTKGPSVFPLAPCSR |
| | | STSESTAALGCLVKDYFPEPVTVSWNSGALTSGVHTFPAVLQSSGLYSLSSVVTVPSSSLGTKTYT |
| | | CNVDHKPSNTKVDKRVESKYGPPCPPCPAPEFLGGPSVFLFPPKPKDTLMISRTPEVTCVVVDVSQ |
| | | EDPEVQFNWYVDGVEVHNAKTKPREEQFNSTYRVVSVLTVLHQDWLNGKEYKCKVSNKGLPSSIE |
| | | KTISKAKGQPREPQVYTLPPSQEEMTKNQVSLTCLVKGFYPSDIAVEWESNGQPENNYKTTPPVLD |
| | | SDGSFFLYSRLTVDKSRWQEGNVFSCSVMHEALHNHYTQKSLSLSLGK |
| 278 | 1D6 LC | QAVLTQPSSLSASPGASASLTCTLRSDINVGAYRIYWYQQKPGSPPRYLLRYKSDSDKQQGSGVSN |
| | | RFSASKDASANAGILHISGLHSEDEADYYCLIWHNSVWVFGGGTKLTVLSQPKAAPSVTLFPPSSEE |
| | | LQANKATLVCLISDFYPGAVTVAWKADSSPVKAGVETTTPSKQSNNKYAASSYLSLTPEQWKSHKS |
| | | YSCQVTHEGSTVEKTVAPAECS |
| 279 | 1E1 IgG1 HC | QVQLQESGGGVVRPGGSLRLSCAASGFTFDDYGMSWVRQAPGKGLEWVSGINWNGGSTGYADS |
| | | VKGRFTISRDNAKNSLYLQMNSLRAEDTAVYYCARGIAVDYWGQGTLVTVSSASTKGPSVFPLAPS |
| | | SKSTSGGTAALGCLVKDYFPEPVTVSWNSGALTSGVHTFPAVLQSSGLYSLSSVVTVPSSSLGTQT |
| | | YICNVNHKPSNTKVDKKVEPKSCDKTHTCPPCPAPELLGGPSVFLFPPKPKDTLMISRTPEVTCVVV |
| | | DVSHEDPEVKFNWYVDGVEVHNAKTKPREEQYNSTYRVVSVLTVLHQDWLNGKEYKCKVSNKAL |
| | | PAPIEKTISKAKGQPREPQVYTLPPSRDELTKNQVSLTCLVKGFYPSDIAVEWESNGQPENNYKTTP |
| | | PVLDSDGSFFLYSKLTVDKSRWQQGNVFSCSVMHEALHNHYTQKSLSLSPGK |
| 280 | 1E1 IgG4 HC | QVQLQESGGGVVRPGGSLRLSCAASGFTFDDYGMSWVRQAPGKGLEWVSGINWNGGSTGYADS |
| | | VKGRFTISRDNAKNSLYLQMNSLRAEDTAVYYCARGIAVDYWGQGTLVTVSSASTKGPSVFPLAPC |
| | | SRSTSESTAALGCLVKDYFPEPVTVSWNSGALTSGVHTFPAVLQSSGLYSLSSVVTVPSSSLGTKT |
| | | YTCNVDHKPSNTKVDKRVESKYGPPCPPCPAPEFLGGPSVFLFPPKPKDTLMISRTPEVTCVVVDV |
| | | SQEDPEVQFNWYVDGVEVHNAKTKPREEQFNSTYRVVSVLTVLHQDWLNGKEYKCKVSNKGLPS |
| | | SIEKTISKAKGQPREPQVYTLPPSQEEMTKNQVSLTCLVKGFYPSDIAVEWESNGQPENNYKTTPP |
| | | VLDSDGSFFLYSRLTVDKSRWQEGNVFSCSVMHEALHNHYTQKSLSLSLGK |
| 281 | 1E1 LC | DVVMTQSPLSLPVTPGEPASISCRSSQSLLHSNGYNYLDWYLQKPGQSPQLLIYLGSNRASGVPDR |
| | | FSGSGSGTDFTLKISRVEAEDVGVYYCMQALQTPWTFGQGTKVEIKRTVAAPSVFIFPPSDEQLKS |
| | | GTASVVCLLNNFYPREAKVQWKVDNALQSGNSQESVTEQDSKDSTYSLSSTLTLSKADYEKHKVY |
| | | ACEVTHQGLSSPVTKSFNRGEC |
| 282 | 1E6 IgG1 HC | QLQLQESGGGVVQPGRSLRLSCAASGFTFSSYAMHWVRQAPGKGLEWVAVISYDGSNKYYADSV |
| | | KGRFTISRDNSKNTLYLQMNSLRAEDTAVYYCARDIPYGGYAHYYYYGMDVWGQGTTVTVSSAST |
| | | KGPSVFPLAPSSKSTSGGTAALGCLVKDYFPEPVTVSWNSGALTSGVHTFPAVLQSSGLYSLSSVV |
| | | TVPSSSLGTQTYICNVNHKPSNTKVDKKVEPKSCDKTHTCPPCPAPELLGGPSVFLFPPKPKDTLMI |
| | | SRTPEVTCVVVDVSHEDPEVKFNWYVDGVEVHNAKTKPREEQYNSTYRVVSVLTVLHQDWLNGK |
| | | EYKCKVSNKALPAPIEKTISKAKGQPREPQVYTLPPSRDELTKNQVSLTCLVKGFYPSDIAVEWESN |
| | | GQPENNYKTTPPVLDSDGSFFLYSKLTVDKSRWQQGNVFSCSVMHEALHNHYTQKSLSLSPGK |
| 283 | 1E6 IgG4 HC | QLQLQESGGGVVQPGRSLRLSCAASGFTFSSYAMHWVRQAPGKGLEWVAVISYDGSNKYYADSV |
| | | KGRFTISRDNSKNTLYLQMNSLRAEDTAVYYCARDIPYGGYAHYYYYGMDVWGQGTTVTVSSAST |
| | | KGPSVFPLAPCSRSTSESTAALGCLVKDYFPEPVTVSWNSGALTSGVHTFPAVLQSSGLYSLSSVV |
| | | TVPSSSLGTKTYTCNVDHKPSNTKVDKRVESKYGPPCPPCPAPEFLGGPSVFLFPPKPKDTLMISR |
| | | TPEVTCVVVDVSQEDPEVQFNWYVDGVEVHNAKTKPREEQFNSTYRVVSVLTVLHQDWLNGKEY |
| | | KCKVSNKGLPSSIEKTISKAKGQPREPQVYTLPPSQEEMTKNQVSLTCLVKGFYPSDIAVEWESNG |
| | | QPENNYKTTPPVLDSDGSFFLYSRLTVDKSRWQEGNVFSCSVMHEALHNHYTQKSLSLSLGK |
| 284 | 1E6 LC | QTVVTQEPSLTVSPGGTVTLTCTSSTGAVTSASYPNWFQQKPGQAPRKLIYDTNNRHSWTPARFS |
| | | GSLLGGKAALTLSGAEPEDEAVYYCFLSYSGAYWIFGGGTHLTVLGQPKAAPSVTLFPPSSEELQA |
| | | NKATLVCLISDFYPGAVTVAWKADSSPVKAGVETTTPSKQSNNKYAASSYLSLTPEQWKSHRSYSC |
| | | QVTHEGSTVEKTVAPTECS |
| 285 | 2C6, 2H6, 3D1 | QVQLVQSGAEVKKPGSSVKVSCKASGGTFSSYAISWVRQAPGQGLEWMGRIIPILGIANYAQKFQG |
| | IgG1 HC | RVTITADKSTSTAYMELSSLRSEDTAVYYCAREERGSYFRAPFDYWGQGTLVTVSSASTKGPSVFP |
| | | LAPSSKSTSGGTAALGCLVKDYFPEPVTVSWNSGALTSGVHTFPAVLQSSGLYSLSSVVTVPSSSL |
| | | GTQTYICNVNHKPSNTKVDKKVEPKSCDKTHTCPPCPAPELLGGPSVFLFPPKPKDTLMISRTPEVT |
| | | CVVVDVSHEDPEVKFNWYVDGVEVHNAKTKPREEQYNSTYRVVSVLTVLHQDWLNGKEYKCKVS |
| | | NKALPAPIEKTISKAKGQPREPQVYTLPPSRDELTKNQVSLTCLVKGFYPSDIAVEWESNGQPENNY |
| | | KTTPPVLDSDGSFFLYSKLTVDKSRWQQGNVFSCSVMHEALHNHYTQKSLSLSPGK |
| 286 | 2C6, 2H6, 3D1 | QVQLVQSGAEVKKPGSSVKVSCKASGGTFSSYAISWVRQAPGQGLEWMGRIIPILGIANYAQKFQG |
| | IgG4 HC | RVTITADKSTSTAYMELSSLRSEDTAVYYCAREERGSYFRAPFDYWGQGTLVTVSSASTKGPSVFP |
| | | LAPCSRSTSESTAALGCLVKDYFPEPVTVSWNSGALTSGVHTFPAVLQSSGLYSLSSVVTVPSSSL |
| | | GTKTYTCNVDHKPSNTKVDKRVESKYGPPCPPCPAPEFLGGPSVFLFPPKPKDTLMISRTPEVTCV |
| | | VVDVSQEDPEVQFNWYVDGVEVHNAKTKPREEQFNSTYRVVSVLTVLHQDWLNGKEYKCKVSNK |
| | | GLPSSIEKTISKAKGQPREPQVYTLPPSQEEMTKNQVSLTCLVKGFYPSDIAVEWESNGQPENNYK |
| | | TTPPVLDSDGSFFLYSRLTVDKSRWQEGNVFSCSVMHEALHNHYTQKSLSLSLGK |
| 287 | 2C6 LC | QAVLTQPSSLSASPGASASLTCTLRSDISVGTYRIYWYQQKPGSPPHYLLRYKSDSDKQQGSGVPS |
| | | RFSGFKDASANAGILLISGLQSEDEADYYCLIWHSNTWVFGGGTKLTVLGQPKAAPSVTLFPPSSEE |
| | | LQANKATLVCLISDFYPGAVTVAWKADSSPVKAGVETTTPSKQSNNKYAASSYLSLTPEQWKSHKS |
| | | YSCQVTHEGSTVEKTVAPTECS |
| 288 | 2H6, 2G9 LC | QAVLTQPSSLSASPGASASLTCTLRSGINVGTYRIYWYQQKPGSPPQYLLRYKSDSDKQQGSGVPS |
| | | RFSGSKDASANAGILLISGLQSEDEADYYCMIWHSSAWVFGGGTKLTVLGQPKAAPSVTLFPPSSE |
| | | ELQANKATLVCLISDFYPGAVTVAWKADSSPVKAGVETTTPSKQSNNKYAASSYLSLTPEQWKSHK |
| | | SYSCQVTHEGSTVEKTVAPTECS |
| 289 | 3D1 LC | QAVLTQPSSLSASPGASASLTCTLRSGIHVGTYRIYWYQQKPGSPPQFLLKYKTDSDKQQGSGVPS |
| | | RFSGSKDASANAGILLISGLQSEDEADYYCMIWHNNAWVFGGGTKLTVLGQPKAAPSVTLFPPSSE |
| | | ELQANKATLVCLISDFYPGAVTVAWKADSSPVKAGVETTTPSKQSNNKYAASSYLSLTPEQWKSHK |
| | | SYSCQVTHEGSTVEKTVAPTECS |
| 290 | 2G9 IgG1 HC | QVTLKESGGGVVQPGRSLRLSCAASGFTFSSYGMHWVRQAPGKGLEWVAVISYDGSNKYYADSV |
| | | KGRFTISRDNSKNTLYLQMNSLRAEDTAVYYCTQGRDYWGQGTLVTVSSASTKGPSVFPLAPSSK |
| | | STSGGTAALGCLVKDYFPEPVTVSWNSGALTSGVHTFPAVLQSSGLYSLSSVVTVPSSSLGTQTYI |
| | | CNVNHKPSNTKVDKKVEPKSCDKTHTCPPCPAPELLGGPSVFLFPPKPKDTLMISRTPEVTCVVVD |
| | | VSHEDPEVKFNWYVDGVEVHNAKTKPREEQYNSTYRVVSVLTVLHQDWLNGKEYKCKVSNKALPA |
| | | PIEKTISKAKGQPREPQVYTLPPSRDELTKNQVSLTCLVKGFYPSDIAVEWESNGQPENNYKTTPPV |
| | | LDSDGSFFLYSKLTVDKSRWQQGNVFSCSVMHEALHNHYTQKSLSLSPGK |
| 291 | 2G9 IgG4 HC | QVTLKESGGGVVQPGRSLRLSCAASGFTFSSYGMHWVRQAPGKGLEWVAVISYDGSNKYYADSV |
| | | KGRFTISRDNSKNTLYLQMNSLRAEDTAVYYCTQGRDYWGQGTLVTVSSASTKGPSVFPLAPCSR |
| | | STSESTAALGCLVKDYFPEPVTVSWNSGALTSGVHTFPAVLQSSGLYSLSSVVTVPSSSLGTKTYT |
| | | CNVDHKPSNTKVDKRVESKYGPPCPPCPAPEFLGGPSVFLFPPKPKDTLMISRTPEVTCVVVDVSQ |
| | | EDPEVQFNWYVDGVEVHNAKTKPREEQFNSTYRVVSVLTVLHQDWLNGKEYKCKVSNKGLPSSIE |
| | | KTISKAKGQPREPQVYTLPPSQEEMTKNQVSLTCLVKGFYPSDIAVEWESNGQPENNYKTTPPVLD |
| | | SDGSFFLYSRLTVDKSRWQEGNVFSCSVMHEALHNHYTQKSLSLSLGK |
| 292 | 2G12 IgG1 HC | QVQLVQSGGGVVQPGRSLRLSCAASGFTFSSYAMHWVRQAPGKGLEWVAVISYDGSNKYYADSV |
| | | KGRFTISRDNSKNTLYLQMNSLRAEDTAVYYCARASRGMDVWGQGTTVTVSSASTKGPSVFPLAP |
| | | SSKSTSGGTAALGCLVKDYFPEPVTVSWNSGALTSGVHTFPAVLQSSGLYSLSSVVTVPSSSLGTQ |
| | | TYICNVNHKPSNTKVDKKVEPKSCDKTHTCPPCPAPELLGGPSVFLFPPKPKDTLMISRTPEVTCVV |
| | | VDVSHEDPEVKFNWYVDGVEVHNAKTKPREEQYNSTYRVVSVLTVLHQDWLNGKEYKCKVSNKA |
| | | LPAPIEKTISKAKGQPREPQVYTLPPSRDELTKNQVSLTCLVKGFYPSDIAVEWESNGQPENNYKTT |
| | | PPVLDSDGSFFLYSKLTVDKSRWQQGNVFSCSVMHEALHNHYTQKSLSLSPGK |
| 293 | 2G12 IgG4 HC | QVQLVQSGGGVVQPGRSLRLSCAASGFTFSSYAMHWVRQAPGKGLEWVAVISYDGSNKYYADSV |
| | | KGRFTISRDNSKNTLYLQMNSLRAEDTAVYYCARASRGMDVWGQGTTVTVSSASTKGPSVFPLAP |
| | | CSRSTSESTAALGCLVKDYFPEPVTVSWNSGALTSGVHTFPAVLQSSGLYSLSSVVTVPSSSLGTK |
| | | TYTCNVDHKPSNTKVDKRVESKYGPPCPPCPAPEFLGGPSVFLFPPKPKDTLMISRTPEVTCVVVD |
| | | VSQEDPEVQFNWYVDGVEVHNAKTKPREEQFNSTYRVVSVLTVLHQDWLNGKEYKCKVSNKGLP |
| | | SSIEKTISKAKGQPREPQVYTLPPSQEEMTKNQVSLTCLVKGFYPSDIAVEWESNGQPENNYKTTP |
| | | PVLDSDGSFFLYSRLTVDKSRWQEGNVFSCSVMHEALHNHYTQKSLSLSLGK |
| 294 | 2G12 LC | DVVMTQSPLSLPVTPGQSASISCRSSQSLVDRDGNTYLTWFHQRPGQSPRRLIFKVSNRDSGVPD |
| | | RFSGSGSGTDFTLRISRVEAEDVGVYYCMQVTHWPRTFGQGTKVEIKRTVAAPSVFIFPPSDEQLK |
| | | SGTASVVCLLNNFYPREAKVQWKVDNALQSGNSQESVTEQDSKDSTYSLSSTLTLSKADYEKHKV |
| | | YACEVTHQGLSSPVTKSFNRGEC |
| 295 | 2H5 IgG1 HC | QVQLQQSGAEVKKPGSSVKVSCKASGGTFSSYAISWVRQAPGQGLEWMGGIIPIFGTANYAQKFQ |
| | | GRVTITADKSTSTAYMELSSLRSEDTAVYYCARARSGSYSLHYWGQGTLVTVSSASTKGPSVFPLA |
| | | PSSKSTSGGTAALGCLVKDYFPEPVTVSWNSGALTSGVHTFPAVLQSSGLYSLSSVVTVPSSSLGT |
| | | QTYICNVNHKPSNTKVDKKVEPKSCDKTHTCPPCPAPELLGGPSVFLFPPKPKDTLMISRTPEVTCV |
| | | VVDVSHEDPEVKFNWYVDGVEVHNAKTKPREEQYNSTYRVVSVLTVLHQDWLNGKEYKCKVSNK |
| | | ALPAPIEKTISKAKGQPREPQVYTLPPSRDELTKNQVSLTCLVKGFYPSDIAVEWESNGQPENNYKT |
| | | TPPVLDSDGSFFLYSKLTVDKSRWQQGNVFSCSVMHEALHNHYTQKSLSLSPGK |
| 296 | 2H5 IgG4 HC | QVQLQQSGAEVKKPGSSVKVSCKASGGTFSSYAISWVRQAPGQGLEWMGGIIPIFGTANYAQKFQ |
| | | GRVTITADKSTSTAYMELSSLRSEDTAVYYCARARSGSYSLHYWGQGTLVTVSSASTKGPSVFPLA |
| | | PCSRSTSESTAALGCLVKDYFPEPVTVSWNSGALTSGVHTFPAVLQSSGLYSLSSVVTVPSSSLGT |
| | | KTYTCNVDHKPSNTKVDKRVESKYGPPCPPCPAPEFLGGPSVFLFPPKPKDTLMISRTPEVTCVVV |
| | | DVSQEDPEVQFNWYVDGVEVHNAKTKPREEQFNSTYRVVSVLTVLHQDWLNGKEYKCKVSNKGL |
| | | PSSIEKTISKAKGQPREPQVYTLPPSQEEMTKNQVSLTCLVKGFYPSDIAVEWESNGQPENNYKTT |
| | | PPVLDSDGSFFLYSRLTVDKSRWQEGNVFSCSVMHEALHNHYTQKSLSLSLGK |
| 297 | 2H5 LC | NFMLTQPHSVSESPGKTVTISCTGSSGSIDSNYVQWYQQRPGSAPTTVIFEDSQRPSGVPDRFSGS |
| | | TDRSSNSASLTISGLKTEDEADYYCQSYDSSNNWVFGGGTKLTVLGQPKAAPSVTLFPPSSEELQA |
| | | NKATLVCLISDFYPGAVTVAWKADSSPVKAGVETTTPSKQSNNKYAASSYLSLTPEQWKSHRSYSC |
| | | QVTHEGSTVEKTVAPAECS |
| 298 | 3F8 IgG1 HC | EVQLVQSGAEVKKPGASVKVSCKASGYTFTSYAMHWVRQAPGQRLEWMGWINAGNGNTKYSQK |
| | | FQGRVTITRDTSASTAYMELSSLRSEDTAVYYCARVLHVLRYFDWPGAFDIWGQGTTVTVSSASTK |
| | | GPSVFPLAPSSKSTSGGTAALGCLVKDYFPEPVTVSWNSGALTSGVHTFPAVLQSSGLYSLSSVVT |
| | | VPSSSLGTQTYICNVNHKPSNTKVDKKVEPKSCDKTHTCPPCPAPELLGGPSVFLFPPKPKDTLMIS |
| | | RTPEVTCVVVDVSHEDPEVKFNWYVDGVEVHNAKTKPREEQYNSTYRVVSVLTVLHQDWLNGKE |
| | | YKCKVSNKALPAPIEKTISKAKGQPREPQVYTLPPSRDELTKNQVSLTCLVKGFYPSDIAVEWESNG |
| | | QPENNYKTTPPVLDSDGSFFLYSKLTVDKSRWQQGNVFSCSVMHEALHNHYTQKSLSLSPGK |
| 299 | 3F8 IgG4 HC | EVQLVQSGAEVKKPGASVKVSCKASGYTFTSYAMHWVRQAPGQRLEWMGWINAGNGNTKYSQK |
| | | FQGRVTITRDTSASTAYMELSSLRSEDTAVYYCARVLHVLRYFDWPGAFDIWGQGTTVTVSSASTK |
| | | GPSVFPLAPCSRSTSESTAALGCLVKDYFPEPVTVSWNSGALTSGVHTFPAVLQSSGLYSLSSVVT |
| | | VPSSSLGTKTYTCNVDHKPSNTKVDKRVESKYGPPCPPCPAPEFLGGPSVFLFPPKPKDTLMISRT |
| | | PEVTCVVVDVSQEDPEVQFNWYVDGVEVHNAKTKPREEQFNSTYRVVSVLTVLHQDWLNGKEYK |
| | | CKVSNKGLPSSIEKTISKAKGQPREPQVYTLPPSQEEMTKNQVSLTCLVKGFYPSDIAVEWESNGQ |
| | | PENNYKTTPPVLDSDGSFFLYSRLTVDKSRWQEGNVFSCSVMHEALHNHYTQKSLSLSLGK |
| 300 | 3F8 LC | QSVLTQPPSASGTPGQRVTISCSGSSSNIGTNYVYWYQQLPGTAPKLLIYINDQRPSGVPDRFSGS |
| | | KSGTSASLALSGLRSEDEADYYCAAWDDSLSGLVFGGGTKLTVLGQPKAAPSVTLFPPSSEELQAN |
| | | KATLVCLISDFYPGAVTVAWKADSSPVKAGVETTTPSKQSNNKYAASSYLSLTPEQWKSHKSYSCQ |
| | | VTHEGSTVEKTVAPAECS |
| 301 | 3F9 IgG1 HC | QVQLVQSGGGVVQPGRSLRLSCATSGFTFSSYGMHWVRQAPGKGLEWVAVISYDGSNKYYADSV |
| | | KGRFTISRDNSKNTLYLQMNSLRAEDTAVYYCARDLVDSSGWYGYYYYYGMDVWGQGTTVTVSS |
| | | ASTKGPSVFPLAPSSKSTSGGTAALGCLVKDYFPEPVTVSWNSGALTSGVHTFPAVLQSSGLYSLS |
| | | SVVTVPSSSLGTQTYICNVNHKPSNTKVDKKVEPKSCDKTHTCPPCPAPELLGGPSVFLFPPKPKDT |
| | | LMISRTPEVTCVVVDVSHEDPEVKFNWYVDGVEVHNAKTKPREEQYNSTYRVVSVLTVLHQDWLN |
| | | GKEYKCKVSNKALPAPIEKTISKAKGQPREPQVYTLPPSRDELTKNQVSLTCLVKGFYPSDIAVEWE |
| | | SNGQPENNYKTTPPVLDSDGSFFLYSKLTVDKSRWQQGNVFSCSVMHEALHNHYTQKSLSLSPGK |
| 302 | 3F9 IgG4 HC | QVQLVQSGGGVVQPGRSLRLSCATSGFTFSSYGMHWVRQAPGKGLEWVAVISYDGSNKYYADSV |
| | | KGRFTISRDNSKNTLYLQMNSLRAEDTAVYYCARDLVDSSGWYGYYYYYGMDVWGQGTTVTVSS |
| | | ASTKGPSVFPLAPCSRSTSESTAALGCLVKDYFPEPVTVSWNSGALTSGVHTFPAVLQSSGLYSLS |
| | | SVVTVPSSSLGTKTYTCNVDHKPSNTKVDKRVESKYGPPCPPCPAPEFLGGPSVFLFPPKPKDTLM |
| | | ISRTPEVTCVVVDVSQEDPEVQFNWYVDGVEVHNAKTKPREEQFNSTYRVVSVLTVLHQDWLNGK |
| | | EYKCKVSNKGLPSSIEKTISKAKGQPREPQVYTLPPSQEEMTKNQVSLTCLVKGFYPSDIAVEWESN |
| | | GQPENNYKTTPPVLDSDGSFFLYSRLTVDKSRWQEGNVFSCSVMHEALHNHYTQKSLSLSLGK |
| 303 | 3F9 LC | EIVLTQSPATLSLSPGERATLSCRASQSVSSYLAWYQQKPGQAPRLLIYDASNRATGIPARFSGSGS |
| | | GTDFTLTISSLEPEDFAVYFCQQRSNWPGTFGQGTKVEIKRTVAAPSVFIFPPSDEQLKSGTASVVC |
| | | LLNNFYPREAKVQWKVDNALQSGNSQESVTEQDSKDSTYSLSSTLTLSKADYEKHKVYACEVTHQ |
| | | GLSSPVTKSFNRGEC |
| 304 | 4G9 IgG1 HC | EVQLVQSGAEVKKPGDSVKVSCKASGYTFTGYYMHWVRQAPGQGLEWMGRINPNSGGTNYAQK |
| | | FQGRVTMTTDTSTSTAYMELRSLRSDDTAVYYCARDLGGATVYWGQGTLVTVSSASTKGPSVFPL |
| | | APSSKSTSGGTAALGCLVKDYFPEPVTVSWNSGALTSGVHTFPAVLQSSGLYSLSSVVTVPSSSLG |
| | | TQTYICNVNHKPSNTKVDKKVEPKSCDKTHTCPPCPAPELLGGPSVFLFPPKPKDTLMISRTPEVTC |
| | | VVVDVSHEDPEVKFNWYVDGVEVHNAKTKPREEQYNSTYRVVSVLTVLHQDWLNGKEYKCKVSN |
| | | KALPAPIEKTISKAKGQPREPQVYTLPPSRDELTKNQVSLTCLVKGFYPSDIAVEWESNGQPENNYK |
| | | TTPPVLDSDGSFFLYSKLTVDKSRWQQGNVFSCSVMHEALHNHYTQKSLSLSPGK |
| 305 | 4G9 IgG4 HC | EVQLVQSGAEVKKPGDSVKVSCKASGYTFTGYYMHWVRQAPGQGLEWMGRINPNSGGTNYAQK |
| | | FQGRVTMTTDTSTSTAYMELRSLRSDDTAVYYCARDLGGATVYWGQGTLVTVSSASTKGPSVFPL |
| | | APCSRSTSESTAALGCLVKDYFPEPVTVSWNSGALTSGVHTFPAVLQSSGLYSLSSVVTVPSSSLG |
| | | TKTYTCNVDHKPSNTKVDKRVESKYGPPCPPCPAPEFLGGPSVFLFPPKPKDTLMISRTPEVTCVV |
| | | VDVSQEDPEVQFNWYVDGVEVHNAKTKPREEQFNSTYRVVSVLTVLHQDWLNGKEYKCKVSNKG |
| | | LPSSIEKTISKAKGQPREPQVYTLPPSQEEMTKNQVSLTCLVKGFYPSDIAVEWESNGQPENNYKTT |
| | | PPVLDSDGSFFLYSRLTVDKSRWQEGNVFSCSVMHEALHNHYTQKSLSLSLGK |
| 306 | 4G9 LC | QAVLTQPSSLSASPGASASLTCTLHSGVNVGTQRIYWYQQKPGSPPRYLVRYKSDSNKQQGSGVP |
| | | SRFSGSKDASANAGILIISGLQSDDEADYYCMIWHNNAWVFGGGTTLTVLGQPKAAPSVTLFPPSSE |
| | | ELQANKATLVCLISDFYPGAVTVAWKADSSPVKAGVETTTPSKQSNNKYAASSYLSLTPEQWKSHK |
| | | SYSCQVTHEGSTVEKAVAPTECS |
| 307 | 5A3 IgG1 HC | EVQLVESGGGLVKPGGSLRLSCAASGFTFSSYSMNWVRQAPGKGLEWVSSISSSSSYIYYADSVK |
| | | GRFTISRDNAKNSLYLQMNSLRAEDTAVYYCARNGAVAGKYFDYWGQGTLVTVSSASTKGPSVFP |
| | | LAPSSKSTSGGTAALGCLVKDYFPEPVTVSWNSGALTSGVHTFPAVLQSSGLYSLSSVVTVPSSSL |
| | | GTQTYICNVNHKPSNTKVDKKVEPKSCDKTHTCPPCPAPELLGGPSVFLFPPKPKDTLMISRTPEVT |
| | | CVVVDVSHEDPEVKFNWYVDGVEVHNAKTKPREEQYNSTYRVVSVLTVLHQDWLNGKEYKCKVS |
| | | NKALPAPIEKTISKAKGQPREPQVYTLPPSRDELTKNQVSLTCLVKGFYPSDIAVEWESNGQPENNY |
| | | KTTPPVLDSDGSFFLYSKLTVDKSRWQQGNVFSCSVMHEALHNHYTQKSLSLSPGK |
| 308 | 5A3 IgG4 HC | EVQLVESGGGLVKPGGSLRLSCAASGFTFSSYSMNWVRQAPGKGLEWVSSISSSSSYIYYADSVK |
| | | GRFTISRDNAKNSLYLQMNSLRAEDTAVYYCARNGAVAGKYFDYWGQGTLVTVSSASTKGPSVFP |
| | | LAPCSRSTSESTAALGCLVKDYFPEPVTVSWNSGALTSGVHTFPAVLQSSGLYSLSSVVTVPSSSL |
| | | GTKTYTCNVDHKPSNTKVDKRVESKYGPPCPPCPAPEFLGGPSVFLFPPKPKDTLMISRTPEVTCV |
| | | VVDVSQEDPEVQFNWYVDGVEVHNAKTKPREEQFNSTYRVVSVLTVLHQDWLNGKEYKCKVSNK |
| | | GLPSSIEKTISKAKGQPREPQVYTLPPSQEEMTKNQVSLTCLVKGFYPSDIAVEWESNGQPENNYK |
| | | TTPPVLDSDGSFFLYSRLTVDKSRWQEGNVFSCSVMHEALHNHYTQKSLSLSLGK |
| 309 | 5A3 LC | DVVMTQSPLSLPVTPGEPASISCRSSQSLLHSNGYNYLDWYLQKPGQSPQLLIYLGSNRASGVPDR |
| | | FSGSGSGTDFTLKISRVEAEDVGVYYCMQALQTPLTFGGGTKVEIKRTVAAPSVFIFPPSDEQLKSG |
| | | TASVVCLLNNFYPREAKVQWKVDNALQSGNSQESVTEQDSKDSTYSLSSTLTLSKADYEKHKLYAC |
| | | EVTHQGLSSPVTKSFNRGEC |
| 310 | 5E8 IgG1 HC | QVQLVQSGGGVVQPGRSLRLSCAASGFTFSSYAMHWVRQAPGKGLEWVAVISYDGSNKYYADSV |
| | | KGRFTISRDNSKNTLYLQMNSLRAEDTAVYYCARDFYPFIVGATSFFDYWGQGTLVTVSSASTKGP |
| | | SVFPLAPSSKSTSGGTAALGCLVKDYFPEPVTVSWNSGALTSGVHTFPAVLQSSGLYSLSSVVTVP |
| | | SSSLGTQTYICNVNHKPSNTKVDKKVEPKSCDKTHTCPPCPAPELLGGPSVFLFPPKPKDTLMISRT |
| | | PEVTCVVVDVSHEDPEVKFNWYVDGVEVHNAKTKPREEQYNSTYRVVSVLTVLHQDWLNGKEYK |
| | | CKVSNKALPAPIEKTISKAKGQPREPQVYTLPPSRDELTKNQVSLTCLVKGFYPSDIAVEWESNGQP |
| | | ENNYKTTPPVLDSDGSFFLYSKLTVDKSRWQQGNVFSCSVMHEALHNHYTQKSLSLSPGK |
| 311 | 5E8 IgG4 HC | QVQLVQSGGGVVQPGRSLRLSCAASGFTFSSYAMHWVRQAPGKGLEWVAVISYDGSNKYYADSV |
| | | KGRFTISRDNSKNTLYLQMNSLRAEDTAVYYCARDFYPFIVGATSFFDYWGQGTLVTVSSASTKGP |
| | | SVFPLAPCSRSTSESTAALGCLVKDYFPEPVTVSWNSGALTSGVHTFPAVLQSSGLYSLSSVVTVP |
| | | SSSLGTKTYTCNVDHKPSNTKVDKRVESKYGPPCPPCPAPEFLGGPSVFLFPPKPKDTLMISRTPE |
| | | VTCVVVDVSQEDPEVQFNWYVDGVEVHNAKTKPREEQFNSTYRVVSVLTVLHQDWLNGKEYKCK |
| | | VSNKGLPSSIEKTISKAKGQPREPQVYTLPPSQEEMTKNQVSLTCLVKGFYPSDIAVEWESNGQPE |
| | | NNYKTTPPVLDSDGSFFLYSRLTVDKSRWQEGNVFSCSVMHEALHNHYTQKSLSLSLGK |
| 312 | 5E8 LC | DIQLTQSPSSLSASVGDRVTITCRASQSISTYINWYQQKPGKAPKLLIYFASSLQSGVPSRFSGGGS |
| | | GTDFTLTISSLQPEDFATYYCQQSYNIPVTFGQGTKVEVTRTVAAPSIFIFPPSDEQLKSGTASVVCL |
| | | LNNFYPREAKVQWKVDNALQSGNSQESVTEQDSKDSTYSLSSTLTLSKADYEKHKVYACEVTHQG |
| | | LSSPVTKSFNRGEC |
| 313 | C001, C008 IgG1 | QVQLVQSGGGVVQPGRSLRLSCAASGFTVSSNYMSWVRQAPGKGLEWVSVIYSGGSTYYADSVK |
| | HC | GRFTISRDNSKNTLYLQMNSLRAEDTAVYYCARGSRAVAGAFDYWGQGTLVTVSSASTKGPSVFP |
| | | LAPSSKSTSGGTAALGCLVKDYFPEPVTVSWNSGALTSGVHTFPAVLQSSGLYSLSSVVTVPSSSL |
| | | GTQTYICNVNHKPSNTKVDKKVEPKSCDKTHTCPPCPAPELLGGPSVFLFPPKPKDTLMISRTPEVT |
| | | CVVVDVSHEDPEVKFNWYVDGVEVHNAKTKPREEQYNSTYRVVSVLTVLHQDWLNGKEYKCKVS |
| | | NKALPAPIEKTISKAKGQPREPQVYTLPPSREEMTKNQVSLTCLVKGFYPSDIAVEWESNGQPENN |
| | | YKTTPPVLDSDGSFFLYSKLTVDKSRWQQGNVFSCSVMHEALHNHYTQKSLSLSPGK |
| 314 | C001, C008 IgG4 | QVQLVQSGGGVVQPGRSLRLSCAASGFTVSSNYMSWVRQAPGKGLEWVSVIYSGGSTYYADSVK |
| | HC | GRFTISRDNSKNTLYLQMNSLRAEDTAVYYCARGSRAVAGAFDYWGQGTLVTVSSASTKGPSVFP |
| | | LAPCSRSTSESTAALGCLVKDYFPEPVTVSWNSGALTSGVHTFPAVLQSSGLYSLSSVVTVPSSSL |
| | | GTKTYTCNVDHKPSNTKVDKRVESKYGPPCPSCPAPEFLGGPSVFLFPPKPKDTLMISRTPEVTCV |
| | | VVDVSQEDPEVQFNWYVDGVEVHNAKTKPREEQFNSTYRVVSVLTVLHQDWLNGKEYKCKVSNK |
| | | GLPSSIEKTISKAKGQPREPQVYTLPPSQEEMTKNQVSLTCLVKGFYPSDIAVEWESNGQPENNYK |
| | | TTPPVLDSDGSFFLYSRLTVDKSRWQEGNVFSCSVMHEALHNHYTQKSLSLSLGK |
| 315 | C001 LC | SSELTQDPAVSVALGQTVSITCQGDSLRNYFAAWYQQKPGQAPLLVIYGKNNRPSGIPDRFSGSSS |
| | | GNTASLTITGAEAEDEADYYCGSRDSSGNHPVFGGGTKVTVLGQPKAAPSVTLFPPSSEELQANKA |
| | | TLVCLISDFYPGAVTVAWKADSSPVKAGVETTTPSKQSNNKYAASSYLSLTPEQWKSHRSYSCQVT |
| | | HEGSTVEKTVAPTECS |
| 316 | C008 LC | YSELTQDPAVSVALGQTVRITCQGDSLRTYYANWYQQKPGQAPVLVMYAKNHRPSGIPDRFSGSS |
| | | SGNTASLTITGAQAEDEADYYCGSRDSSGNHPVFGGGTKVTVLGQPKAAPSVTLFPPSSEELQANK |
| | | ATLVCLISDFYPGAVTVAWKADSSPVKAGVETTTPSKQSNNKYAASSYLSLTPEQWKSHRSYSCQV |
| | | THEGSTVEKTVAPTECS |
| 317 | C010 IgG1 HC | QVQLQQSGPGLVKPSQTLSVTCAISGDSVSSNSAAWNWFRQSPSRGLEWLGRTYYRSKWYSDYA |
| | | VSVKSRITINPDTSKNQFSLQLNSVTPEDTAVYYCARDVRAARAFDIWGQGTMVTVSSASTKGPSV |
| | | FPLAPSSKSTSGGTAALGCLVKDYFPEPVTVSWNSGALTSGVHTFPAVLQSSGLYSLSSVVTVPSS |
| | | SLGTQTYICNVNHKPSNTKVDKKVEPKSCDKTHTCPPCPAPELLGGPSVFLFPPKPKDTLMISRTPE |
| | | VTCVVVDVSHEDPEVKFNWYVDGVEVHNAKTKPREEQYNSTYRVVSVLTVLHQDWLNGKEYKCK |
| | | VSNKALPAPIEKTISKAKGQPREPQVYTLPPSREEMTKNQVSLTCLVKGFYPSDIAVEWESNGQPEN |
| | | NYKTTPPVLDSDGSFFLYSKLTVDKSRWQQGNVFSCSVMHEALHNHYTQKSLSLSPGK |
| 318 | C010 IgG4 HC | QVQLQQSGPGLVKPSQTLSVTCAISGDSVSSNSAAWNWFRQSPSRGLEWLGRTYYRSKWYSDYA |
| | | VSVKSRITINPDTSKNQFSLQLNSVTPEDTAVYYCARDVRAARAFDIWGQGTMVTVSSASTKGPSV |
| | | FPLAPCSRSTSESTAALGCLVKDYFPEPVTVSWNSGALTSGVHTFPAVLQSSGLYSLSSVVTVPSS |
| | | SLGTKTYTCNVDHKPSNTKVDKRVESKYGPPCPSCPAPEFLGGPSVFLFPPKPKDTLMISRTPEVT |
| | | CVVVDVSQEDPEVQFNWYVDGVEVHNAKTKPREEQFNSTYRVVSVLTVLHQDWLNGKEYKCKVS |
| | | NKGLPSSIEKTISKAKGQPREPQVYTLPPSQEEMTKNQVSLTCLVKGFYPSDIAVEWESNGQPENN |
| | | YKTTPPVLDSDGSFFLYSRLTVDKSRWQEGNVFSCSVMHEALHNHYTQKSLSLSLGK |
| 319 | C010 LC | QAVLTQPSSLSASPGASASLTCTLRSGINVGTYRIYWYQQKPGSPPQYLLRYKSDSDKQQGSGVPS |
| | | RFSGSKDASANAGILVISGLQSEDEADYYCLIWHNRGWVFGGGTKVTVLGQPKAAPSVTLFPPSSE |
| | | ELQANKATLVCLISDFYPGAVTVAWKADSSPVKAGVETTTPSKQSNNKYAASSYLSLTPEQWKSHR |
| | | SYSCQVTHEGSTVEKTVAPTECS |
| 320 | C023, C046 IgG1 | EVQLVESGGGLVQPGRPLRLSCAASGFTFSSYAMSWVRQAPGKGLEWVSSISSGGSTYYADSVR |
| | HC | GRFTISRDDSKDTLYLQMNSLRAEDTAVYYCARTGVGGAFDIWGQGTMVTVSSASTKGPSVFPLAP |
| | | SSKSTSGGTAALGCLVKDYFPEPVTVSWNSGALTSGVHTFPAVLQSSGLYSLSSVVTVPSSSLGTQ |
| | | TYICNVNHKPSNTKVDKKVEPKSCDKTHTCPPCPAPELLGGPSVFLFPPKPKDTLMISRTPEVTCVV |
| | | VDVSHEDPEVKFNWYVDGVEVHNAKTKPREEQYNSTYRVVSVLTVLHQDWLNGKEYKCKVSNKA |
| | | LPAPIEKTISKAKGQPREPQVYTLPPSREEMTKNQVSLTCLVKGFYPSDIAVEWESNGQPENNYKTT |
| | | PPVLDSDGSFFLYSKLTVDKSRWQQGNVFSCSVMHEALHNHYTQKSLSLSPGK |
| 321 | C023, C046 IgG4 | EVQLVESGGGLVQPGRPLRLSCAASGFTFSSYAMSWVRQAPGKGLEWVSSISSGGSTYYADSVR |
| | HC | GRFTISRDDSKDTLYLQMNSLRAEDTAVYYCARTGVGGAFDIWGQGTMVTVSSASTKGPSVFPLAP |
| | | CSRSTSESTAALGCLVKDYFPEPVTVSWNSGALTSGVHTFPAVLQSSGLYSLSSVVTVPSSSLGTK |
| | | TYTCNVDHKPSNTKVDKRVESKYGPPCPSCPAPEFLGGPSVFLFPPKPKDTLMISRTPEVTCVVVD |
| | | VSQEDPEVQFNWYVDGVEVHNAKTKPREEQFNSTYRVVSVLTVLHQDWLNGKEYKCKVSNKGLP |
| | | SSIEKTISKAKGQPREPQVYTLPPSQEEMTKNQVSLTCLVKGFYPSDIAVEWESNGQPENNYKTTP |
| | | PVLDSDGSFFLYSRLTVDKSRWQEGNVFSCSVMHEALHNHYTQKSLSLSLGK |
| 322 | C023 LC | DVVMTQSPLSLPVTPGEPASISCRSSHSLLYDNRYNYLDWYLQRPGQSPQLLIYLGSTRASGVPDR |
| | | FSGSGSGTDFTLKISRVEAEDIGVYYCMQAVQTPIIFGQGTRLEIKRTVAAPSVFIFPPSDEQLKSGT |
| | | ASVVCLLNNFYPREAKVQWKVDNALQSGNSQESVTEQDSKDSTYSLSSTLTLSKADYEKHKVYAC |
| | | EVTHQGLSSPVTKSFNRGEC |
| 323 | C025 IgG1 HC | EVQLLESGGGLVQPGGSLRLSCAASGFTFSSYAMSWVRQAPGKGLEWVSTIYYAGSNTYYADSVK |
| | | GRFTISRDNSKNTLYLQMNSLRAEDTAVYYCAKGYYTFDYWGQGTLVTVSSASTKGPSVFPLAPSS |
| | | KSTSGGTAALGCLVKDYFPEPVTVSWNSGALTSGVHTFPAVLQSSGLYSLSSVVTVPSSSLGTQTYI |
| | | CNVNHKPSNTKVDKKVEPKSCDKTHTCPPCPAPELLGGPSVFLFPPKPKDTLMISRTPEVTCVVVD |
| | | VSHEDPEVKFNWYVDGVEVHNAKTKPREEQYNSTYRVVSVLTVLHQDWLNGKEYKCKVSNKALPA |
| | | PIEKTISKAKGQPREPQVYTLPPSREEMTKNQVSLTCLVKGFYPSDIAVEWESNGQPENNYKTTPPV |
| | | LDSDGSFFLYSKLTVDKSRWQQGNVFSCSVMHEALHNHYTQKSLSLSPGK |
| 324 | C025 IgG4 HC | EVQLLESGGGLVQPGGSLRLSCAASGFTFSSYAMSWVRQAPGKGLEWVSTIYYAGSNTYYADSVK |
| | | GRFTISRDNSKNTLYLQMNSLRAEDTAVYYCAKGYYTFDYWGQGTLVTVSSASTKGPSVFPLAPCS |
| | | RSTSESTAALGCLVKDYFPEPVTVSWNSGALTSGVHTFPAVLQSSGLYSLSSVVTVPSSSLGTKTY |
| | | TCNVDHKPSNTKVDKRVESKYGPPCPSCPAPEFLGGPSVFLFPPKPKDTLMISRTPEVTCVVVDVS |
| | | QEDPEVQFNWYVDGVEVHNAKTKPREEQFNSTYRVVSVLTVLHQDWLNGKEYKCKVSNKGLPSSI |
| | | EKTISKAKGQPREPQVYTLPPSQEEMTKNQVSLTCLVKGFYPSDIAVEWESNGQPENNYKTTPPVL |
| | | DSDGSFFLYSRLTVDKSRWQEGNVFSCSVMHEALHNHYTQKSLSLSLGK |
| 325 | C025 LC | QDPDVSVALGQTVRITCHGDSLRSSYASWYQQKPGQAPVLVFFGKNNRPSGIPDRFSGSSSGDTA |
| | | SLTITGAQAEDEADYYCHSRDTSDNHLRVFGGGTKLTVLGQPKAAPSVTLFPPSSEELQANKATLV |
| | | CLISDFYPGAVTVAWKADSSPVKAGVETTTPSKQSNNKYAASSYLSLTPEQWKSHRSYSCQVTHE |
| | | GSTVEKTVAPTECS |
| 326 | C040 IgG1 HC | EVQLVESGGGLVRPGRSLRLSCRASGLTLSDYGMHWVRQAPGKGLEWVSGISGSGSNTYYTDSV |
| | | KGRFTISRDNAKNSLYLQMNSLRAEDTAVYFCARSVRGAFDNWGQGTMVTVSSASTKGPSVFPLA |
| | | PSSKSTSGGTAALGCLVKDYFPEPVTVSWNSGALTSGVHTFPAVLQSSGLYSLSSVVTVPSSSLGT |
| | | QTYICNVNHKPSNTKVDKKVEPKSCDKTHTCPPCPAPELLGGPSVFLFPPKPKDTLMISRTPEVTCV |
| | | VVDVSHEDPEVKFNWYVDGVEVHNAKTKPREEQYNSTYRVVSVLTVLHQDWLNGKEYKCKVSNK |
| | | ALPAPIEKTISKAKGQPREPQVYTLPPSREEMTKNQVSLTCLVKGFYPSDIAVEWESNGQPENNYKT |
| | | TPPVLDSDGSFFLYSKLTVDKSRWQQGNVFSCSVMHEALHNHYTQKSLSLSPGK |
| 327 | C040 IgG4 HC | EVQLVESGGGLVRPGRSLRLSCRASGLTLSDYGMHWVRQAPGKGLEWVSGISGSGSNTYYTDSV |
| | | KGRFTISRDNAKNSLYLQMNSLRAEDTAVYFCARSVRGAFDNWGQGTMVTVSSASTKGPSVFPLA |
| | | PCSRSTSESTAALGCLVKDYFPEPVTVSWNSGALTSGVHTFPAVLQSSGLYSLSSVVTVPSSSLGT |
| | | KTYTCNVDHKPSNTKVDKRVESKYGPPCPSCPAPEFLGGPSVFLFPPKPKDTLMISRTPEVTCVVV |
| | | DVSQEDPEVQFNWYVDGVEVHNAKTKPREEQFNSTYRVVSVLTVLHQDWLNGKEYKCKVSNKGL |
| | | PSSIEKTISKAKGQPREPQVYTLPPSQEEMTKNQVSLTCLVKGFYPSDIAVEWESNGQPENNYKTT |
| | | PPVLDSDGSFFLYSRLTVDKSRWQEGNVFSCSVMHEALHNHYTQKSLSLSLGK |
| 328 | C040 LC | SSELTQDPAVSVALGQTVRITCQGDSLRRFFASWYQQRPGQAPILVIYGKNNRPSGIPDRFSGSRS |
| | | GNTASLTIVGAQADDEADYYCSSRDSSRDRLVFGGGTKLTVLGQPKAAPSVTLFPPSSEELQANKA |
| | | TLVCLISDFYPGAVTVAWKADSSPVKAGVETTTPSKQSNNKYAASSYLSLTPEQWKSHRSYSCQVT |
| | | HEGSTVEKTVAPTECS |
| 329 | C046 LC | QSVLTQPPSVSAAPGQKVTISCSGSSSNIGDNYVSWYQQLPGTAPKVLIYGNNNRPSGIPDRFSGS |
| | | KSGTSASLAISGLQSEDEADYYCATWDDSLKGYVFGTGTQLTVLGQPKAAPSVTLFPPSSEELQAN |
| | | KATLVCLISDFYPGAVTVAWKADSSPVKAGVETTTPSKQSNNKYAASSYLSLTPEQWKSHRSYSCQ |
| | | VTHEGSTVEKTVAPTECS |
| 330 | C117 IgG1 HC | QVQLQQSGPGLVKPSQTLSLTCAISGDSVSSNSAAWNWIRQSPSRGLEWLGRTYYRSKWYNDYA |
| | | VSVKSRITINPDTSKNQFSLQLNSVTPEDTAVYYCARGIVGAFDYWGQGTLVTVSSASTKGPSVFPL |
| | | APSSKSTSGGTAALGCLVKDYFPEPVTVSWNSGALTSGVHTFPAVLQSSGLYSLSSVVTVPSSSLG |
| | | TQTYICNVNHKPSNTKVDKKVEPKSCDKTHTCPPCPAPELLGGPSVFLFPPKPKDTLMISRTPEVTC |
| | | VVVDVSHEDPEVKFNWYVDGVEVHNAKTKPREEQYNSTYRVVSVLTVLHQDWLNGKEYKCKVSN |
| | | KALPAPIEKTISKAKGQPREPQVYTLPPSREEMTKNQVSLTCLVKGFYPSDIAVEWESNGQPENNYK |
| | | TTPPVLDSDGSFFLYSKLTVDKSRWQQGNVFSCSVMHEALHNHYTQKSLSLSPGK |
| 331 | C117 IgG4 HC | QVQLQQSGPGLVKPSQTLSLTCAISGDSVSSNSAAWNWIRQSPSRGLEWLGRTYYRSKWYNDYA |
| | | VSVKSRITINPDTSKNQFSLQLNSVTPEDTAVYYCARGIVGAFDYWGQGTLVTVSSASTKGPSVFPL |
| | | APCSRSTSESTAALGCLVKDYFPEPVTVSWNSGALTSGVHTFPAVLQSSGLYSLSSVVTVPSSSLG |
| | | TKTYTCNVDHKPSNTKVDKRVESKYGPPCPSCPAPEFLGGPSVFLFPPKPKDTLMISRTPEVTCVV |
| | | VDVSQEDPEVQFNWYVDGVEVHNAKTKPREEQFNSTYRVVSVLTVLHQDWLNGKEYKCKVSNKG |
| | | LPSSIEKTISKAKGQPREPQVYTLPPSQEEMTKNQVSLTCLVKGFYPSDIAVEWESNGQPENNYKTT |
| | | PPVLDSDGSFFLYSRLTVDKSRWQEGNVFSCSVMHEALHNHYTQKSLSLSLGK |
| 332 | C117 LC | SSELTQDPAVSVALGQTVRITCQGDSLRSYHASWYQQKPGQAPVLVIYGRNNRPSGIPDRFSGSTS |
| | | GNTASLTITGAQAEDEADYYCKSRDSSGSLSYVFGTGTKVTVLGQPKAAPSVTLFPPSSEELQANK |
| | | ATLVCLISDFYPGAVTVAWKADSSPVKAGVETTTPSKQSNNKYAASSYLSLTPEQWKSHRSYSCQV |
| | | THEGSTVEKTVAPTECS |
| 333 | Human CNX | MEGKWLLCMLLVLGTAIVEAHDGHDDDVIDIEDDLDDVIEEVEDSKPDTTAPPSSPKVTYKAPVPTG |
| | isoform 1 | EVYFADSFDRGTLSGWILSKAKKDDTDDEIAKYDGKWEVEEMKESKLPGDKGLVLMSRAKHHAISA |
| | (UniProt: P27824- | KLNKPFLFDTKPLIVQYEVNFQNGIECGGAYVKLLSKTPELNLDQFHDKTPYTIMFGPDKCGEDYKL |
| | 1, v2) | HFIFRHKNPKTGIYEEKHAKRPDADLKTYFTDKKTHLYTLILNPDNSFEILVDQSVVNSGNLLNDMTP |
| | | PVNPSREIEDPEDRKPEDWDERPKIPDPEAVKPDDWDEDAPAKIPDEEATKPEGWLDDEPEYVPD |
| | | PDAEKPEDWDEDMDGEWEAPQIANPRCESAPGCGVWQRPVIDNPNYKGKWKPPMIDNPSYQGI |
| | | WKPRKIPNPDFFEDLEPFRMTPFSAIGLELWSMTSDIFFDNFIICADRRIVDDWANDGWGLKKAADG |
| | | AAEPGVVGQMIEAAEERPWLWVVYILTVALPVFLVILFCCSGKKQTSGMEYKKTDAPQPDVKEEEE |
| | | EKEEEKDKGDEEEEGEEKLEEKQKSDAEEDGGTVSQEEEDRKPKAEEDEILNRSPRNRKPRRE |
| 334 | Human CNX | MADRRTPTPFAGCRLPRQRRARDASQVSAPGTRRIMEGKWLLCMLLVLGTAIVEAHDGHDDDVIDI |
| | isoform 2 | EDDLDDVIEEVEDSKPDTTAPPSSPKVTYKAPVPTGEVYFADSFDRGTLSGWILSKAKKDDTDDEIA |
| | (UniProt: P27824- | KYDGKWEVEEMKESKLPGDKGLVLMSRAKHHAISAKLNKPFLFDTKPLIVQYEVNFQNGIECGGAY |
| | 2) | VKLLSKTPELNLDQFHDKTPYTIMFGPDKCGEDYKLHFIFRHKNPKTGIYEEKHAKRPDADLKTYFTD |
| | | KKTHLYTLILNPDNSFEILVDQSVVNSGNLLNDMTPPVNPSREIEDPEDRKPEDWDERPKIPDPEAV |
| | | KPDDWDEDAPAKIPDEEATKPEGWLDDEPEYVPDPDAEKPEDWDEDMDGEWEAPQIANPRCESA |
| | | PGCGVWQRPVIDNPNYKGKWKPPMIDNPSYQGIWKPRKIPNPDFFEDLEPFRMTPFSAIGLELWS |
| | | MTSDIFFDNFIICADRRIVDDWANDGWGLKKAADGAAEPGVVGQMIEAAEERPWLWVVYILTVALPV |
| | | FLVILFCCSGKKQTSGMEYKKTDAPQPDVKEEEEEKEEEKDKGDEEEEGEEKLEEKQKSDAEEDG |
| | | GTVSQEEEDRKPKAEEDEILNRSPRNRKPRRE |
| 335 | Human CNX | MKESKLPGDKGLVLMSRAKHHAISAKLNKPFLFDTKPLIVQYEVNFQNGIECGGAYVKLLSKTPELNL |
| | isoform 3 | DQFHDKTPYTIMFGPDKCGEDYKLHFIFRHKNPKTGIYEEKHAKRPDADLKTYFTDKKTHLYTLILNP |
| | (UniProt: P27824- | DNSFEILVDQSVVNSGNLLNDMTPPVNPSREIEDPEDRKPEDWDERPKIPDPEAVKPDDWDEDAPA |
| | 3) | KIPDEEATKPEGWLDDEPEYVPDPDAEKPEDWDEDMDGEWEAPQIANPRCESAPGCGVWQRPVI |
| | | DNPNYKGKWKPPMIDNPSYQGIWKPRKIPNPDFFEDLEPFRMTPFSAIGLELWSMTSDIFFDNFIICA |
| | | DRRIVDDWANDGWGLKKAADGAAEPGVVGQMIEAAEERPWLWVVYILTVALPVFLVILFCCSGKKQ |
| | | TSGMEYKKTDAPQPDVKEEEEEKEEEKDKGDEEEEGEEKLEEKQKSDAEEDGGTVSQEEEDRKP |
| | | KAEEDEILNRSPRNRKPRRE |
| 336 | Human CNX | MEGKWLLCMLLVLGTAIVEA |
| | isoform 1 signal | |
| | peptide | |
| 337 | Human CNX | HDGHDDDVIDIEDDLDDVIEEVEDSKPDTTAPPSSPKVTYKAPVPTGEVYFADSFDRGTLSGWILSK |
| | lumenal domain | AKKDDTDDEIAKYDGKWEVEEMKESKLPGDKGLVLMSRAKHHAISAKLNKPFLFDTKPLIVQYEVNF |
| | | QNGIECGGAYVKLLSKTPELNLDQFHDKTPYTIMFGPDKCGEDYKLHFIFRHKNPKTGIYEEKHAKR |
| | | PDADLKTYFTDKKTHLYTLILNPDNSFEILVDQSVVNSGNLLNDMTPPVNPSREIEDPEDRKPEDWD |
| | | ERPKIPDPEAVKPDDWDEDAPAKIPDEEATKPEGWLDDEPEYVPDPDAEKPEDWDEDMDGEWEA |
| | | PQIANPRCESAPGCGVWQRPVIDNPNYKGKWKPPMIDNPSYQGIWKPRKIPNPDFFEDLEPFRMT |
| | | PFSAIGLELWSMTSDIFFDNFIICADRRIVDDWANDGWGLKKAADGAAEPGVVGQMIEAAEERP |
| 338 | Human CNX | WLWVVYILTVALPVFLVILFC |
| | transmembrane | |
| | domain | |
| 339 | Human CNX | CSGKKQTSGMEYKKTDAPQPDVKEEEEEKEEEKDKGDEEEEGEEKLEEKQKSDAEEDGGTVSQE |
| | cytoplasmic | EEDRKPKAEEDEILNRSPRNRKPRRE |
| | domain | |
| 340 | Human CNX lectin | KAPVPTGEVYFADSFDRGTLSGWILSKAKKDDTDDEIAKYDGKWEVEEMKESKLPGDKGLVLMSR |
| | domain 1 | AKHHAISAKLNKPFLFDTKPLIVQYEVNFQNGIECGGAYVKLLSKTPELNLDQFHDKTPYTIMFGPDK |
| | | CGEDYKLHFIFRHKNPKTGIYEEKHAKRPDADLKTYFTDKKTHLYTLILNPDNSFEILVDQSVVNSGN |
| | | LLNDMTPPVN |
| 341 | Human CNX P | PSREIEDPEDRKPEDWDERPKIPDPEAVKPDDWDEDAPAKIPDEEATKPEGWLDDEPEYVPDPDA |
| | domain | EKPEDWDEDMDGEWEAPQIANPRCESAPGCGVWQRPVIDNPNYKGKWKPPMIDNPSYQGIWKP |
| | | RKIPNPDFFEDL |
| 342 | Human CNX lectin | EPFRMTPFSAIGLELWSMTSDIFFDNFIICADRRIVDDWANDGWGL |
| | domain 2 | |
| 343 | Mature human | HDGHDDDVIDIEDDLDDVIEEVEDSKPDTTAPPSSPKVTYKAPVPTGEVYFADSFDRGTLSGWILSK |
| | CNX isoform 1 | AKKDDTDDEIAKYDGKWEVEEMKESKLPGDKGLVLMSRAKHHAISAKLNKPFLFDTKPLIVQYEVNF |
| | | QNGIECGGAYVKLLSKTPELNLDQFHDKTPYTIMFGPDKCGEDYKLHFIFRHKNPKTGIYEEKHAKR |
| | | PDADLKTYFTDKKTHLYTLILNPDNSFEILVDQSVVNSGNLLNDMTPPVNPSREIEDPEDRKPEDWD |
| | | ERPKIPDPEAVKPDDWDEDAPAKIPDEEATKPEGWLDDEPEYVPDPDAEKPEDWDEDMDGEWEA |
| | | PQIANPRCESAPGCGVWQRPVIDNPNYKGKWKPPMIDNPSYQGIWKPRKIPNPDFFEDLEPFRMT |
| | | PFSAIGLELWSMTSDIFFDNFIICADRRIVDDWANDGWGLKKAADGAAEPGVVGQMIEAAEERPWL |
| | | WVVYILTVALPVFLVILFCCSGKKQTSGMEYKKTDAPQPDVKEEEEEKEEEKDKGDEEEEGEEKLE |
| | | EKQKSDAEEDGGTVSQEEEDRKPKAEEDEILNRSPRNRKPRRE |
| 344 | Mouse CNX | MEGKWLLCLLLVLGTAAVEAHDGHDDDAIDIEDDLDDVIEEVEDSKSKSDASTPPSPKVTYKAPVPT |
| | (UniProt: P35564- | GEVYFADSFDRGSLSGWILSKAKKDDTDDEIAKYDGKWEVDEMKETKLPGDKGLVLMSRAKHHAIS |
| | 1, v1) | AKLNKPFLFDTKPLIVQYEVNFQNGIECGGAYVKLLSKTAELSLDQFHDKTPYTIMFGPDKCGEDYKL |
| | | HFIFRHKNPKTGVYEEKHAKRPDADLKTYFTDKKTHLYTLILNPDNSFEILVDQSVVNSGNLLNDMTP |
| | | PVNPSREIEDPEDRKPEDWDERPKIADPDAVKPDDWDEDAPSKIPDEEATKPEGWLDDEPEYIPDP |
| | | DAEKPEDWDEDMDGEWEAPQIANPKCESAPGCGVWQRPMIDNPNYKGKWKPPMIDNPNYQGIW |
| | | KPRKIPNPDFFEDLEPFKMTPFSAIGLELWSMTSDIFFDNFIISGDRRVVDDWANDGWGLKKAADGA |
| | | AEPGVVLQMLEAAEERPWLWVVYILTVALPVFLVILFCCSGKKQSNAMEYKKTDAPQPDVKDEEGK |
| | | EEEKNKRDEEEEEEKLEEKQKSDAEEDGVTGSQDEEDSKPKAEEDEILNRSPRNRKPRRE |
| 345 | Mouse CNX | MEGKWLLCLLLVLGTAAVEA |
| | signal peptide | |
| 346 | Mouse CNX | HDGHDDDAIDIEDDLDDVIEEVEDSKSKSDASTPPSPKVTYKAPVPTGEVYFADSFDRGSLSGWILS |
| | lumenal domain | KAKKDDTDDEIAKYDGKWEVDEMKETKLPGDKGLVLMSRAKHHAISAKLNKPFLFDTKPLIVQYEVN |
| | | FQNGIECGGAYVKLLSKTAELSLDQFHDKTPYTIMFGPDKCGEDYKLHFIFRHKNPKTGVYEEKHAK |
| | | RPDADLKTYFTDKKTHLYTLILNPDNSFEILVDQSVVNSGNLLNDMTPPVNPSREIEDPEDRKPEDW |
| | | DERPKIADPDAVKPDDWDEDAPSKIPDEEATKPEGWLDDEPEYIPDPDAEKPEDWDEDMDGEWEA |
| | | PQIANPKCESAPGCGVWQRPMIDNPNYKGKWKPPMIDNPNYQGIWKPRKIPNPDFFEDLEPFKMT |
| | | PFSAIGLELWSMTSDIFFDNFIISGDRRVVDDWANDGWGLKKAADGAAEPGVVLQMLEAAEERP |
| 347 | Mouse CNX | WLWVVYILTVALPVFLVILFC |
| | transmembrane | |
| | domain | |
| 348 | Mouse CNX | CSGKKQSNAMEYKKTDAPQPDVKDEEGKEEEKNKRDEEEEEEKLEEKQKSDAEEDGVTGSQDEE |
| | cytoplasmic | DSKPKAEEDEILNRSPRNRKPRRE |
| | domain | |
| 349 | Mouse CNX lectin | KAPVPTGEVYFADSFDRGSLSGWILSKAKKDDTDDEIAKYDGKWEVDEMKETKLPGDKGLVLMSR |
| | domain 1 | AKHHAISAKLNKPFLFDTKPLIVQYEVNFQNGIECGGAYVKLLSKTAELSLDQFHDKTPYTIMFGPDK |
| | | CGEDYKLHFIFRHKNPKTGVYEEKHAKRPDADLKTYFTDKKTHLYTLILNPDNSFEILVDQSVVNSGN |
| | | LLNDMTPPVN |
| 350 | Mouse CNX P | PSREIEDPEDRKPEDWDERPKIADPDAVKPDDWDEDAPSKIPDEEATKPEGWLDDEPEYIPDPDAE |
| | domain | KPEDWDEDMDGEWEAPQIANPKCESAPGCGVWQRPMIDNPNYKGKWKPPMIDNPNYQGIWKPR |
| | | KIPNPDFFEDL |
| 351 | Mouse CNX lectin | EPFKMTPFSAIGLELWSMTSDIFFDNFIISGDRRVVDDWANDGWGL |
| | domain 2 | |
| 352 | Mature mouse | HDGHDDDAIDIEDDLDDVIEEVEDSKSKSDASTPPSPKVTYKAPVPTGEVYFADSFDRGSLSGWILS |
| | CNX | KAKKDDTDDEIAKYDGKWEVDEMKETKLPGDKGLVLMSRAKHHAISAKLNKPFLFDTKPLIVQYEVN |
| | | FQNGIECGGAYVKLLSKTAELSLDQFHDKTPYTIMFGPDKCGEDYKLHFIFRHKNPKTGVYEEKHAK |
| | | RPDADLKTYFTDKKTHLYTLILNPDNSFEILVDQSVVNSGNLLNDMTPPVNPSREIEDPEDRKPEDW |
| | | DERPKIADPDAVKPDDWDEDAPSKIPDEEATKPEGWLDDEPEYIPDPDAEKPEDWDEDMDGEWEA |
| | | PQIANPKCESAPGCGVWQRPMIDNPNYKGKWKPPMIDNPNYQGIWKPRKIPNPDFFEDLEPFKMT |
| | | PFSAIGLELWSMTSDIFFDNFIISGDRRVVDDWANDGWGLKKAADGAAEPGVVLQMLEAAEERPWL |
| | | WVVYILTVALPVFLVILFCCSGKKQSNAMEYKKTDAPQPDVKDEEGKEEEKNKRDEEEEEEKLEEK |
| | | QKSDAEEDGVTGSQDEEDSKPKAEEDEILNRSPRNRKPRRE |
| 353 | Human CRT | MLLSVPLLLGLLGLAVAEPAVYFKEQFLDGDGWTSRWIESKHKSDFGKFVLSSGKFYGDEEKDKGL |
| | (UniProt: P27797- | QTSQDARFYALSASFEPFSNKGQTLVVQFTVKHEQNIDCGGGYVKLFPNSLDQTDMHGDSEYNIM |
| | 1, v1) | FGPDICGPGTKKVHVIFNYKGKNVLINKDIRCKDDEFTHLYTLIVRPDNTYEVKIDNSQVESGSLEDD |
| | | WDFLPPKKIKDPDASKPEDWDERAKIDDPTDSKPEDWDKPEHIPDPDAKKPEDWDEEMDGEWEP |
| | | PVIQNPEYKGEWKPRQIDNPDYKGTWIHPEIDNPEYSPDPSIYAYDNFGVLGLDLWQVKSGTIFDNF |
| | | LITNDEAYAEEFGNETWGVTKAAEKQMKDKQDEEQRLKEEEEDKKRKEEEEAEDKEDDEDKDEDE |
| | | EDEEDKEEDEEEDVPGQAKDEL |
| 354 | Human CRT | MLLSVPLLLGLLGLAVA |
| | signal peptide | |
| 355 | Human CRT N- | EPAVYFKEQFLDGDGWTSRWIESKHKSDFGKFVLSSGKFYGDEEKDKGLQTSQDARFYALSASFE |
| | domain | PFSNKGQTLVVQFTVKHEQNIDCGGGYVKLFPNSLDQTDMHGDSEYNIMFGPDICGPGTKKVHVIF |
| | | NYKGKNVLINKDIRCKDDEFTHLYTLIVRPDNTYEVKIDNSQVESGSLEDDWDFLPPKKIKDPDASKP |
| | | EDWDERAKIDDPTDSKPEDWDKPEHIPDPDAKKPEDWDEEMDGEWEPPVIQNPEYKGEWKPRQI |
| | | DNPDYKGTWIHPEIDNPEYSPDPSIYAYDNFGVLGLDLWQVKSGTIFDNFLITNDEAYAEEFGNETW |
| | | GVTKAAEKQ |
| 356 | Human CRT C- | MKDKQDEEQRLKEEEEDKKRKEEEEAEDKEDDEDKDEDEEDEEDKEEDEEEDVPGQAKDEL |
| | domain | |
| 357 | Human CRT lectin | EPAVYFKEQFLDGDGWTSRWIESKHKSDFGKFVLSSGKFYGDEEKDKGLQTSQDARFYALSASFE |
| | domain 1 | PFSNKGQTLVVQFTVKHEQNIDCGGGYVKLFPNSLDQTDMHGDSEYNIMFGPDICGPGTKKVHVIF |
| | | NYKGKNVLINKDIRCKDDEFTHLYTLIVRPDNTYEVKIDNSQVESGSLEDDWD |
| 358 | Human CRT lectin | DPSIYAYDNFGVLGLDLWQVKSGTIFDNFLITNDEAYAEEFGNETWGVTKAAEKQ |
| | domain 2 | |
| 359 | Human CRT P | FLPPKKIKDPDASKPEDWDERAKIDDPTDSKPEDWDKPEHIPDPDAKKPEDWDEEMDGEWEPPVI |
| | domain | QNPEYKGEWKPRQIDNPDYKGTWIHPEIDNPEYSP |
| 360 | Mature human | EPAVYFKEQFLDGDGWTSRWIESKHKSDFGKFVLSSGKFYGDEEKDKGLQTSQDARFYALSASFE |
| | CRT | PFSNKGQTLVVQFTVKHEQNIDCGGGYVKLFPNSLDQTDMHGDSEYNIMFGPDICGPGTKKVHVIF |
| | | NYKGKNVLINKDIRCKDDEFTHLYTLIVRPDNTYEVKIDNSQVESGSLEDDWDFLPPKKIKDPDASKP |
| | | EDWDERAKIDDPTDSKPEDWDKPEHIPDPDAKKPEDWDEEMDGEWEPPVIQNPEYKGEWKPRQI |
| | | DNPDYKGTWIHPEIDNPEYSPDPSIYAYDNFGVLGLDLWQVKSGTIFDNFLITNDEAYAEEFGNETW |
| | | GVTKAAEKQMKDKQDEEQRLKEEEEDKKRKEEEEAEDKEDDEDKDEDEEDEEDKEEDEEEDVPG |
| | | QAKDEL |
| 361 | C008 epitope | YEVNFQNGIECGGAY |
| | region 1 | |
| 362 | C008 epitope | WEAPQIANPRC |
| | region 2 | |
| 363 | C008 composite | YEVNFQNGIECGGAYVKLLSKTPELNLDQFHDKTPYTIMFGPDKCGEDYKLHFIFRHKNPKTGIYEE |
| | epitope region | KHAKRPDADLKTYFTDKKTHLYTLILNPDNSFEILVDQSVVNSGNLLNDMTPPVNPSREIEDPEDRKP |
| | | EDWDERPKIPDPEAVKPDDWDEDAPAKIPDEEATKPEGWLDDEPEYVPDPDAEKPEDWDEDMDG |
| | | EWEAPQIANPRC |
| 364 | 1E1 epitope | VYFADSFDRGTLSG |
| | region 1 | |
| 365 | 1E1 epitope | ADSFDRGTLSGWILSKAKKDDTDDE |
| | region 2 | |
| 366 | 1E1 epitope | VDQSVVNSGNLLNDMTPPVNPSRE |
| | region 3 | |
| 367 | 1E1 epitope | DERPKIPDPEAVKPDDWDEDAPAKIPDEE |
| | region 4 | |
| 368 | 1E1 epitope | WEAPQIANPRCESAPGCGV |
| | region 5 | |
| 369 | 1E1 epitope | WQRPVIDNPNYKGKWKPPMIDNPSYQGIWKPRKIPNPDF |
| | region 6 | |
| 370 | 1E1 epitope | WANDGWGLKKAADGAAEPGVVGQMIE |
| | region 7 | |
| 371 | 1E1 composite | VYFADSFDRGTLSGWILSKAKKDDTDDEIAKYDGKWEVEEMKESKLPGDKGLVLMSRAKHHAISAK |
| | epitope region | LNKPFLFDTKPLIVQYEVNFQNGIECGGAYVKLLSKTPELNLDQFHDKTPYTIMFGPDKCGEDYKLH |
| | | FIFRHKNPKTGIYEEKHAKRPDADLKTYFTDKKTHLYTLILNPDNSFEILVDQSVVNSGNLLNDMTPP |
| | | VNPSREIEDPEDRKPEDWDERPKIPDPEAVKPDDWDEDAPAKIPDEEATKPEGWLDDEPEYVPDP |
| | | DAEKPEDWDEDMDGEWEAPQIANPRCESAPGCGVWQRPVIDNPNYKGKWKPPMIDNPSYQGIW |
| | | KPRKIPNPDFFEDLEPFRMTPFSAIGLELWSMTSDIFFDNFIICADRRIVDDWANDGWGLKKAADGA |
| | | AEPGVVGQMIE |
| 372 | 1E1 epitope | VYFADSFDRGTLSGWILSKAKKDDTDDE |
| | regions 1 and 2 | |
| 373 | 1E1 epitope | WEAPQIANPRCESAPGCGVWQRPVIDNPNYKGKWKPPMIDNPSYQGIWKPRKIPNPDF |
| | regions 5 and 6 | |
| 374 | ER-2Lec | AATGTAGTCTTATGCAATACTCTTGTAGTCTTGCAACATGGTAACGATGAGTTAGCAACATGCCT |
| | sequence | TACAAGGAGAGAAAAAGCACCGTGCATGCCGATTGGTGGAAGTAAGGTGGTACGATCGTGCCT |
| | | TATTAGGAAGGCAACAGACGGGTCTGACATGGATTGGACGAACCACTGAATTGCCGCATTGCA |
| | | GAGATATTGTATTTAAGTGCCTAGCTCGATACATAAACGGGTCTCTCTGGTTAGACCAGATCTG |
| | | AGCCTGGGAGCTCTCTGGCTAACTAGGGAACCCACTGCTTAAGCCTCAATAAAGCTTGCCTTG |
| | | AGTGCTTCAAGTAGTGTGTGCCCGTCTGTTGTGTGACTCTGGTAACTAGAGATCCCTCAGACCC |
| | | TTTTAGTCAGTGTGGAAAATCTCTAGCAGTGGCGCCCGAACAGGGACTTGAAAGCGAAAGGGA |
| | | AACCAGAGGAGCTCTCTCGACGCAGGACTCGGCTTGCTGAAGCGCGCACGGCAAGAGGCGAG |
| | | GGGCGGCGACTGGTGAGTACGCCAAAAATTTTGACTAGCGGAGGCTAGAAGGAGAGAGATGG |
| | | GTGCGAGAGCGTCAGTATTAAGCGGGGGAGAATTAGATCGCGATGGGAAAAAATTCGGTTAAG |
| | | GCCAGGGGGAAAGAAAAAATATAAATTAAAACATATAGTATGGGCAAGCAGGGAGCTAGAACG |
| | | ATTCGCAGTTAATCCTGGCCTGTTAGAAACATCAGAAGGCTGTAGACAAATACTGGGACAGCTA |
| | | CAACCATCCCTTCAGACAGGATCAGAAGAACTTAGATCATTATATAATACAGTAGCAACCCTCTA |
| | | TTGTGTGCATCAAAGGATAGAGATAAAAGACACCAAGGAAGCTTTAGACAAGATAGAGGAAGAG |
| | | CAAAACAAAAGTAAGACCACCGCACAGCAAGCGGCCGCTGATCTTCAGACCTGGAGGAGGAG |
| | | ATATGAGGGACAATTGGAGAAGTGAATTATATAAATATAAAGTAGTAAAAATTGAACCATTAGGA |
| | | GTAGCACCCACCAAGGCAAAGAGAAGAGTGGTGCAGAGAGAAAAAAGAGCAGTGGGAATAGG |
| | | AGCTTTGTTCCTTGGGTTCTTGGGAGCAGCAGGAAGCACTATGGGCGCAGCGTCAATGACGCT |
| | | GACGGTACAGGCCAGACAATTATTGTCTGGTATAGTGCAGCAGCAGAACAATTTGCTGAGGGC |
| | | TATTGAGGCGCAACAGCATCTGTTGCAACTCACAGTCTGGGGCATCAAGCAGCTCCAGGCAAG |
| | | AATCCTGGCTGTGGAAAGATACCTAAAGGATCAACAGCTCCTGGGGATTTGGGGTTGCTCTGG |
| | | AAAACTCATTTGCACCACTGCTGTGCCTTGGAATGCTAGTTGGAGTAATAAATCTCTGGAACAG |
| | | ATTTGGAATCACACGACCTGGATGGAGTGGGACAGAGAAATTAACAATTACACAAGCTTAATAC |
| | | ACTCCTTAATTGAAGAATCGCAAAACCAGCAAGAAAAGAATGAACAAGAATTATTGGAATTAGAT |
| | | AAATGGGCAAGTTTGTGGAATTGGTTTAACATAACAAATTGGCTGTGGTATATAAAATTATTCAT |
| | | AATGATAGTAGGAGGCTTGGTAGGTTTAAGAATAGTTTTTGCTGTACTTTCTATAGTGAATAGAG |
| | | TTAGGCAGGGATATTCACCATTATCGTTTCAGACCCACCTCCCAACCCCGAGGGGACCCGACA |
| | | GGCCCGAAGGAATAGAAGAAGAAGGTGGAGAGAGAGACAGAGACAGATCCATTCGATTAGTGA |
| | | ACGGATCTCGACGGTATCGGTTAACTTTTAAAAGAAAAGGGGGGATTGGGGGGTACAGTGCAG |
| | | GGGAAAGAATAGTAGACATAATAGCAACAGACATACAAACTAAAGAATTACAAAAACAAATTACA |
| | | AAAATTCAAAATTTTATCGATAAGCTTGGGAGTTCCGCGTTACATAACTTACGGTAAATGGCCCG |
| | | CCTGGCTGACCGCCCAACGACCCCCGCCCATTGACGTCAATAATGACGTATGTTCCCATAGTA |
| | | ACGCCAATAGGGACTTTCCATTGACGTCAATGGGTGGAGTATTTACGGTAAACTGCCCACTTGG |
| | | CAGTACATCAAGTGTATCATATGCCAAGTACGCCCCCTATTGACGTCAATGACGGTAAATGGCC |
| | | CGCCTGGCATTATGCCCAGTACATGACCTTATGGGACTTTCCTACTTGGCAGTACATCTACGTA |
| | | TTAGTCATCGCTATTACCATGGTGATGCGGTTTTGGCAGTACATCAATGGGCGTGGATAGCGGT |
| | | TTGACTCACGGGGATTTCCAAGTCTCCACCCCATTGACGTCAATGGGAGTTTGTTTTGGCACCA |
| | | AAATCAACGGGACTTTCCAAAATGTCGTAACAACTCCGCCCCATTGACGCAAATGGGGGTAG |
| | | GCGTGTACGGTGGGAGGTCTATATAAGCAGAGCTCGTTTAGTGAACCGTCAGATCGCCTGGAG |
| | | ACGCCATCCACGCTGTTTTGACCTCCATAGAAGACACCGGGACCGATCCAGCCTCCGGACTCT |
| | | AGAGGATCCCTACCGGTGATATCCTCGAGACCatggctacaggctcccggacgtccctgctcctggctt |
| | | ttggcctgctctgcctgccctggcttcaagagggcagtgccttcccaaccattcccttatccGTGAGCA |
| | | AGGGCGAGGAGCTGTTCACCGGGGTGGTGCCCATCCTGGTCGAGCTGGACGGCGACGTAAACGGCCACA |
| | | AGTTCAGCGTGTCCGGCGAGGGCGAGGGCGATGCCACCTACGGCAAGCTGACCCTGAAGTTCATCTGCA |
| | | CCACCGGCAAGCTGCCCGTGCCCTGGCCCACCCTCGTGACCACCTTCACCTACGGCGTGCAGTGCTTCG |
| | | CCCGCTACCCCGACCACATGAAGCAGCACGACTTCTTCAAGTCCGCCATGCCCGAAGGCTACG |
| | | TCCAGGAGCGCACCATCTTCTTCAAGGACGACGGCAACTACAAGACCCGCGCCGAGGTGAAG |
| | | TTCGAGGGCGACACCCTGGTGAACCGCATCGAGCTGAAGGGCATCGACTTCAAGGAGGACGG |
| | | CAACATCCTGGGGCACAAGCTGGAGTACAACTACAACAGCCACAAGGTCTATATCACCGCCGA |
| | | CAAGCAGAAGAACGGCATCAAGGTGAACTTCAAGACCCGCCACAACATCGAGGACGGCAGCG |
| | | TGCAGCTCGCCGACCACTACCAGCAGAACACCCCCATCGGCGACGGCCCCGTGCTGCTGCCC |
| | | GACAACCACTACCTGAGCACCCAGTCCGCCCTGAGCAAAGACCCCAACGAGAAGCGCGATCA |
| | | CATGGTCCTGCTGGAGTTCGTGACCGCCGCCGGGATCACTCTCGGCATGGACGAGCTGTACA |
| | | AGGGCTCGAGCCCATCAACAAGTTTGTACAAAAAAGCAGGCTccGATAACGGGAAGCCTATCCC |
| | | TAACCCTCTCCTCGGTCTCGATTCTACGgagttaagggttccagaccatcaggatatagcttttggggc |
| | | cttgcagcagggaactaactgcctcgacactttgggacactttgctgatggtgtggttggagtttatga |
| | | atgtcacaatgctgggggaaaccaggaatgggccttgacgaaggagaagtcggtgaagcacatggattt |
| | | gtgccttactgtggtggaccgggcaccgggctctcttataaagctgcagggctgccgagaaaatgacag |
| | | cagacagaaatgggaacagatcgagggcaactccaagctgaggcacgtgggcagcaacctgtgcctgga |
| | | cagtcgcacggccaagagcgggggcctaagcgtggaggtgtgtggcccggccctttcgcagcagtggaa |
| | | gttcacgctcaacctgcagcagGCACAGGGAGCCCAAGGGAGATCCGATAACGGGAAGCCTATCCCTAA |
| | | CCCTCTCCTCGGTCTCGATTCTACGgagttaagggttccagaccatcaggatatagcttttggggcctt |
| | | gcagcagggaactaactgcctcgacactttgggacactttgctgatggtgtggttggagtttatgaatg |
| | | tcacaatgctgggggaaaccaggaatgggccttgacgaaggagaagtcggtgaagcacatggatttgtg |
| | | ccttactgtggtggaccgggcaccgggctctcttataaagctgcagggctgccgagaaaatgacagcag |
| | | acagaaatgggaacagatcgagggcaactccaagctgaggcacgtgggcagcaacctgtgcctggacag |
| | | tcgcacggccaagagcgggggcctaagcgtggaggtgtgtggcccggccctttcgcagcagtggaagtt |
| | | cacgctcaacctgcagcagAAAGACGAGCTGtagGACCCAGCTTTCTTGTACAAAGTGGTTGATATCCA |
| | | GCACAGTGGCGGCCGCTCGAGTCTAGAGGGCCCGCGGTTCGAAGGTAAGCCTATCCCTAACCCTCTCCT |
| | | CGGTCTCGATTCTACGCGTACCGGTTAGTAATGATCGACAATCAACCTCTGGATTACAAAATTTGTGAA |
| | | AGATTGACTGGTATTCTTAACTATGTTGCTCCTTTTACGCTATGTGGATACGCTGCTTTAATGCCTTTG |
| | | TATCATGCTATTGCTTCCCGTATGGCTTTCATTTTCTCCTCCTTGTATAAATCCTGGTTGCTGTCTCTT |
| | | TATGAGGAGTTGTGGCCCGTTGTCAGGCAACGTGGCGTGGTGTGCACTGTGTTTGCTGACGCAACCCCC |
| | | ACTGGTTGGGGCATTGCCACCACCTGTCAGCTCCTTTCCGGGACTTTCGCTTTCCCCCTCCCTATTGCC |
| | | ACGGCGGAACTCATCGCCGCCTGCCTTGCCCGCTGCTGGACAGGGGCTCGGCTGTTGGGCACTGACAAT |
| | | TCCGTGGTGTTGTCGGGGAAGCTGACGTCCTTTCCATGGCTGCTCGCCTGTGTTGCCACCTGGATT |
| | | CTGCGCGGGACGTCCTTCTGCTACGTCCCTTCGGCCCTCAATCCAGCGGACCTTCCTTCCCGC |
| | | GGCCTGCTGCCGGCTCTGCGGCCTCTTCCGCGTCTTCGCCTTCGCCCTCAGACGAGTCGGAT |
| | | CTCCCTTTGGGCCGCCTCCCCGCCTGGCGATGGTACCGGTGTGGAAAGTCCCCAGGCTCCCC |
| | | AGCAGGCAGAAGTATGCAAAGCATGCATCTCAATTAGTCAGCAACCAGGTGTGGAAAGTCCCC |
| | | AGGCTCCCCAGCAGGCAGAAGTATGCAAAGCATGCATCTCAATTAGTCAGCAACCATAGTCCC |
| | | GCCCCTAACTCCGCCCATCCCGCCCCTAACTCCGCCCAGTTCCGCCCATTCTCCGCCCCATGG |
| | | CTGACTAATTTTTTTTATTTATGCAGAGGCCGAGGCCGCCTCTGCCTCTGAGCTATTCCAGAAG |
| | | TAGTGAGGAGGCTTTTTTGGAGGCCTAGGCTTTTGCAAAAAGCTCCCGGGAGCTTGTATATCCA |
| | | TTTTCGGATCTGATCAGCACGTGTTGACAATTAATCATCGGCATAGTATATCGGCATAGTATAAT |
| | | ACGACAAGGTGAGGAACTAAACCATGGCCAAGCCTTTGTCTCAAGAAGAATCCACCCTCATTGA |
| | | AAGAGCAACGGCTACAATCAACAGCATCCCCATCTCTGAAGACTACAGCGTCGCCAGCGCAGC |
| | | TCTCTCTAGCGACGGCCGCATCTTCACTGGTGTCAATGTATATCATTTTACTGGGGGACCTTGT |
| | | GCAGAACTCGTGGTGCTGGGCACTGCTGCTGCTGCGGCAGCTGGCAACCTGACTTGTATCGT |
| | | CGCGATCGGAAATGAGAACAGGGGCATCTTGAGCCCCTGCGGACGGTGCCGACAGGTGCTTC |
| | | TCGATCTGCATCCTGGGATCAAAGCCATAGTGAAGGACAGTGATGGACAGCCGACGGCAGTTG |
| | | GGATTCGTGAATTGCTGCCCTCTGGTTATGTGTGGGAGGGCTAAGCACAATTCGAGCTCGGTA |
| | | CCTTTAAGACCAATGACTTACAAGGCAGCTGTAGATCTTAGCCACTTTTTAAAAGAAAAGGGGG |
| | | GACTGGAAGGGCTAATTCACTCCCAACGAAGACAAGATCTGCTTTTTGCTTGTACTGGGTCTCT |
| | | CTGGTTAGACCAGATCTGAGCCTGGGAGCTCTCTGGCTAACTAGGGAACCCACTGCTTAAGCC |
| | | TCAATAAAGCTTGCCTTGAGTGCTTCAAGTAGTGTGTGCCCGTCTGTTGTGTGACTCTGGTAAC |
| | | TAGAGATCCCTCAGACCCTTTTAGTCAGTGTGGAAAATCTCTAGCAGTAGTAGTTCATGTCATCT |
| | | TATTATTCAGTATTTATAACTTGCAAAGAAATGAATATCAGAGAGTGAGAGGAACTTGTTTATTG |
| | | CAGCTTATAATGGTTACAAATAAAGCAATAGCATCACAAATTTCACAAATAAAGCATTTTTTTCAC |
| | | TGCATTCTAGTTGTGGTTTGTCCAAACTCATCAATGTATCTTATCATGTCTGGCTCTAGCTATCC |
| | | CGCCCCTAACTCCGCCCATCCCGCCCCTAACTCCGCCCAGTTCCGCCCATTCTCCGCCCCATG |
| | | GCTGACTAATTTTTTTTATTTATGCAGAGGCCGAGGCCGCCTCGGCCTCTGAGCTATTCCAGAA |
| | | GTAGTGAGGAGGCTTTTTTGGAGGCCTAGGGACGTACCCAATTCGCCCTATAGTGAGTCGTAT |
| | | TACGCGCGCTCACTGGCCGTCGTTTTACAACGTCGTGACTGGGAAAACCCTGGCGTTACCCAA |
| | | CTTAATCGCCTTGCAGCACATCCCCCTTTCGCCAGCTGGCGTAATAGCGAAGAGGCCCGCACC |
| | | GATCGCCCTTCCCAACAGTTGCGCAGCCTGAATGGCGAATGGGACGCGCCCTGTAGCGGCGC |
| | | ATTAAGCGCGGCGGGTGTGGTGGTTACGCGCAGCGTGACCGCTACACTTGCCAGCGCCCTAG |
| | | CGCCCGCTCCTTTCGCTTTCTTCCCTTCCTTTCTCGCCACGTTCGCCGGCTTTCCCCGTCAAGC |
| | | TCTAAATCGGGGGCTCCCTTTAGGGTTCCGATTTAGTGCTTTACGGCACCTCGACCCCAAAAAA |
| | | CTTGATTAGGGTGATGGTTCACGTAGTGGGCCATCGCCCTGATAGACGGTTTTTCGCCCTTTGA |
| | | CGTTGGAGTCCACGTTCTTTAATAGTGGACTCTTGTTCCAAACTGGAACAACACTCAACCCTAT |
| | | CTCGGTCTATTCTTTTGATTTATAAGGGATTTTGCCGATTTCGGCCTATTGGTTAAAAAATGAGC |
| | | TGATTTAACAAAAATTTAACGCGAATTTTAACAAAATATTAACGCTTACAATTTAGGTGGCACTTT |
| | | TCGGGGAAATGTGCGCGGAACCCCTATTTGTTTATTTTTCTAAATACATTCAAATATGTATCCGC |
| | | TCATGAGACAATAACCCTGATAAATGCTTCAATAATATTGAAAAAGGAAGAGTATGAGTATTCAA |
| | | CATTTCCGTGTCGCCCTTATTCCCTTTTTTGCGGCATTTTGCCTTCCTGTTTTTGCTCACCCAGA |
| | | AACGCTGGTGAAAGTAAAAGATGCTGAAGATCAGTTGGGTGCACGAGTGGGTTACATCGAACT |
| | | GGATCTCAACAGCGGTAAGATCCTTGAGAGTTTTCGCCCCGAAGAACGTTTTCCAATGATGAGC |
| | | ACTTTTAAAGTTCTGCTATGTGGCGCGGTATTATCCCGTATTGACGCCGGGCAAGAGCAACTCG |
| | | GTCGCCGCATACACTATTCTCAGAATGACTTGGTTGAGTACTCACCAGTCACAGAAAAGCATCT |
| | | TACGGATGGCATGACAGTAAGAGAATTATGCAGTGCTGCCATAACCATGAGTGATAACACTGCG |
| | | GCCAACTTACTTCTGACAACGATCGGAGGACCGAAGGAGCTAACCGCTTTTTTGCACAACATG |
| | | GGGGATCATGTAACTCGCCTTGATCGTTGGGAACCGGAGCTGAATGAAGCCATACCAAACGAC |
| | | GAGCGTGACACCACGATGCCTGTAGCAATGGCAACAACGTTGCGCAAACTATTAACTGGCGAA |
| | | CTACTTACTCTAGCTTCCCGGCAACAATTAATAGACTGGATGGAGGCGGATAAAGTTGCAGGAC |
| | | CACTTCTGCGCTCGGCCCTTCCGGCTGGCTGGTTTATTGCTGATAAATCTGGAGCCGGTGAGC |
| | | GTGGGTCTCGCGGTATCATTGCAGCACTGGGGCCAGATGGTAAGCCCTCCCGTATCGTAGTTA |
| | | TCTACACGACGGGGAGTCAGGCAACTATGGATGAACGAAATAGACAGATCGCTGAGATAGGTG |
| | | CCTCACTGATTAAGCATTGGTAACTGTCAGACCAAGTTTACTCATATATACTTTAGATTGATTTAA |
| | | AACTTCATTTTTAATTTAAAAGGATCTAGGTGAAGATCCTTTTTGATAATCTCATGACCAAAATCC |
| | | CTTAACGTGAGTTTTCGTTCCACTGAGCGTCAGACCCCGTAGAAAAGATCAAAGGATCTTCTTG |
| | | AGATCCTTTTTTTCTGCGCGTAATCTGCTGCTTGCAAACAAAAAAACCACCGCTACCAGCGGTG |
| | | GTTTGTTTGCCGGATCAAGAGCTACCAACTCTTTTTCCGAAGGTAACTGGCTTCAGCAGAGCGC |
| | | AGATACCAAATACTGTTCTTCTAGTGTAGCCGTAGTTAGGCCACCACTTCAAGAACTCTGTAGC |
| | | ACCGCCTACATACCTCGCTCTGCTAATCCTGTTACCAGTGGCTGCTGCCAGTGGCGATAAGTC |
| | | GTGTCTTACCGGGTTGGACTCAAGACGATAGTTACCGGATAAGGCGCAGCGGTCGGGCTGAA |
| | | CGGGGGGTTCGTGCACACAGCCCAGCTTGGAGCGAACGACCTACACCGAACTGAGATACCTA |
| | | CAGCGTGAGCTATGAGAAAGCGCCACGCTTCCCGAAGGGAGAAAGGCGGACAGGTATCCGGT |
| | | AAGCGGCAGGGTCGGAACAGGAGAGCGCACGAGGGAGCTTCCAGGGGGAAACGCCTGGTAT |
| | | CTTTATAGTCCTGTCGGGTTTCGCCACCTCTGACTTGAGCGTCGATTTTTGTGATGCTCGTCAG |
| | | GGGGGCGGAGCCTATGGAAAAACGCCAGCAACGCGGCCTTTTTACGGTTCCTGGCCTTTTGCT |
| | | GGCCTTTTGCTCACATGTTCTTTCCTGCGTTATCCCCTGATTCTGTGGATAACCGTATTACCGCC |
| | | TTTGAGTGAGCTGATACCGCTCGCCGCAGCCGAACGACCGAGCGCAGCGAGTCAGTGAGCGA |
| | | GGAAGCGGAAGAGCGCCCAATACGCAAACCGCCTCTCCCCGCGCGTTGGCCGATTCATTAAT |
| | | GCAGCTGGCACGACAGGTTTCCCGACTGGAAAGCGGGCAGTGAGCGCAACGCAATTAATGTG |
| | | AGTTAGCTCACTCATTAGGCACCCCAGGCTTTACACTTTATGCTTCCGGCTCGTATGTTGTGTG |
| | | GAATTGTGAGCGGATAACAATTTCACACAGGAAACAGCTATGACCATGATTACGCCAAGCGCG |
| | | CAATTAACCCTCACTAAAGGGAACAAAAGCTGGAGCTGCAAGCTT |
| 375 | siRNA target | CAAGAGUGGUCCUAGGAGAUU |
| | sequence for CNX | |
| | (CNX) | |
| 376 | siRNA target | GGAAUAGUCCCAUUAGCAA |
| | sequence for | |
| | PDIA3 | |
| 377 | siRNA target | GGGCAAGGACUUACUUAUU |
| | sequence for | |
| | PDIA3 | |
| 378 | siRNA target | AGACCCAAAUAUCGUCAUA |
| | sequence for | |
| | PDIA3 | |
| 379 | siRNA target | AGGAGUUCUCGCGUGAUG |
| | sequence for | |
| | PDIA3 | |
| 380 | siRNA target | GAACGAGUAUGAUGAUAAU |
| | sequence for | |
| | PDIA3 | |
| 381 | siRNA target | GGACAAGACUGUGGCAUAU |
| | sequence for | |
| | PDIA3 | |
| 382 | siRNA target | GGCAAGGACUUACUUAUUG |
| | sequence for | |
| | PDIA3 | |
| 383 | siRNA target | UGAUAAAGAUGCCUCUAUA |
| | sequence for | |
| | PDIA3 | |
| 384 | HDX detected | VQSGGGVVQPGRS |
| | peptide sequence | |
| | 1 | |
| 385 | HDX detected | YADSVKGRF |
| | peptide sequence | |
| | 2 | |
| 386 | HDX detected | SRAVAGAFDYWGQGTLVT |
| | peptide sequence | |
| | 3 | |
| 387 | HDX detected | VMYAKNHRPSGIPDRFSGSSSGNTASL |
| | peptide sequence | |
| | 4 | |
| 388 | HDX detected | YYVGSRDSSGNHPVFGGGTKVTLV |
| | peptide sequence | |
| | 5 | |
| 389 | HDX detected | ADSFDRGTLSG |
| | peptide sequence | |
| | 6 | |
| 390 | HDX detected | YVKLLSKTPELNL |
| | peptide sequence | |
| | 7 | |
| 391 | HDX detected | DEDAPAKIPDEEATKPEG |
| | peptide sequence | |
| | 8 | |
| 392 | HDX detected | WVRQAPGKGLEWVSGINWNGGSTGYAD |
| | peptide sequence | |
| | 9 | |
| 393 | HDX detected | WVSIGNWNGGSTGYADSVKGRF |
| | peptide sequence | |
| | 10 | |
| 394 | HDX detected | VTVSSASTKGPSVFPLAPSSKSTSGGTAAL |
| | peptide sequence | |
| | 11 | |
| 395 | HDX detected | VKDYFPEPVTVSWNSGALTSGVHTFPAVL |
| | peptide sequence | |
| | 12 | |
| 396 | HDX detected | VVDVSHEDPEVKFNW |
| | peptide sequence | |
| | 13 | |
| 397 | HDX detected | TVLHQDWLNGKEYKCKVSNKALPAPIE |
| | peptide sequence | |
| | 14 | |
| 398 | HDX detected | VLHQDWLNGKEYKCKVS |
| | peptide sequence | |
| | 15 | |
| 399 | HDX detected | KTISKAKGQPREPQVYTLPPSRDELTKNQVSL |
| | peptide sequence | |
| | 16 | |
| 400 | HDX detected | YSKLTVDKSRWQQGNV |
| | peptide sequence | |
| | 17 | |
| 401 | HDX detected | TLKISRVEAEDVG |
| | peptide sequence | |
| | 18 | |
| 402 | HDX detected | VFIFPPSDEQLKSGTAS |
| | peptide sequence | |
| | 19 | |
| 403 | HDX detected | VCLLNNFYPREAKVQWKVDNALQSGNSQE |
| | peptide sequence | |
| | 20 | |
| 404 | HDX detected | EVTHQGLSSPVTKSFNRGEC |
| | peptide sequence | |
| | 21 | |
| 405 | HDX detected | WEAPQIANPRCESAPCGV |
| | peptide sequence | |
| | 22 | |
| 406 | HDX detected | WANDGWGLKKAADDGAAEPGVVGQMIE |
| | peptide sequence | |
| | 23 | |
| 407 | HDX detected | QNGIECGGAYYVKLLSKTPELNLDQFHDKTPYT |
| | peptide sequence | |
| | 24 | |
| 408 | HDX detected | TPTTVGPTTVGSTTVG |
| | peptide sequence | |
| | 25 | |
| 409 | Linker sequence 1 | GGGGSGGGGSGGGGS |
| 410 | Linker sequence 2 | GGGS |
| 411 | Linker sequence 3 | GGGGS |
| 412 | Linker sequence 4 | GGGSG |
| 413 | Linker sequence 5 | GGGGSG |
| 414 | Linker sequence 6 | GGGGS |
| 415 | Linker sequence 7 | GGGGSGGGGSGGGGSGGGGS |
| 416 | Linker sequence 8 | GGGGSGGGGSGGGGSGGGGSGGGGSGGGGS |

TABLE A

| | Column A | | | Column B | | |
|---|---|---|---|---|---|---|
| | VH | | | VL | | |
| Clone | HC-CDR1 | HC-CDR2 | HC-CDR3 | LC-CDR1 | LC-CDR2 | LC-CDR3 |
| 1D3 | SEQ ID NO: 2 | SEQ ID NO: 3 | SEQ ID NO: 4 | SEQ ID NO: 10 | SEQ ID NO: 11 | SEQ ID NO: 12 |
| 1D6 | SEQ ID NO: 18 | SEQ ID NO: 19 | SEQ ID NO: 20 | SEQ ID NO: 25 | SEQ ID NO: 26 | SEQ ID NO: 27 |
| 1E1 | SEQ ID NO: 33 | SEQ ID NO: 34 | SEQ ID NO: 35 | SEQ ID NO: 41 | SEQ ID NO: 42 | SEQ ID NO: 43 |
| 1E6 | SEQ ID NO: 48 | SEQ ID NO: 3 | SEQ ID NO: 49 | SEQ ID NO: 53 | SEQ ID NO: 54 | SEQ ID NO: 55 |
| 2C6 | SEQ ID NO: 61 | SEQ ID NO: 62 | SEQ ID NO: 63 | SEQ ID NO: 68 | SEQ ID NO: 26 | SEQ ID NO: 69 |
| 2H6 | SEQ ID NO: 61 | SEQ ID NO: 62 | SEQ ID NO: 63 | SEQ ID NO: 73 | SEQ ID NO: 26 | SEQ ID NO: 74 |
| 3D1 | SEQ ID NO: 61 | SEQ ID NO: 62 | SEQ ID NO: 63 | SEQ ID NO: 78 | SEQ ID NO: 79 | SEQ ID NO: 80 |
| 2G9 | SEQ ID NO: 2 | SEQ ID NO: 3 | SEQ ID NO: 83 | SEQ ID NO: 73 | SEQ ID NO: 26 | SEQ ID NO: 74 |
| 2G12 | SEQ ID NO: 48 | SEQ ID NO: 3 | SEQ ID NO: 86 | SEQ ID NO: 89 | SEQ ID NO: 11 | SEQ ID NO: 90 |
| 2H5 | SEQ ID NO: 61 | SEQ ID NO: 95 | SEQ ID NO: 96 | SEQ ID NO: 101 | SEQ ID NO: 102 | SEQ ID NO: 103 |
| 3F8 | SEQ ID NO: 108 | SEQ ID NO: 109 | SEQ ID NO: 110 | SEQ ID NO: 115 | SEQ ID NO: 116 | SEQ ID NO: 117 |
| 3F9 | SEQ ID NO: 2 | SEQ ID NO: 3 | SEQ ID NO: 122 | SEQ ID NO: 125 | SEQ ID NO: 126 | SEQ ID NO: 127 |
| 4G9 | SEQ ID NO: 132 | SEQ ID NO: 133 | SEQ ID NO: 134 | SEQ ID NO: 139 | SEQ ID NO: 140 | SEQ ID NO: 80 |
| 5A3 | SEQ ID NO: 146 | SEQ ID NO: 147 | SEQ ID NO: 148 | SEQ ID NO: 41 | SEQ ID NO: 42 | SEQ ID NO: 153 |
| 5E8 | SEQ ID NO: 48 | SEQ ID NO: 3 | SEQ ID NO: 156 | SEQ ID NO: 158 | SEQ ID NO: 159 | SEQ ID NO: 160 |
| C001 | SEQ ID NO: 166 | SEQ ID NO: 167 | SEQ ID NO: 168 | SEQ ID NO: 171 | SEQ ID NO: 172 | SEQ ID NO: 173 |
| C008 | SEQ ID NO: 166 | SEQ ID NO: 167 | SEQ ID NO: 168 | SEQ ID NO: 179 | SEQ ID NO: 180 | SEQ ID NO: 173 |
| C010 | SEQ ID NO: 185 | SEQ ID NO: 186 | SEQ ID NO: 187 | SEQ ID NO: 73 | SEQ ID NO: 26 | SEQ ID NO: 194 |
| C023 | SEQ ID NO: 48 | SEQ ID NO: 199 | SEQ ID NO: 200 | SEQ ID NO: 205 | SEQ ID NO: 42 | SEQ ID NO: 206 |
| C025 | SEQ ID NO: 48 | SEQ ID NO: 211 | SEQ ID NO: 212 | SEQ ID NO: 216 | SEQ ID NO: 172 | SEQ ID NO: 217 |
| C040 | SEQ ID NO: 222 | SEQ ID NO: 223 | SEQ ID NO: 224 | SEQ ID NO: 229 | SEQ ID NO: 172 | SEQ ID NO: 230 |
| C046 | SEQ ID NO: 48 | SEQ ID NO: 199 | SEQ ID NO: 200 | SEQ ID NO: 235 | SEQ ID NO: 236 | SEQ ID NO: 237 |
| C117 | SEQ ID NO: 185 | SEQ ID NO: 243 | SEQ ID NO: 244 | SEQ ID NO: 248 | SEQ ID NO: 249 | SEQ ID NO: 250 |

TABLE B

| | Column A | | | |
|---|---|---|---|---|
| | VH | | | |
| Clone | HC-FR1 | HC-FR2 | HC-FR3 | HC-FR4 |
| 1D3 | SEQ ID NO: 5 | SEQ ID NO: 6 | SEQ ID NO: 7 | SEQ ID NO: 8 |
| 1D6 | SEQ ID NO: 21 | SEQ ID NO: 22 | SEQ ID NO: 23 | SEQ ID NO: 8 |
| 1E1 | SEQ ID NO: 36 | SEQ ID NO: 37 | SEQ ID NO: 38 | SEQ ID NO: 39 |
| 1E6 | SEQ ID NO: 50 | SEQ ID NO: 6 | SEQ ID NO: 7 | SEQ ID NO: 51 |
| 2C6 | SEQ ID NO: 64 | SEQ ID NO: 65 | SEQ ID NO: 66 | SEQ ID NO: 39 |
| 2H6 | SEQ ID NO: 64 | SEQ ID NO: 65 | SEQ ID NO: 66 | SEQ ID NO: 39 |
| 3D1 | SEQ ID NO: 64 | SEQ ID NO: 65 | SEQ ID NO: 66 | SEQ ID NO: 39 |
| 2G9 | SEQ ID NO: 84 | SEQ ID NO: 6 | SEQ ID NO: 7 | SEQ ID NO: 39 |
| 2G12 | SEQ ID NO: 87 | SEQ ID NO: 6 | SEQ ID NO: 7 | SEQ ID NO: 51 |
| 2H5 | SEQ ID NO: 97 | SEQ ID NO: 98 | SEQ ID NO: 66 | SEQ ID NO: 39 |
| 3F8 | SEQ ID NO: 111 | SEQ ID NO: 112 | SEQ ID NO: 113 | SEQ ID NO: 51 |
| 3F9 | SEQ ID NO: 123 | SEQ ID NO: 6 | SEQ ID NO: 7 | SEQ ID NO: 51 |
| 4G9 | SEQ ID NO: 135 | SEQ ID NO: 136 | SEQ ID NO: 137 | SEQ ID NO: 39 |
| 5A3 | SEQ ID NO: 149 | SEQ ID NO: 150 | SEQ ID NO: 151 | SEQ ID NO: 39 |
| 5E8 | SEQ ID NO: 87 | SEQ ID NO: 6 | SEQ ID NO: 7 | SEQ ID NO: 39 |
| C001 | SEQ ID NO: 87 | SEQ ID NO: 169 | SEQ ID NO: 7 | SEQ ID NO: 39 |
| C008 | SEQ ID NO: 87 | SEQ ID NO: 169 | SEQ ID NO: 7 | SEQ ID NO: 39 |
| C010 | SEQ ID NO: 188 | SEQ ID NO: 189 | SEQ ID NO: 190 | SEQ ID NO: 8 |
| C023 | SEQ ID NO: 201 | SEQ ID NO: 202 | SEQ ID NO: 203 | SEQ ID NO: 8 |
| C025 | SEQ ID NO: 213 | SEQ ID NO: 214 | SEQ ID NO: 7 | SEQ ID NO: 39 |
| C040 | SEQ ID NO: 225 | SEQ ID NO: 226 | SEQ ID NO: 227 | SEQ ID NO: 8 |
| C046 | SEQ ID NO: 201 | SEQ ID NO: 202 | SEQ ID NO: 203 | SEQ ID NO: 8 |
| C117 | SEQ ID NO: 245 | SEQ ID NO: 246 | SEQ ID NO: 190 | SEQ ID NO: 39 |

| | Column B | | | |
|---|---|---|---|---|
| | VL | | | |
| Clone | LC-FR1 | LC-FR2 | LC-FR3 | LC-FR4 |
| 1D3 | SEQ ID NO: 13 | SEQ ID NO: 14 | SEQ ID NO: 15 | SEQ ID NO: 16 |
| 1D6 | SEQ ID NO: 28 | SEQ ID NO: 29 | SEQ ID NO: 30 | SEQ ID NO: 31 |
| 1E1 | SEQ ID NO: 44 | SEQ ID NO: 45 | SEQ ID NO: 46 | SEQ ID NO: 16 |
| 1E6 | SEQ ID NO: 56 | SEQ ID NO: 57 | SEQ ID NO: 58 | SEQ ID NO: 59 |
| 2C6 | SEQ ID NO: 28 | SEQ ID NO: 70 | SEQ ID NO: 71 | SEQ ID NO: 31 |
| 2H6 | SEQ ID NO: 28 | SEQ ID NO: 75 | SEQ ID NO: 76 | SEQ ID NO: 31 |
| 3D1 | SEQ ID NO: 28 | SEQ ID NO: 81 | SEQ ID NO: 76 | SEQ ID NO: 31 |
| 2G9 | SEQ ID NO: 28 | SEQ ID NO: 75 | SEQ ID NO: 76 | SEQ ID NO: 31 |
| 2G12 | SEQ ID NO: 91 | SEQ ID NO: 92 | SEQ ID NO: 93 | SEQ ID NO: 16 |
| 2H5 | SEQ ID NO: 104 | SEQ ID NO: 105 | SEQ ID NO: 106 | SEQ ID NO: 31 |
| 3F8 | SEQ ID NO: 118 | SEQ ID NO: 119 | SEQ ID NO: 120 | SEQ ID NO: 31 |
| 3F9 | SEQ ID NO: 128 | SEQ ID NO: 129 | SEQ ID NO: 130 | SEQ ID NO: 16 |
| 4G9 | SEQ ID NO: 141 | SEQ ID NO: 142 | SEQ ID NO: 143 | SEQ ID NO: 144 |
| 5A3 | SEQ ID NO: 44 | SEQ ID NO: 45 | SEQ ID NO: 46 | SEQ ID NO: 154 |
| 5E8 | SEQ ID NO: 161 | SEQ ID NO: 162 | SEQ ID NO: 163 | SEQ ID NO: 164 |
| C001 | SEQ ID NO: 174 | SEQ ID NO: 175 | SEQ ID NO: 176 | SEQ ID NO: 177 |
| C008 | SEQ ID NO: 181 | SEQ ID NO: 182 | SEQ ID NO: 183 | SEQ ID NO: 177 |
| C010 | SEQ ID NO: 28 | SEQ ID NO: 75 | SEQ ID NO: 197 | SEQ ID NO: 177 |
| C023 | SEQ ID NO: 44 | SEQ ID NO: 207 | SEQ ID NO: 208 | SEQ ID NO: 209 |
| C025 | SEQ ID NO: 218 | SEQ ID NO: 219 | SEQ ID NO: 220 | SEQ ID NO: 31 |
| C040 | SEQ ID NO: 231 | SEQ ID NO: 232 | SEQ ID NO: 233 | SEQ ID NO: 31 |
| C046 | SEQ ID NO: 238 | SEQ ID NO: 239 | SEQ ID NO: 240 | SEQ ID NO: 241 |
| C117 | SEQ ID NO: 231 | SEQ ID NO: 251 | SEQ ID NO: 252 | SEQ ID NO: 253 |

TABLE C

| | Column A | Column B |
|---|---|---|
| Clone | VH | VL |
| 1D3 | SEQ ID NO: 1 | SEQ ID NO: 9 |
| 1D6 | SEQ ID NO: 17 | SEQ ID NO: 24 |
| 1E1 | SEQ ID NO: 32 | SEQ ID NO: 40 |
| 1E6 | SEQ ID NO: 47 | SEQ ID NO: 52 |
| 2C6 | SEQ ID NO: 60 | SEQ ID NO: 67 |
| 2H6 | SEQ ID NO: 60 | SEQ ID NO: 72 |
| 3D1 | SEQ ID NO: 60 | SEQ ID NO: 77 |
| 2G9 | SEQ ID NO: 82 | SEQ ID NO: 72 |
| 2G12 | SEQ ID NO: 85 | SEQ ID NO: 88 |
| 2H5 | SEQ ID NO: 94 | SEQ ID NO: 100 |
| 3F8 | SEQ ID NO: 107 | SEQ ID NO: 114 |
| 3F9 | SEQ ID NO: 121 | SEQ ID NO: 124 |
| 4G9 | SEQ ID NO: 131 | SEQ ID NO: 138 |
| 5A3 | SEQ ID NO: 145 | SEQ ID NO: 152 |
| 5E8 | SEQ ID NO: 155 | SEQ ID NO: 157 |
| C001 | SEQ ID NO: 165 | SEQ ID NO: 170 |
| C008 | SEQ ID NO: 165 | SEQ ID NO: 178 |
| C010 | SEQ ID NO: 184 | SEQ ID NO: 191 |
| C023 | SEQ ID NO: 198 | SEQ ID NO: 204 |
| C025 | SEQ ID NO: 210 | SEQ ID NO: 215 |
| C040 | SEQ ID NO: 221 | SEQ ID NO: 228 |
| C046 | SEQ ID NO: 198 | SEQ ID NO: 234 |
| C117 | SEQ ID NO: 242 | SEQ ID NO: 247 |

TABLE D

| Antigen | | | |
|---|---|---|---|
| Binding | | Column A | Column B |
| Molecule | Name | Heavy Chain | Light Chain |
| [1] | 1D3 IgG1 | SEQ ID NO: 273 | SEQ ID NO: 275 |
| [2] | 1D6 IgG1 | SEQ ID NO: 276 | SEQ ID NO: 278 |
| [3] | 1E1 IgG1 | SEQ ID NO: 279 | SEQ ID NO: 281 |
| [4] | 1E6 IgG1 | SEQ ID NO: 282 | SEQ ID NO: 284 |
| [5] | 2C6 IgG1 | SEQ ID NO: 285 | SEQ ID NO: 287 |
| [6] | 2H6 IgG1 | SEQ ID NO: 285 | SEQ ID NO: 288 |
| [7] | 3D1 IgG1 | SEQ ID NO: 285 | SEQ ID NO: 289 |
| [8] | 2G9 IgG1 | SEQ ID NO: 290 | SEQ ID NO: 288 |
| [9] | 2G12 IgG1 | SEQ ID NO: 292 | SEQ ID NO: 294 |
| [10] | 2H5 IgG1 | SEQ ID NO: 295 | SEQ ID NO: 297 |
| [11] | 3F8 IgG1 | SEQ ID NO: 298 | SEQ ID NO: 300 |
| [12] | 3F9 IgG1 | SEQ ID NO: 301 | SEQ ID NO: 303 |
| [13] | 4G9 IgG1 | SEQ ID NO: 304 | SEQ ID NO: 306 |
| [14] | 5A3 IgG1 | SEQ ID NO: 307 | SEQ ID NO: 309 |
| [15] | 5E8 IgG1 | SEQ ID NO: 310 | SEQ ID NO: 312 |
| [16] | C001 IgG1 | SEQ ID NO: 313 | SEQ ID NO: 315 |
| [17] | C008 IgG1 | SEQ ID NO: 313 | SEQ ID NO: 316 |
| [18] | C010 IgG1 | SEQ ID NO: 317 | SEQ ID NO: 319 |
| [19] | C023 IgG1 | SEQ ID NO: 320 | SEQ ID NO: 322 |
| [20] | C025 IgG1 | SEQ ID NO: 323 | SEQ ID NO: 325 |
| [21] | C040 IgG1 | SEQ ID NO: 326 | SEQ ID NO: 328 |
| [22] | C046 IgG1 | SEQ ID NO: 320 | SEQ ID NO: 329 |
| [23] | C117 IgG1 | SEQ ID NO: 330 | SEQ ID NO: 332 |
| [24] | 1D3 IgG4 | SEQ ID NO: 274 | SEQ ID NO: 275 |
| [25] | 1D6 IgG4 | SEQ ID NO: 277 | SEQ ID NO: 278 |
| [26] | 1E1 IgG4 | SEQ ID NO: 280 | SEQ ID NO: 281 |
| [27] | 1E6 IgG4 | SEQ ID NO: 283 | SEQ ID NO: 284 |
| [28] | 2C6 IgG4 | SEQ ID NO: 286 | SEQ ID NO: 287 |
| [29] | 2H6 IgG4 | SEQ ID NO: 286 | SEQ ID NO: 288 |
| [30] | 3D1 IgG4 | SEQ ID NO: 286 | SEQ ID NO: 289 |
| [31] | 2G9 IgG4 | SEQ ID NO: 291 | SEQ ID NO: 288 |
| [32] | 2G12 IgG4 | SEQ ID NO: 293 | SEQ ID NO: 294 |
| [33] | 2H5 IgG4 | SEQ ID NO: 296 | SEQ ID NO: 297 |
| [34] | 3F8 IgG4 | SEQ ID NO: 299 | SEQ ID NO: 300 |
| [35] | 3F9 IgG4 | SEQ ID NO: 302 | SEQ ID NO: 303 |
| [36] | 4G9 IgG4 | SEQ ID NO: 305 | SEQ ID NO: 306 |
| [37] | 5A3 IgG4 | SEQ ID NO: 308 | SEQ ID NO: 309 |
| [38] | 5E8 IgG4 | SEQ ID NO: 311 | SEQ ID NO: 312 |
| [39] | C001 IgG4 | SEQ ID NO: 314 | SEQ ID NO: 315 |
| [40] | C008 IgG4 | SEQ ID NO: 314 | SEQ ID NO: 316 |
| [41] | C010 IgG4 | SEQ ID NO: 318 | SEQ ID NO: 319 |
| [42] | C023 IgG4 | SEQ ID NO: 321 | SEQ ID NO: 322 |
| [43] | C025 IgG4 | SEQ ID NO: 324 | SEQ ID NO: 325 |
| [44] | C040 IgG4 | SEQ ID NO: 327 | SEQ ID NO: 328 |
| [45] | C046 IgG4 | SEQ ID NO: 321 | SEQ ID NO: 329 |
| [46] | C117 IgG4 | SEQ ID NO: 331 | SEQ ID NO: 332 |

The present disclosure includes the combination of the aspects and preferred features described except where such a combination is clearly impermissible or expressly avoided.

The section headings used herein are for organizational purposes only and are not to be construed as limiting the subject matter described.

Aspects and embodiments of the present disclosure will now be illustrated, by way of example, with reference to the accompanying figures. Further aspects and embodiments will be apparent to those skilled in the art. All documents mentioned in this text are incorporated herein by reference.

Throughout this specification, including the claims which follow, unless the context requires otherwise, the word ‘comprise,’ and variations such as ‘comprises’ and ‘comprising,’ will be understood to imply the inclusion of a stated integer or step or group of integers or steps but not the exclusion of any other integer or step or group of integers or steps.

As used herein, an amino acid sequence or a region of a polypeptide which ‘corresponds’ to a specified reference amino acid sequence or region of a polypeptide has at least 60%, e.g. one of at least 65%, 70%, 75%, 80%, 85%, 90%, 91%, 92%, 93%, 94%, 95%, 96%, 97%, 98%, 99% or 100% sequence identity to the amino acid sequence of the amino acid sequence/polypeptide/region. An amino acid sequence/region/position of a polypeptide/amino acid sequence which ‘corresponds’ to a specified reference amino acid sequence/region/position of a polypeptide/amino acid sequence can be identified by sequence alignment of the subject sequence to the reference sequence, e.g. using sequence alignment software such as ClustalOmega (Söding, J. 2005, Bioinformatics 21, 951-960).

It must be noted that, as used in the specification and the appended claims, the singular forms ‘a,’ ‘an,’ and ‘the’ include plural referents unless the context clearly dictates otherwise. Ranges may be expressed herein as from ‘about’ one particular value, and/or to ‘about’ another particular value. When such a range is expressed, another embodiment includes from the one particular value and/or to the other particular value. Similarly, when values are expressed as approximations, by the use of the antecedent ‘about,’ it will be understood that the particular value forms another embodiment.

Where a nucleic acid sequence is disclosed herein, the reverse complement thereof is also expressly contemplated.

Methods described herein may preferably be performed in vitro. The term ‘in vitro’ is intended to encompass procedures performed with cells in culture whereas the term ‘in vivo’ is intended to encompass procedures with/on intact multi-cellular organisms.

### BRIEF DESCRIPTION OF THE FIGURES

Embodiments and experiments illustrating the principles of the present disclosure will now be discussed with reference to the accompanying figures.

FIG. 1. Binding ELISA data for 15 Fab supernatant clones to the antigen protein of biotinylated recombinant human CANX protein fused with the Fc region of human IgG1 at the C-terminus (HuCANX_hFc). The 15 Fab clones were tested in a binding ELISA assay to assess their antigen binding to biotinylated HUCANX with a human Fc tag, and detected by goat-anti-human Fab-HRP (horse radish peroxidase).

FIG. 2. Polyclonal phage ELISA data after panning round 1, 2, 3 against purified fragment of human Calnexin containing Amino acids 1-481 fused to human FC region (FC-CNX). Detection of bound polyclonal phages to FC, FC-CNX or BSA coated wells was performed with an anti-M13-HRP antibody.

FIG. 3. Monoclonal phage ELISA with indicated clones tested against coated wells with FC-CNX, FC or BSA. Detection was performed with an anti-M13 HRP.

FIG. 4. (A) Binding avidity ELISA of 15 IgG1s to the antigen protein of biotinylated HuCANX_hFc. The 15 antibody clones were tested in a binding ELISA assay to assess binding to target protein biotinylated HuCANX with a human Fc tag, and detected by streptavidin-HRP, with an irrelevant IgG1 used as a negative control antibody. (B) Binding affinity ELISA of 15 IgG1s to the antigen protein of HuCANX_His. The 15 antibody clones were tested in a binding ELISA assay to assess binding to target protein HuCANX with a His tag, and detected by anti-His-HRP, with an irrelevant IgG1 used as a negative control antibody.

FIG. 5. Binding avidity ELISA data for 15 IgG1 antibody clones to the antigen protein of CNX-His. The 15 antibody clones were tested in a binding ELISA assay to assess binding to CNX-His using an anti-human Fc-HRP for detection. An irrelevant human IgG1 was used as a negative control antibody and CNX ab10286 (Abcam) was used as a positive control. The results are split into two graphs with controls for comparison. The table on the right-hand side of the figure depicts estimated EC50 for each antibody.

FIG. 6. Binding affinity of 15 IgG1s to the antigen protein of MsCANX_His was measured by Bio-Layer Interferometry. The table depicts derived equilibrium dissociation constant (KD), association constant (Kₒₙ) and dissociation rate constant (K_{dis}) for IgG tested antibodies. 2H5 and 5A3 could not be determined.

FIG. 7. Binding affinity of 5 IgG1s to the antigen protein of Human CNX_His was measured by Bio-Layer Interferometry. The table depicts derived equilibrium dissociation constant (KD), association constant (Kₒₙ) and dissociation rate constant (K_{dis}) for IgG tested antibodies.

FIG. 8. Binding ELISA test of 8 recombinant scFV clones to antigen protein of HUCANX_hFc (100 ng and 10 ng) and BSA control coated well. The 8 clones were tested in a binding ELISA assay with detection using an anti-myc tag HRP.

FIG. 9. Binding avidity of scFV clone 8 to the antigen protein human FC-CNX. Measurement performed with Bio-Layer Interferometry. The table depicts derived equilibrium dissociation constant (KD), association constant (Kₒₙ) and dissociation rate constant (K_{dis}) for scFV clone 8.

FIG. 10. Epitope binding of the 15 antibody clones by Bio-Layer Interferometry (BLI) analysis. Light grey highlighted boxes are antibodies with non-overlapping epitope on the HUCANX_His protein. Dark grey highlighted boxes are antibodies share at least partially overlapping epitopes with the other antibodies.

FIG. 11. HDX of scFv clone 8 upon presence of FC-CNX. Each point refers to a detected peptide sequence with mass spectrometry-oriented N terminal (left) to C-terminal (right). Differences in deuterons between respective time of deuterium labeled sample with unlabelled sample are scored on Y axis. Figure includes SEQ ID NOs 384-388.

FIG. 12. HDX of FC-CNX upon presence of scFv clone8. Each point refers to a detected peptide sequence with mass spectrometry-oriented N terminal (left) to C-terminal (right). Differences in deuterons between respective time of deuterium labeled sample with unlabelled sample are scored on Y axis. Figure includes SEQ ID NOs 389-391.

FIG. 13. HDX of Heavy chain of 1E1 upon presence of human CNX-His. Each point refers to a detected peptide sequence with mass spectrometry-oriented N terminal (left) to C-terminal (right). Differences in deuterons between respective time of deuterium labeled sample with unlabelled sample are scored on Y axis Figure includes SEQ ID NOs 392-400.

FIG. 14. HDX of Light chain of Igg1 1E1 upon presence of human CNX-His. Each point refers to a detected peptide sequence with mass spectrometry-oriented N terminal (left) to C-terminal (right). Differences in deuterons between respective time of deuterium labeled sample with unlabelled sample are scored on Y axis. Figure includes SEQ ID NOs 401-404.

FIG. 15. HDX of human CNX-His upon presence of Igg 1E1. Each point refers to a detected peptide sequence with mass spectrometry-oriented N terminal (left) to C-terminal (right). Differences in deuterons between respective time of deuterium labeled sample with unlabelled sample are scored on Y axis. Figure includes SEQ ID NOs 405-407.

FIG. 16. Localisation of IgG antibodies in immunofluorescence settings. Auto contrast images of Immunofluorescent staining of MDA-231 cells with indicated human IgG1 antibodies and a secondary detection with anti-human coupled alexa Fluorophore and hoechst nuclear stain.

FIG. 17. CNX Immunofluorescence staining with IgG. Representative Images of polyclonal Huh7 CNX-mcherry cells (right image pair) stained with indicated human IgG1 antibodies and a secondary detection with anti-human coupled alexa fluorophore (left image pair). Graph of quantification using Intensity produced in CNX-mcherry cells subtracted to intensity produced by antibody in Huh7 cells is shown.

FIG. 18. CNX Immunofluorescence staining with scFV. Images of Huh7 CNX mcherry cells co-stained with indicated scFv and a secondary anti-myc coupled alexa fluorophore detection. R² correlation of cytoplasmic intensity of Huh7 CNX-Mcherry cells intensity with scFV detected anti-myc intensity was calculated and is indicated on the right.

FIG. 19. Immunoblotting test of antibodies: detection of CNX/CRT presence from nitrocellulose membranes with transferred proteins from SDS PAGE loaded with cell extracts from HeLa cells untreated or transfected with siRNA against CNX or CRT. Each indicated IgG1 was incubated on membranes and bound antibody was detected anti-human HRP secondary antibody (top and middle). An anti actin loading control is shown for top row. A test of IgG1 clone 8 for detection of CNX/CRT with membranes containing transferred proteins from SDS PAGE loaded with cell extracts from MDA-231 ERG2 and MDA-231 ERG2 CNX−/− is shown on the right. Immunoblotting with control CNX antibody ab238078 and CRT antibody ab92516 are presented.

FIG. 20. Fluorescent gelatin layered assay with Huh7 Cells seeded 48H in the presence of IgG control or scFV clone 8 at 10 ug/ml. Representative Fluorescent gelatin area and respective associated hoechst images are shown along with a graph showing a normalized calculated degraded area per cell in several fields of view.

FIG. 21. Fluorescent gelatin layered assay with Huh7 Cells seeded 48H in the presence of IgG control, ab22595 CNX antibody, scFV clone 8 or indicated IgG1 at 20 ug/ml. Representative Fluorescent gelatin area and respective associated Hoechst images are shown along with a graph showing a normalized calculated degraded area per cell in several fields of view. Error bars indicate standard error of the mean (SEM).

FIG. 22. Collagen DQ degradation assay with 3T3 v-Src cells seeded 48H in the presence of IgG control, ab22595 anti CNX, 1E1 IgG4 at 20 ug/ml. Representative fluorescent images showing DQ signal and hoechst nuclear stain. For each image there is a fixed settings converted DQ fluorescence image in black and white. A graph showing Mean+/−SEM of a normalized calculated degraded area per cell in several wells is shown along with statistical significance in relation to IgG control (**).

FIG. 23. Collagen DQ degradation assay with 3T3 v-Src cells seeded 48H in the presence of IgG control, 1E1, 4G9, 1D6 and 2G9 IgG1 at 20, 2 or 0.2 ug/ml. Representative fluorescent images showing DQ signal and hoechst nuclear stain. For each image there is a fixed settings converted DQ fluorescence image in black and white. A graph showing Mean+/−SEM of a normalized calculated degraded area per cell in several wells is shown along with statistical significance in relation to IgG control (* indicates p=≤0.05; **p=≤0.01; ***p=≤0.001; ****p=≤0.0001).

FIG. 24. Fluorescent gelatin layered assay with Huh7 Cells seeded 48H in the presence of IgG control or indicated 1gG4 at 20, 2 or 0.2 ug/ml. Representative Fluorescent gelatin area and respective associated Hoechst images are shown along with a graph showing Mean+/−SEM of a normalized calculated degraded area per cell in several fields of view for 3 biological replicates.

FIG. 25. In vivo visualization of liver tumor growth in different treated groups at day 0 and day 24 is shown in the images. Quantification of total photon flux from liver tumor expressing oncogenic shp53/Nras/Luciferase is shown in the top-right graph. Survival analysis of mice injected with CNX antibodies compared to Control is shown in the bottom-right graph.

FIG. 26. Survival analysis of mouse model of breast cancer metastasis to the lung by tail-vein injection of MDA-MB-231 ER-G2 tagged GFP cells supplemented by an anti-CNX antibody or a control antibody injection is shown in the top-right graph. Quantification of the number of nodules in each condition (the mean+/−standard deviation) is shown in the top-right graph and visually represented by the lower images.

FIG. 27. Comparison of subcutaneous tumor growth at day 10 in mice injected with both NIH/3T3vSrc (right flank) and NIN/3T3vSrc CNX CALR KO (left flank) is demonstrated in the image taken at day 10 (above) and the graph (below).

FIG. 28. (A) Immunoblot analysis of human IgG to shows that CNX antibodies significantly accumulate in 3T3vSrc tumor tissues as compared to other tissues. (B) Histopathological analyses of NIH/3T3vSrc tumor from mice treated with CNX antibodies (middle column) or Ctrl IgG1 (left column) as compared with NIH/3T3vSrc CNX CALR KO tumor. (C) Co-staining immunohistofluorescence analysis of Vimentin (fibroblast marker), human IgG1 and TUNEL (cell death) in NIH/3T3Vrc tumor and liver samples from mouse either treated with α-CNX IgG1 or Control IgG1. Scale bars, 500 μm.

FIG. 29. Impact of scFV clone8 in CAIA model: (A) Treatment schedule of collagen antibody induced arthritis setup in C57BL6 mouse model: anti-collagen antibody is injected on day 0 and is followed by an LPS stimulation injection at day 3, then scFV clone 8 was injected intraperitoneally at 100 ug per mouse at day 3, 5, 7 and 9. Control mice received PBS instead of scFV clone 8. Mice were sacrificed on day 10. (B) Variation in paws thickness was measured across the different time point and statistical significant day differences are shown (* indicates p=≤0.05; **p=≤0.01; ***p=≤0.001). (C) Arthritis score measured at day 7 and day 10 for control and scFV injected mouse groups. Statistical significance is shown at day 10 (*).

FIG. 30. Impact of IgG4 1E1 in CAIA model: (A) Treatment schedule of collagen antibody induced arthritis setup in C57BL6 mouse model: anti-collagen antibody is injected on day 0 and is followed by an LPS stimulation injection at day 3, then IgG4 1E1 was injected intraperitoneally at 250 ug per mouse at day 3, 5, 7. Control mice received PBS instead of 1 IgG4 1E1. Mice were sacrificed on day 10. (B) Variation in paws thickness was measured across the different time point and statistical significant day differences are shown (* indicates p=≤0.05; **p=≤0.01; ***p=≤0.001).

FIG. 31. O-glycosylation is enhanced in human samples of rheumatoid arthritis and osteoarthritis, indicating that high levels of O-glycosylation correlate with a diseased state. Panel A shows representative images of immunohistofluorescence staining of nuclei with Hoechst (upper panel) and O-GalNAc glycans (Tn glycans) stained with Vicia villosa lectin (VVL, lower panel) on human tissue microarray (TMA) containing joint tissues from healthy subjects (Normal) and patients with osteoarthritis (OA), rheumatoid arthritis (RA) or Psoriasis Arthritis (PSA). Scale bar, 5 μm. Panel B shows a quantification graph of Tn glycan levels in individual tissue cores. Osteoarthritis patients displayed higher O-GalNAc glycan levels than healthy subjects, while most rheumatoid arthritis patients and two Psoriasis arthritis patients showed higher O-GalNAc glycan levels than healthy individuals. Data are mean±SEM and combined from two different tissue microarray (TMA) slides consisting of tissue sections from 21 osteoarthritis patients, 18 rheumatoid arthritis patients, 6 psoriasis arthritis patients and 7 healthy subjects. Individual data points represent the raw integrated density of Vicia villosa lectin (VVL) staining normalized to that of nuclear staining of individual subjects. Box and whisker plots show all values, boxes extend from the 25^{th} to 75^{th} percentiles, and error bars span max to min values *, p<0.05, ****, p<0.0001, NS: not significant (one-way ANOVA, Kruskal-Wallis test).

FIG. 32. Tn glycan levels are enhanced in arthritis induced mice and correlated with disease severity. This indicates that high Tn glycan levels correlate with a diseased state. Panel A shows the results of haematoxylin and eosin (HE) histology of synovial tissues obtained from control mice (day 0) or those injected with Collagen type II antibody induced arthritis (CAIA) at day 7, 10 and 14. S: synovium; B: bone, P: pannus, (*): infiltration of immune cells in the synovial sub-lining, arrows: bone erosion. Panel B shows representative immunofluorescence staining images with Vicia villosa lectin (VVL; upper panel) and nuclei (lower panel) showing the time-course dependent increase of VVL staining in the pannus tissues of CAIA mice from day 7 to day 10. Scale bar, 50 μm. Panels C and D show the results of evaluation of clinical scores (C) and quantification of total Tn levels in the synovium (D) of CAIA mice from day 0 to day 14. In panel C, data are mean of arthritic scores of 4 mice per time point. In panel D, individual data points represent mean Tn levels of individual joints, two joints on the front paws of each animal are computed. Box and whisker plots show all values, boxes extend from the 25^{th} to 75^{th} percentiles, and error bars span max to min values. *, p<0.05; **, p<0.01; NS, not significant (one-way ANOVA).

FIG. 33. Synovial cells in collagen type II antibody induced arthritis (CAIA) mice display signs of GALA pathway activation. This indicates in a diseased model, the GALA pathway is active in synovial cells, thereby making the GALA pathway a therapeutic target. Panels A and B show images of co-staining of Vicia villosa lectin (VVL; A) or GALNT2 (B) with endoplasmic reticulum (ER) resident protein CNX (CNX), showing markedly increased Tn glycan levels in the synovium of arthritis induced mice. Scale bar 50 μm. Zoomed images show VVL staining or GALNT2 enzymes co-localized with CNX in arthritis joints, indicating the GALA activation state. Zoom scale 4x; S: synovial membrane; B: bone; arrows show either Golgi (in untreated mice) or ER staining patterns (in CAIA mice) of VVL or GALNT2.

FIG. 34. Synovial fibroblasts (SF) are the major cell type displaying GALA activation in Collagen type II antibody induced arthritis (CAIA) mice, indicating that in a diseased model, the GALA pathway is active in synovial cells and thereby making the GALA pathway a therapeutic target. Co-staining of Vicia villosa lectin (bottom right panel) with fibroblast marker vimentin (bottom left panel), immune cell marker anti-CD45 (top right panel) and nuclei (top left panel) shows relative distribution of immune cells (circles), fibroblasts (rectangles) in the pannus tissues of CAIA mice and their Tn expression levels. Scale bar, 50 μm.

FIG. 35. Synovial lining fibroblasts from both osteoarthritis and rheumatoid arthritis patients display strong GALA activation, indicating that in a diseased model, the GALA pathway is active in synovial cells and thereby making the GALA pathway a therapeutic target. Panel A shows the results of haematoxylin and eosin (H&E) histology of synovial tissues obtained from rheumatoid arthritis and osteoarthritis patients. “*” indicates Infiltration of immune cells in the sub-lining of RA synovium; SL: synovial lining. Scale bar, 100 μm. Panel B shows representative immunofluorescence images of osteoarthritis (top panel) and rheumatoid arthritis (bottom panel) synovium showing strong Tn glycan levels in the lining synovial fibroblasts (SF), as identified by FAPα (arrowheads) and sparse Tn staining in immune cells identified by CD45 in the sub-lining (divided by a dashed line). Scale bar, 50 μm.

FIG. 36. Primary synovial fibroblasts (SF) from osteoarthritis and rheumatoid arthritis patients induces strong GALA activation in response to stimulation with arthritis-driving cytokines and cartilage extracellular matrix (ECM). This shows that the GALA pathway is responsible for the symptoms and disease progression of arthritis. Panel A shows fluorescence-activated cell sorting (FACS) dot plots showing high purity (≥90%) of primary synovial fibroblasts cultures established from healthy subjects (HCSF), osteoarthritis (OASF) and rheumatoid arthritis (RASF) patients. Panel B shows representative images of Helix pomatia lectin (HPL) staining show higher Tn glycan levels in both osteoarthritis synovial fibroblasts and rheumatoid arthritis compared synovial fibroblasts to HCSF under basal conditions. The magnitudes are accelerated after priming those cells with arthritis driven cytokines including IL1β and TNFα (CYTO) and cartilage extracellular matrix. Scare bar, 20 μm. Panel C shows the results of the quantification of Helix pomatia lectin (HPL) levels in HCSF, OASF and RASF under basal conditions and stimulation with CYTO only (no coating) or in combination with cartilage extracellular matrix or collagen type I extracellular matrix. Data are mean±SEM of two 2 independent experiments. *p<0.05, ***≤0.001, ****p<0.0001, NS: not significant (Two-way ANOVA).

FIG. 37. GALA activation in synovial fibroblasts drives cartilage extracellular matrix degradation, showing that the GALA pathway is responsible for the symptoms and disease progression of arthritis. Panel A shows a strategy to inhibit GALA in synovial fibroblasts by stably transfecting SW982 synovial fibroblasts with a construct expressing doxycycline (DOX) inducible 2 lectin domains of GALNT2 in the ER (ER-2Lec). Panel B shows representative images of the results of matrix degradation activity of SW982 cells and ER-2Lec expressing SW982 cells after stimulation with rheumatoid arthritis associated cytokines (CYTO; IL1β and TNFα). Arrows show degraded matrices. Panel C shows a quantification graph showing decreased matrix degradation activity in GALA inhibited SW982 cells after stimulation with CYTO (IL1β and TNFα). Data correspond to the mean±SEM and are representative for three independent experiments. Each data point represents the total degraded area (μm) per nuclear per well. ***, p<0.001, ****p<0.0001 (One-way ANOVA). Figure includes SEQ ID NO:408.

FIG. 38. ER-2Lec expression in synovial fibroblasts in vivo suppresses GALA activation, showing the efficacy of ER-2Lec expression in treating arthritis or alleviating symptoms associated with arthritis. Representative images are shown indicating that ER-2Lec is mainly expressed in synovial fibroblasts identified by EGFP positive stains (arrowheads) in the synovium of Col6a1Cre ER-2Lec mice and that such cells display reduced VVL staining, indicating the inhibition of GALA. B: bone, S: synovium. Scale bar, 50 μm.

FIG. 39. The effect of GALA inhibition by ER-2Lec expression in reducing paw swelling in CAIA mice, showing the efficacy of ER-2Lec expression in treating arthritis or alleviating symptoms associated with arthritis. Panel A shows representative images and panel B shows quantification. Paw thickness analysis shows alleviated swelling levels on both front and hind paws of Col6a1Cre ER-2Lec mice at day 7 post arthritis induction as compared to Col6a1Cre control mice. Data are mean±SEM of two 2 independent experiments, n=5 mice per group. **, p<0.01 (one way ANOVA).

FIG. 40. GALA inhibition by ER-2Lec expression in synovial fibroblasts (SF) reduces clinical scores in CAIA mice, which in turn indicates that ER-2Lec expression in effective in treating arthritis on a clinical scale. Measurement of clinical scores shows reduced arthritis severity in Col6a1Cre ER-2Lec mice at day 7 post arthritis induction as compared to Col6a1Cre control mice. Data are mean±SEM of two 2 independent experiments, n=5 mice per group. P=0.09 (non-parametric t test, Mann-Whitney test).

FIG. 41. GALA inhibition by ER-2Lec expression in synovial fibroblasts protects CAIA mice from cartilage degradation, showing the efficacy of ER-2Lec expression in treating arthritis or alleviating symptoms associated with arthritis. Panels A and B show representative images showing Alcian blue (AB) (A) and Safranin-O (SO) (B) staining in untreated Col6a1Cre mice or arthritis induced Col6a1Cre and Col6a1Cre ER-2Lec mice at day 7. Panels C and D show results of the quantification of positive AB (C) and SO (D) staining areas (scale bars). The data showed that arthritis induced cartilage matrix degradation is restored in GALA inhibition mice (Col6aCre ER-2Lec). Each data point represents an average of positive staining area per mm2 articular cartilage from three different metacarpophalangeal joints of one animal. Data are shown as mean±SEM. *, p<0.05, **, p<0.01; **, p<0.001 (one way ANOVA test).

FIG. 42. GALA activates O-glycosylation of CNX (CNX) in arthritis-primed synovial fibroblasts. This shows the effect of the GALA pathway in the diseased state, and identifies CNX as a therapeutic target. Panel A and B shows the blot results of co-immunoprecipitation using VVL lectin (A) and a quantification graph (B) showing that levels of O-GalNAc glycosylated CNX (CNX) are enhanced in SW982 cells after stimulation with arthritis associated cytokines (CYTO) and cartilage extracellular matrix (ECM) but reduced in doxycycline- (DOX) inducible ER-2Lec expressing SW982 cells. Actin was used as a loading control. In Panel B, data are shown as mean±SEM and representative of 3 independent experiments. *, p<0.05; **, p<0.01 (one way ANOVA test).

FIG. 43. GALA induces cell surface exposure of CNX (CNX) in arthritic-primed synovial fibroblasts. This shows the effect of the GALA pathway in the diseased state, and identifies CNX as a therapeutic target. Panels A and B show flow cytometry histogram plots (A) and a quantification graph (B) showing an increased proportion of synovial fibroblasts cells expressing surface CNX (CNX) after stimulation with arthritis induced cytokines (CYTO) and cartilage extracellular matrix (ECM). The induction of cell surface CNX is diminished in ER-2Lec expressing SF cells. In B, data are shown as mean±SEM and representative of 2 independent experiments. **, p<0.01; ***, p<0.001 (one-way ANOVA test).

FIG. 44. CNX surface exposure is enhanced in primary synovial fibroblasts from both osteoarthritis (OA) and rheumatoid arthritis (RA) patients, thus identifying CNX as a therapeutic target in the treatment of arthritis. Panels A and B show flow cytometry histogram plots (A) and a quantification graph (B) showing higher proportions of osteoarthritis synovial fibroblasts (OASF) or rheumatoid arthritis synovial fibroblasts (RASF) cells positive for surface CNX compared to that of healthy control synovial fibroblasts (HCSF) under basal condition or stimulation with arthritis driven cytokines (CYTO) and cartilage extracellular matrix (ECM). In Panel B, data are shown as mean±SEM and representative of 2 independent experiments. *, p<0.05; ***, p<0.001 (one-way ANOVA test).

FIG. 45. Disulfide bonds are abundantly present in cartilage extracellular matrix (ECM). This identifies the CNX:PDIA3 complex (that is the reducing effect of the complex on disulfide bonds) as a therapeutic target. Representative immunofluorescence staining images show staining of collagen fibres containing Collagen type III (Col III), Collagen type I (Col I) and Fibronectin in cartilage extracellular matrix (ECM). Collagen disulfide bonds are chemically reduced using TCEP which can be detected by staining with OX133 antibodies or left untreated (UT). Scale bar, 5 μm.

FIG. 46. Blockage of CNX (CNX) reduces cartilage degradation activity of primary synovial fibroblasts from osteoarthritis patients, thus identifying CNX as a therapeutic target in the treatment of arthritis. Panel A and B show representative images (A) and a quantification graph (B) showing decreased matrix degradation activity in primary fibroblasts isolated from osteoarthritis patients after incubation with anti-CNX antibodies or isotype control antibodies. Data correspond to the mean±SEM and representative data for three independent experiments. Each data point represents the total degraded area (μm) per nuclear per well. ***, p<0.001 (one-way ANOVA).

FIG. 47. Treatment with antibodies against CNX (CNX) reduces paw swelling in CAIA mice, showing the efficacy of the use of anti-CNX antibodies in treating arthritis. Panel A shows a schematic of the antibody treatment scheme. Panel B shows representative photographs of CAIA mice treated with anti-CNX antibodies or left untreated at day 10. Panel C shows a line graph plotting paw thickness measurement, which shows reduced paw swelling in CAIA animals after injecting with anti-CNX antibodies. Data represent mean±SEM and n=4-5 mice per group. *, p<0.05 (two-way ANOVA test).

FIG. 48. Treatment with antibodies against CNX (CNX) reduces arthritis severity in CAIA mice, thus showing the efficacy of the use of anti-CNX antibodies in treating arthritis. Clinical scores of CAIA mice at day 10 following treatment with isotype control antibodies or anti-CNX antibodies. Data are mean±SEM, n=4-5 mice per group. P-value is estimated by non-parametric t-test (Mann-Whitney test).

FIG. 49. Treatment with antibodies against CNX (CNX) protects CAIA mice from cartilage degradation, thus showing the efficacy of the use of anti-CNX antibodies in treating arthritis or preventing the worsening of arthritis or a symptom of arthritis. Panels A and B show representative histological images showing Alcian blue (AB) (A) and Safranin-O (SO) (B) staining (arrow bars) in CAIA mice treated with isotype control or anti-CNX antibodies. Panels C and D show quantification graphs show increased AB and SO staining areas in CAIA mice treated with anti-CNX antibodies as compared to those treated with isotype control antibodies. Individual data points represent average of positive staining area per mm² articular cartilage from individual metacarpophalangeal joints of one animal, n=4-5 mice per group. Data are shown as mean±SEM. *, p<0.05; **, p<0.01 (Mann-Whitney test).

FIG. 50. Anti-CNX (CNX) antibodies are accumulated in the synovium of CAIA mice. Representative immunofluorescence images show co-staining of VVL and anti-CNX antibodies (arrow heads) in CAIA mice injected with anti-CNX antibodies or isotype control antibodies at day 10. This shows that the accumulation of anti-CNX antibodies indicates that their ability to bind and target the CAIA mice synovium, while, in contrast, there is no binding with the control isotype antibodies. This indicates that CNX is expressed by cells in the synovium of CAIA mice, and that the cells in the synovium can specifically be targeted by anti-CNX antibodies for therapy. B: bone, S: synovium. Scale bar, 50 μm.

FIG. 51. Results of screening for GALA targets and validation of its effect on primary arthritic synovial fibroblasts. Panel A shows a schematic diagram of high content screen to select GALA targets and their blocking modalities. Synovial fibroblast (SF) cells (SW982) were cultured on a quenched fluorescent cartilage matrix component (DQ-Collagen) in the presence of GALA target blockers (antibodies or siRNA). The degradation activity of GALA in synovial fibroblasts results in fluorescence signal increases (control panel), while synovial fibroblasts treated with blockers could inhibit degradation activity. Panel B shows the results of staining with VVL showing high Tn glycan expression outside Golgi (identified by Golgi marker, Giantin), indicating GALA activation in primary synovial fibroblasts isolated from an osteoarthritis patient (OASF). Scale bar, 5 μm. Panel C shows a quantification graph showing decreased matrix degradation activity in primary synovial fibroblasts, isolated from an osteoarthritis patient (OASF), treated with anti-CNX and anti-MMP14 antibodies. Each data point represents the ratio of raw integrated density of degraded DQ-Collagen to nuclear per field of view. Data are mean±SEM, n=20 field of views per group. *, p<0.05, **, p<0.01; NS, not significant (one way ANOVA test).

FIG. 52. Characterisation of mice injected with anti-CNX or isotype control antibodies. This data shows comparable body weight changes between the two. A: Body weight changes of CAIA mice treated with isotype control or anti-CNX antibodies. B and C: Representative histological images and quantification of Safranin-O (SO) staining areas (B) in CAIA mice treated with isotype control or anti-CNX antibodies. Individual data points represent average of positive staining area per mm2 articular cartilage from individual metacarpophalangeal joints of one animal, n=4-5 mice per group. (C) Data are shown as mean±SEM. *, p<0.05; **, p<0.01 (Mann-Whitney test).

FIG. 53. O-GalNAc (Tn) glycans tissue analysis in synovial tissues from both arthritis patients and arthritis induced animals. A: Representative Immunohistofluorescence staining of O-GalNAc glycans with VVL lectin on human tissue microarray (TMA) sections from Osteoarthritis (OA), psoriasis (PSA), rheumatoid arthritis (RA) and health subjects (Normal). Magnification 10×, scale bar, 500 μm. B: HE histology (upper panel) and immunohistochemistry staining with VVL lectin (lower panel) on collagen type II antibody induced arthritis (CAIA) mice at day 7 or those left untreated (UNT).

FIG. 54. OA synovial tissue analysis and primary synovial fibroblasts purification metric. A: (Left) H&E histology of synovial tissues obtained from OA patients. Scale bar, 100 μm; SL: synovial lining. (Right) Representative immunofluorescence images of OA synovium stained with VVL/CD45 and FAPα. Lining synovial fibroblasts are identified by FAPα (arrowheads). Immune cells are identified by CD45. Sublining layer and lining layers boundaries are defined with dash line. Scale bar, 50 μm. B: FACS dot plots showing high purity (≥90%) of primary SF cultures established from healthy subjects (HCSF), OA (OASF) and RA (RASF) patients.

FIG. 55. GALA activation causes damage to cartilage in CAIA mice. A: Representative images of Safranin-O (SO) (B) staining in untreated Col6a1Cre mice or arthritis induced Col6a1Cre and Col6a1Cre ER-2Lec mice at day 7. Scale bars, 100 μm. B and C: Quantification of SO staining thickness (B) and total positive staining area (C). Arthritis induced cartilage matrix degradation is indicated with arrowheads. Each data point represents an average of positive staining area per mm2 articular cartilage from three different metacarpophalangeal joints of one animal. Data are shown as mean±SEM. p<0.01; ****, p<0.0001 (one way ANOVA test).

FIG. 56. Binding data for monoclonal anti-CNX antibody clone 2G9. Data were generated using an ELISA assay. The assay revealed high specificity for CNX (left bar) for clone 2G9 and the commercial polyclonal antibody ab10286, with little binding to BSA coated wells (right bar). The negative control hIgG did not bind significantly to CNX. Data correspond to the mean±SEM and representative data for three independent experiments.

FIG. 57. Binding data for monoclonal anti-CNX antibody clone 2G9, based on ELISA essays performed with serial dilutions of antibodies 2G9 and ab10286. Data suggest higher affinity and/or avidity for 2GP compared to commercial polyclonal antibody ab10286.

FIG. 58. ECM degradation assay data demonstrating the ECM degradation ability of monoclonal anti-CNX antibody clone 2G9. Panels A and B show representative images (A) and a quantification graph (B) showing 2G9 was able to reduce by 90% ECM degradation compared to untreated controls. Data correspond to the mean±SEM and representative data for three independent experiments.

FIG. 59. ECM degradation assay data. 2G9 in IgG4 format is also able to block ECM degradation. Data correspond to the mean±SEM and representative data for three independent experiments. ***, p<0.001 (one-way ANOVA).

FIG. 60. Impact of use of IgG1 anti-CNX antibodies on expansion of Huh7 Spheroid in Matrigel. (A) Representative reconstructed brightfield Image of 96 well with Huh7 Spheroids at Day 1 and Day 12 after indicated treatment. Continuous line delineate spheroid area at Day 1. Dashed line delineate the same spheroid area as Day1 but indicate an expansion in size at Day 12. Dashed dotted line delineate the same spheroid area as Day1 but indicate a shrinking in size at Day 12. IgG1 was used at 10 μg per ml (B) Lineplot of spheroid size change at Day 1 and Day 12 for indicated Treatment. Each Line depicts a spheroid monitored at same well and image position at Day 1 and Day 12. (C) Violin and Boxplot of spheroid growth at Day12 relative to Day 1. P value indicates significant difference in growth of spheroid in 1E1 treated condition vs control IgG1. Day 1 was set at 100%.

FIG. 61. (A) Schematic representation of the fluorescent gelatin ECM degradation assay. Degraded fluorescent gelatin on the coverslip will appear as a dark shadow and the degraded area was quantified. (B) Representative images of the degraded areas of gelatin and nuclei with various antibody treatments. Dox- refers to SW982 ERG1 cells that were not induced with doxycycline while Dox+ refers to cells induced with 1 ug/ml doxycycline. All cells that were treated with 10 ug/ml antibodies were under doxycycline induction. Ctl hIgG1 corresponds to SW982 cells treated with an unrelated human IgG1. ab22595 and ab92573 are commercial antibodies targeting CNX. Two experimental replicates were performed. (C) Quantification of the degraded ECM area per nuclei in presence of various antibody treatments. Each point represents one imaged field. The results are a combination of two experimental replicates.

FIG. 62. (A) Paw thickness variation in CAIA mice treated with 1E1 or control PBS from day 0 to day 17. (B) Unpaired t-test demonstrates significant difference in change in paw thickness between control mice and 1E1 treated mice from day 0 to day 17.

FIG. 63. (A) Paw thickness variation in CAIA mice treated with 2G9 or control IgG1 from day 0 to day 17. (B) Unpaired t-test demonstrates significant difference in change in paw thickness between control mice and 2G9 treated mice from day 0 to day 17.

FIG. 64. (A) Representative images of the degraded areas of gelatin and nuclei by HUH7 (top) and SW982 ERG1 (bottom) cells. Cells were treated with various antibodies in 2 ug/ml, 1 ug/ml and 0.2 ug/ml doses for two days. SW982 ERG1 cells were induced with 1 ug/ml doxycycline to activate GALA. Ctl hIgG1 corresponds to cells treated with an unrelated human IgG1. ab22595 is a commercial antibody targeting CNX. (B) Quantification of the degraded ECM area per nuclei of HUH7 (left) and SW982 ERG1 (right) treated with various antibodies in 2 ug/ml, 1 ug/ml and 0.2 ug/ml doses for two days. Each point represents one imaged field.

FIG. 65. Immunoblot analysis of human IgG1 shows that CNX antibodies significantly accumulate in HepG2-Luc tumor tissues as compared to Control.

FIG. 66. Immunoblot analysis of human IgG1 shows that CNX antibodies significantly accumulate in Hep3B tumor tissues as compared to Control.

FIG. 67. Immunoblot analysis of human IgG1 shows that CNX antibodies significantly accumulate in Huh7-Luc tumor tissues as compared to Control.

FIG. 68. Identification of the amount of CNX antibodies, named 1E1, accumulated in various tumors (HepG2Luc, Hep3B and Huh7Luc) from NSG mice treated with either 1E1 or Control IgG1 (EBOLA) for three doses at 30 mg/kg every 2 days. (A) Western blot of 25 μg tumor/well with serial igG1 dilution. (B) Standard curves generated via Image J analysis using western blot intensity of H and L chain normalised background. (C) Amount of IgG1 in 1 μg tumor tissue (Mean+SD.).

FIG. 69. (A) In vivo visualization of subcutaneous tumor growth in different treated groups at day 0, day 7 and day 20 is shown in the images. Nude mice carrying tumors were treated with CNX antibodies (1E1) compared to IgG1 Control at 15 mg/kg per dose. (B) Quantification of total photon flux from HepG2Luc tumors expressing Luciferase. (C) Quantification of total photon flux from Huh7Luc tumors expressing Luciferase, Mean+SD.

FIG. 70. (A) In vivo visualization of subcutaneous tumor growth in different treated groups at day 0, day 18 and day 27 is shown in the images. NSG mice carrying tumors were treated with CNX antibodies (1E1 or 3D1) compared to IgG1 Control at 15 mg/kg per dose. (B) Quantification of total photon flux from HepG2Luc tumors (at 1 million cells). (C) Quantification of total photon flux from HepG2Luc tumors expressing Luciferase (at 5 million cells), Mean+SD.

FIG. 71. (A) Schematic of in vivo imagining experiment. (B) In vivo bioluminescence imaging of α-Calnexin/1E1 Alexa Fluor®680-conjugated in mice bearing in HepG2Luc tumors. (C) In vivo fluorescence Imaging of α-Calnexin/1E1 Alexa Fluor®680-conjugated in mice bearing in HepG2Luc tumors. (D) Ex vivo imaging of Calnexin/1E1 Alexa Fluor®680-conjugated accumulation in tumor versus organs at 5 days post injection.

### EXAMPLES

In the following Examples, the inventors describe the generation of novel CNX-specific antibodies and their biophysical and functional characterisation.

### Example 1: Identification of Human Monoclonal Antibodies

15 Clones Identified from a Human Fab Library

Recombinant human CANX protein fused with the Fc region of human IgG1 at the C-terminus (HuCANX_hFc) was used to isolate binders from a library of Fab sequences, using phage display technology. 15 unique clones were isolated from 475 clones initially identified. These 15 clones showed high Fab supernatant binding signals to HuCANX_hFc in ELISA, which were then cloned into IgG format for further characterisation and given the following identifiers: 1D3, 1D6, 1E1, 1E6, 2C6, 2G9, 2G12, 2H5, 2H6, 3D1, 3F8, 3F9, 4G9, 5A3, 5E8.

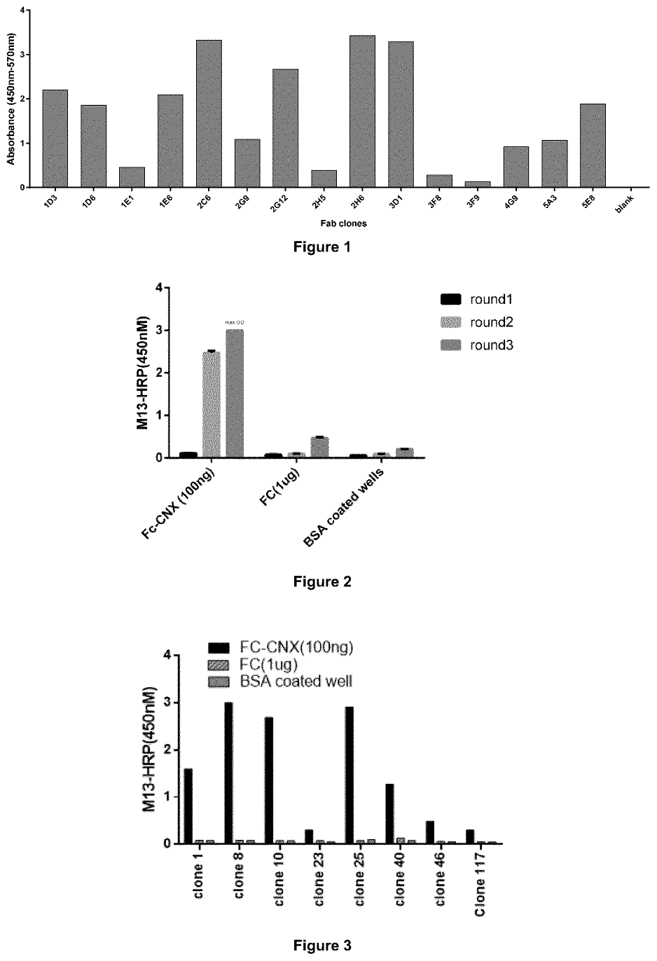

The results of binding ELISA using Fab supernatants for the 15 unique clones are shown in FIG. 1. The 15 Fabs clones were reformatted into human IgG via PCR cloning of the variable regions of the heavy and light chains from the phagemid and insertion in the pTT5 vector.

8 Clones Identified from a Human scFv Library

Naïve library ETC-H1 (Dorfmueller et al Sci Rep 6, 21661, 2016) was used and is based on a human naive repertoire in single chain Fv (scFv) format with N terminal VH fused with 3×(GGGGS) (SEQ ID NO:409) linker to C-terminal VL. ETC-H1 has a library complexity established from 10¹⁰ transformed independent clones.

Panning was performed with 3 rounds of selection/amplification. A human antibody phage library (ETC-H1) was used and designed to contain phagemid pIT2 expressing sequences of amplified human VH Igg domain fused to a 15 Amino acid linker (GGGGS)3 (SEQ ID NO:409), fused to amplified human VL Igg domains followed by a his tag a myc tag and pIII phage minor coat protein. Phagemid library was transformed into TG1 bacterial cells. The TG1 bacterial library was transduced with M13K07 helper phages to facilitate production of phages expressing scFv on their surface via PIII minor coat protein, here referred to as the scFv phage library. A purified fragment of human CNX containing Amino acids 1-481 fused to human FC region was used as antigen (FC-CNX) for panning human scFv phage library (Sinobiological). 1 μg FC-CNX was diluted in bicarbonate/carbonate antigen coating buffer (100 mM NaHCO3 pH9.6) in a well of 96 well maxisorp plate overnight. Distinct wells were also coated with either 1 μg human Fc-region in 100 mM NaHCO3 pH9.6 or a mix of 1% dry milk 1% BSA in PBS. Next day excess was removed, and the surface of all wells were blocked with 2% dry milk in PBS for 2 hours. Excess volume in wells was then removed and washed manually 3× with PBS 0.05% Tween. 10¹⁰ phages from the scFv library diluted in PBS 1% milk/BSA were added to milk/BSA blocking well for 2 hours. Phage excess volume was then transferred to FC coated well for 2H. Excess phage volume was then transferred to FC-CNX coated well for 2H. Excess of phage was then discarded and FC-CNX wells were washed with PBS 0.05% Tween with 1 minute incubation and repeated for a total of 5 wash. Phages attached to FC-CNX were eluted with trypsin solution at 10 μg per ml with shaking at 37° C. for 30 minutes. TG1 growing bacteria at OD600 nm=0.4 were transduced with eluted phages and selected on YT agar ampicillin 1% glucose agar plates overnight. Pooled bacterial colonies were grown in YT ampicillin 1% glucose and transduced with M13K07 helper phages, selected and induced for phage expression in YT ampicillin kanamycin 0.1% glucose overnight at 30° C. Phages were collected from supernatant media and precipitated for 2H with ⅕th volume of 20% PEG-6000/2.5 M NaCl on ice. After centrifugation, phage pellet was resuspended in PBS and used for the next round of panning. Subsequent rounds of panning followed the same methodology but have specifically 10 washes of 1 min with PBS 0.05% tween for round 2 or 15 washes of 1 min for round 3 for FC-CNX/phages washing step.

Polyclonal Phage populations from each round were tested by phage ELISA to measure the enrichment in positive binders to FC-CNX (100 ng/well) vs FC (1 μg/well) vs BSA coated wells. Binding of phages to coated antigen was detected via M13-HRP (horse radish peroxidase) which binds the major phage coat protein. The ELISA analysis revealed a marked enrichment in the library from rounds 2 and 3 binding to FC-CNX (FIG. 2).

Single colonies recovered from the third round of panning were evaluated with production of phages in multiwell plates and tested for presence of binding to FC-CNX coated well, absence of binding to FC coated well and absence of binding to BSA coated well in a Phage ELISA using anti-M13 HRP detection antibody (FIG. 3). Indicated clones showed specific enrichment against FC-CNX coated wells but not FC coated or BSA control wells.

Frequency of clone sequences detection across 160 clones tested: clone 1:2x, clone 8:8x, clone 10:1x, clone 23:1x, clone 25:1x, clone 40:12x, clone46:1x, clone 117:2x.

For IgG1, a nucleotide sequence encoding Clone8 VH domain (SEQ ID NO:165) was inserted into pFUSEss-CHIg-hG1 (Invivogen) after IL2 signal sequence. For IgG4, a nucleotide sequence encoding Clone8 VH domain was inserted into pFUSEss-CHIg-hG4(invivogen) after a nucleotide sequence encoding IL-2 signal sequence. Clone8 antibody light chains were produced by introducing a nucleotide sequence encoding Clone8 VL domain (SEQ ID NO:178) into pFUSE2ss-CLIg-hI2 (Invivogen) after a nucleotide sequence encoding IL2 signal sequence. Co-transfection of pFUSEss-CHIg-hG1/pFUSE2ss-CLIg-hI2 or pFUSEss-CHIg-hG4/pFUSE2ss-CLIg-hI2 in Expi293/ExpiCHO cells was used to produce Clone8 in human IgG1/IgG4 framework.

### Example 2: Binding Affinities of Human Monoclonal Antibodies

Binding Affinities of Fab and scFv Derived Antibodies

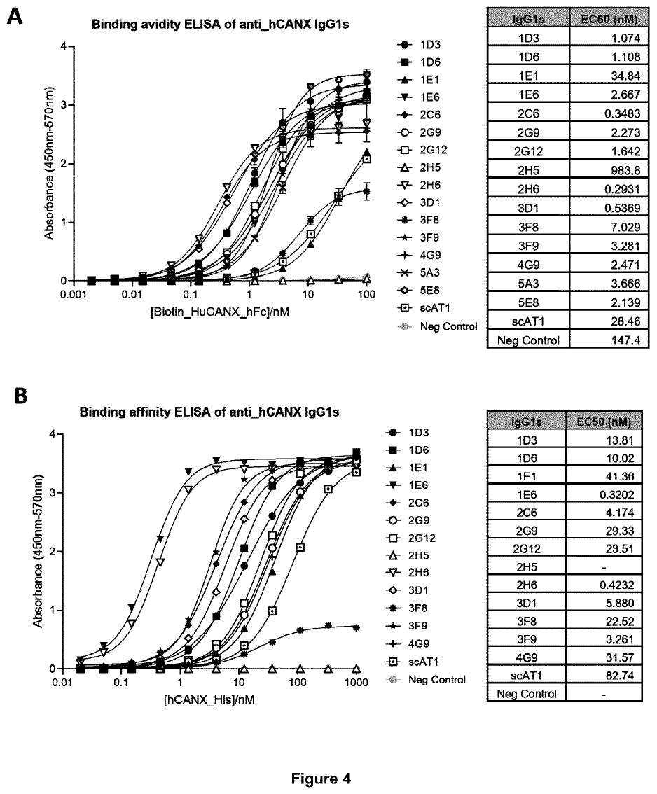

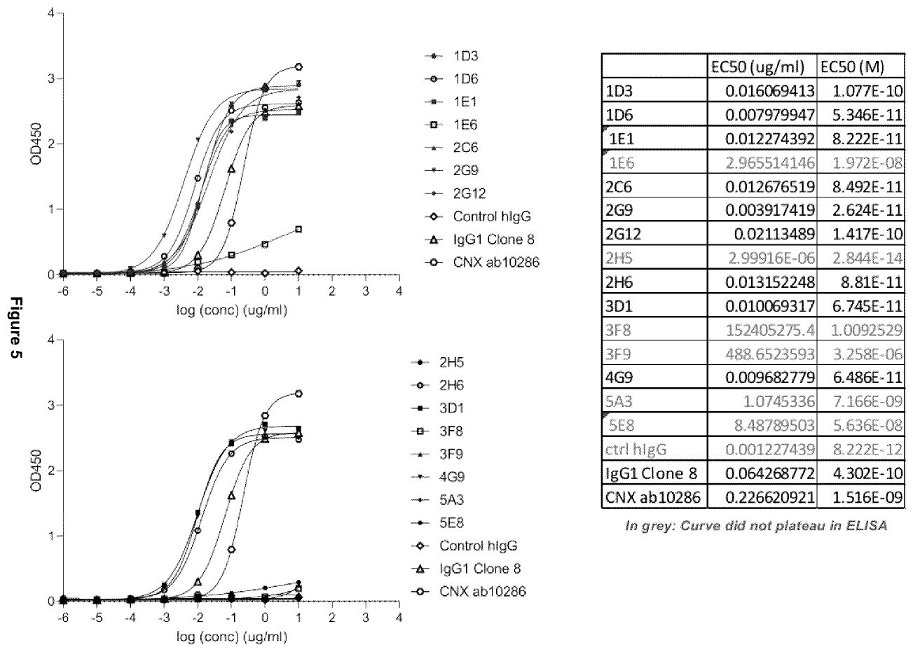

The 15 antibody clones were tested using ELISA against biotinylated recombinant HuCANX_hFc protein to assess binding avidity for the target, with an irrelevant IgG1 used as a negative control antibody. In brief, the 15 antibody clones were coated at 2 μg/ml onto the 96-well ELISA plates overnight at 4° C. After blocking with casein for 2 hours, the biotinylated antigen was added at different concentrations (3 times serial dilutions) and incubated for 1 hour. The wells were then washed with PBST and detected by HRP conjugated streptavidin. With the exception of clone 2H5, all the other 15 clones showed dose dependent antigen binding for HuCANX_hFc (FIG. 4a). 13 antibody clones were also tested in ELISA against recombinant HuCANX_His protein to assess binding affinity for the target, with an irrelevant IgG1 used as a negative control antibody. In brief, the 15 antibody clones were coated at 10 nM onto the 96-well ELISA plates overnight at 4° C. After blocking with casein for 2 hours, the antigen was added at different concentrations (3 times serial dilutions) and incubated for 1 hour. The wells were then washed with PBST and detected by HRP conjugated anti-His tag antibody. With the exception of clone 2H5, all the other clones showed dose dependent antigen binding for HuCANX_His (FIG. 4b).

CNX-his antigen (Genscript) was diluted in bicarbonate/carbonate antigen coating buffer (100 mM NaHCO₃) and coated on maxisorp microplates shaking overnight at 4 C. The wells were subsequently blocked with 5% milk in PBS-Tween (PBST) for 2 hours at room temperature. The wells were washed 3 times with PBST before the addition of antibodies at several indicated dilution in 2% BSA-PBST and incubated for 1 hour at 37 C. The wells were washed 3 times with PBST. The specific anti-human secondary antibody conjugated with horseradish peroxidase was diluted and added to wells with a subsequent incubation for 1 hour at 37° C. Then wells were washed 3 times with PBST. Addition of TMB solution was performed and incubated at room temperature for 30 mins or until desired color change was attained. Addition of 0.1 M HCL to stop reaction was then added and measurement of absorbance at 450 nm on a microplate reader was then followed. ELISA was performed with IgG1 Fab formatted antibodies and scFv clone 8 converted to IgG1 at indicated dilution (log scale in μg/ml) against constant CNX-his antigen coated on Maxisorp plate. CNX ab10286 (abcam) and a human Igg1 unrelated were used as positive and negative control respectively. 4 parameters logistic curve fit were used to calculate EC50. 1E6, 2H5, 3F8, 3F9, 5A3 and 5E8 showed poor binding whereas the remaining antibodies produced EC50>10⁻¹⁰ M (FIG. 5).

To measure the affinity of selected antibodies, the Biolayer Interferometry (BLI) assay was used. Briefly, the tested antibody was coated on the tip of a fiber optic sensor. Then, the sensor was incubated in a solution containing a CNX fragment (CNX-his). The binding changes the thickness of the molecular complex, affecting the interference patterns with the light passing through the detector. The speed of change and range of variation allows the derivation of an association constant, kon, between the antibody and the target protein (CNX). After equilibrium was achieved, the sensor was transferred in a medium free of CNX and a dissociation constant (k_{dis}) can be measured.

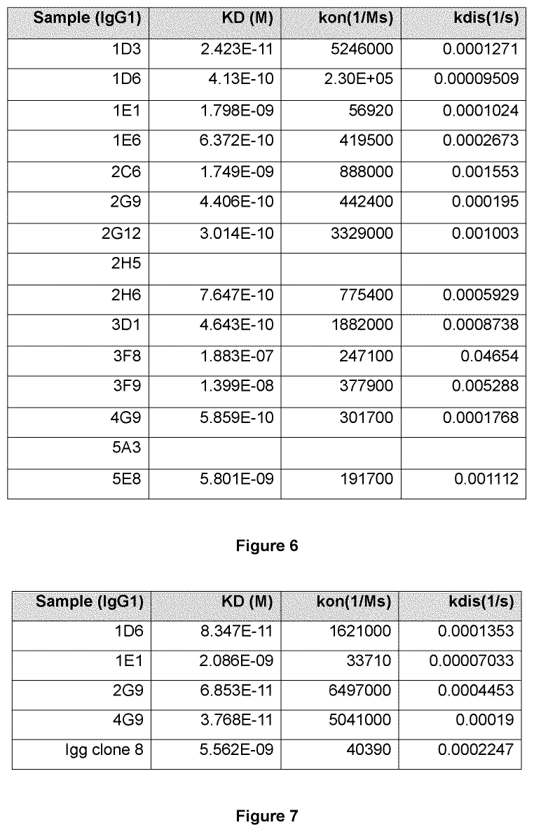

The binding affinity of the 15 antibody clones to recombinant mouse CANX protein with a polyhistidine tag at the C-terminus (MsCANX_His) was measured by Bio-Layer Interferometry. The 15 antibody clones were loaded and captured by the anti-human IgG Fc capture (AHC) biosensors (FIG. 6). The biosensors were dipped into a range of MsCANX_His protein concentrations (from 200 nM to 0.39 nM, in 2-fold dilutions), the kinetics of the association and dissociation were then recorded.

The binding affinity of the 5 antibody clones (including Igg clone 8) to recombinant human CNX protein with a polyhistidine tag at the C-terminus (HuCANX_His) was further measured by Bio-Layer Interferometry (FIG. 7). The 5 antibody clones were loaded and captured by the anti-human IgG Fc capture (AHC) biosensors. The biosensors were dipped into a range of HuCANX_His protein concentrations (from 200 nM to 3.13 nM, in 2-fold dilutions), the kinetics of the association and dissociation were then recorded.

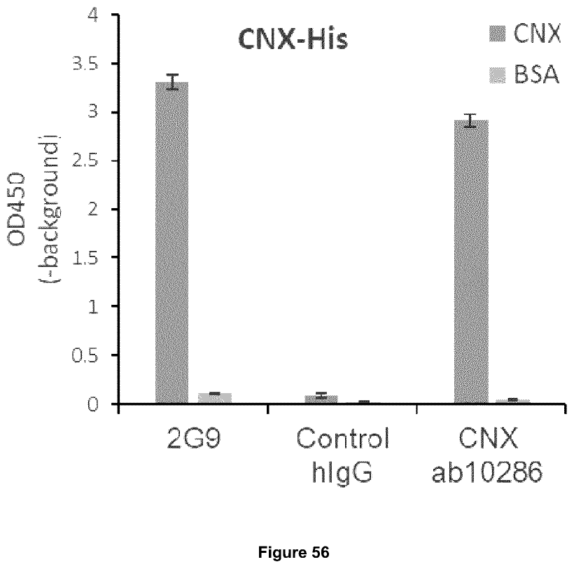

In further experiments, binding of antibody 2G9 binding was investigated. Purified His-tagged human CNX was adsorbed and coated on plastic wells; control wells were coated with BSA. 2G9, a control human IgG or the commercial monoclonal antibody ab10286 were then added to the plates at 10 μg/ml. The results revealed high specificity for CNX for both antibodies, with little binding to BSA coated wells (FIG. 56). The negative control hIgG did not bind significantly to CNX.

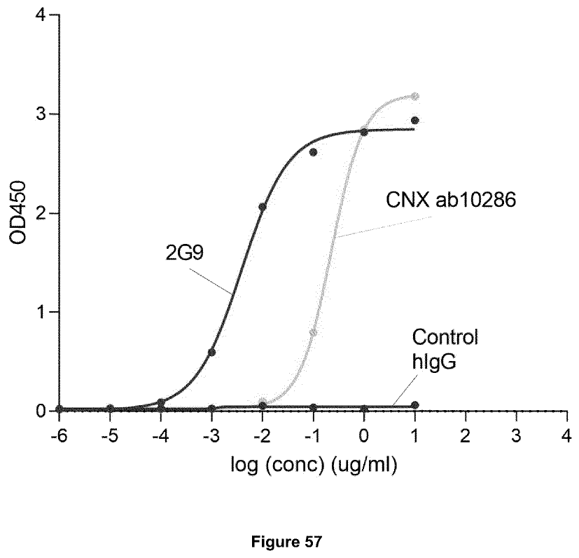

To quantitatively compare 2G9 and ab10286, the ELISA assay was repeated using serial dilutions of the antibodies (FIG. 57). 2G9 displayed saturated binding at 0.1 μg/ml, with a calculated EC50 of ~3.10e⁻⁶ μg/ml. This concentration is lower than the commercial monoclonal antibody ab10286, suggesting a higher affinity and/or avidity.

Binding Affinities of scFv Derived Antibodies

FC-CNX antigen protein was diluted in bicarbonate/carbonate antigen coating buffer (100 mM NaHCO₃) and coated on maxisorp microplates shaking overnight at 4 C. The wells were subsequently blocked with 5% milk in PBS-Tween (PBST) for 2 hours at room temperature. The wells were washed 3 times with PBST before the addition of scFv antibodies in 2% BSA-PBST and incubated for 1 hour at 37C. The wells were washed 3 times with PBST. The specific anti-myc secondary antibody conjugated with horseradish peroxidase (scFv constructs have a myc tag in C terminal of their sequence) was diluted and added to wells with a subsequent incubation for 1 hour at 37C. Wash 3 times with PBST. Addition of TMB solution was performed and incubation at room temperature for 30 mins or until desired color change was attained. Addition of 0.1 M HCL and measurement of absorbance at 450 nm on a microplate reader was then followed.

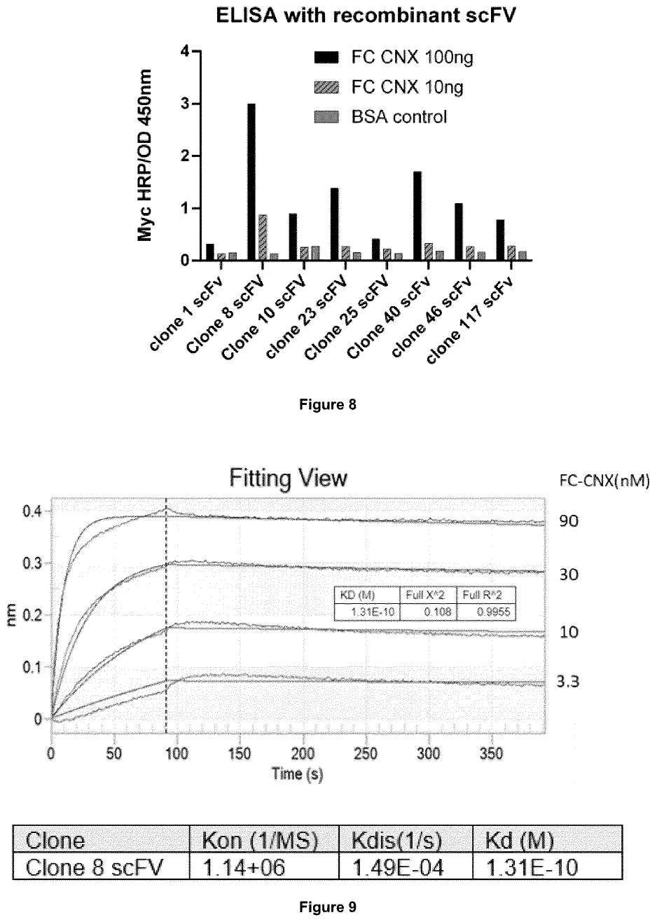

scFV format Clone 1, 8, 10, 23, 25, 40, 46, 117 showed an increased signal in FC-CNX as compared to BSA coated wells (FIG. 8). Only clone 8 scFv showed association with lower amount of FC-CNX antigen at 10 ng coated well.

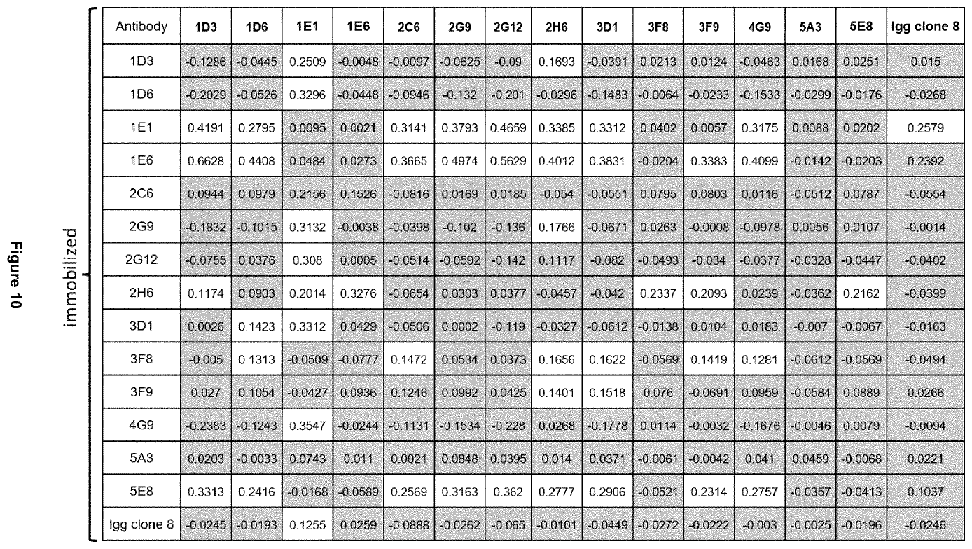

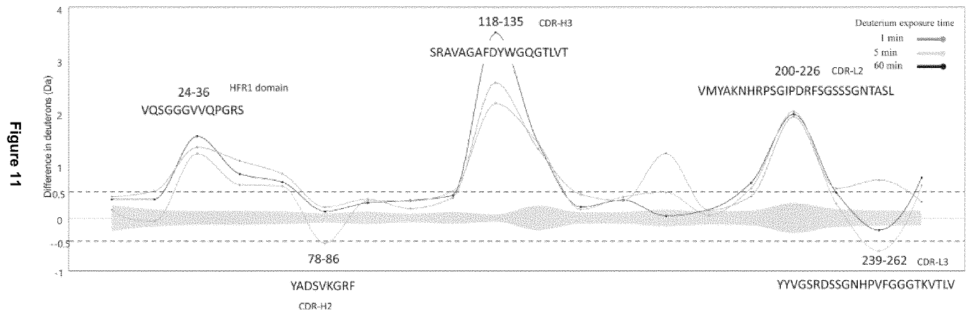

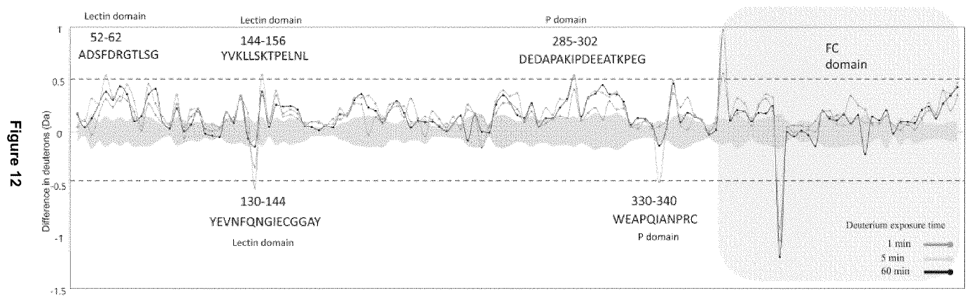

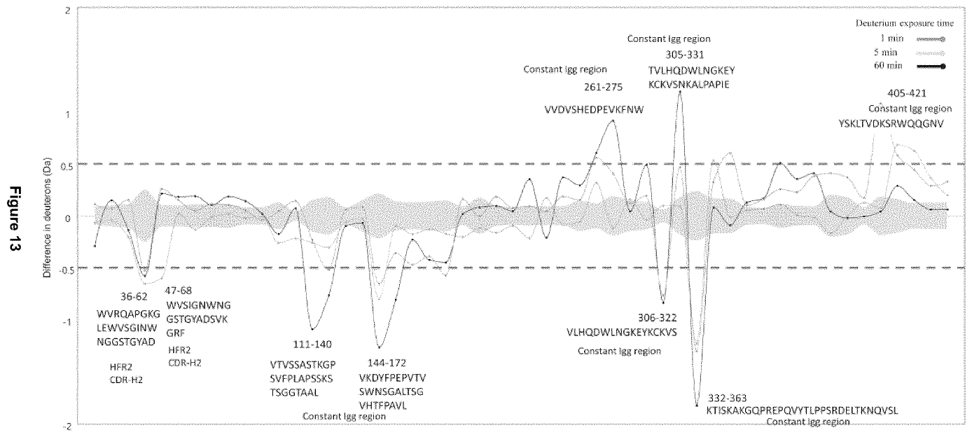

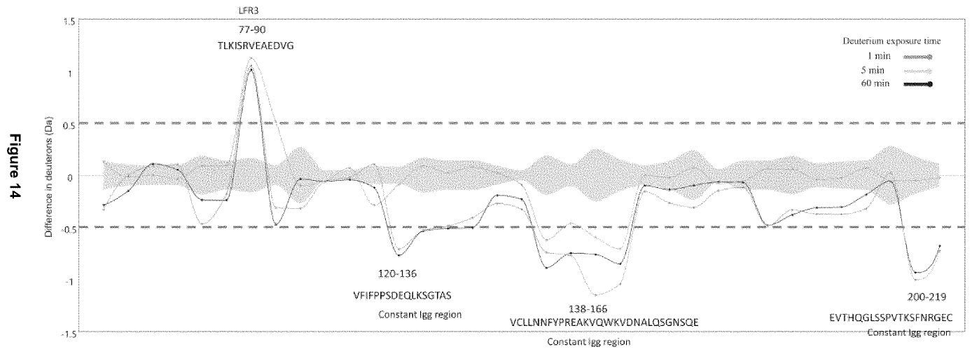

Constant quantity of recombinant scFv clone 8 was captured on Octet (Fortebio) penta-His sensor tips and was submerged in solution of FC-CNX (90, 30, 10 and 3.3 nM) in 1× kinetic buffer (Fortebio) for association and was then submerged in 1K kinetic buffer solution for dissociation (FIG. 9). Curve fitting was performed with octet software to report indicated rate.

### Epitope Mapping of CNX Antibodies

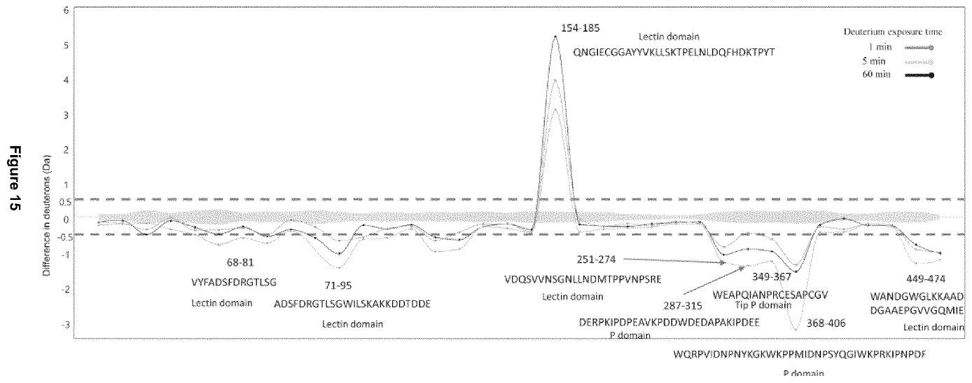

Epitope binning of 15 antibody clones was performed using a classical sandwich assay. A selected antibody was immobilized onto AR2G biosensors, then dipped into antigen protein of HuCANX_His solution and then dipped into a panel of 15 different antibodies. An irrelevant antibody was included as control. Results are shown in FIG. 10.

scFv Clone 8 and IgG1 1E1 were tested with hydrogen/deuterium exchange coupled with mass spectrometry (HDX) against FC-CNX (sinobiological) or a recombinant CNX-histidine tag (genscript custom recombinant production) respectively. Each candidate is deuterium labeled (for 0, 1, 5 or 60 minutes) alone or in a complex with antigen. Deuterium labeling occurs on accessible surfaces. Therefore, no labelling is present on inaccessible protein domains engaged in interaction with an antigen (reduced deuterion difference). Dynamic change of conformation at certain protein locations can be inferred when deuterium labeling is more pronounced in more exposed regions upon association (increased deuterion difference). Upon trypsin digestion of these samples, peptides can be detected in mass spectrometry and referenced back by mass to the original antigen or antibody known sequence of samples to detect abundance and deuterium presence.

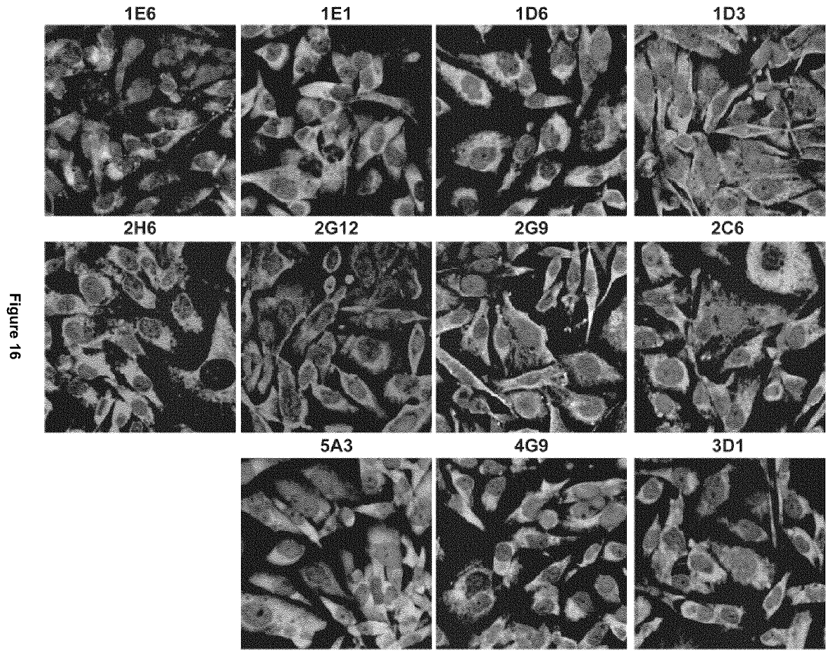

scFv clone8 was investigated for association with FC-CNX. Decreased Deuterium exchange for indicated peptides suggested association of CDR-H2 (AA 78-86) and CDR-L3 (AA 239-262) of scFv clone 8 with FC-CNX. Increased deuterium exchange suggested conformational change of HFR1 (AA 24-36), CDR-H3 (AA-118-135) and CDR-L2 (AA 200-226) of scFv clone 8 upon binding to FC-CNX (FIG. 11).

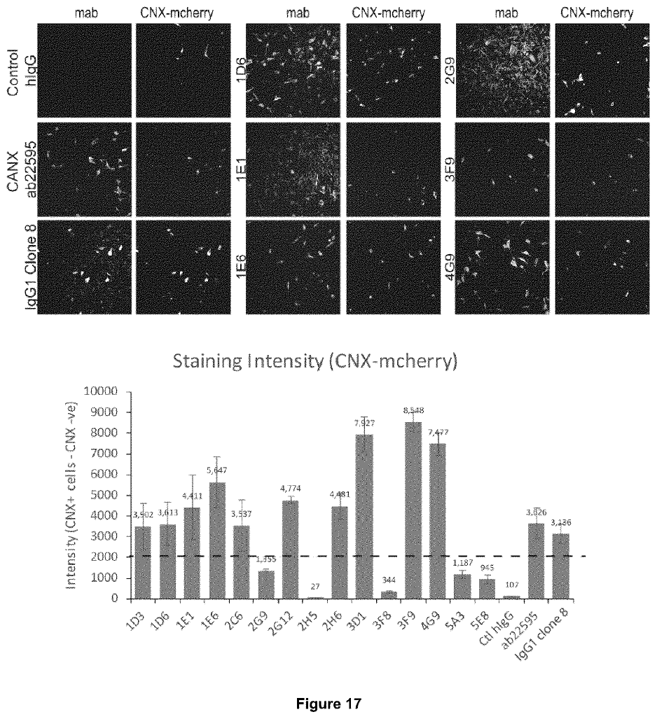

Decreased deuterium exchange of Lectin domain (AA 130-144) and P domain of FC_CNX (AA 330-340) suggested association of these respective domains with scFv clone 8 (FIG. 12). Increased deuterium exchange at lectin domain (AA 52-62, 144-156,) and P domain (AA 285-302) suggested conformational change of these respective domains upon engagement to scFv clone8.

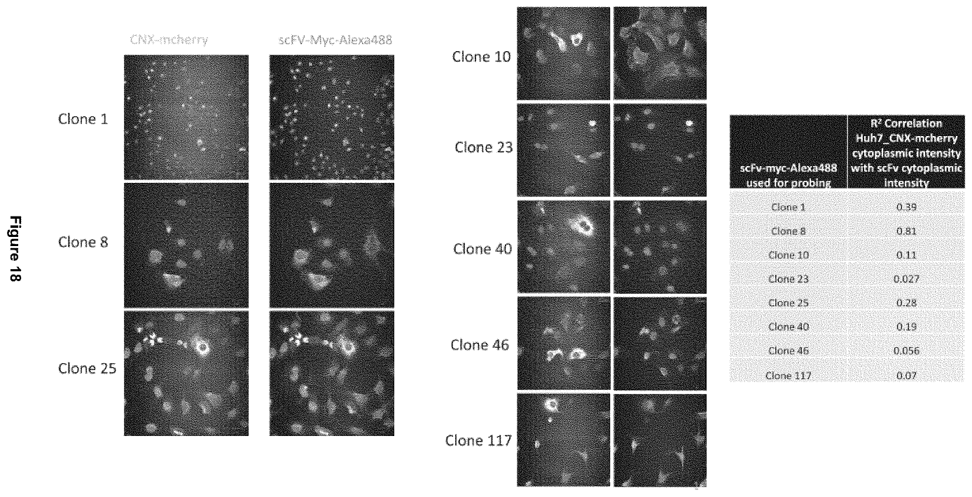

IgG1 1E1 was analysed in HDX for association with CNX-His. Decreased deuterium exchange of CDR-H2/HFR2 (AA36-62, 47-68) of Heavy Chain of 1E1 suggested association of this domain with human CNX-His (FIG. 13).

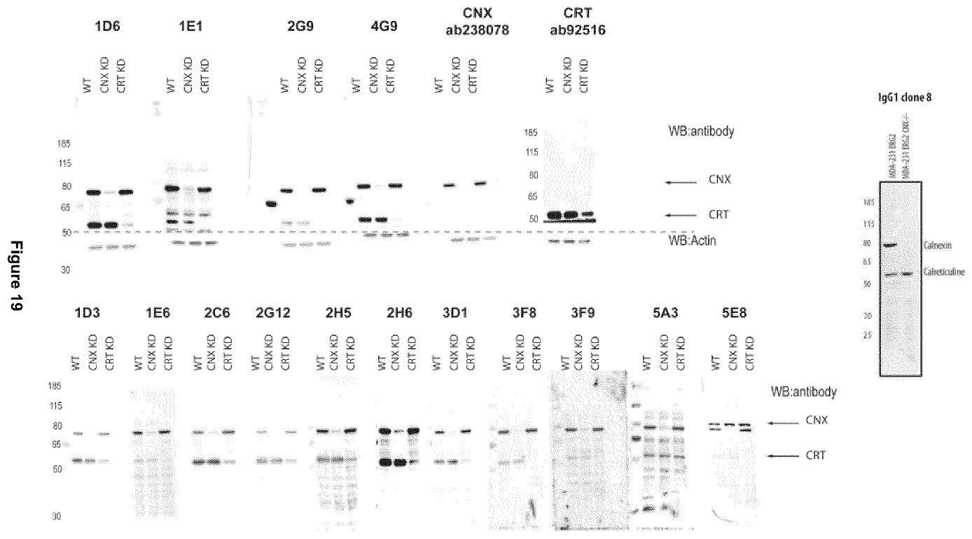

Increased deuterium exchange of LFR3 (AA70-90) of light Chain of 1E1 IgG1 suggested conformational change of this domain upon association with human CNX-His (FIG. 14).

Decreased deuterium exchange of lectin domain (AA68-81, 71-95, 251-274, 449-474), P domain (AA287-315) and tip of P domain (AA349-367) of human CNX-His suggested association of these domains with IgG1 1E1. Increased deuterium exchange of lectin domain (AA154-185) suggested conformational change of this domain upon association with human CNX-His (FIG. 15).

### Example 3: Immunofluorescence and Western Blot Analysis of CNX Antibodies

### Immunofluorescence Staining Shows Specific ER-Like Pattern for CNX Antibodies

Using MDA-231 cells, paraformaldehyde fixed cells were prepared. Permeabilization, blocking with calf serum, and incubation of IgG1 CNX antibodies at 10 μg/ml for 1 hour and used a secondary anti human coupled alexa fluorophore antibody along with Hoechst nuclear stain was performed. CNX and CRT being ER localized commonly produce a reticular stain throughout the cells but not at the nucleus. As expected, staining reminiscent of ER like pattern for 1D3, 1D6, 1E1, 1E6, 2C6, 2G9, 2G12, 2H6, 3D1, 4G9, 5A3 but not for 2H5, 3F8, 3F9 and 5E8 was confirmed (FIG. 16).

Immunofluorescence from Huh7 transfected with CNX-mcherry fluorescent protein was compared with fluorescent signals produced by human IgG1 CNX antibodies (including IgG1 clone 8). A secondary anti-human antibody conjugated to Alexa fluorophore 647 was used. Colocalization on images was seen for CNX-mcherry signal with control CNX antibody ab22595, IgG1 clone 8, 1D6, 1E1, 1E6, 3F9, 4G9 but not negative control human IgG (FIG. 17). The signal obtained from normal Huh7 without CNX mCherry was used to normalize the signal obtained with Huh7 CNX-mcherry. After calculation and subtraction of signal from control cells, increased signal could be obtained in range of ab22595 (Abcam) CNX antibody positive control for 1D3, 1D6, 1E1, 1E6, 2C6, 2G12, 2H6, 3D1, 3F9, 4G9, IgG1 clone 8 (FIG. 17). Thus, these specific antibodies could detect CNX overexpression in CNX mCherry cells in immunofluorescence application settings.

Huh7 permeabilized cells carrying stable CNX-mcherry were also subjected to immunofluorescence assay with scFv antibodies and a secondary antibody anti-Myc conjugated to Alexa488. Clone 1, clone 8 and 25 scFv did colocalize with CNX mcherry signal produced by the cells and showed ER-like pattern localisation (FIG. 18). Clone 8 showed the best cytoplasmic colocalization index (R2) out of all scFv tested. Therefore, clone1, clone 8 and clone 25 in scFv format detect CNX overexpression in Huh7 CNX mcherry cells in immunofluorescence application settings.

### Western Blot Analysis Shows Specificity for CNX and CRT

All Fabs converted to IgG1 were tested for western blotting detection using Hela extract from cells untreated or subjected to 72H knock down with an siRNA against CNX or CRT (CRT). After SDS-PAGE and transfer to nitrocellulose membranes, membranes were blocked with BSA and incubated with 1 μg/ml antibody solution overnight, following multiple washes, an anti-human HRP secondary antibody was used for detection of tested antibodies. Positive control CNX antibody ab238078 (Abcam) produced a specific band at 80 kDa which was decreased upon CNX siRNA knock down. Positive control CRT antibody ab92516 (Abcam) produced a specific band at 50 kDa marker size which was decreased upon CRT knock down (FIG. 19). All IgG1 antibodies tested showed specificity to CNX and CRT except 5A3 and 5E8 who had specificity to CNX but no confirmed reactivity to CRT. Additional test of IgG1 clone 8 showed specificity in western blot to CNX (50 Kda) and CRT (80 Kda) when using cell extracts from human MDA-231 cells and MDA-231 CNX−/− cells.

Therefore, it is shown with western blot application settings that IgG1 of 1D6, 1E1, 2G9, 4G9, 1D3, 1E6, 2C6, 2G12, 2H5, 2H6, 3D1, 3F8, 3F9 and clone 8 detect CNX and CRT. It is additionally shown that IgG1 of 5A3 and 5E8 detect CNX in western blot application settings.

### Example 4: ECM Protective Activities of CNX Antibodies

### Assays of ECM: Fluorescence Gelatin Method/DQ Collagen Method

The fluorescence gelatin layered assay measures the degradation of fluorescently labeled gelatin by cells. A commercial solution of gelatin (2%) can be labeled with 5-Carboxy-X-Rhodamine, Succinimidyl Ester. The labeled gelatin was then transferred on sterile coverslips to create a thin layer, which was stabilized by glutaraldehyde fixation. Finally, a solution of rat tail collagen was used to coat the coverslips, creating a thin layer of collagen on top of the gelatin. Then, the coverslips are transferred in culture vessels and cells with degradative activity (for instance human hepatocellular carcinoma Huh7) are seeded and incubated for 48 h to allow for degradation to occur. After fixation, the coverslips are stained with nuclear stain hoechst to permit the counting of cells. The coverslips are imaged on a confocal microscope with at least 10 fields per coverslip. The images are then analyzed using ImageJ. A threshold was defined manually to reveal the surface of degraded gelatin and the total area per field was measured. In parallel, the number of nuclei was calculated, and the final result was normalized to the cells in each field.

The collagen DQ degradation assay uses a mix of rat tail I collagen and quenched fluorescent DQ collagen type I which is coated and polymerised on the bottom of a 384 well optical grade plate. 3t3-vSrc mouse cell lines are seeded on top of this collagen layer for a period of 48 to 72h. When cells degrade collagen, they release DQ dye which is not quenched anymore and emit fluorescence. Quantification of fluorescent area of DQ signal from live cells with high content imaging can be normalized to nuclei count to obtain a degraded area/cell.

### Several Antibodies Show High ECM Protective Activities

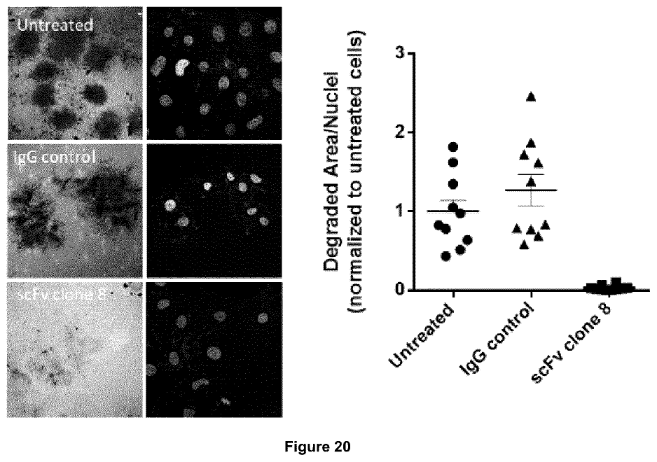

Incubation of untreated cells, negative IgG control antibody or clone 8 in scFv format at 10 μg/ml was performed on Huh7 cells seeded on fluorescent gelatin/collagen for 48H. Incubation of scFv clone 8 tested at 10 μg/ml for 48H in Huh7 cells produced a reduced degraded area as compared to untreated or IgG negative control treated cells (FIG. 20).

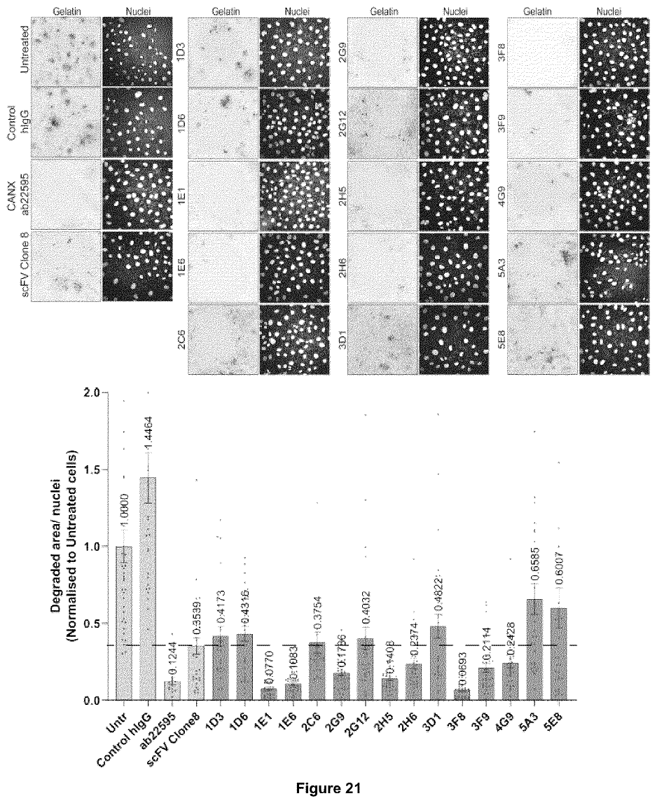

Incubation of all IgG1 CNX antibodies in tandem with clone 8 in scFv format at 20 μg/ml along with positive control ab22595 (Abcam) or a negative control IgG1 was performed on Huh7 cells seeded on fluorescent gelatin for 48H (FIG. 21). High gelatin/collagen degraded area was detected from untreated cells or negative control IgG1 whereas ab22595 positive control showed minimal degraded area. Test of scFv clone 8 produced a reduced degraded area as compared to untreated cells. All IgG1 antibodies tested showed reduced degraded area but among IgG1: 1E1, 1E6, 2G9, 2H5, 2H6, 3F8, 3F9 and 4G9 showed the most reduced degraded area as compared to scFv clone 8.

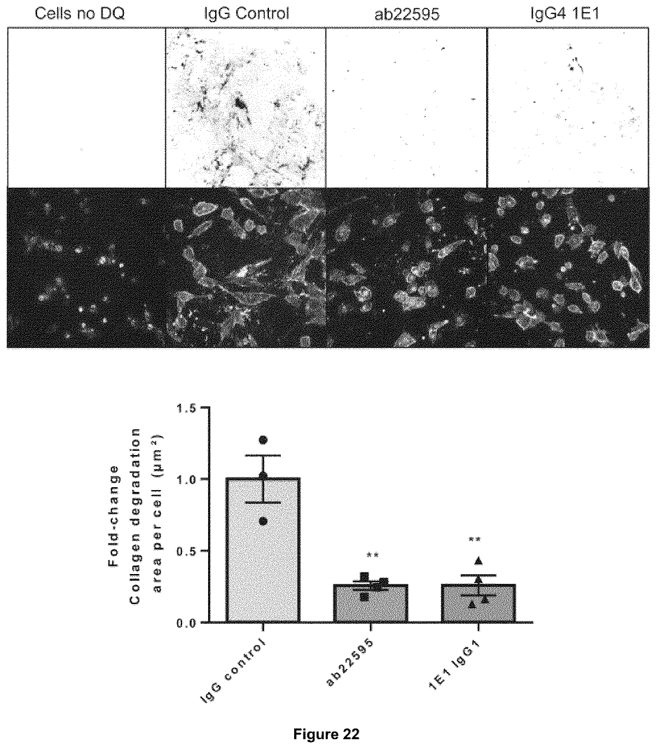

Test of inhibition of degradation was also performed with Collagen DQ degradation assay. IgG negative control treated 3T3-v-Src mouse cells produced DQ released fluorescent collagen as revealed by green fluorescent signal (FIG. 22). Treatment of cells 48H with positive CNX control ab22595 or IgG4 1E1 at 20 μg per ml inhibited significantly the release of DQ collagen green signal.

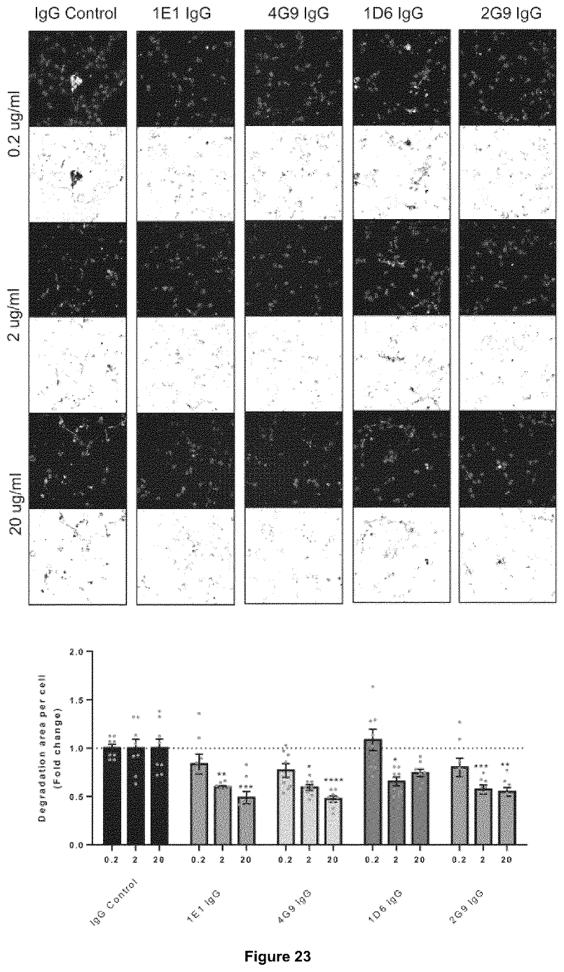

1E1, 4G9, 1D7 and 2G9 IgG were tested using DQ collagen assay with 3T3-v-Src mouse cells for 48H at different dilution 20, 2 and 0.2 μg/ml (FIG. 23). IgG negative control treated 3T3-v-Src mouse cells produced DQ released fluorescent collagen as revealed by green fluorescent signal. Treating 3t3-vSrc cells with 1E1, 4G9, 2G9 and 1D6 IgG at 20 μg/ml showed a statistically significant reduction in DQ degradation. Treating 3t3-vSrc cells with 1E1, 4G9, 2G9 at 2 μg/ml showed a statistically significant reduction in DQ degradation.

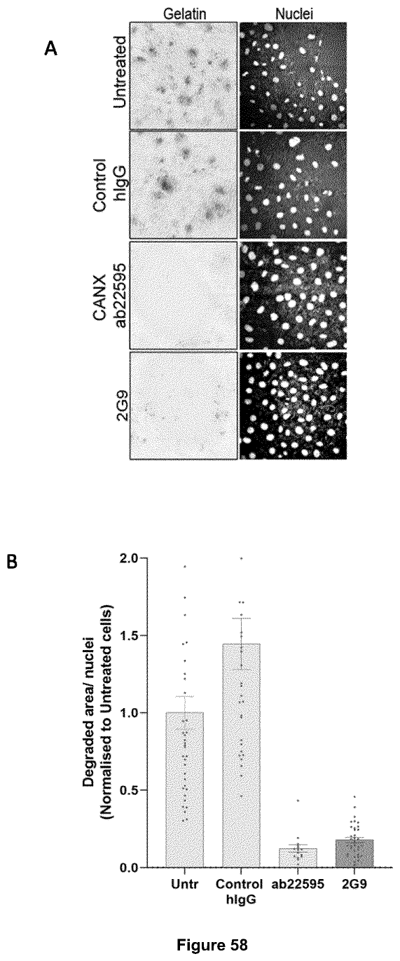

In further experiments, the ability of 2G9 in IgG1 format to prevent ECM degradation was evaluated using the fluorescent gelatin layered assay, as described above. The results are shown in FIGS. 58A and 58B. While the control IgG had no effect on matrix degradation or even seemed to stimulate it, 2G9 was able to reduce by 90% ECM degradation compared to untreated controls (FIG. 58B).

### IgG4 Forms of Antibodies Also Protect ECM

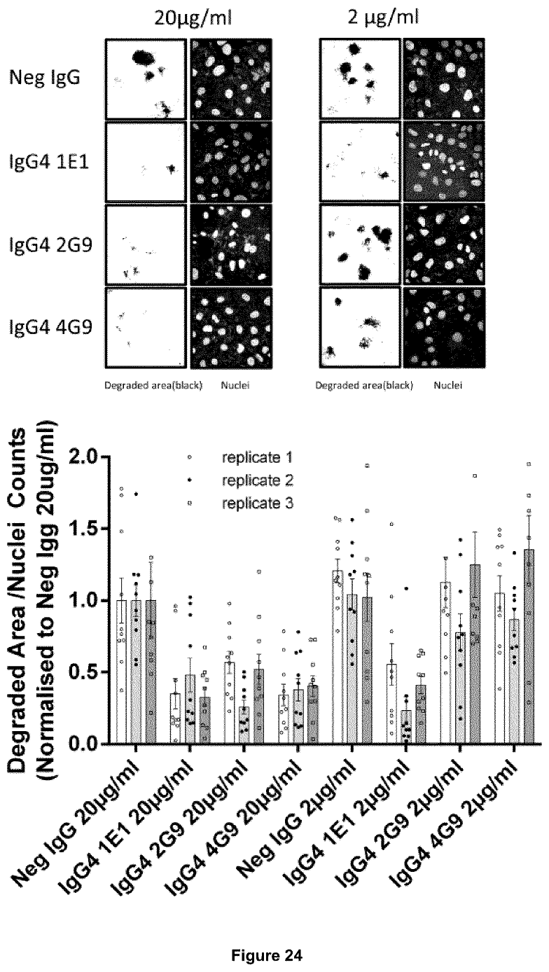

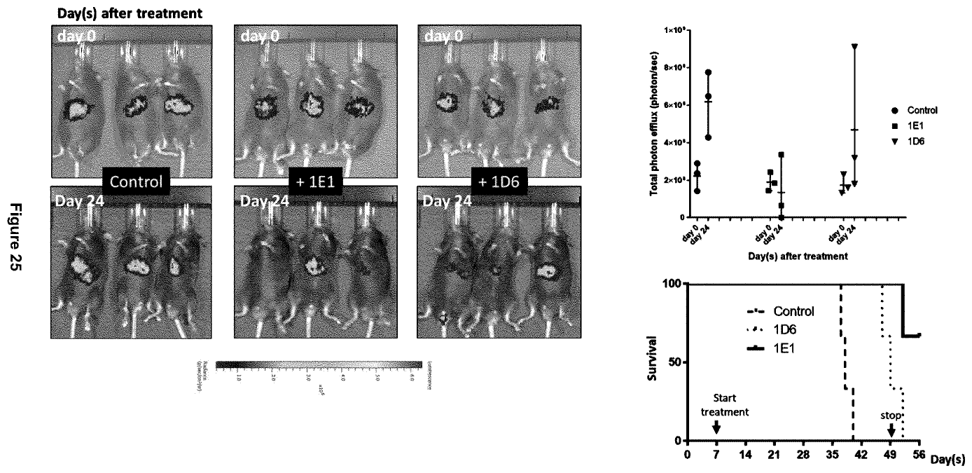

Isotype switched Igg4 1E1, 2G9 and 4G9 was tested with fluorescent gelatin layered assay at 2 dilutions: 20 μg/ml and 2 μg/ml on Huh7 Cells for 48 hours (FIG. 24). A reduction in degraded area was observed with IgG4 1E1, 2G9 and 4G9 as compared to negative IgG control at 20 μg/ml but only 1E1 showed ability to reduce degraded area as compared to negative IgG control when tested at 2 μg/ml.

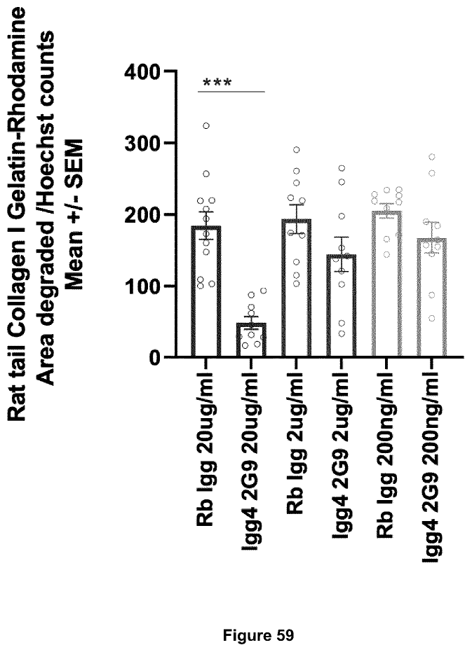

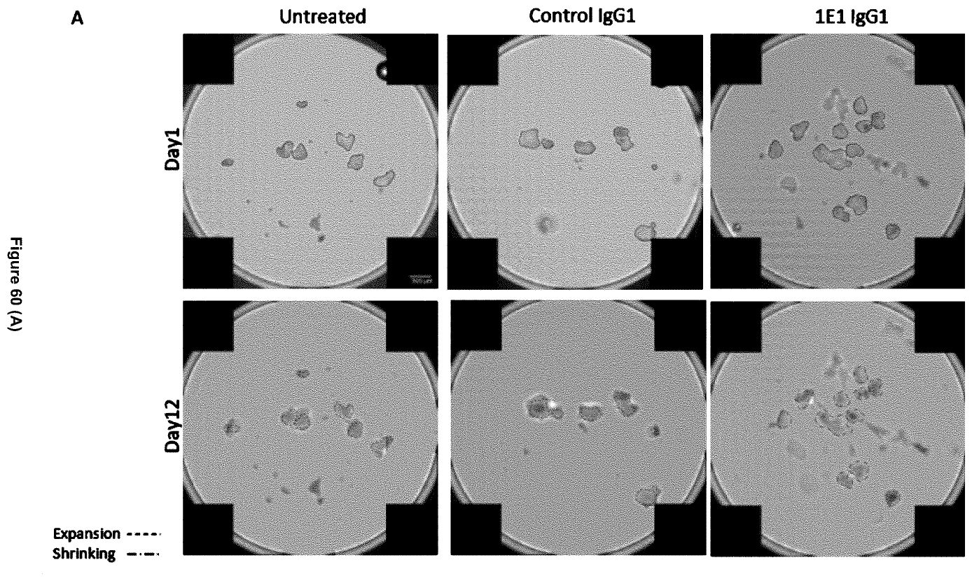

In further experiments, the ability of 2G9 to prevent ECM degradation in the fluorescent gelatin layered assay in the IgG4 format was evaluated. The results are shown in FIG. 59. 2G9 IgG4 was able to protect ECM degradation, with the effect most apparent at 20 μg/ml.

### Example 5: Anti-CNX Antibodies Reduce Tumor Growth and Metastasis, and Reduce Arthritis Pathology

### 5.1 CNX Antibodies Reduce Liver Tumor Growth

Hydrodynamic tail-vein (TV) injection is performed in 5-6-week-old C57BL/6J mice to deliver Sleeping Beauty (SB) plasmid system expressing both oncogenic Nras-G12V, Luciferase and a shRNA targeting the tumor suppressor gene p53. Each animal was injected only once. Plasmids were prepared using an EndoFree Maxi kit (Qiagen). The transposon/transposase mixture was diluted in lactated Ringer's solution (BRAun) in a volume corresponding to 10% of the body weight of the mouse being injected. Each animal received approximately 10 μg of transposase-encoding plasmid (pPGK-SB13) and 30 μg of pT2/shp53/PGK/Nras/Luciferase plasmid. Animals being injected were not anaesthetized but immobilized with a plastic restrainer. Sterile single-use 27-gauge needles were used. Injection was completed via the lateral tail vein in less than 8 s. Mice at 7 day post-injection (d.p.i.) of Nras/shp53-induced liver tumors were used for treatment. Mice were given an intraperitoneal (i.p.) injection of anti-CNX IgG1 antibody (250 μg per mouse) or anti-EBOLA IgG1 as control. Repeat i.p. injections of the antibodies were given every 2-3 days. Mice were monitored twice a week for general health and tumor burden. At desired time points, mice were imaged by IVIS® Spectrum imaging system. Prior to imaging, the animal will be placed in an induction chamber and anesthetized by isoflurane (oxygen at 1-2 L/min and isoflurane at 2-3%). When the animal is in a moderately deep plane of anesthesia, it is injected IP with 150 mg of luciferin per kg of body weight (typically, 100 to 200 μL of luciferin substrate at 50 mg/mL concentration in PBS per animal). Incubation time is about 10 minutes. In vivo images will be acquired at 1s exposure time and analyzed with the IVIS Living Image software package.

In vivo visualization of liver tumor growth in different treated groups at day 0 and day 24 was performed to assess the ability of CNX antibodies to reduce tumor growth. Quantification of total photon flux from liver tumor expressing oncogenic shp53/Nras/Luciferase was used to generate quantitative data (FIG. 25). Survival analysis of mice injected with CNX antibodies was also compared to Control (FIG. 25). Data show a reduction of tumour growth and improvements in survival data for CNX antibodies (1E1 and 1D6) compared with the control.

### 5.2 CNX Antibodies Reduce Cancer Cell Metastasis to the Lungs

MDA ER-G2 cells (2 million per mouse) were injected in the tail vein of 6-week-old nude BALB/c mice. At 7 d.p.i., mice were given an i.v. injection of anti-CNX IgG1 antibodies (1E1, 1D6 250 μg per mouse) or anti-EBOLA IgG1 as control. Mice were given i.v. injections of antibodies every 3 days. Mice were closely monitored and necropsied at desired time points.

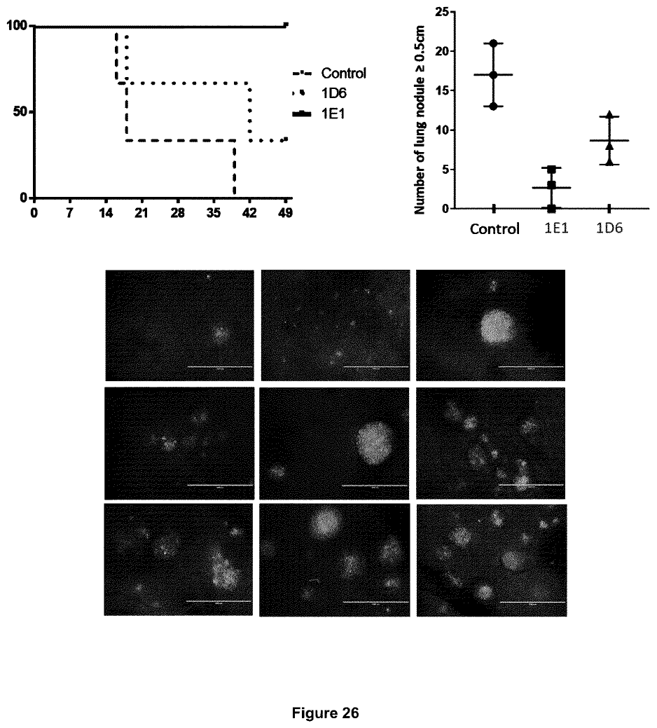

Results show an improved rate of survival at 49 days for CNX antibodies (1E1 and 1D6) compared with the control (FIG. 26). It was also shown that the number of lung nodules in mice treated with CNX antibodies (1E1 and 1D6) is reduced compared with the control treated mice (FIG. 26).

### 5.3 CNX Antibodies Accumulate in Tumors In Vivo

This assay employs the injection of NIH/3T3vSrc versus NIH/3T3vSrc CNX CALR knock-out (KO). Subcutaneous injection of 0.1 to 0.2 ml ~5×106 cells mixed with Matrigel Basement Membrane Matrix (thaw on ice) at a ratio 2:1. The tumor cells were implanted into the flank of mice, and tumor growth was monitored by calipering. At day 10 post injection, mice were given an i.p. injection of anti-CNX 1E1 IgG1 antibodies (750 μg per mouse). Mice were given i.p. injections of antibodies every 2 days for 3 times. Mice were sacrificed at 16 h after the last injection and different tissues were collected for anti-CNX accumulation analysis.

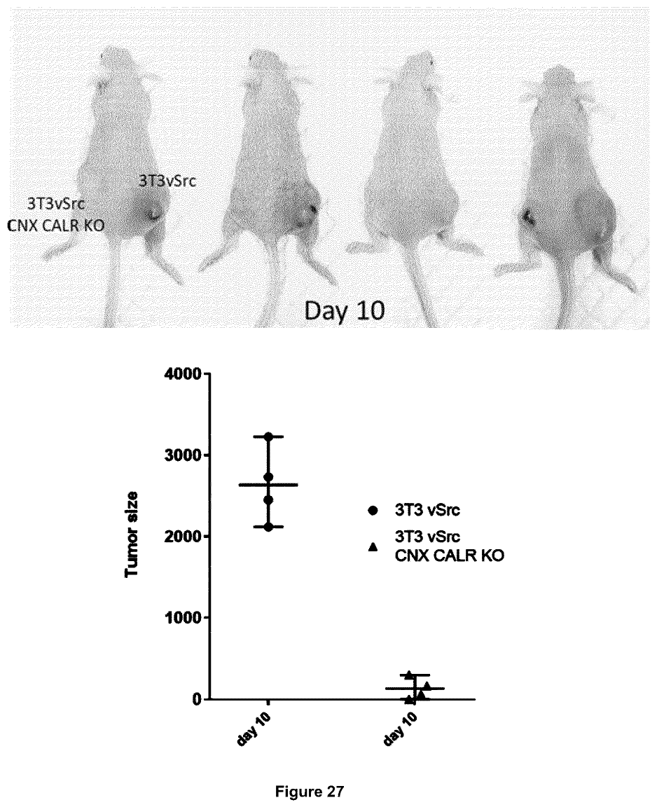

Comparison of subcutaneous tumor growth at day 10 in mice injected with both NIH/3T3vSrc (right flank) and NIH/3T3vSrc CNX CALR KO (left flank) was performed, with results being shown in FIG. 27. Subcutaneous tumor growth was considerably lower in the left flank (NIN/3T3vSrc CNX CALR KO) compared with the right flank (control: NIH/3T3vSrc) (FIG. 27).

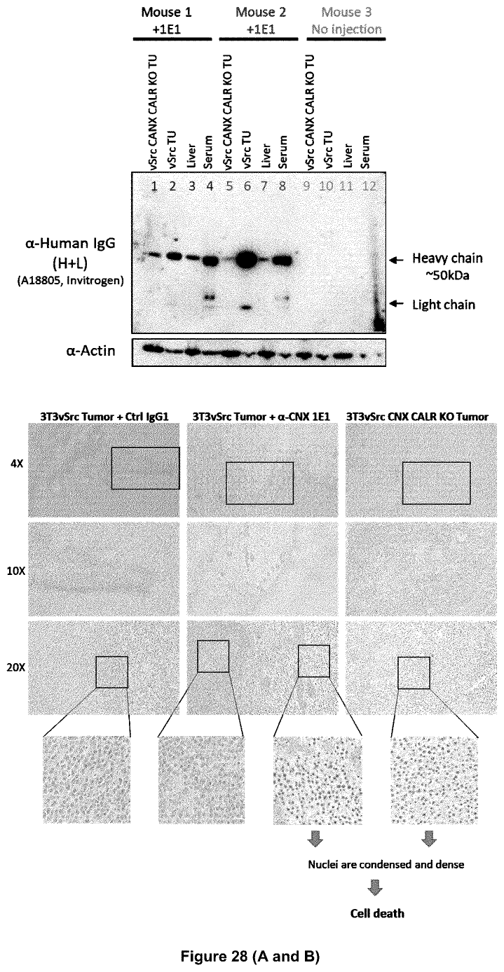

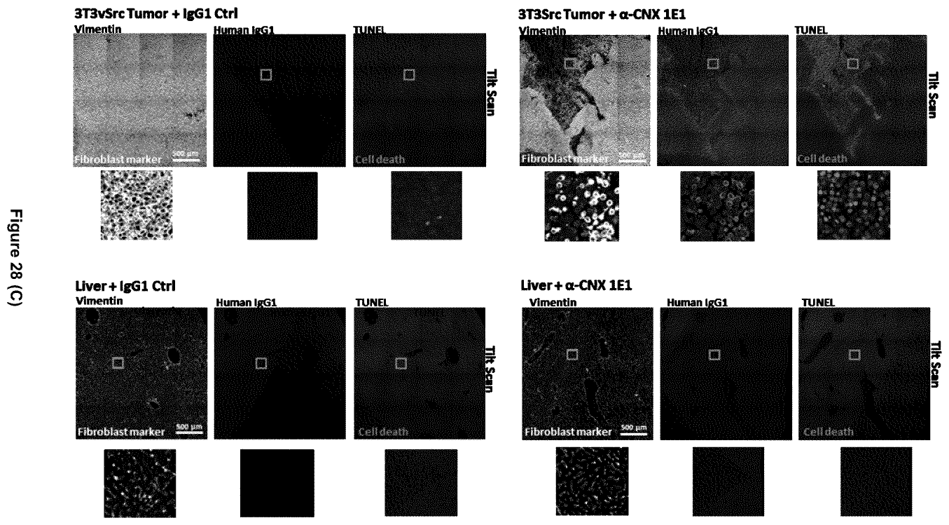

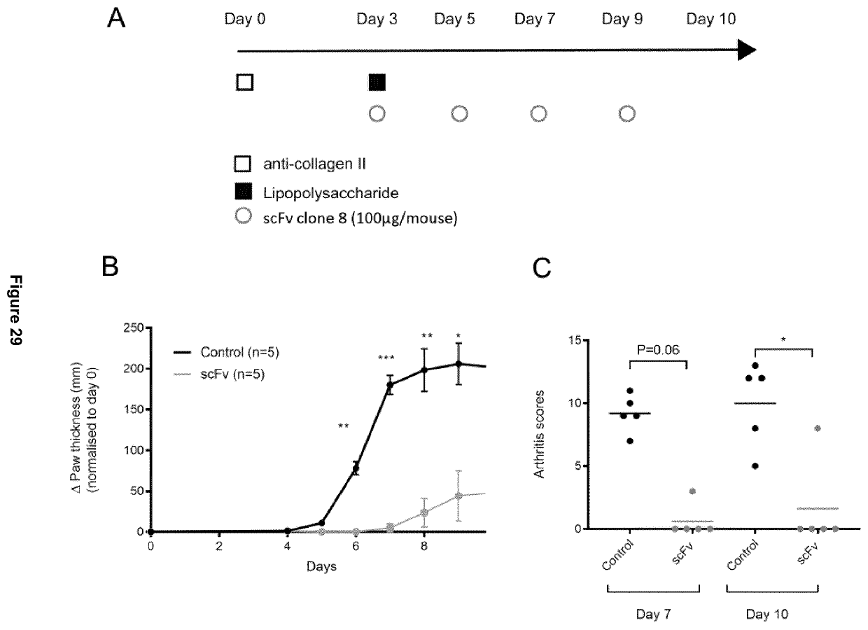

Immunoblot analysis of human IgG shows that CNX antibodies significantly accumulate in 3T3vSrc tumor tissues as compared to other tissues (FIG. 28).

### 5.4 Anti-CNX Antibodies Reduce Arthritis Symptoms

scFV clone 8 was analysed in the CAIA mouse model. C57BL6 mice received an injection of collagen antibody on day 0 and were then injected with LPS on Day 3 and received few hours later on Day 3 an intraperitoneal injection of recombinant scFv clone 8 at 100 μg/mouse or PBS in control situation (FIG. 29A). The scFv was further injected at day 5, and 7 and 9. At day 6 and 7. Control mice developed an increased thickness in the paws characteristic of inflamed joints in control mice. The scFv clone 8 injected mouse showed no detectable thickness increase at day 6 and 7 (FIG. 29B). At day 7 arthritis score reached almost significant difference for scFv group as compared to control group (P=0.06) (FIG. 29C). At day 8 and 9 control and scFV injected mouse developed increased thickness in paws but scFv group displayed significantly less thickness in the paws as compared to control group (FIG. 29B). At Day 10, the scFv group showed a significantly reduced arthritis score as compared to control group (FIG. 29C).

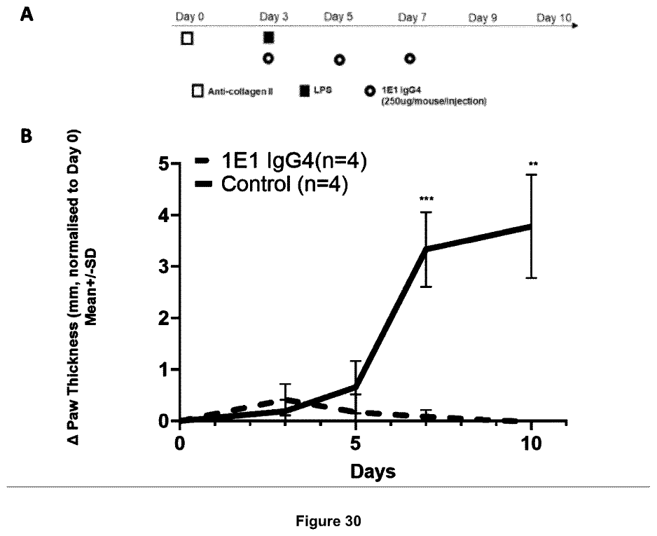

We tested IgG4 1E1 in the CAIA mouse model. C57BL6 mice received an injection of collagen antibody on day 0 and were then injected with LPS on Day 3 and received few hours later on Day 3 an intraperitoneal injection of recombinant scFv clone 8 at 250 μg/mouse or PBS in control situation (FIG. 30A). IgG4 was further injected at day 5, and 7. At day 5, 7 and 10 control mice developed an increased thickness in the paws characteristic of inflamed joints in control mice. IgG4 1E1 injected mouse showed minimal paws thickness increase at day 7 and 10 (FIG. 30B).

### Example 6: Therapeutic Utility of Anti-CNX Antibodies for the Treatment/Prevention of Diseases Characterised by Cartilage Degradation

### 6.1 Materials and Methods

### Patient Samples, Cell Lines and Mouse Strains

Patient samples: synovial tissue specimens were obtained from patients with rheumatoid arthritis (RA) or osteoarthritis (OA), who were undergoing joint replacement surgery at the Tan Tock Seng Hospital (Singapore). The procedures were approved by the Ethics Committee of The National Healthcare Group domain specific review board under protocol no. 2018/00980. All patients were given written consent and met the diagnostic criteria for rheumatoid arthritis or osteoarthritis.

Cell lines: The SW982 cells (ATCC HTB-93) are synovial fibroblast cells derived from synovium of a patient with synovial sarcoma. SW982 cells were maintained in Leibovitz's L-15 Medium (Gibco; ThermoFisher Scientific) supplemented with 10% (v/v) foetal calf serum (FCS) and 1% (w/v) penicillin/streptomycin (Gibco; ThermoFisher Scientific) at 37° C. in a free gas exchange with atmospheric air. The SW982 cells were engineered to stably express a doxycycline-inducible gene encoding the ER-2Lec using the Sleeping Beauty transposon system.

Mice: Col6a1Cre mice expressing collagen type VI promoter driven on the C57BL/6J background were provided by G. Bressan (University of Milano, Milano, Italy). It is noted that comparable murine models known in the art can be used as a background to generate the mice required according to the present disclosure. ER-2Lec mice on the same background expressing an endoplasmic reticulum (ER)-localized double-lectin domain (ER-2Lec) under the control of a loxP-flanked STOP cassette were custom generated according to specifics provided by the inventors by Ozgene. Col6a1Cre ER-2Lec mice were generated by crossing Col6a1Cre mice with ER-2Lec mice, resulting in mice expressing ER-2Lec in the mesenchymal cell lineages. All animals were bred and maintained under specific pathogen-free conditions in micro-isolator cages with access to food and water at Biological Resource Centre (ASTAR, Singapore). Experiments were performed using age- and sex-matched animals and complied with guidelines approved by the Animal Ethics Committees at Biological Research Centre (ASTAR, Singapore) under the protocol IACUC no. 201548.

Isolation of Primary Synovial Fibroblasts (SF) from Human Tissues

Synovial tissues from osteoarthritis (OA) and rheumatoid arthritis (RA) patients were obtained at the time of synovectomy or synovial biopsy. Following excision, the tissues were immediately minced and digested in collagenase IV (1 mg/ml, Gibco) in Dulbecco's modified Eagle medium (DMEM) for 1.5 hours at 37° C. with gentle agitation. The mixture was passed through a 70 μm mesh cell strainer and cell pellet was obtained after centrifugation at 250 g for 10 minutes. Human synovial fibroblasts from osteoarthritis and rheumatoid arthritis patients (OASF and RASF, respectively) were used between passages 3 and 9 (Rosengren et al., 2007, Methods in Molecular Medicine, Vol. 135: Arthritis Research, Volume 1). The purity of culture was confirmed by staining with fibroblast cell identity marker CD90 prior to performing experiments. Normal human synovial fibroblasts were derived from synovial tissues from a healthy human donor (HCSF) and obtained from the Cell Applications, Inc. HCSF were cultured in complete in DMEM supplemented with 10% (v/v) foetal calf serum (FCS) and 1% (w/v) penicillin/streptomycin (Gibco; ThermoFisher Scientific) at 37° C. in a humidified atmosphere containing 5% CO₂.

### Reagents

Antibodies: Anti-CNX (ab10286, ab22595), Anti-Vimentin (ab92547), anti-beta actin (ab8226) antibodies were purchased from Abcam. Agarose-bound Vicia villosa Lectin (VVL, AL-1233) was purchased from Vector Laboratories. PE.C7 conjugated anti-CD45 and PE conjugated anti-CD90 were purchased from Biolegend. Anti-FAPα was purchased from R&D systems. Anti-NEM OX133 antibody was purchased from Absolute Antibody. Anti-rabbit IgG-HRP antibody and anti-mouse IgG-horse radish peroxidase (HRP) antibody were purchased from GE Healthcare Life Sciences.

Plasmids: The plasmid expressing doxycycline inducible ER-2Lec was generated as previously described (Gill et al. 2013, PNAS, 2013, E3152-E3161). The resulting vectors, together with the pPGK-SB13 expressing Sleeping Beauty 208 transposase, were used to transfect SW982 cells.

### Immunofluorescence Staining

Formalin-fixed, paraffin-embedded tissue sections were deparaffinized in xylene substitute buffer (Sub-X, Leica Biosystems) and rehydrated. For mouse joint tissue and tissue microarray (provitro AG, Berlin, Germany), antigen retrieval was performed by immersion in Epitope Retrieval Solution pH 6 (Leica Biosystems) and incubated at 60° C. in an oven for 18 hours. For human tissue specimens, antigen retrieval was performed using Epitope Retrieval Solution pH 6 (Leica Biosystems) in a pressure chamber (2100 Retriever, Akribis Scientific Limited, WA16 0JG, GB). Sections were washed twice with phosphate buffered saline (PBS) and immersed in a blocking buffer (5% horse serum, 1% Triton X100). After 1 hour, sections were incubated overnight at 4° C. with a primary antibody mix containing primary antibodies including rabbit anti-CD45, rabbit anti-Vimentin, rabbit anti-CNX, rat anti-FAPα, or biotin conjugated Vicia villosa lectin (VVL). Samples were washed three times with blocking buffer and incubated for 2 hours with the corresponding secondary antibodies including anti-rat Alexa Fluor647, anti-rabbit Alexa Fluor 594-conjugated or Alexa Fluor488 streptavidin (ThermoFisher Scientific, 1:400). After washing, cell nuclei were stained with Hoechst 33342 (ThermoFisher Scientific; 1:1000) for 5 minutes before mounting. All images were captured using the same settings on the LSM-700 (Zeiss) confocal microscope. Raw integrated density of VVL and nuclear signals were measured by FIJI image calculator.

### Collagen Antibody-Induced Arthritis

On day 0, mice were injected with an antibody cocktail mix containing five different monoclonal antibody clones against collagen type II proteins (Chondrex Inc.) at a dose of 2 mg/mouse (200 μl) via intraperitoneal (IP) injections. On day 3, animals were injected with LPS at 50 μg/mouse via IP injections (100 μl). From day 3 on, arthritis severity was assessed daily by measuring paw thickness using a digital calliper. Assessment was also done by a blinded researcher using a qualitative clinical scoring system provided by Chondrex, Inc.

### Histology Analysis and Cartilage Extracellular Matrix Staining

After removing skin, both front and hind paws were formalin-fixed and embedded in paraffin. Tissues were de-paraffinized and rehydrated as described above in the above immunofluorescent staining protocol. Tissue sections were stained with Haematoxylin and Eosin (HE), Safranin-O (SO), and Alcian Blue (AB) stains. Slides were scanned at 20× using a Leica SCN400 slide scanner (Leica Microsystems, Germany). Images were exported to Slidepath Digital Image Hub (Leica Microsystems, Germany) for viewing. Selected regions were analysed using Measure Stained Area Assay of Slidepath Tissue Image Analysis 2.0 software (Leica Microsystems, Germany). A quantitative analysis of cartilage extracellular matrix (ECM) staining areas was performed using FIJI image calculator.

### Flow Cytometry

Skin from the feet was removed and the joints were cut 3 mm above the heel. To avoid contamination with the bone marrow, bone marrow cavity in the tibia was thoroughly flushed with Hank's balanced salt solution (HBSS). The joints were cut into small pieces and incubated in digestion buffer (1 mg/ml collagenase IV and 1 mg/ml of DNase I in HBSS) for 60 minutes at 37° C. Cells released during the digestion were filtered through 70 μm cell strainers; erythrocytes were lysed using a red blood lysis buffer (BD Biosciences). The cells were stained with live/dead Aqua (Invitrogen) viability dyes, incubated with Fc Block at 1:50 (BD Biosciences), and stained with fluorochrome-conjugated antibodies including PE-conjugated anti-CD90 (Biolegend), PECy7-conjugated anti-CD45 (Biolegend) and FITC-conjugated Vicia villosa lectin (VVL; Life Technologies) for 30 minutes. For intracellular staining, cells were fixed by using BD Cytofix/Cytoperm solution (BD Biosciences). Fixed cells were permeabilized in 1×BD Perm/Wash buffer (BD Biosciences) prior staining with FITC conjugated Vicia villosa lectin (VVL). Samples were acquired on FACS BD LSRII and analysed using Kaluza softwares.

Western Blot and VVL-Coimmunoprecipitation (VVL-CoIP) Cells were seeded at 2×10⁵ cell/ml in 10 cm dishes pre-coated with 2 mg/ml cartilage extracellular matrix (ECM; Xylyx Bio.) and left to rest overnight. After stimulation with 100 μg/ml TNFα (PeproTech) and 100 μg/ml IL-1β (PeproTech) for 24 hours, cells were harvested and lysed in a low-stringency RIPA lysis buffer (50 mM Tris, 200 mM NaCl, 0.5% NP-40, Complete and PhoStop inhibitor [Roche Applied Science]) and lysed for 30 minutes at 4° C. Lysates were then clarified by centrifugation at 13000 g for 10 minutes at 4° C. Clarified tissue lysates were incubated with agarose-bound Vicia villosa lectin (VVL) beads (Vector Laboratories) overnight at 4° C. Beads were washed three times with RIPA lysis buffer, and the precipitated proteins were eluted in 2×LDS sample buffer containing 50 mM DTT. Lysates were boiled at 95° C. for 5 minutes and separated by SDS-PAGE electrophoresis using 4-12% Bis-Tris 80 NuPage gels (Invitrogen) at 180 V for 70 minutes. Samples were then transferred on nitrocellulose membranes using iBlot transfer system (Invitrogen) and blocked using 3% bovine serum albumin (BSA) dissolved in TBST (tris-buffered saline (TBS) and Polysorbate 20 (also known as Tween 20)-50 mM Tris, 150 mM NaCl and 0.1% Tween-20) for 1 hour at room temperature. The nitrocellulose membranes were then incubated with primary antibodies (1/1000 diluted in 3% BSA-TBST) overnight at 4° C. The next day, membranes were washed three times with TBST and incubated with secondary antibodies conjugated with horseradish peroxidase (HRP) for 2 hours at room temperature. Membranes were washed three more times with TBST before electrochemiluminescence (ECL) exposure.

### Matrix Degradation Assay

Red gelatine coverslips were prepared as previously described (Ros et al., Nat Cell Biol, 2020, vol 22, November 2020, 1371-1381). The coverslips were coated with 0.2 mg/ml cartilage extracellular matrix (ECM; Xylyx Bio) for 3 hours at 37° C. Synovial fibroblasts cells (including SW982 cells, osteoarthritis synovial fibroblasts (OASF), rheumatoid arthritis synovial fibroblasts (RASF) and healthy human donor synovial fibroblasts (HCSF)) were seeded at 5×10⁴ cells/ml/well in 24-well plates overnight. Cells were then stimulated with 100 μg/ml TNFα and 100 μg/ml IL-1β. After 24 hours, the cells were fixed with 4% paraformaldehyde (PFA) and stained for nuclear using Hoechst 33342 (Life Technologies). Stained coverslips were mounted onto glass microscope slides and 10 to 30 images were acquired for each condition. The normalized area of matrix degradation relative to the number of cells was measured by using ImageJ software as previously described. (Martin et al., J Vis Expr, 2012, vol 66, e4119) Briefly, the area of degradation using the fluorescent gelatine images after thresholding and the same threshold were applied for all images. The cell counter tool was used to count the number of nuclei and the area of gelatine degradation per total number of cells was calculated. Experiments were done in three biological replicates.

### Statistical Analyses

GraphPad Prism (version 8.4.3, GraphPad Software, CA, USA) was used for statistical analyses and graphical preparation. Data analysis was performed by one-way (Kruskal Wallis test), two-way ANOVA (Tukey's multiple comparisons test) or Mann-Whitney test as indicated. Differences were considered statistically significant for p-values <0.05.

### 6.2 Results

### Enhanced O-Glycosylation in Rheumatoid Arthritis and Osteoarthritis Synovium

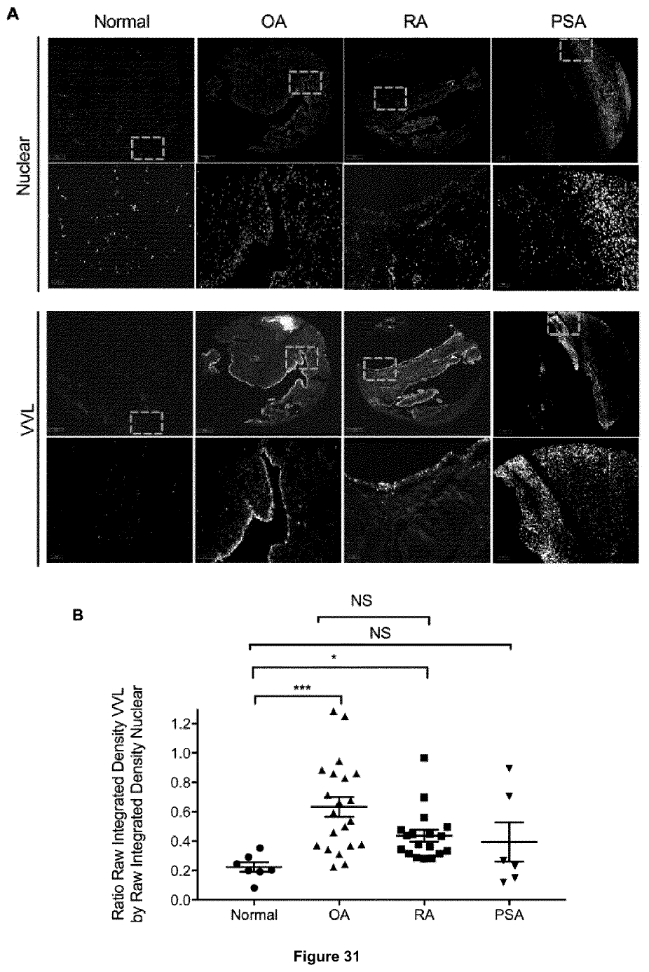

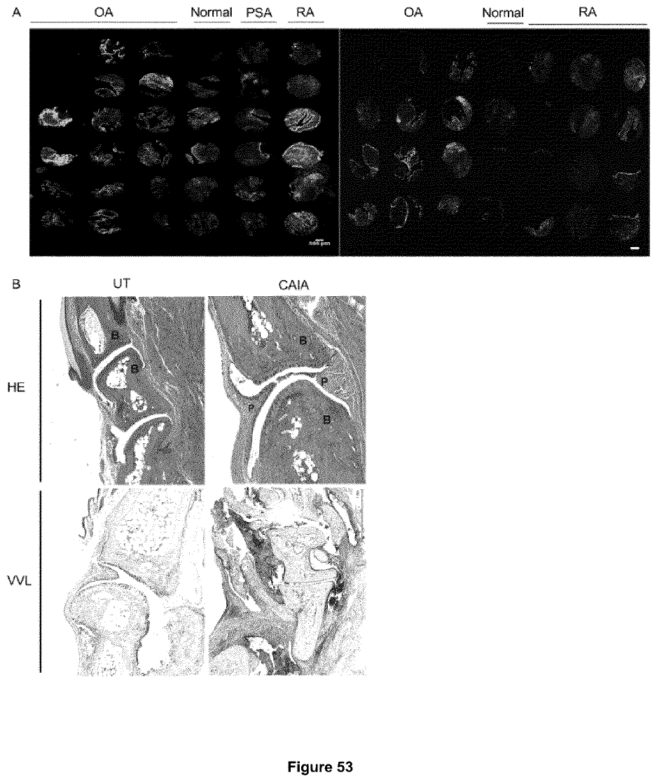

A hallmark of GALA is increased cellular levels of Tn (T nouvelle), the O-glycan formed by the addition of a GalNac to a Ser or Thr residue. Tn can be detected by Tn-binding proteins such as Vicia villosa Lectin (VVL) and Helix Pomatia Lectin (HPL) (Gill, et al., Proc. Natl. Acad. Sci. U.S.A 110, E3152-61 2013). We analysed microarrays of joint tissues by immunofluorescence using VVL and counterstained for DNA. A distinct increase was detected in Tn samples in 18/21 samples from OA patients, 2/6 samples from patients with Psoriasis arthritis and 9/18 samples of RA patients (FIG. 31A and FIG. 53). The integrated fluorescence intensity of VVL staining was quantified and normalised to the DNA signal intensity as a marker of cell density (FIG. 31B). While healthy patient samples showed little variation, most samples of RA and OA samples displayed enhanced Tn levels, with in some areas with up to seven-fold increase of VVL signal.

To further characterise GALA in arthritis, a mouse model of RA based on Collagen Antibody Induced Arthritis (CAIA) was used. Briefly, animals were injected with an antibody against collagen type II, then 3 days later with lipopolysaccharide (LPS) and developed symptoms starting at day 5. Initial immunohistochemistry analysis revealed a marked increase in the levels of Tn in and around the joint in animals with arthritis (FIG. 53).

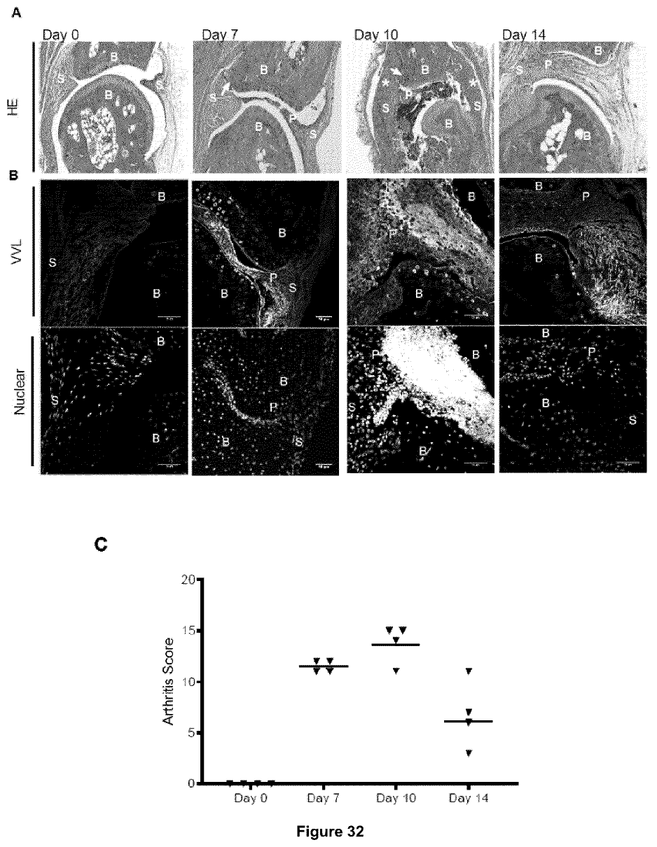

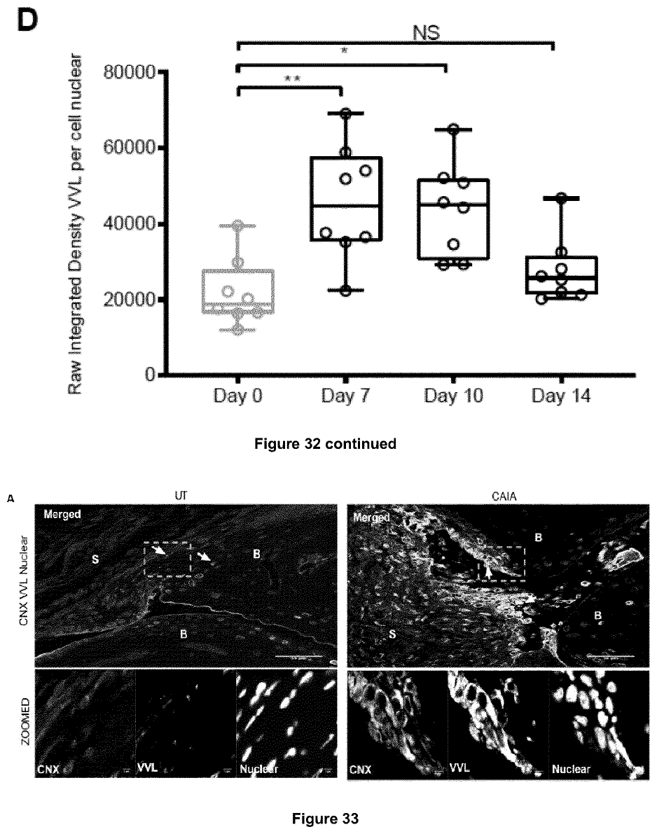

In the CAIA model, the symptoms took about 7 days to reach a peak and lasted for 10 days before slowly decreasing. Animals were sampled on day 0, 7, 10 and 14 and used immunofluorescence staining to quantify Tn levels. Histologically, a pannus invading the joint cavity was apparent on day 7 and increased in size with the influx of immune cells on day 10. By day 14, the amount of immune cells had drastically diminished but synovial tissue remained in the joint cavity (FIGS. 32A and 32B). High levels of Tn were observed in cells invading the joint cavity at day 7 and persisted till day 10 (FIGS. 32C and 32D). By day 14, cellular Tn levels had subsided in a large fraction of the animals. Some Tn staining was observed in fibrous material that was devoid of cells, and this staining did not decrease significantly at day 14.

High Tn Levels in the Arthritic Synovium are Consistent with GALA Activation

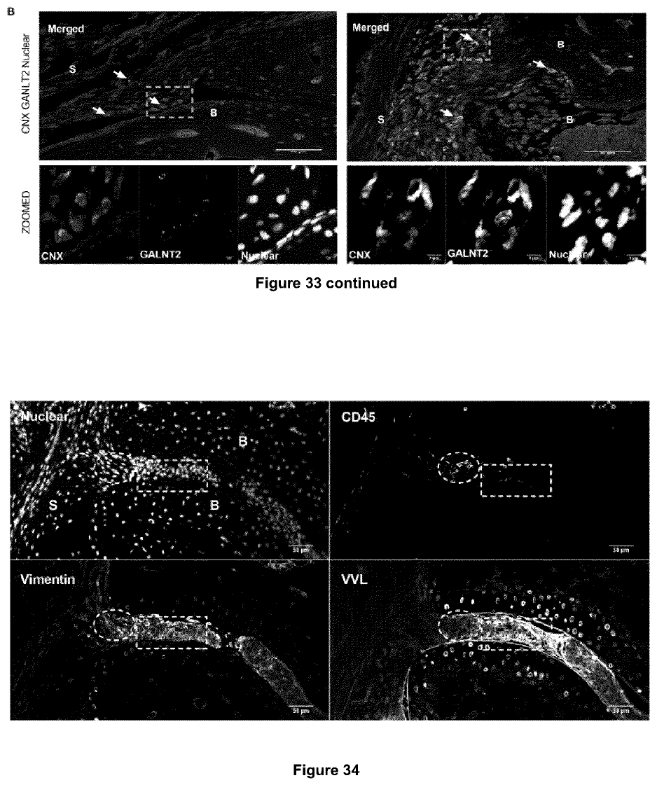

High cellular Tn levels can be induced by the GALA pathway, which is characterised by abundant Tn signal in the ER. Because the ER is distributed throughout the cell body, GALA activation typically shows up as an increased diffuse signal (Bard et al., Trends Cell Biol. 26, 379-388, 2016). CAIA synovium samples were co-stained for Tn and CNX, an ER marker (FIG. 33A). In untreated samples, Tn and CNX staining were clearly separated, with Tn concentrated in a perinuclear pattern consistent with the Golgi. By contrast, in day 7 CAIA samples, Tn staining was elevated, filling the cellular space and colocalizing with the ER marker CNX, strongly suggestive of GALA induction. To corroborate GALA induction in the CAIA context, GALNT2 staining was employed, a ubiquitously expressed GALNT transferase previously shown to be relocalized to ER in cancer with GALA activation. A similar change in localisation from perinuclear in untreated sample to ER pattern in CAIA sample with colocalization with CNX was observed (FIG. 33B). Results suggest the relocation of GALNT2 from the Golgi to ER as the basis of Tn levels increase in CAIA context. These results are in line with the previously reported description of GALA phenotype in breast and liver cancer (Gill, et al., Proc. Natl. Acad. Sci. U.S.A 110, E3152-61, 2013; Nguyen, et al., Cancer Cell. 32, 639-653.e6, 2017).

### Synovial Fibroblasts are the Major Cell Type Displaying GALA

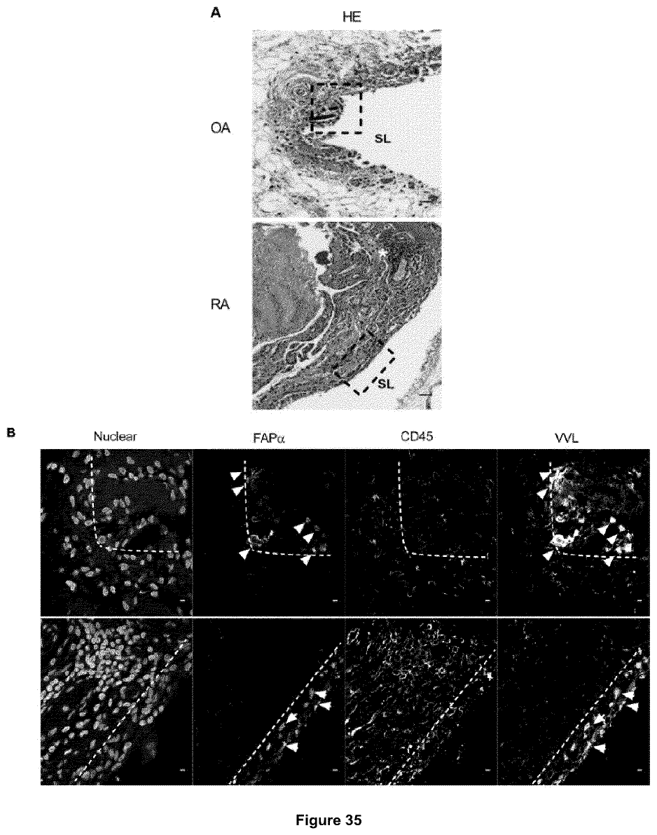

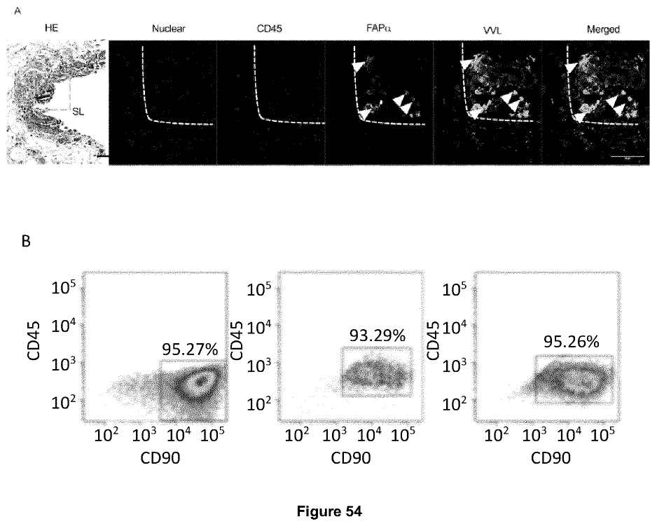

The synovium in RA disease is a complex tissue comprising immune cells and synovial fibroblasts (Choy et al., Rheumatology. 51 Suppl 5, v3-11, 2012). To establish which cell type displayed increased GALA, 7d mouse CAIA joint samples were co-stained with VVL, CD45 a marker of immune cells and vimentin a marker of fibroblasts. Vimentin positive cells were observed at the forefront of the invading panus (FIG. 34). CD45 positive cells were typically clustered and located behind the invading front of the pannus. Remarkably, Tn positive cells in the pannus largely co-stained with vimentin positive region and not CD45 positive region. This result suggests GALA activation in synovial fibroblast (FIG. 34). This result was validated in human samples of RA and OA, using the Fibroblast Activated Protein alpha (FAPα) as a marker of synovial lining fibroblasts (Bauer et al. Arthritis Res. Ther. 8, R171, 2006). The FAPα positive cells formed a layer located at the edge of the pannus, with a larger layer of CD45 positive cells behind in RA sample (FIG. 35A, 35B and FIG. 54A). In OA samples, the number of CD45 cells was reduced compared to RA samples but FAPα cells displayed similar VVL staining (FIG. 54). Strikingly, VVL staining co-localised exclusively with FAPα in both RA and OA conditions. Thus, lining synovial fibroblasts are the major cells displaying GALA activation in OA and RA synovium.

### Stimulation of SF by Cytokines and ECM Induces Higher GALA Levels

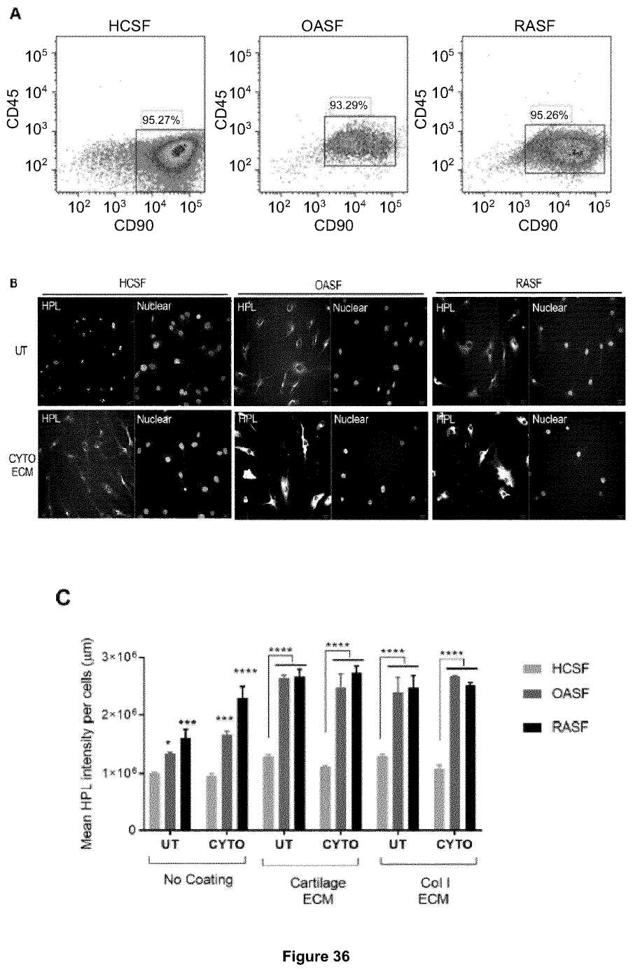

To understand how GALA is activated in vivo, primary human SFs derived from patients were analysed. FACS analysis using CD90 and CD45 as markers of SFs and immune cells respectively established the >90% purity of the cell preparations used (FIG. 54B). High content imaging analysis of Tn level in these SF patient derived cells was performed with HPL staining. Seeding cells on plastic wells showed increased levels of Tn in OA and even more in RA cells as compared to healthy SF (FIG. 36). Then, SFs were stimulated with TNFα and IL1β cytokines, which are thought to drive disease progression in RA (Kagari and Shimozato. J. Immunol. 169, 1459-1466, 2002). Individual cytokines had a relatively limited effect on GALA activation, however the combination of both cytokines (labelled CYTO) induced a 2 fold increase in Tn cellular levels. Interestingly, the effect was marked in RASF and more limited in OASF and almost nonexistent in healthy SFs (HCSF).

Next, it was considered whether cartilage ECM proteins might contribute to the activation of SF. Exposing SF to cartilage ECM activated GALA by up to three fold in both OASF and RASF. By contrast, HCSF cells were almost non-responsive. The combination of CYTO and ECM had an additive effect on Tn levels (FIG. 36). The stimulation was not specific for cartilage ECM as the use of rat tail derived collagen I induced a similar activation (FIG. 36).

Under unstimulated conditions, Tn staining was exclusively detected in the Golgi of HCSF, whereas OASF and RASF displayed additional ER-like Tn staining (FIG. 36). Upon stimulation with a combination of CYTO and cartilage ECM, the ER-localized Tn staining was enhanced in both OASF and RASF but not HCSF (FIG. 36).

To summarize, this analysis revealed that OA and RA patients' SFs have elevated GALA compared to healthy SFs in cell culture. RA SFs activate further GALA in response to cytokines, while both RA and OA SFs activate GALA upon exposure to ECM. By contrast, healthy SFs had very limited GALA responses, suggesting the pathway is primed for activation in patients' cells.

### Inhibition of GALA in SF Reduces ECM Degradation and Arthritis In Vivo

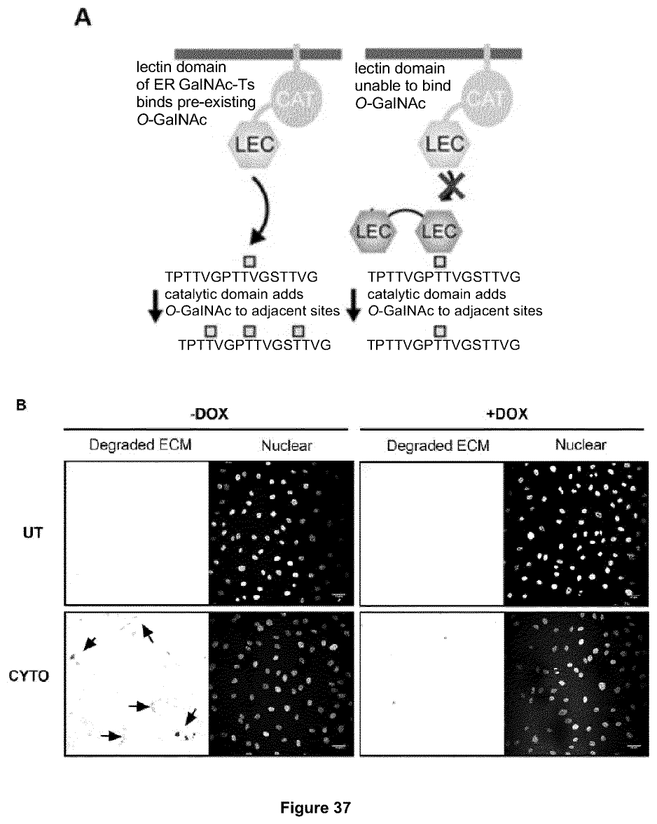

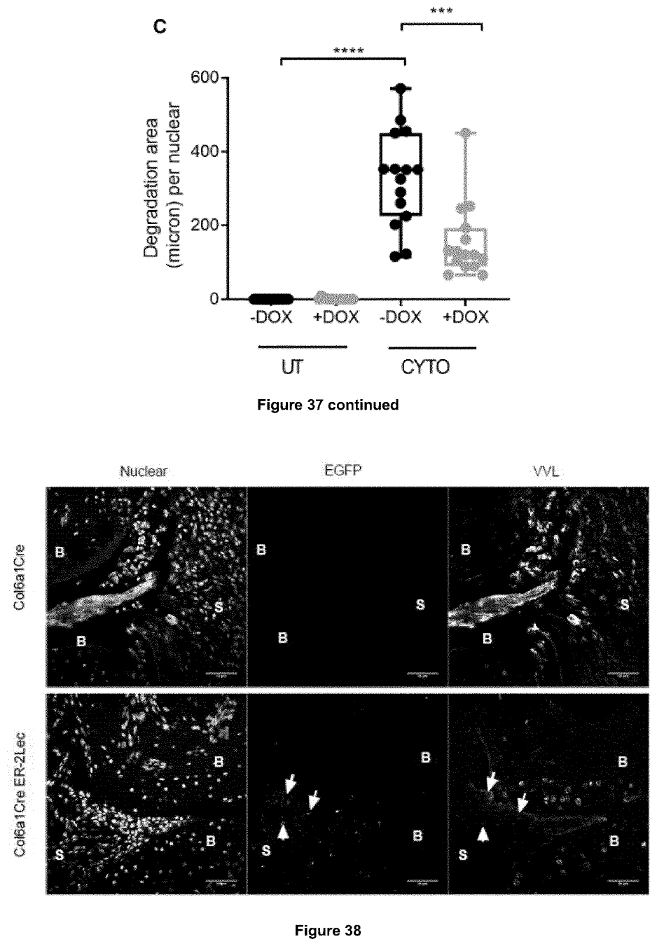

As GALA drives ECM degradation in cancer cells, it was assessed whether it is also implicated in cartilage ECM degradation by SFs. It was found that the human synovial sarcoma SW982 cells are able to degrade collagen (in a sandwich assay with fluorescent gelatin previously described) (Ros et al., Nat. Cell Biol. 22, 1371-1381, 2020). It has been described previously the ER-2Lec chimeric protein, composed of an ER-targeting sequence and a fusion of two lectins of GALNT2 (Gill, et al., Proc. Natl. Acad. Sci. U.S.A 110, E3152-61 2013). ER-2Lec inhibits specifically the activity of GALA by interfering with ER O-glycosylation. A stable SW982 transfectant with ER-2Lec under a Doxycycline inducible promoter system was generated. Similarly to RA SFs, SW982 cells stimulated with the CYTO mix were more active at degrading the ECM (FIGS. 37B and 37C). However, when ER-2Lec expression was induced, the degradation was reduced significantly, by more than 2-fold (FIGS. 37B and 37C).

To assess whether ER-2Lec could reduce arthritis in vivo, a transgenic mouse line with ER-2Lec with a Lox cassette was generated. The transgenic line was crossed with mice expressing Cre under the collagen VI alpha1 (Col6a1) promoter. Collagen VI is expressed by joint mesenchymal cells, and in particular SFs (Danks et al., Annals of the Rheumatic Diseases. 75, 2016, pp. 1187-1195; Armaka, et al., J. Exp. Med. 205, 331-337, 2008). Thus, this genetic cross is expected to result in ER-2Lec being expressed mostly in SFs and reduce Tn levels in arthritic mice. CAIA was induced in these mice and monitored ER-2Lec-GFP expression and Tn levels in the pannus after 7 days. Tn was markedly reduced, consistent with GALA inhibition (FIG. 38).

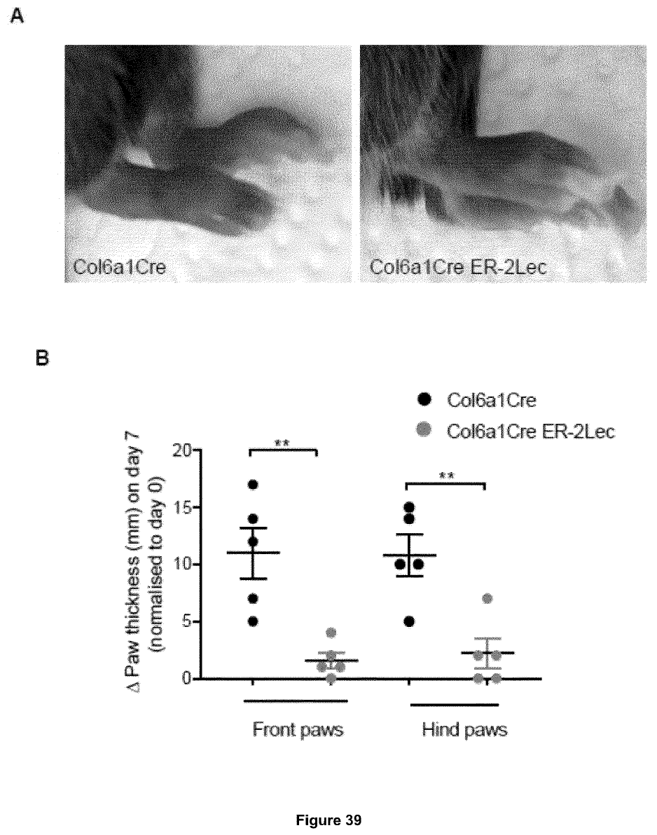

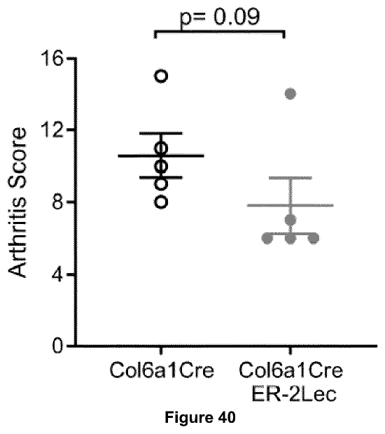

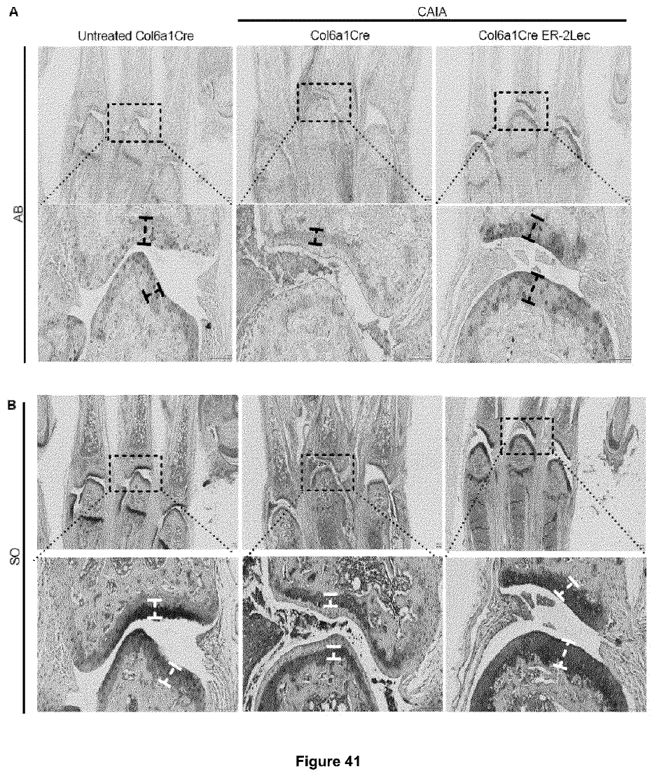

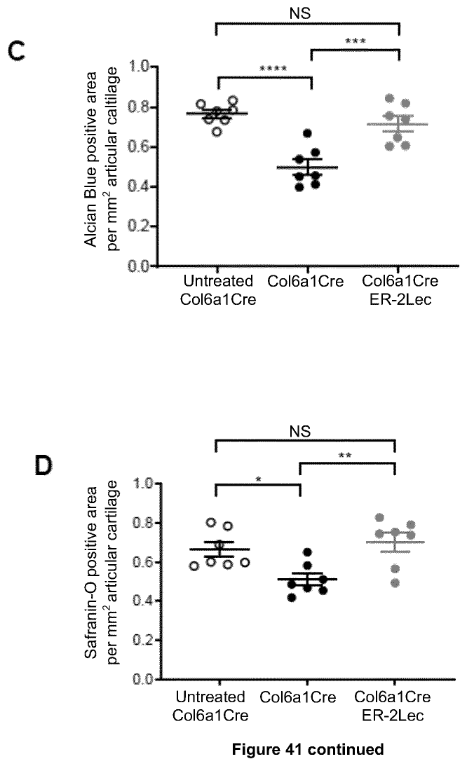

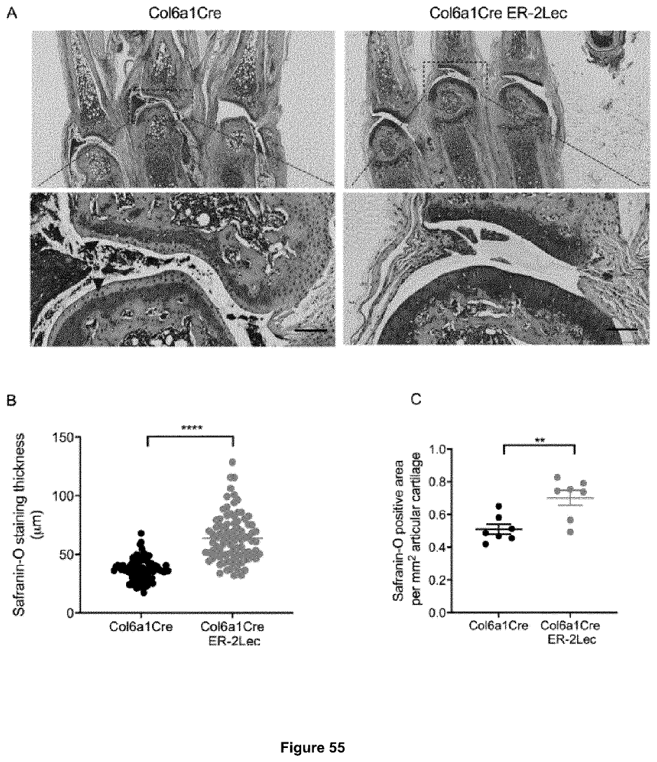

RA symptoms were monitored in Col6a1Cre ER-2Lec animals and in Cre expressing controls. The ER-2Lec expressing animals displayed a marked reduction of swelling in the paws (FIGS. 39A and 39B). Change in paw thickness overtime was monitored and significant reduced thickness in ER-2Lec animals as compared to controls was observed. The internationally defined arthritis score was used in a blinded evaluation and observed a consistent reduction of symptoms in Col6a1Cre ER-2Lec animals (FIG. 40). Histological analysis was performed on day 7 using H&E and the alcian blue (AB) and safranin-O (SO) stains, which are commonly used to reveal the cartilage fraction of joints. H&E staining of Col6a1Cre ER-2Lecjoints showed a reduction of pannus size, consistent with the reduction of swelling (FIG. 41). Interestingly, the infiltration of immune cells seemed much reduced in the ER-2Lec expressing animals (FIG. 41 and FIG. 55A). The AB positive region was also significantly preserved in CAIA Col6a1Cre ER-2Lec animals (FIG. 41). Significant change was also obtained after quantification of SO stains (FIG. 55). Taken together, results demonstrate that inhibiting GALA with the ER-2Lec protein in SF can limit arthritis disease progression.

### GALA Activates CNX Glycosylation and Surface Exposure in SF

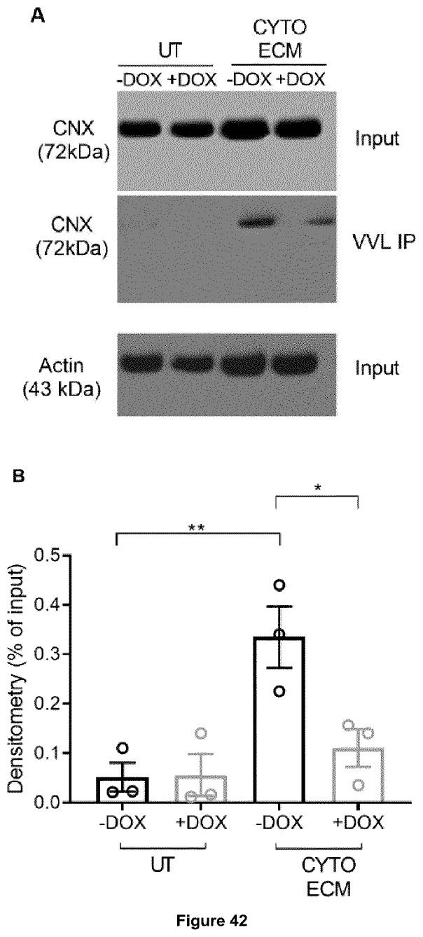

CNX was recently described as a glycosylation target and effector of GALA (Ros et al., Nat. Cell Biol. 22, 1371-1381, 2020). Upon glycosylation, CNX is translocated at the cell surface and, in conjunction with PDIA3, mediates the cleavage of disulfide bonds in ECM proteins. This reductive activity is essential for matrix degradation by cancer cells (Ros et al., Nat. Cell Biol. 22, 1371-1381, 2020). In SW982 SF, it was found that CNX is hyper-glycosylated by ~6 fold after stimulation with cytokines and ECM (FIGS. 42A and 42B). This glycosylation was GALA dependent as expression of ER-2Lec was able to significantly reduce CNX glycosylation.

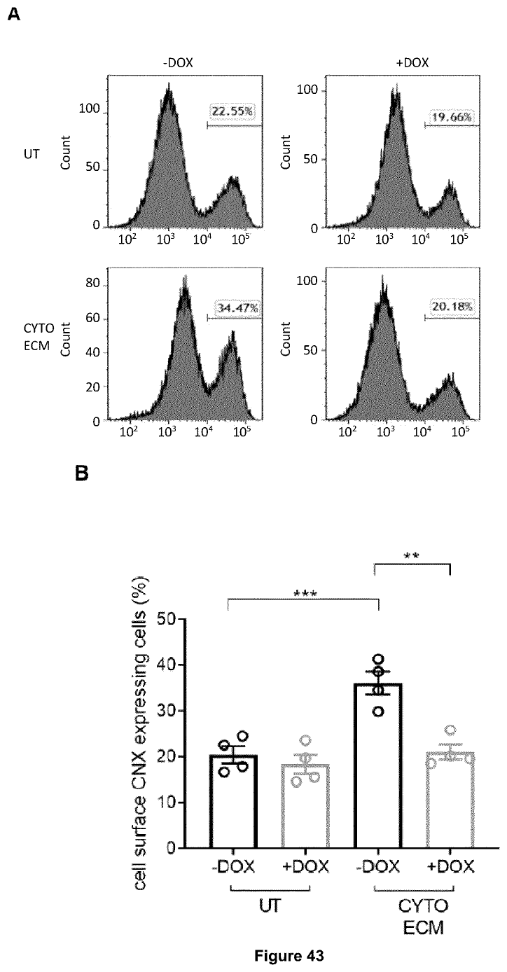

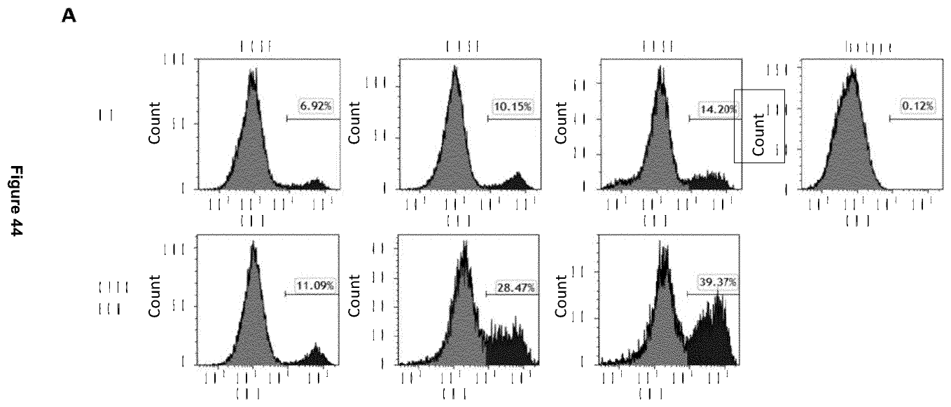

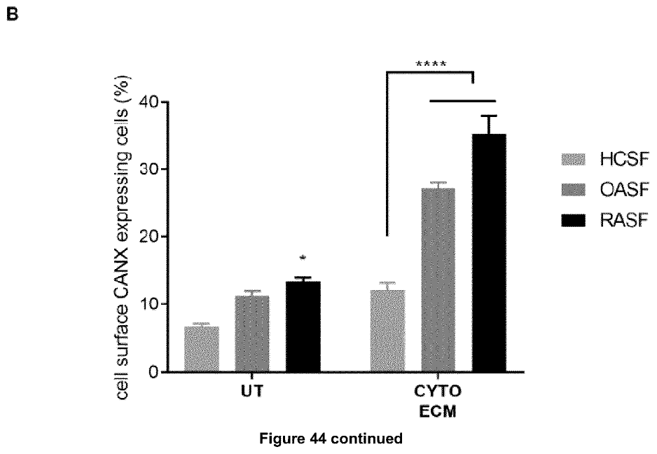

In addition, it was found, using FACS, that CNX surface expression increased significantly by about 10% after stimulation of SW982 SF cells with CYTO and ECM (FIGS. 43A and 43B). Strikingly, the increase in cell surface CNX signal was fully repressed by ER-2Lec expression (FIGS. 44A and 44B).

In SFs from healthy controls (HCSF), the proportion of cell surface CNX positive cells was only ~7% and stimulation with cytokines and ECM increased it slightly (FIGS. 44A and 44B). By contrast, SF cells from the patient suffering from RA (RASF) or OA (OASF) displayed significantly increased levels and were more sensitive to stimulation, with a threefold increase in the percentage of cells with cell surface CNX (FIGS. 44A and 44B). Overall, these results indicate that CNX glycosylation and its cell surface exposure is enhanced in arthritic SFs and is dependent on GALA.

### Anti-CNX Antibodies Prevent Arthritic Symptoms in CAIA Mice

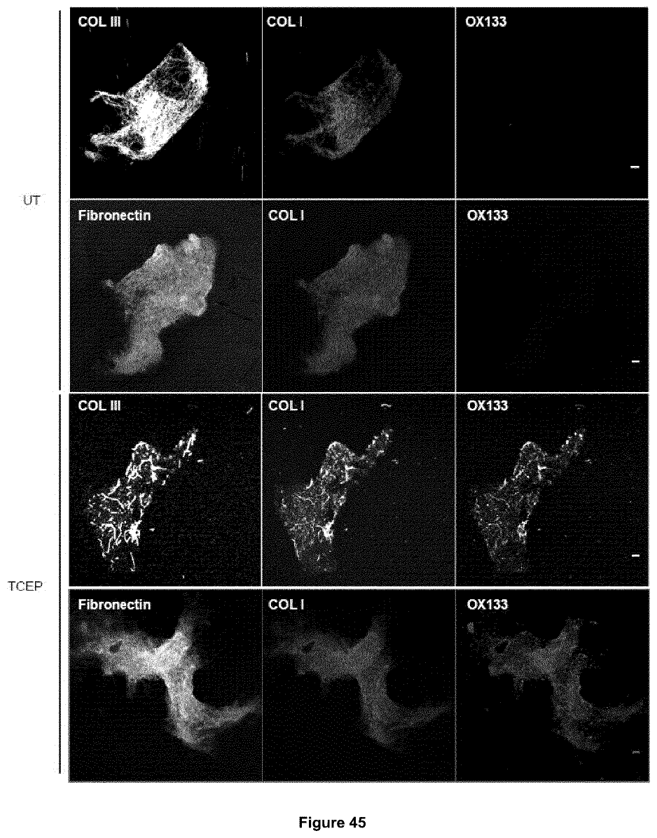

It has been previously shown that antibodies against CNX can block ECM degradation by preventing the necessary reduction of disulfide bonds (Ros et al., Nat. Cell Biol. 22, 1371-1381, 2020). It was hypothesized that blocking CNX would similarly prevent cartilage ECM degradation. The presence of disulfide bonds in cartilage ECM was assessed using a previously described method (Ros et al., Nat. Cell Biol. 22, 1371-1381, 2020). Briefly, cartilage ECM was reduced with TCEP, then exposed to N-Ethylmaleimide (NEM) and then treated with the anti-OX133 antibody. As described previously with liver ECM, an abundant OX133 signal colocalizing with collagen 3/collagen 1 and fibronectin/collagen 1 fibers was observed, suggesting cartilage ECM is heavily cross-linked with disulfide bonds (FIG. 45).

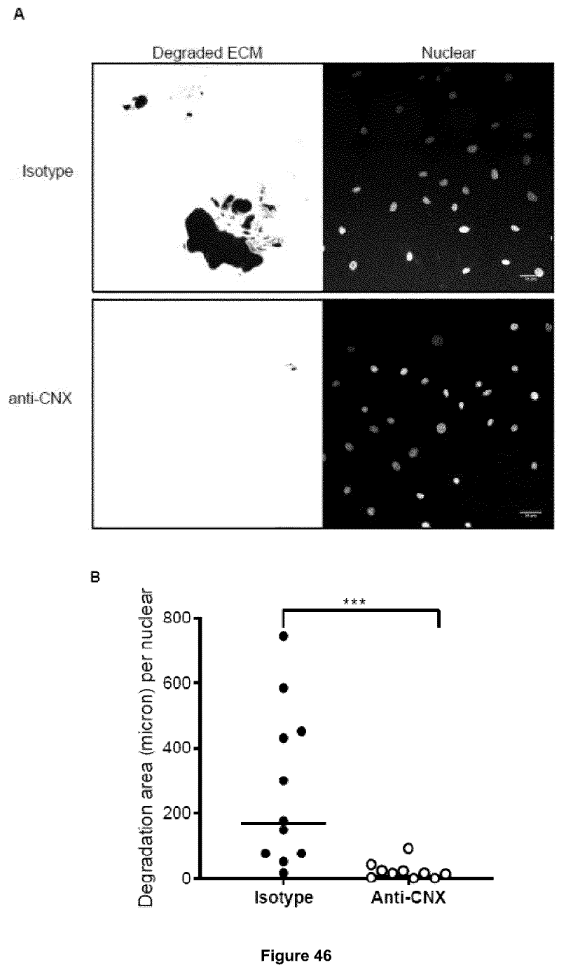

The effect of anti-CNX antibodies on ECM degradation was then assessed. OASF cells seeded on cartilage ECM covering fluorescent gelatin were allowed to degrade ECM overnight. The addition of a polyclonal anti-CNX antibody blocked this degradative activity (FIGS. 46A and 46B).

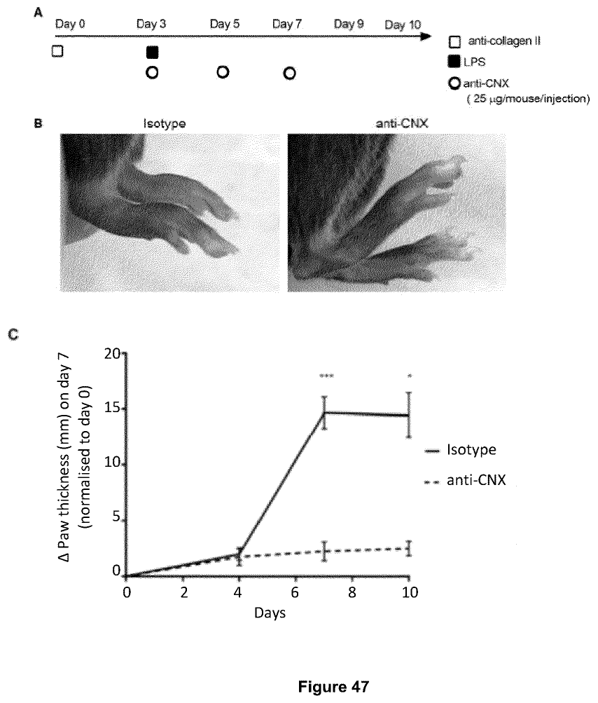

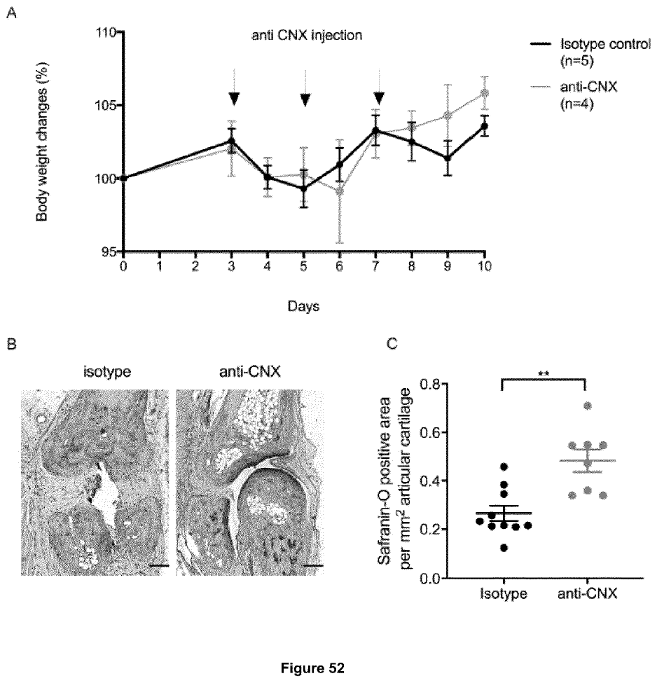

Encouraged by these results, animals were next treated with the anti-CNX antibody. The weight of animals receiving three injections with the antibody over 10 days was monitored; no weight loss was detected (FIG. 52). Next, CAIA animals were treated with the anti-CNX antibody, injecting 25 μg every two days from day 3 till day 7 after initiation of CAIA (FIG. 47A). Paw thickness was monitored at regular intervals and measured arthritic score at day 10. Strikingly, the paws exhibited reduced swelling in anti-CNX treated animals as compared to control animals treated with an isotype antibody (FIG. 47B). While some redness and swelling in the fingers still occurred, raising the arthritic score, the mean score of treated animals was half of CAIA control animals (FIG. 47C). This is reminiscent of the results obtained with ER-2Lec expression.

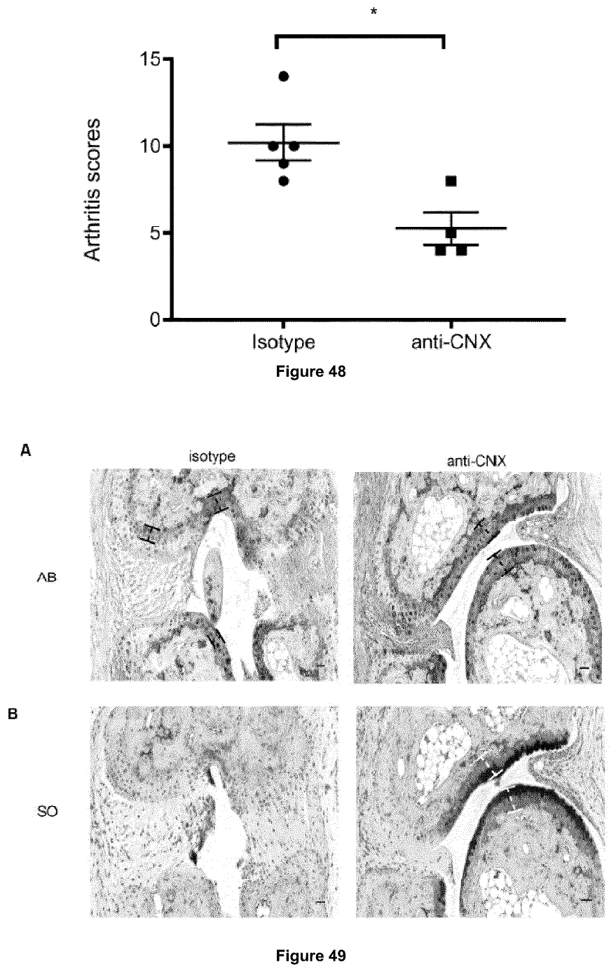

At the histological level, the reduction in SO positive cartilage was very pronounced in control animals at day 10 (FIG. 48). In addition, the synovium had adhered to the underlying bone, possibly indicating the onset of bone remodelling. By contrast, anti-CNX treated animals had a well-preserved joint cavity with abundant cartilage remaining (FIG. 48).

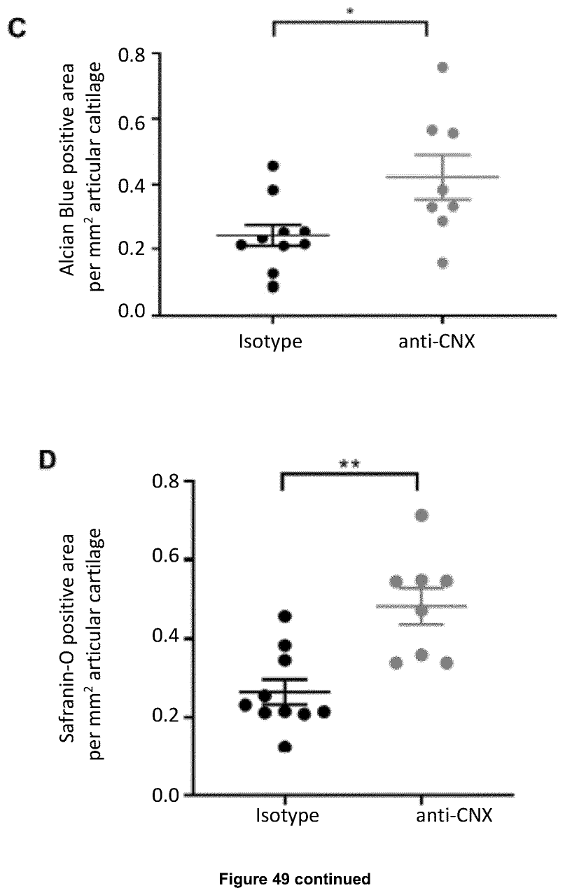

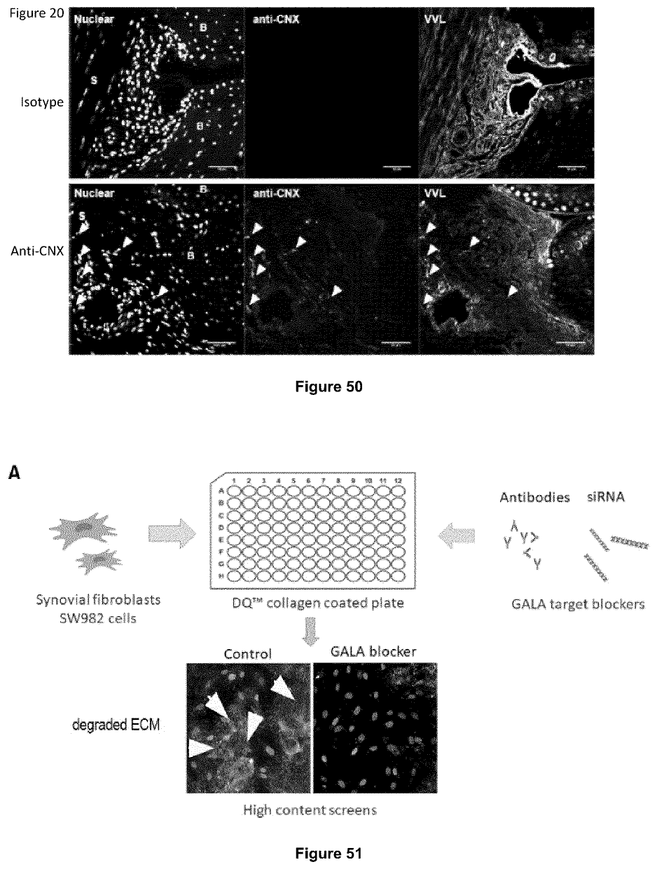

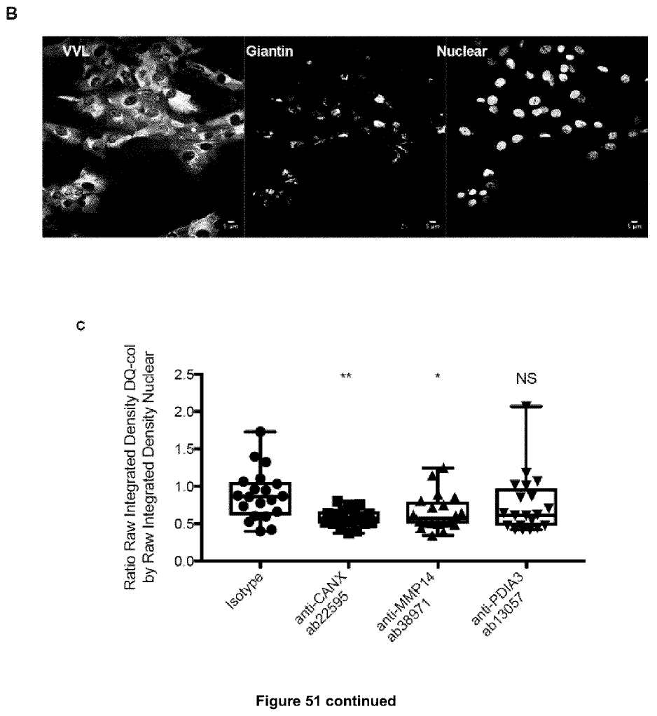

The working hypothesis is that the CNX antibody bound to lining synovial fibroblasts and inhibited their degradative activity. To test whether the antibody had indeed interacted with these cells, the joints of treated animals were stained with an anti-rabbit IgG. A signal in cells of the synovium of animals treated with anti-CNX antibody was clearly detectable, where no signal appeared in animals treated with a control rabbit IgG (FIG. 49C).

Overall, these results indicate that inhibiting CNX results in a potent inhibition of cartilage ECM degradation and could form the basis of an arthritis therapeutics.

### 6.3 Discussion

In this study, it is shown that arthritic synovial fibroblasts have markedly up-regulated GalNac O-glycosylation compared to healthy counterparts. This increase is due to the activation of the GALA pathway, with relocation of GALNTs from the Golgi to the ER.

In cancer cells, activation of EGF-R and in particular the Src kinase drive GALA (Gill et al., J. Cell Biol. 189, 843-858, 2010; Chia et al., PLoS One. 14, e0214118, 2019). Other signaling molecules, such as the ERK8 kinase, constitutively and dynamically inhibit the pathway (Chia et al., Elife. 3, e01828, 2014). In vitro, RA and OA patient derived fibroblasts have moderately elevated GALA levels compared to healthy human SFs. However, RA fibroblasts activate GALA in response to an IL-1 beta and TNF-alpha cytokine mixture. Interestingly, RA SFs response is more marked than normal or OA SFs, suggesting RA SFs have been primed to respond to these cytokines. IL-1 has been reported to activate the tyrosine kinase Src (Mon et al., Oncol. Lett. 13, 955-960, 2017), suggesting a possible link between the cytokine and GALA.

Exposure to ECM strongly activates GALA in both OA and RA fibroblasts, more readily than in the healthy control cells. It was proposed previously that adhesion of SFs to elements of cartilage ECM is involved in arthritis development (Pap et al., Arthritis Res. 2, 361-367, 2000). Injection of fibronectin in joints leads to the degradation of cartilage proteoglycans (Homandberg, et al., J. Rheumatol. 20, 1378-1382, 1993). Integrins, the fibronectin receptors, are also activators of the Src kinase (Shattil, Trends Cell Biol. 15, 399-403, 2005; Huveneers, and Danen, J. Cell Sci. 122, 1059-1069, 2009). Thus, a signaling cascade could link external ECM signals to integrins, Src and then GALA, activating ECM degradation (Gill et al., J. Cell Biol. 189, 843-858, 2010). This hypothetical cascade would feed a pathological positive feedback loop. It remains unclear why GALA response to ECM in healthy SFs is much more limited. SFs from arthritic joints have been proposed to be epigenetically primed for degradation (Nygaard et al., Nat. Rev. Rheumatol. 16, 316-333 (2020). Indeed, OASF and RASF display comparable global methylation profiles, which are distinct from SF from healthy subjects (Nakano et al., Ann. Rheum. Dis. 72, 110-117, 2013). Some differences were identified in genes involved in PDGF and EGF signalling, also regulators of GALA (Chia et al., PLoS One. 14, e0214118, 2019). Thus, an epigenetic priming could include higher propensity to activate GALA. In addition, GALA glycosylation is probably synergistic with other regulatory mechanisms. For instance, increased levels of CNX were found in the synovial tissues of arthritic mice, consistent with gene expression data reported in previous studies (Broeren et al., PLoS One. 11, e0167076, 2016; Nzeusseu Toukap, et al., Arthritis Rheum. 56, 1579-1588, 2007). GALNT1, 3 and 5 were found upregulated in these studies and increased expression of GALNT1 and 2 was observed. Whether activated by immune signals or by ECM proteins, GALA glycosylation may not be activated continuously during arthritis. Indeed, in the CAIA mouse model, the levels of GALA significantly decreased at day 10, preceded by the full recovery of the animals (at day 14 or later). In patient samples, GALA is detectable in samples of OA, RA and psoriasis arthritis, but a significant fraction of samples display low GALA levels. This suggests that GALA is only fully activated during the active ECM degradative phase of the disease, a phase that would correspond to flares in patients. By contrast, in phases of remission, there is less ECM degradation and correspondingly low GALA levels. Among the targets of GALA glycosylation is MMP14, a cell surface protease that degrades collagen fibers and activates other MMPs (Nguyen, et al., Cancer Cell. 32, 639-653.e6, 2017, Gialeli, et al., FEBS J. 278, 16-27, 2011). MMP14 is one of the MMPs involved in arthritis, and activates MMP-2 and 13 (Rose and Kooyman, Dis. Markers. 2016, 4895050, 2016). MMP14 O-glycosylation is essential for its protease activity and occurs in a low complexity region of the protein in the form of a cluster: six or more amino-acids are modified with GalNAc or more complex O-glycans (Nguyen et al., Cancer Cell. 32, 639-653.e6 (2017).

CNX also displays a clustered glycosylation pattern, located in the N-terminal region (Ros et al., Nat. Cell Biol. 22, 1371-1381, 2020). Clustered glycosylation is a frequent feature of GalNac glycosylation, exemplified in mucin proteins. The ER-2Lec chimeric protein inhibits this clustered glycosylation (Gill et al., Proc. Natl. Acad. Sci. U.S.A 110, E3152-61, 2013). As in cancer cells, ER-2Lec reduced the levels of Tn signal in SFs. Thus, it is likely to inhibit at least partially MMP14 and CNX glycosylation inhibiting ECM degradation by SFs in vitro and in vivo. As GALNTs act on thousands of proteins and preliminary, unpublished data indicate that GALA affects many proteins, additional glycoproteins may be involved in the pathological activity of SFs and affected by ER-2Lec (Steentoft, et al., Nat. Methods. 8, 977-982, 2011).

ER-2Lec activated by Cre under a Collagen type VI promoter results in CAIA-treated mice being protected from the loss of cartilage. ER-2Lec expression was mostly restricted to SFs, with no expression detected in immune cells. Interestingly, ER-2Lec expression reduced swelling and inflammation of the joint. Effective protection of the cartilage ECM might reduce SFs activation, preventing release of cytokines and thus inflammation. The fact that anti-CNX antibody treatment also reduces inflammation supports this explanation.

The role of CNX in the degradation of ECM was only recently established. In complex with PDIA3, CNX participates in the reduction of disulfide bonds in liver ECM proteins (Ros et al., Nat. Cell Biol. 22, 1371-1381, 2020). Disulfide bonds, like other cross-linking bonds, prevent the action of proteases (Philp et al., Am. J. Respir. Cell Mol. Biol. 58, 594-603, 2018). The cartilage ECM contains an abundance of disulfide bonds. Anti-CNX antibodies blocked matrix degradation by SFs in vitro and provided a significant protection of cartilage ECM in animals.

Other strategies to inhibit synoviocytes have been developed, such as targeting the adhesion molecule Cadherin 11 (Lee, et al., Science. 315, 1006-1010, 2007; Kiener, et al., Arthritis Rheum. 60, 1305-1310, 2009). More recently, targeting the tyrosine phosphatase PTPRS at the cell surface of SFs has also been shown to protect the cartilage in RA mice (Svensson, et al., Sci Adv. 6, eaba4353, 2020). In addition, the targeting of MMPs has been researched for several decades and specific MMP inhibitors such as Trocade have demonstrated protective effects against RA and OA in animal models (Lewis et al. Br. J. Pharmacol. 121, 540-546 (1997; Brewster, et al., Arthritis Rheum. 41, 1639-1644, 1998). Poor tolerability of these compounds led to clinical trial failures (Close. Ann. Rheum. Dis. 60 Suppl 3, iii62-7 (2001). To date, while some progress has been made, inhibiting MMP therapeutically remains relatively challenging (Fields. Cells. 8. 2019, doi:10.3390/cells8090984). Targeting the CNX-ERp57 complex with antibodies may represent a more attractive approach as less toxicity is expected.

Overall, the data opens perspectives for biomarker discovery and a new therapeutic approach targeting CNX with antibodies. More generally, results suggest that activation of O-glycosylation through GALA is a critical control switch for ECM degradation in synovial fibroblasts as in cancer cells, indicating the broad pathological relevance of the pathway.

### 6.4 References to Example 6

1. D. J. Gill, J. Chia, J. Senewiratne, F. Bard, Regulation of O-glycosylation through Golgi-to-ER relocation of initiation enzymes. J. Cell Biol. 189, 843-858 (2010).
2. D. J. Gill, K. M. Tham, J. Chia, S. C. Wang, C. Steentoft, H. Clausen, E. A. Bard-Chapeau, F. A. Bard, Initiation of GalNAc-type O-glycosylation in the endoplasmic reticulum promotes cancer cell invasiveness. Proc. Natl. Acad. Sci. U.S.A 110, E3152-61 (2013).
3. F. Bard, J. Chia, Cracking the Glycome Encoder: Signaling, Trafficking, and Glycosylation. Trends Cell Biol. 26, 379-388 (2016).
4. A. T. Nguyen, J. Chia, M. Ros, K. M. Hui, F. Saltel, F. Bard, Organelle Specific O-Glycosylation Drives MMP14 Activation, Tumor Growth, and Metastasis. Cancer Cell. 32, 639-653.e6 (2017).
5. M. Ros, A. T. Nguyen, J. Chia, S. Le Tran, X. Le Guezennec, R. McDowall, S. Vakhrushev, H. Clausen, M. J. Humphries, F. Saltel, F. A. Bard, ER-resident oxidoreductases are glycosylated and trafficked to the cell surface to promote matrix degradation by tumour cells. Nat. Cell Biol. 22, 1371-1381 (2020).
6. J. Chia, F. Tay, F. Bard, The GalNAc-T Activation (GALA) Pathway: Drivers and markers. PLoS One. 14, e0214118 (2019).
7. J. Chia, K. M. Tham, D. J. Gill, E. A. Bard-Chapeau, F. A. Bard, ERK8 is a negative regulator of O-GalNAc glycosylation and cell migration. Elife. 3, e01828 (2014).

### Example 7: Anti-CNX Antibodies Limit the Size Expansion of Cancer Cell Spheroids

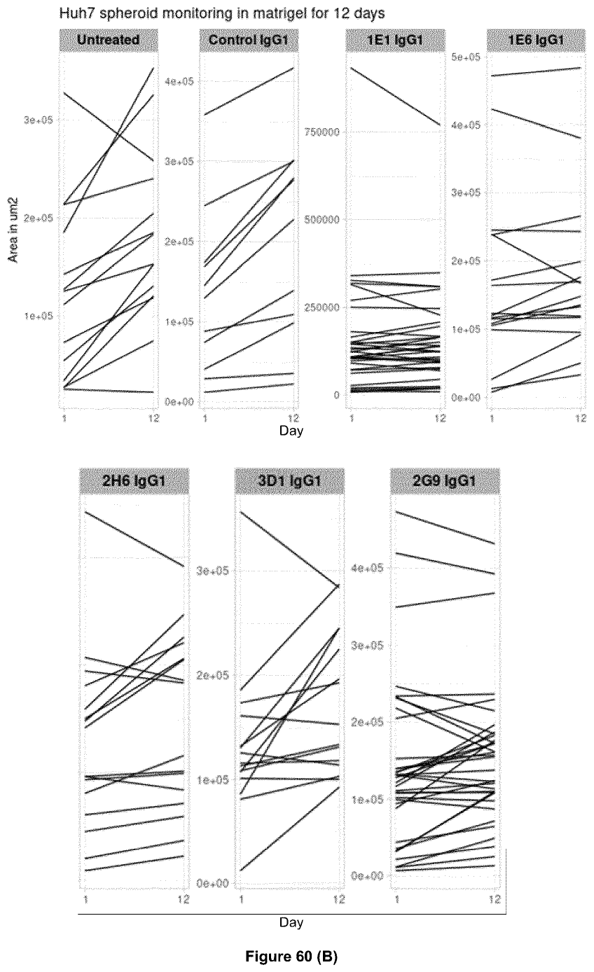

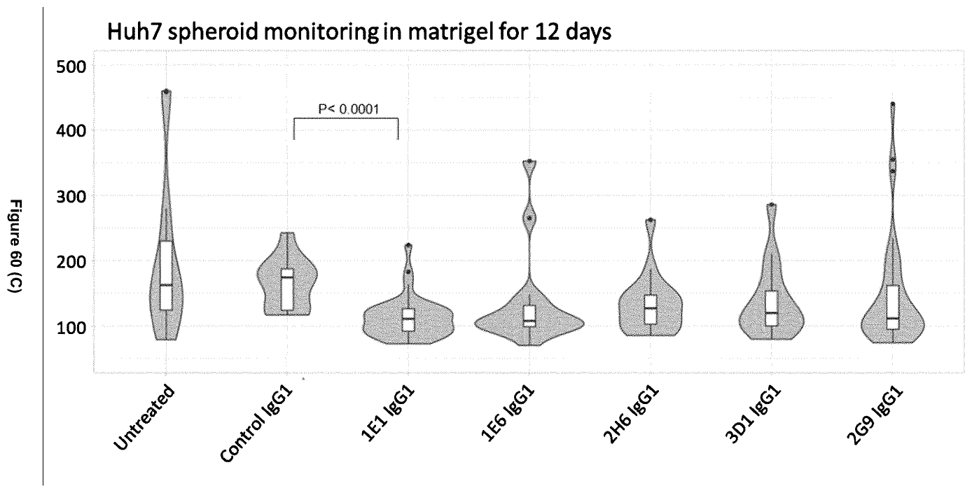

Using a conventional hanging drop method, Huh7 spheroids were generated during a period of 4 days. Spheroids were collected and embedded in growth factor reduced Matrigel, in 96 well plates. Images of the Huh7 spheroids were captured in this initial stage by performing all well surface image acquisition across a depth of 1 mm and using maximal projection of the planes (FIG. 60A). Calnexin IgG1 antibodies or negative control antibodies were administered at 10 μg/ml, with media and antibodies being replaced every 3 days. After 12 days, images were recorded of the spheroids by performing all well image acquisition across a depth of 1 mm and using maximal projection of the planes.

Spheroid location was matched from initial Day 1 to Day 12 and changes in spheroid area and relative growth were calculated (FIGS. 60B and 60C). Untreated and control IgG1 treated spheroids showed clear size expansion after 12 days. Anti-CNX antibodies showed different trends—the majority of anti 1E1 treated spheroids reduced in size. Lineplot analysis showed a marked increase in slope for most of spheroids in Untreated and Control IgG1, whereas most of 1E1 spheroids produced flat lineplots with minimal change of state between Day 1 and Day 12 due to the limitation of spheroid size expansion. The lineplots for 1E6 and 2G9 also looked noticeably different to the Untreated and Control IgG1 groups, suggesting an effect in limiting limit the size expansion of cancer cell spheroids.

Accessing relative growth change for spheroid at Day 12 as compared to Day1 showed significant difference for 1E1 as compared to control IgG1, with some non-significant differences seen for ither anti-CNX antibodies (FIG. 60C).

### Example 8: Anti-CNX Antibodies Demonstrate ECM Protection Against Synovial Fibroblasts and Prevent Arthritic Symptoms

### ECM Protection Assay in Synovial Fibroblast Cell Line SW982

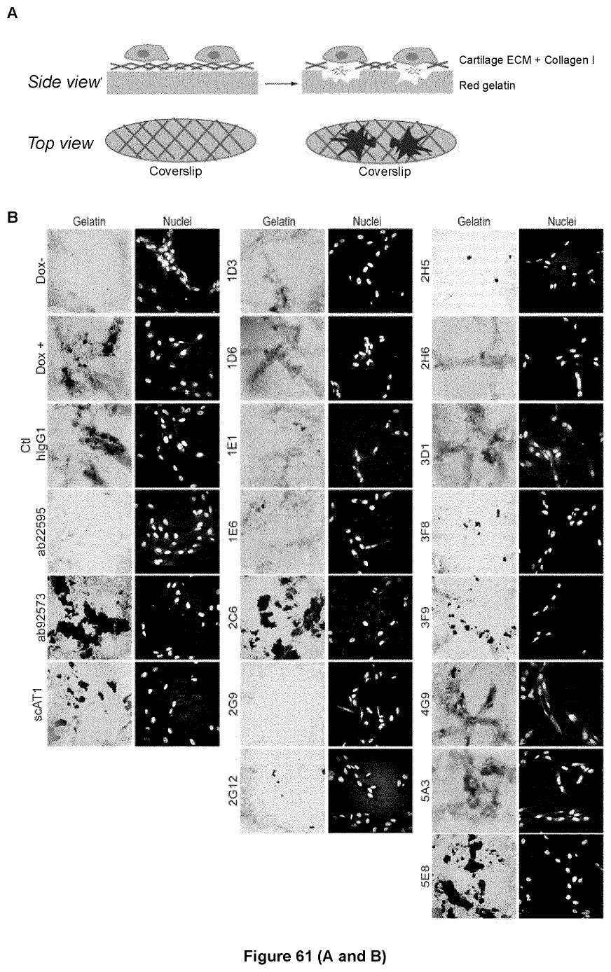

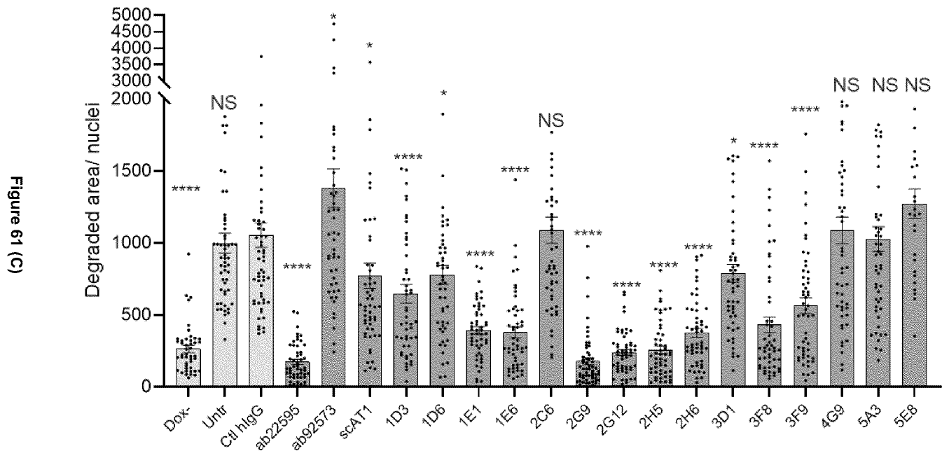

The ECM protection assay measures cell-mediated degradation of fluorescent gelatin beneath a layer of cartilage ECM and rat tail Collagen I. Briefly, 2% fluorescently labelled gelatin was coated on sterile coverslips and stabilised with 0.05% glutaraldehyde fixation. A mixture of 0.1 mg/ml Cartilage ECM (Xylyx Bio) together with 0.5 mg/ml rat tail collagen I (Corning) was then coated as a thin layer on top of the fluorescent gelatin. Synovial cell line SW982 ER-G1 cells were then seeded on these coverslips in the presence of 0.1 ug/ml doxycycline induction to stimulate ER-G1 expression. ER-G1 expression stimulates GALA activation in these cells. The cells were allowed to degrade the ECM gelatin coverslips for 48 hours with and without presence of antibodies (FIG. 61A). After 48 hours, the gelatin coverslips were fixed with 4% paraformaldehyde and stained with fluorescent Hoescht for nuclei counting. The coverslips were imaged on a confocal microscope with at least 25 fields per coverslip. The images were analysed by ImageJ to quantify the area of gelatin degradation and the number of nuclei per field. The final analysis output was the area of degradation per nuclei.

The 15 IgG1 antibodies derived from the Fab library, and commercial CNX antibodies, were characterised on SW982 ERG1 cells (FIGS. 61B and 61C). 10 μg/ml of antibody was added to treat the cells. Quantification showed that GALA activation by doxycycline treatment resulted in 3-fold increase in ECM degradation compared to non-doxycycline induced cells (Dox-). Commercial anti-CNX monoclonal antibody ab92573 did not inhibit ECM degradation. Commercial polyclonal anti-CNX antibody CNX ab22595 blocks ECM degradation in SW982 ERG1 cells (~84% reduction), in a way that is similar to that previously observed in HUH7 cells (Example 4). Various human CNX antibodies showed different extent of ECM protection activity with about 7-fold difference between 2G9 and 5E8.

Treatment with 1E1 Prevent Arthritic Symptoms in Rheumatoid Arthritis CAIA Mouse Model

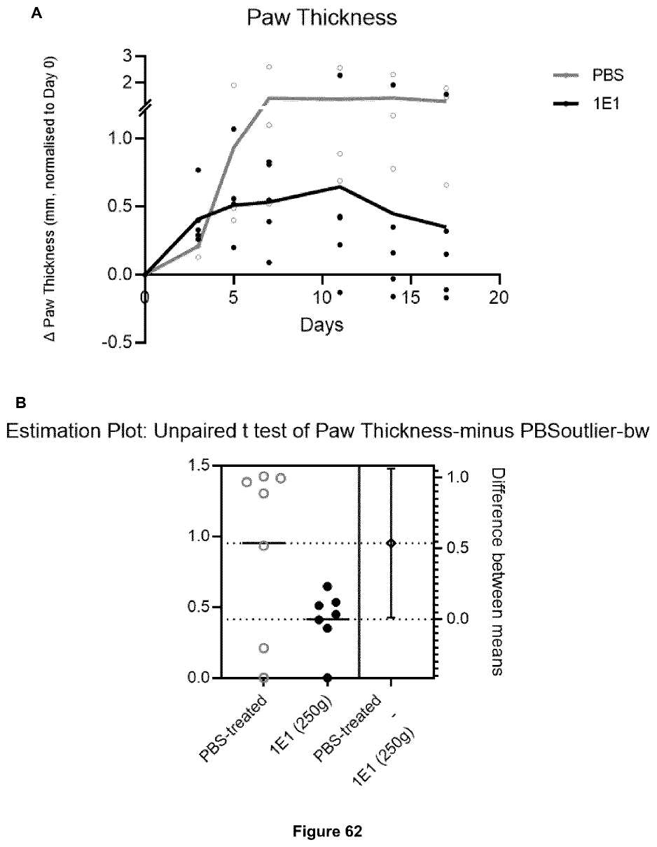

DBA/J mice were induced with 0.5 mg of anti-Collagen II antibody (Chondrex) for 3 days before starting antibody treatment. 5 mice were treated with 250 ug of 1E1 and 3 mice were injected with PBS as a control every 2 days. The paw thickness, an indicator of arthritic swelling, was monitored every 2 days over a course of 15 days. The change in paw thickness with respect to day 0 was quantified for each animal (FIG. 62A). The paws exhibited significantly reduced swelling in 1E1 treated mice compared to control mice (FIG. 62B).

Treatment with 2G9 Prevent Arthritic Symptoms in Rheumatoid Arthritis CAIA Mouse Model

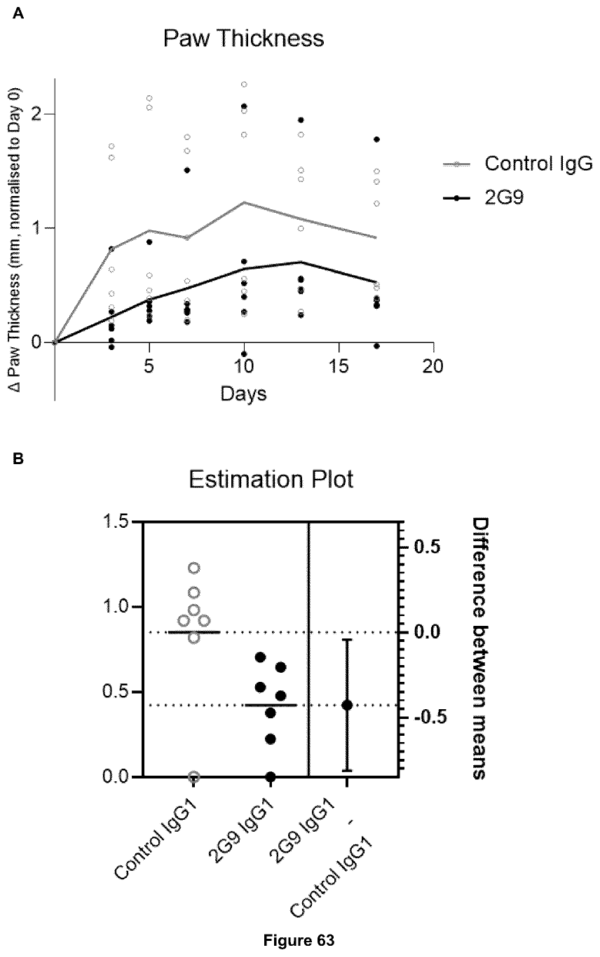

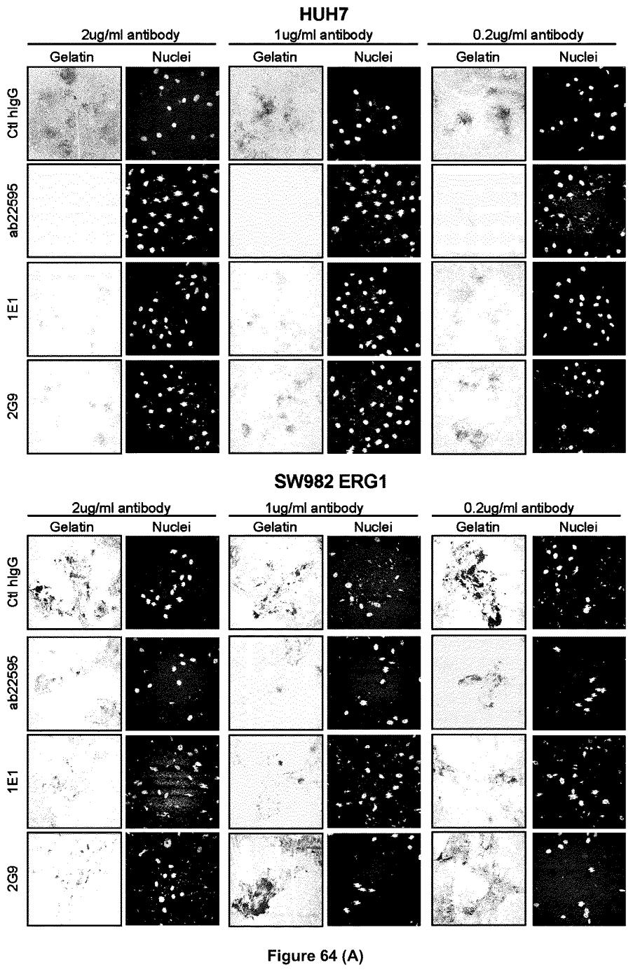

DBA/J mice were induced with 0.5 mg of anti-Collagen II antibody (Chondrex) for 3 days before starting antibody treatment. 5 mice were treated with 250 ug of 2G9 and 3 mice were injected with Control IgG1 every 2 days. The paw thickness, an indicator of arthritic swelling, was monitored every 2 days over a course of 18 days. The change in paw thickness with respect to day 0 was quantified for each animal (FIG. 63A). The paws exhibited significantly reduced swelling in 2G9 treated mice compared to control mice (FIG. 63B).

### Example 9: Dose Response of Anti-CNX Antibodies

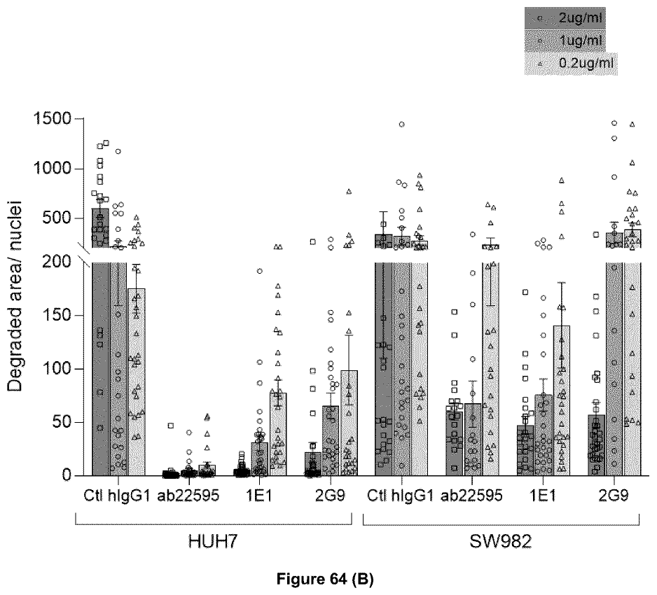

Anti-CNX antibodies 1E1 and 2G9 were further characterised in wildtype HUH7 and SW982 ERG1 cells. Lower doses of 2 μg/ml, 1 μg/ml and 0.2 μg/ml antibodies were tested to observe the dose response to inhibition of ECM degradation. The cells, in the presence of various antibodies at different concentrations, were allowed to degrade the ECM gelatin coverslips for 48 hours before fixation for imaging. The Example 8 method of the ECM protection assay was used for this experiment. Results are shown in FIG. 64. A dose response is clearly visible for both 1E1 and 2G9 antibodies in both cancer (HUH7) and synovial (SW982 ERG1) cell lines based on histological (FIG. 64A) and quantification data (FIG. 64B).

### Example 10: Anti-CNX Antibodies Accumulate in Tumors and Block Tumor Growth

### Accumulation of Antibodies in Tumor Cells:

The accumulation of anti-CNX antibodies (clones 1E1 and 3D1) in different tumor types was investigated through immunoblot analysis. Specifically, the ability of the antibodies to accumulate in HepG2, Hep3B, and Huh7 subcutaneous tumor cell lines was assessed.

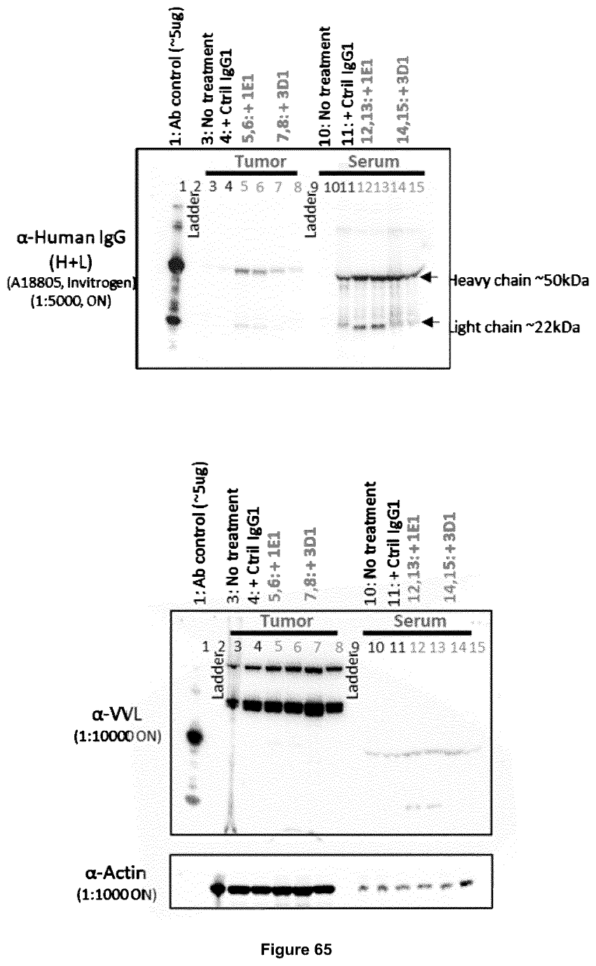

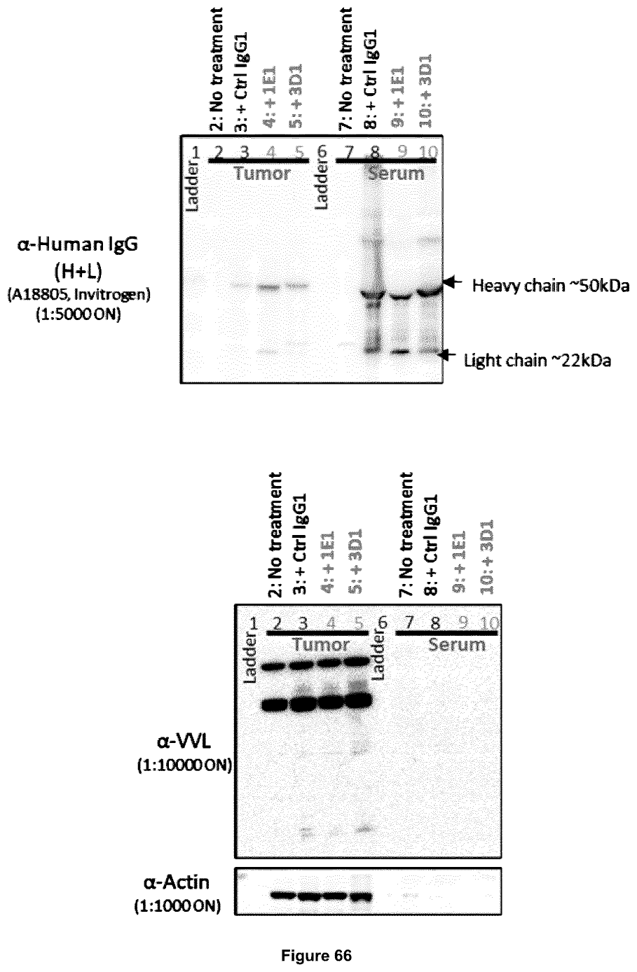

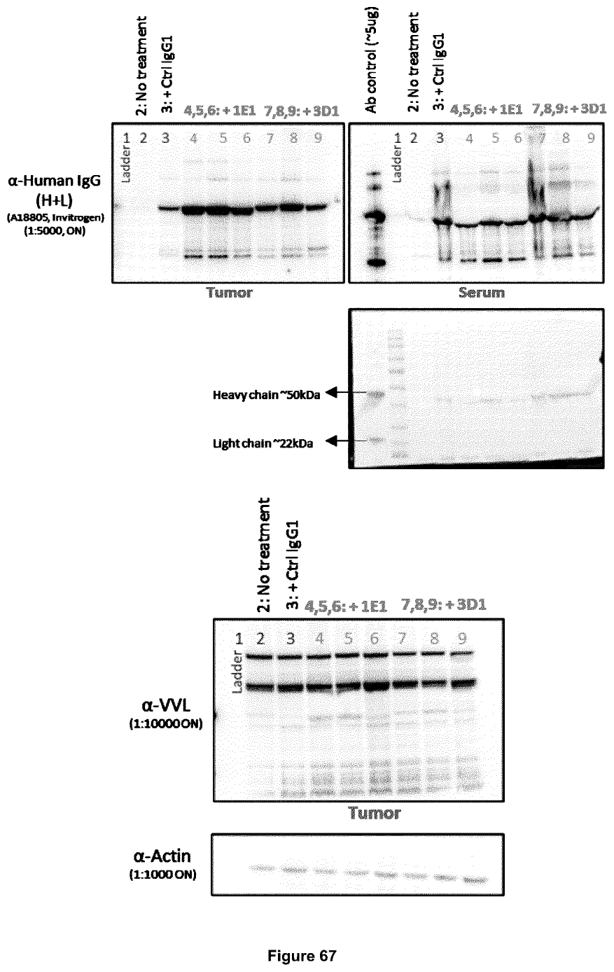

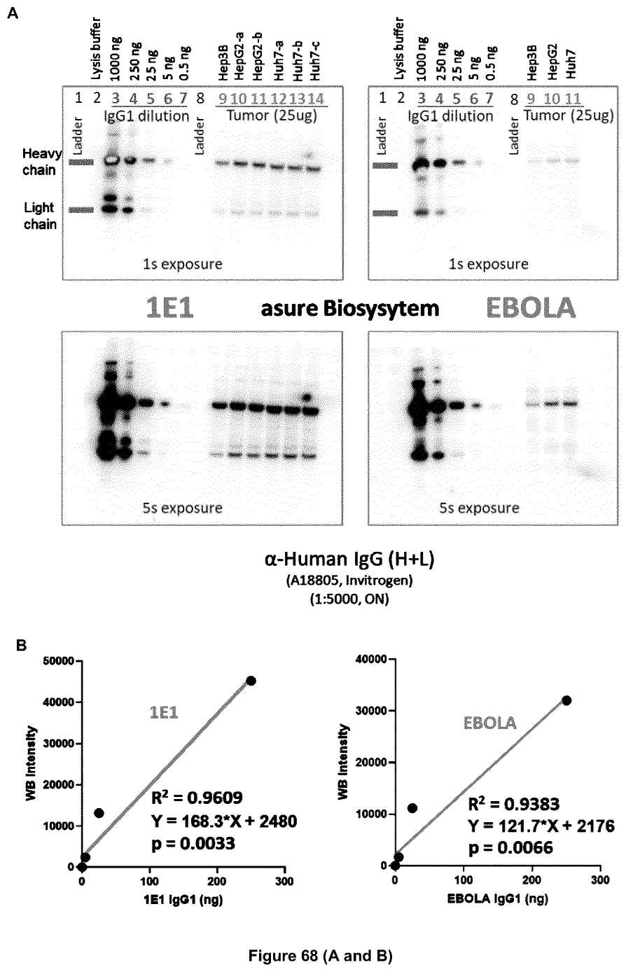

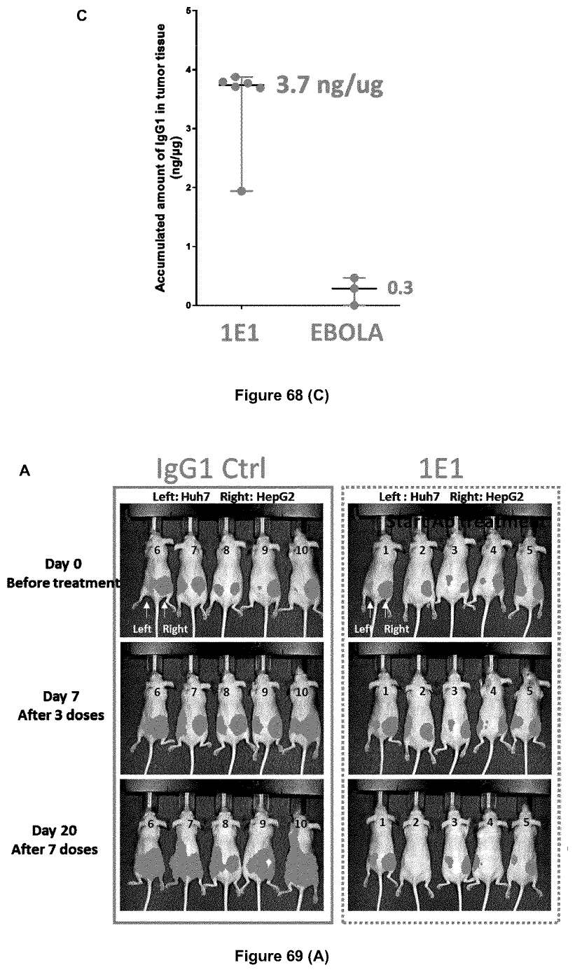

The anti-CNX antibodies (clones 1E1 and 3D1) were shown to significantly accumulate in HepG2-Luc, Hep3B, and Huh7-Luc tumor tissues, compared to relevant controls (FIGS. 65-67).

### Anti-CNX Antibodies Block Tumor Growth In Vivo

The ability of anti-CNX antibodies to reduce or inhibit tumor growth in vivo was assessed in nude mice and NSG mice. A nude mouse is a laboratory mouse from a strain with a genetic mutation that causes a deteriorated or absent thymus, resulting in an inhibited immune system due to a greatly reduced number of T cells. NSG branded mice lack mature T cells, B cells, and natural killer (NK) cells. NSG branded mice are also deficient in multiple cytokine signaling pathways, and they have many defects in innate immunity.

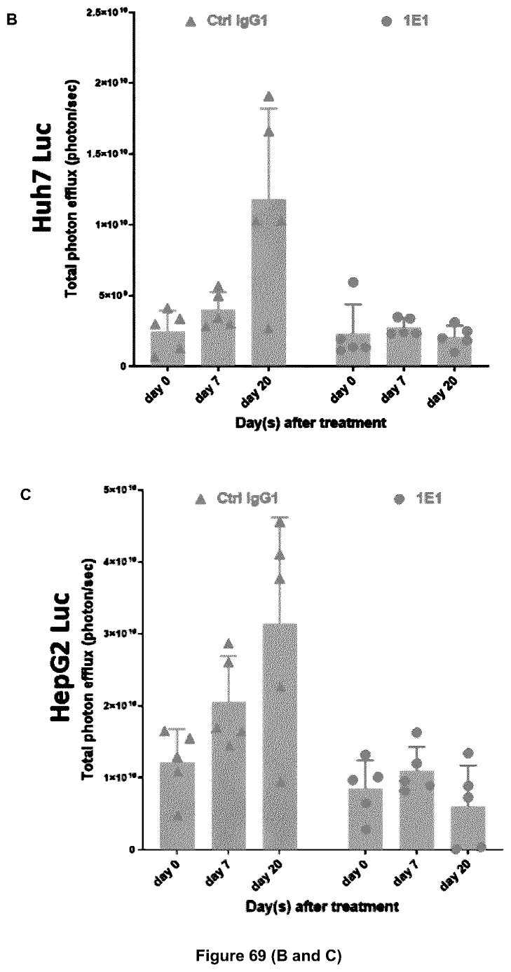

Nude mice carrying tumors were treated with anti-CNX antibodies (1E1) compared to IgG1 Control at 15 mg/kg per dose. Quantification of total photon flux from HepG2Luc and Huh7Luc tumors expressing Luciferase is shown in FIG. 69. It can be seen that the growth of both Huh7 and HepG2 tumours was significantly inhibited by the anti-CNX antibodies.

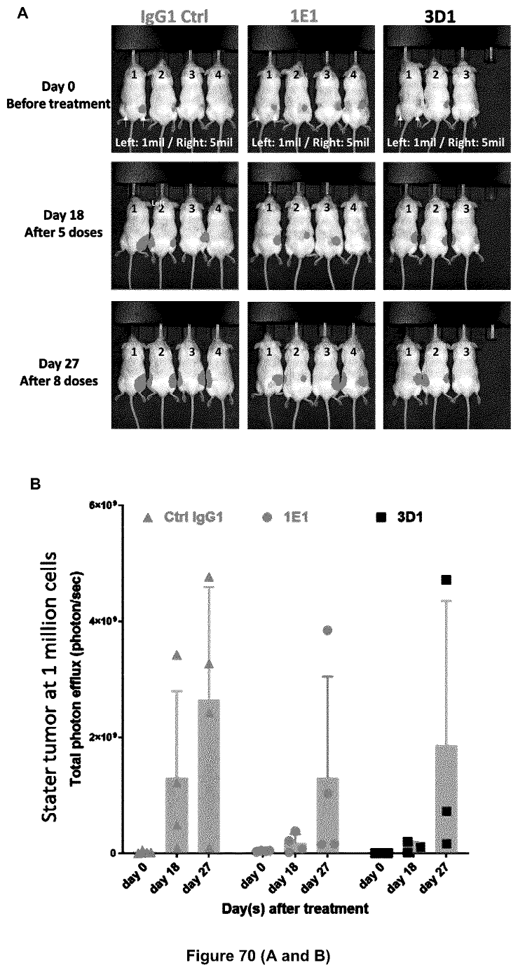

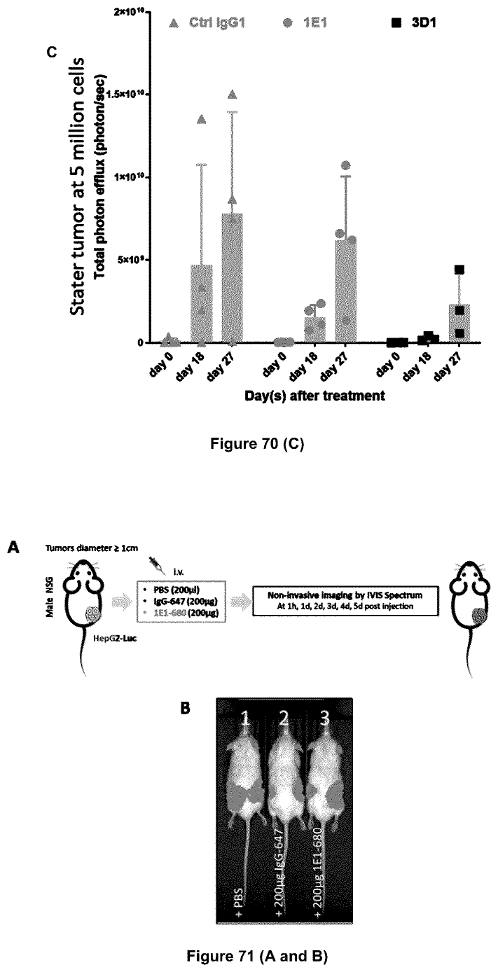

NSG mice carrying tumors were treated with CNX antibodies (1E1 or 3D1) compared to IgG1 Control at 15 mg/kg per dose. Quantification of total photon flux from HepG2Luc tumors expressing Luciferase is shown in FIG. 70. Again, it can be seen that the growth of HepG2 tumours was inhibited by the anti-CNX antibodies.

### Example 11: Imaging of α-Calnexin/1E1 Alexa Fluor®680

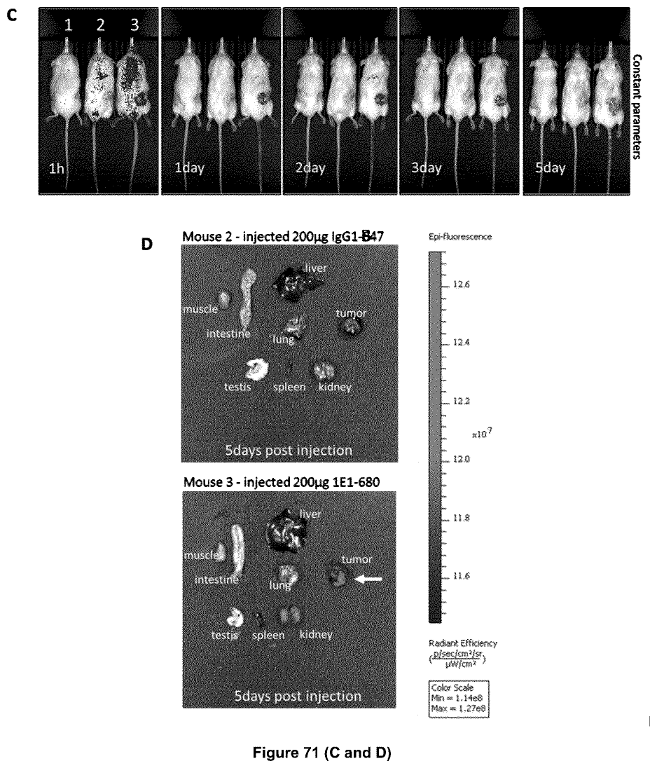

In vivo Imaging of α-Calnexin/1E1 Alexa Fluor®680-conjugated in mice bearing in HepG2Luc tumors was performed according to the schematic of FIG. 71A.

Subcutaneous tumors were generated by HepG2-Luc cell injection in left (1×10⁶) and right (5×10⁶) flanks of NSG mice. When HepG2Luc tumors reached about 1 cm in diameter, they were used for imaging of α-Calnexin/1E1 Alexa Fluor®680. Tail-vein injections were performed to deliver 200 μg of IgG-647 (mouse 2) or 1E1-680 (Mouse 3) diluted in PBS and compared with mouse 1 injected with 200 μl PBS (negative control). Before the imaging session, surrounding tumor regions to be imaged were shaved with clippers and depilatory cream.

Both in vivo bioluminescent imaging (FIG. 71B) and fluorescence imaging of α-Calnexin/1E1 Alexa Fluor® 680-conjugated (FIG. 71C) in mice bearing in HepG2Luc tumors was performed using the IVIS Spectrum In Vivo Imaging System (Perkin Elmer). Constant parameters were applied for fluorescence imaging at Ex675/Em720 filter, fluorescence level: Low, binning Factor: 8 and f Number: 8, exposure time: 2s, and analyzed using Living Image software. Images show that the α-Calnexin/1E1 Alexa Fluor®680-conjugated mainly accumulated in tumors.

After the last time point of in vivo imaging at 5 days post injection, organs were removed from euthanized mice and placed in cold PBS in a petri dish before imaged ex vivo using constant parameters were applied for fluorescence imaging at Ex605/Em700 filter, fluorescence level: Low, binning Factor: 8 and f Number: 8, exposure time: 1s.

Ex vivo imaging of Calnexin/1E1 Alexa Fluor®680-conjugated accumulation in tumor versus organs at 5 days post injection was performed, with results being shown in FIG. 71D. This ex vivo fluorescence imaging reveals the presence of IgG-647 and 1E1-680 in organs and tumors from mice in FIG. 71C at 5 days post injection. The arrow highlights the strong fluorescent signal of Calnexin/1E1 Alexa Fluor®680-conjugated in the HepG2 tumor of mouse 3, but not in other organs.

1. An antigen-binding molecule, optionally isolated, which binds to CNX.

2. The antigen-binding molecule according to claim 1, wherein the antigen-binding molecule inhibits extracellular matrix (ECM) degradation.

3. The antigen-binding molecule according to claim 1 or claim 2, wherein the antigen-binding molecule comprises:
(a)
(i) a heavy chain variable (VH) region incorporating the following CDRs:
HC-CDR1 having the amino acid sequence of SEQ ID NO:166
HC-CDR2 having the amino acid sequence of SEQ ID NO:167
HC-CDR3 having the amino acid sequence of SEQ ID NO:168; and
(ii) a light chain variable (VL) region incorporating the following CDRs:
LC-CDR1 having the amino acid sequence of SEQ ID NO:179
LC-CDR2 having the amino acid sequence of SEQ ID NO:180
LC-CDR3 having the amino acid sequence of SEQ ID NO:173; or
(b)
(i) a heavy chain variable (VH) region incorporating the following CDRs:
HC-CDR1 having the amino acid sequence of SEQ ID NO:33
HC-CDR2 having the amino acid sequence of SEQ ID NO:34
HC-CDR3 having the amino acid sequence of SEQ ID NO:35; and
(ii) a light chain variable (VL) region incorporating the following CDRs:
LC-CDR1 having the amino acid sequence of SEQ ID NO:41
LC-CDR2 having the amino acid sequence of SEQ ID NO:42
LC-CDR3 having the amino acid sequence of SEQ ID NO:43; or
(c)
(i) a heavy chain variable (VH) region incorporating the following CDRs:
HC-CDR1 having the amino acid sequence of SEQ ID NO:2
HC-CDR2 having the amino acid sequence of SEQ ID NO:3
HC-CDR3 having the amino acid sequence of SEQ ID NO:4; and
(ii) a light chain variable (VL) region incorporating the following CDRs:
LC-CDR1 having the amino acid sequence of SEQ ID NO:10
LC-CDR2 having the amino acid sequence of SEQ ID NO:11
LC-CDR3 having the amino acid sequence of SEQ ID NO:12; or
(d)
(i) a heavy chain variable (VH) region incorporating the following CDRs:
HC-CDR1 having the amino acid sequence of SEQ ID NO:18
HC-CDR2 having the amino acid sequence of SEQ ID NO:19
HC-CDR3 having the amino acid sequence of SEQ ID NO:20; and
(ii) a light chain variable (VL) region incorporating the following CDRs:
LC-CDR1 having the amino acid sequence of SEQ ID NO:25
LC-CDR2 having the amino acid sequence of SEQ ID NO:26
LC-CDR3 having the amino acid sequence of SEQ ID NO:27; or
(e)
(i) a heavy chain variable (VH) region incorporating the following CDRs:
HC-CDR1 having the amino acid sequence of SEQ ID NO:48
HC-CDR2 having the amino acid sequence of SEQ ID NO:3
HC-CDR3 having the amino acid sequence of SEQ ID NO:49; and
(ii) a light chain variable (VL) region incorporating the following CDRs:
LC-CDR1 having the amino acid sequence of SEQ ID NO:53
LC-CDR2 having the amino acid sequence of SEQ ID NO:54
LC-CDR3 having the amino acid sequence of SEQ ID NO:55; or
(f)
(i) a heavy chain variable (VH) region incorporating the following CDRs:
HC-CDR1 having the amino acid sequence of SEQ ID NO:61
HC-CDR2 having the amino acid sequence of SEQ ID NO:62
HC-CDR3 having the amino acid sequence of SEQ ID NO:63; and
(ii) a light chain variable (VL) region incorporating the following CDRs:
LC-CDR1 having the amino acid sequence of SEQ ID NO:68
LC-CDR2 having the amino acid sequence of SEQ ID NO:26
LC-CDR3 having the amino acid sequence of SEQ ID NO:69; or
(g)
(i) a heavy chain variable (VH) region incorporating the following CDRs:
HC-CDR1 having the amino acid sequence of SEQ ID NO:61
HC-CDR2 having the amino acid sequence of SEQ ID NO:62
HC-CDR3 having the amino acid sequence of SEQ ID NO:63; and
(ii) a light chain variable (VL) region incorporating the following CDRs:
LC-CDR1 having the amino acid sequence of SEQ ID NO:73
LC-CDR2 having the amino acid sequence of SEQ ID NO:26
LC-CDR3 having the amino acid sequence of SEQ ID NO:74; or
(h)
(i) a heavy chain variable (VH) region incorporating the following CDRs:
HC-CDR1 having the amino acid sequence of SEQ ID NO:61
HC-CDR2 having the amino acid sequence of SEQ ID NO:62
HC-CDR3 having the amino acid sequence of SEQ ID NO:63; and
(ii) a light chain variable (VL) region incorporating the following CDRs:
LC-CDR1 having the amino acid sequence of SEQ ID NO:78
LC-CDR2 having the amino acid sequence of SEQ ID NO:79
LC-CDR3 having the amino acid sequence of SEQ ID NO:80; or
(i)
(i) a heavy chain variable (VH) region incorporating the following CDRs:
HC-CDR1 having the amino acid sequence of SEQ ID NO:2
HC-CDR2 having the amino acid sequence of SEQ ID NO:3
HC-CDR3 having the amino acid sequence of SEQ ID NO:83; and
(ii) a light chain variable (VL) region incorporating the following CDRs:
LC-CDR1 having the amino acid sequence of SEQ ID NO:73
LC-CDR2 having the amino acid sequence of SEQ ID NO:26
LC-CDR3 having the amino acid sequence of SEQ ID NO:74; or
(j)
(i) a heavy chain variable (VH) region incorporating the following CDRs:
HC-CDR1 having the amino acid sequence of SEQ ID NO:48
HC-CDR2 having the amino acid sequence of SEQ ID NO:3
HC-CDR3 having the amino acid sequence of SEQ ID NO:86; and
(ii) a light chain variable (VL) region incorporating the following CDRs:
LC-CDR1 having the amino acid sequence of SEQ ID NO:89
LC-CDR2 having the amino acid sequence of SEQ ID NO:11
LC-CDR3 having the amino acid sequence of SEQ ID NO:90; or
(k)
(i) a heavy chain variable (VH) region incorporating the following CDRs:
HC-CDR1 having the amino acid sequence of SEQ ID NO:61
HC-CDR2 having the amino acid sequence of SEQ ID NO:95
HC-CDR3 having the amino acid sequence of SEQ ID NO:96; and
(ii) a light chain variable (VL) region incorporating the following CDRs:
LC-CDR1 having the amino acid sequence of SEQ ID NO:101
LC-CDR2 having the amino acid sequence of SEQ ID NO:102
LC-CDR3 having the amino acid sequence of SEQ ID NO:103; or
(l)
(i) a heavy chain variable (VH) region incorporating the following CDRs:
HC-CDR1 having the amino acid sequence of SEQ ID NO:108
HC-CDR2 having the amino acid sequence of SEQ ID NO:109
HC-CDR3 having the amino acid sequence of SEQ ID NO:110; and
(ii) a light chain variable (VL) region incorporating the following CDRs:
LC-CDR1 having the amino acid sequence of SEQ ID NO:115
LC-CDR2 having the amino acid sequence of SEQ ID NO:116
LC-CDR3 having the amino acid sequence of SEQ ID NO:117; or
(m)
(i) a heavy chain variable (VH) region incorporating the following CDRs:
HC-CDR1 having the amino acid sequence of SEQ ID NO:2
HC-CDR2 having the amino acid sequence of SEQ ID NO:3
HC-CDR3 having the amino acid sequence of SEQ ID NO:122; and
(ii) a light chain variable (VL) region incorporating the following CDRs:
LC-CDR1 having the amino acid sequence of SEQ ID NO:125
LC-CDR2 having the amino acid sequence of SEQ ID NO:126
LC-CDR3 having the amino acid sequence of SEQ ID NO:127; or
(n)
(i) a heavy chain variable (VH) region incorporating the following CDRs:
HC-CDR1 having the amino acid sequence of SEQ ID NO:132
HC-CDR2 having the amino acid sequence of SEQ ID NO:133
HC-CDR3 having the amino acid sequence of SEQ ID NO:134; and
(ii) a light chain variable (VL) region incorporating the following CDRs:
LC-CDR1 having the amino acid sequence of SEQ ID NO:139
LC-CDR2 having the amino acid sequence of SEQ ID NO:140
LC-CDR3 having the amino acid sequence of SEQ ID NO:80; or
(o)
(i) a heavy chain variable (VH) region incorporating the following CDRs:
HC-CDR1 having the amino acid sequence of SEQ ID NO:146
HC-CDR2 having the amino acid sequence of SEQ ID NO:147
HC-CDR3 having the amino acid sequence of SEQ ID NO:148; and
(ii) a light chain variable (VL) region incorporating the following CDRs:
LC-CDR1 having the amino acid sequence of SEQ ID NO:41
LC-CDR2 having the amino acid sequence of SEQ ID NO:42
LC-CDR3 having the amino acid sequence of SEQ ID NO:153; or
(p)
(i) a heavy chain variable (VH) region incorporating the following CDRs:
HC-CDR1 having the amino acid sequence of SEQ ID NO:48
HC-CDR2 having the amino acid sequence of SEQ ID NO:3
HC-CDR3 having the amino acid sequence of SEQ ID NO:156; and
(ii) a light chain variable (VL) region incorporating the following CDRs:
LC-CDR1 having the amino acid sequence of SEQ ID NO:158
LC-CDR2 having the amino acid sequence of SEQ ID NO:159
LC-CDR3 having the amino acid sequence of SEQ ID NO:160; or
(q)
(i) a heavy chain variable (VH) region incorporating the following CDRs:
HC-CDR1 having the amino acid sequence of SEQ ID NO:166
HC-CDR2 having the amino acid sequence of SEQ ID NO:167
HC-CDR3 having the amino acid sequence of SEQ ID NO:168; and
(ii) a light chain variable (VL) region incorporating the following CDRs:
LC-CDR1 having the amino acid sequence of SEQ ID NO:171
LC-CDR2 having the amino acid sequence of SEQ ID NO:172
LC-CDR3 having the amino acid sequence of SEQ ID NO:173; or
(r)
(i) a heavy chain variable (VH) region incorporating the following CDRs:
HC-CDR1 having the amino acid sequence of SEQ ID NO:185
HC-CDR2 having the amino acid sequence of SEQ ID NO:186
HC-CDR3 having the amino acid sequence of SEQ ID NO:187; and
(ii) a light chain variable (VL) region incorporating the following CDRs:
LC-CDR1 having the amino acid sequence of SEQ ID NO:73
LC-CDR2 having the amino acid sequence of SEQ ID NO:26
LC-CDR3 having the amino acid sequence of SEQ ID NO:194; or
(s)
(i) a heavy chain variable (VH) region incorporating the following CDRs:
HC-CDR1 having the amino acid sequence of SEQ ID NO:48
HC-CDR2 having the amino acid sequence of SEQ ID NO:199
HC-CDR3 having the amino acid sequence of SEQ ID NO:200; and
(ii) a light chain variable (VL) region incorporating the following CDRs:
LC-CDR1 having the amino acid sequence of SEQ ID NO:205
LC-CDR2 having the amino acid sequence of SEQ ID NO:42
LC-CDR3 having the amino acid sequence of SEQ ID NO:206; or
(t)
(i) a heavy chain variable (VH) region incorporating the following CDRs:
HC-CDR1 having the amino acid sequence of SEQ ID NO:48
HC-CDR2 having the amino acid sequence of SEQ ID NO:211
HC-CDR3 having the amino acid sequence of SEQ ID NO:212; and
(ii) a light chain variable (VL) region incorporating the following CDRs:
LC-CDR1 having the amino acid sequence of SEQ ID NO:216
LC-CDR2 having the amino acid sequence of SEQ ID NO:172
LC-CDR3 having the amino acid sequence of SEQ ID NO:217; or
(u)
(i) a heavy chain variable (VH) region incorporating the following CDRs:
HC-CDR1 having the amino acid sequence of SEQ ID NO:222
HC-CDR2 having the amino acid sequence of SEQ ID NO:223
HC-CDR3 having the amino acid sequence of SEQ ID NO:224; and
(ii) a light chain variable (VL) region incorporating the following CDRs:
LC-CDR1 having the amino acid sequence of SEQ ID NO:229
LC-CDR2 having the amino acid sequence of SEQ ID NO:172
LC-CDR3 having the amino acid sequence of SEQ ID NO:230; or
(v)
(i) a heavy chain variable (VH) region incorporating the following CDRs:
HC-CDR1 having the amino acid sequence of SEQ ID NO:48
HC-CDR2 having the amino acid sequence of SEQ ID NO:199
HC-CDR3 having the amino acid sequence of SEQ ID NO:200; and
(ii) a light chain variable (VL) region incorporating the following CDRs:
LC-CDR1 having the amino acid sequence of SEQ ID NO:235
LC-CDR2 having the amino acid sequence of SEQ ID NO:236
LC-CDR3 having the amino acid sequence of SEQ ID NO:237; or
(w)
(i) a heavy chain variable (VH) region incorporating the following CDRs:
HC-CDR1 having the amino acid sequence of SEQ ID NO:185
HC-CDR2 having the amino acid sequence of SEQ ID NO:243
HC-CDR3 having the amino acid sequence of SEQ ID NO:244; and
(ii) a light chain variable (VL) region incorporating the following CDRs:
LC-CDR1 having the amino acid sequence of SEQ ID NO:248
LC-CDR2 having the amino acid sequence of SEQ ID NO:249
LC-CDR3 having the amino acid sequence of SEQ ID NO:250.

4. The antigen-binding molecule according to any one of claims 1 to 3, wherein the antigen-binding molecule comprises:
a VH region comprising an amino acid sequence having at least 70% sequence identity to the amino acid sequence of SEQ ID NO:165, 32, 1, 17, 47, 60, 82, 85, 94, 107, 121, 131, 154, 155, 184, 198, 210, 221 or 242; and
a VL region comprising an amino acid sequence having at least 70% sequence identity to the amino acid sequence of SEQ ID NO:178, 40, 9, 24, 52, 67, 72, 77, 88, 100, 114, 124, 138, 152, 157, 170, 191, 204, 215, 228, 234 or 247; optionally wherein the antigen-binding molecule comprises:
(i) a VH region comprising an amino acid sequence having at least 70% sequence identity to the amino acid sequence of SEQ ID NO:165; and
a VL region comprising an amino acid sequence having at least 70% sequence identity to the amino acid sequence of SEQ ID NO:178;
or
(ii) a VH region comprising an amino acid sequence having at least 70% sequence identity to the amino acid sequence of SEQ ID NO:32; and
a VL region comprising an amino acid sequence having at least 70% sequence identity to the amino acid sequence of SEQ ID NO:40;
or
(iii) a VH region comprising an amino acid sequence having at least 70% sequence identity to the amino acid sequence of SEQ ID NO:1; and
a VL region comprising an amino acid sequence having at least 70% sequence identity to the amino acid sequence of SEQ ID NO:9;
or
(iv) a VH region comprising an amino acid sequence having at least 70% sequence identity to the amino acid sequence of SEQ ID NO:17; and
a VL region comprising an amino acid sequence having at least 70% sequence identity to the amino acid sequence of SEQ ID NO:24;
or
(v) a VH region comprising an amino acid sequence having at least 70% sequence identity to the amino acid sequence of SEQ ID NO:47; and
a VL region comprising an amino acid sequence having at least 70% sequence identity to the amino acid sequence of SEQ ID NO:52;
or
(vi) a VH region comprising an amino acid sequence having at least 70% sequence identity to the amino acid sequence of SEQ ID NO:60; and
a VL region comprising an amino acid sequence having at least 70% sequence identity to the amino acid sequence of SEQ ID NO:67;
or
(vii) a VH region comprising an amino acid sequence having at least 70% sequence identity to the amino acid sequence of SEQ ID NO:60; and
a VL region comprising an amino acid sequence having at least 70% sequence identity to the amino acid sequence of SEQ ID NO:72;
or
(viii) a VH region comprising an amino acid sequence having at least 70% sequence identity to the amino acid sequence of SEQ ID NO:60; and
a VL region comprising an amino acid sequence having at least 70% sequence identity to the amino acid sequence of SEQ ID NO:77;
or
(ix) a VH region comprising an amino acid sequence having at least 70% sequence identity to the amino acid sequence of SEQ ID NO:; and
a VL region comprising an amino acid sequence having at least 70% sequence identity to the amino acid sequence of SEQ ID NO:;
or
(x) a VH region comprising an amino acid sequence having at least 70% sequence identity to the amino acid sequence of SEQ ID NO:82; and
a VL region comprising an amino acid sequence having at least 70% sequence identity to the amino acid sequence of SEQ ID NO:72;
or
(xi) a VH region comprising an amino acid sequence having at least 70% sequence identity to the amino acid sequence of SEQ ID NO:85; and
a VL region comprising an amino acid sequence having at least 70% sequence identity to the amino acid sequence of SEQ ID NO:88;
or
(xii) a VH region comprising an amino acid sequence having at least 70% sequence identity to the amino acid sequence of SEQ ID NO:94; and
a VL region comprising an amino acid sequence having at least 70% sequence identity to the amino acid sequence of SEQ ID NO:100;
or
(xiii) a VH region comprising an amino acid sequence having at least 70% sequence identity to the amino acid sequence of SEQ ID NO:107; and
a VL region comprising an amino acid sequence having at least 70% sequence identity to the amino acid sequence of SEQ ID NO:114;
or
(xiv) a VH region comprising an amino acid sequence having at least 70% sequence identity to the amino acid sequence of SEQ ID NO:121; and
a VL region comprising an amino acid sequence having at least 70% sequence identity to the amino acid sequence of SEQ ID NO:124;
or
(xv) a VH region comprising an amino acid sequence having at least 70% sequence identity to the amino acid sequence of SEQ ID NO:131; and
a VL region comprising an amino acid sequence having at least 70% sequence identity to the amino acid sequence of SEQ ID NO:138;
or
(xvi) a VH region comprising an amino acid sequence having at least 70% sequence identity to the amino acid sequence of SEQ ID NO:145; and
a VL region comprising an amino acid sequence having at least 70% sequence identity to the amino acid sequence of SEQ ID NO:152;
or
(xvii) a VH region comprising an amino acid sequence having at least 70% sequence identity to the amino acid sequence of SEQ ID NO:155; and
a VL region comprising an amino acid sequence having at least 70% sequence identity to the amino acid sequence of SEQ ID NO:157;
or
(xviii) a VH region comprising an amino acid sequence having at least 70% sequence identity to the amino acid sequence of SEQ ID NO:165; and
a VL region comprising an amino acid sequence having at least 70% sequence identity to the amino acid sequence of SEQ ID NO:170;
or
(xix) a VH region comprising an amino acid sequence having at least 70% sequence identity to the amino acid sequence of SEQ ID NO:184; and
a VL region comprising an amino acid sequence having at least 70% sequence identity to the amino acid sequence of SEQ ID NO:191;
or
(xx) a VH region comprising an amino acid sequence having at least 70% sequence identity to the amino acid sequence of SEQ ID NO:198; and
a VL region comprising an amino acid sequence having at least 70% sequence identity to the amino acid sequence of SEQ ID NO:204;
or
(xxi) a VH region comprising an amino acid sequence having at least 70% sequence identity to the amino acid sequence of SEQ ID NO:210; and
a VL region comprising an amino acid sequence having at least 70% sequence identity to the amino acid sequence of SEQ ID NO:215;
or
(xxii) a VH region comprising an amino acid sequence having at least 70% sequence identity to the amino acid sequence of SEQ ID NO:221; and
a VL region comprising an amino acid sequence having at least 70% sequence identity to the amino acid sequence of SEQ ID NO:228;
or
(xxiii) a VH region comprising an amino acid sequence having at least 70% sequence identity to the amino acid sequence of SEQ ID NO:198; and
a VL region comprising an amino acid sequence having at least 70% sequence identity to the amino acid sequence of SEQ ID NO:234;
or
(xxiv) a VH region comprising an amino acid sequence having at least 70% sequence identity to the amino acid sequence of SEQ ID NO:242; and
a VL region comprising an amino acid sequence having at least 70% sequence identity to the amino acid sequence of SEQ ID NO:247.

5. The antigen-binding molecule according to any one of claims 1 to 4, wherein the antigen-binding molecule binds to CNX via contact with: (a) one or more amino acid residues of the region of CNX corresponding to the region shown in SEQ ID NO:363, optionally wherein the antigen-binding molecule binds to CNX via contact with one or more amino acid residues of the region of CNX corresponding to the region shown in SEQ ID NO:361 or 362; or (b) one or more amino acid residues of the region of CNX corresponding to the region shown in SEQ ID NO:371, optionally wherein the antigen-binding molecule binds to CNX via contact with one or more amino acid residues of the region of CNX corresponding to the region shown in SEQ ID NO:364, 365, 366, 367, 368, 369, 370, 372, or 373.

6. The antigen-binding molecule according to any one of claims 1 to 5, wherein the antigen-binding molecule binds to CRT.

7. The antigen-binding molecule according to any one of claims 1 to 6, wherein the antigen-binding molecule binds to human CNX and mouse CNX.

8. The antigen-binding molecule according to any one of claims 1 to 8, wherein the antigen-binding molecule is a multispecific antigen-binding molecule, and wherein the antigen-binding molecule further comprises an antigen-binding domain which binds to an antigen other than CNX.

9. The antigen-binding molecule according to claim 8, wherein the multispecific antigen-binding molecule is a bispecific T cell engager (BiTE).

10. A chimeric antigen receptor (CAR) comprising an antigen-binding molecule according to any one of claims 1 to 9.

11. An antibody-drug conjugate (ADC) comprising an antigen-binding molecule according to any one of claims 1 to 9 and a drug moiety.

12. A nucleic acid, or a plurality of nucleic acids, optionally isolated, encoding an antigen-binding molecule according to any one of claims 1 to 9, a CAR according to claim 10.

13. An expression vector, or a plurality of expression vectors, comprising a nucleic acid or a plurality of nucleic acids according to claim 12.

14. A cell comprising an antigen-binding molecule according to any one of claims 1 to 9, a CAR according to claim 10, an ADC according to claim 11, a nucleic acid or a plurality of nucleic acids according to claim 12, or an expression vector or a plurality of expression vectors according to claim 13.

15. A method comprising culturing a cell according to claim 14 under conditions suitable for expression of an antigen-binding molecule or CAR by the cell.

16. A composition comprising an antigen-binding molecule according to any one of claims 1 to 9, a CAR according to claim 10, an ADC according to claim 11, a nucleic acid or a plurality of nucleic acids according to claim 12, an expression vector or a plurality of expression vectors according to claim 13, or a cell according to claim 14, and a pharmaceutically acceptable carrier, diluent, excipient or adjuvant.

17. An antigen-binding molecule according to any one of claims 1 to 9, a CAR according to claim 10, an ADC according to claim 11, a nucleic acid or a plurality of nucleic acids according to claim 12, an expression vector or a plurality of expression vectors according to claim 13, a cell according to claim 14, or a composition according to claim 16, for use in a method of medical treatment or prophylaxis.

18. An antigen-binding molecule according to any one of claims 1 to 9, a CAR according to claim 10, an ADC according to claim 11, a nucleic acid or a plurality of nucleic acids according to claim 12, an expression vector or a plurality of expression vectors according to claim 13, a cell according to claim 14, or a composition according to claim 16, for use in a method of treatment or prevention of a disease/condition characterised by extracellular matrix (ECM) degradation.

19. An antigen-binding molecule according to any one of claims 1 to 9, a CAR according to claim 10, an ADC according to claim 11, a nucleic acid or a plurality of nucleic acids according to claim 12, an expression vector or a plurality of expression vectors according to claim 13, a cell according to claim 14, or a composition according to claim 16, for use in a method of treatment or prevention of a cancer.

20. The antigen-binding molecule, CAR, ADC, nucleic acid or plurality of nucleic acids, expression vector or plurality of expression vectors, cell or composition for use according to claim 19, wherein the cancer is selected from: liver cancer, breast cancer, oral cancer, oral squamous cell carcinoma, sarcoma, lung cancer, prostate cancer, bladder cancer, renal cancer, melanoma, pancreatic cancer, endometrial cancer, colorectal cancer, ovarian cancer, cervical cancer, brain cancer, bile duct cancer, testicular cancer, and thyroid cancer.

21. An antigen-binding molecule according to any one of claims 1 to 9, a CAR according to claim 10, an ADC according to claim 11, a nucleic acid or a plurality of nucleic acids according to claim 12, an expression vector or a plurality of expression vectors according to claim 13, a cell according to claim 14, or a composition according to claim 16, for use in a method of treatment or prevention of cartilage degradation, or a disease/condition characterised by cartilage degradation.

22. The antigen-binding molecule, CAR, nucleic acid or plurality of nucleic acids, expression vector or plurality of expression vectors, cell or composition for use according to claim 21, wherein the disease/condition characterised by cartilage degradation is selected from: a joint disorder, arthritis, osteoarthritis, psoriasis arthritis, rheumatoid arthritis, juvenile arthritis, post-traumatic arthritis, gout, chondrocalcinosis, fibromyalgia, costochondritis, osteochondritis dissecans, cartilage damage and polychondritis.

23. Use of antigen-binding molecule according to any one of claims 1 to 9 to deplete or increase killing of cells expressing CNX.

24. An in vitro complex, optionally isolated, comprising an antigen-binding molecule according to any one of claims 1 to 9 bound to CNX.

25. A method for detecting CNX in a sample, comprising contacting a sample containing, or suspected to contain, CNX with an antigen-binding molecule according to any one of claims 1 to 9, and detecting the formation of a complex of the antigen-binding molecule with CNX.

26. A method of selecting or stratifying a subject for treatment with a CNX-targeted agent, the method comprising contacting, in vitro, a sample from the subject with an antigen-binding molecule according to any one of claims 1 to 9 and detecting the formation of a complex of the antigen-binding molecule with CNX.

27. Use of an antigen-binding molecule according to any one of claims 1 to 9 as an in vitro or in vivo diagnostic or prognostic agent.
